(19) United States
(12) Patent Application Publication
(10) Pub. No.: US 2011/0312898 A1
(43) Pub. Date: Dec. 22, 2011

(54) PROPHYLACTIC / THERAPEUTIC AGENT FOR CANCER

(75) Inventor: Hisanori Matsui, Kanagawa-ken (JP)

(73) Assignee: Takeda Pharmaceutical Company Limited, Chuo-ku (JP)

(21) Appl. No.: 13/142,414

(22) PCT Filed: Dec. 28, 2009
(86) PCT No.: PCT/JP2009/071919

§ 371 Date: Aug. 30, 2011

Related U.S. Application Data
(60) Provisional application No. 61/203,887, filed on Dec. 29, 2008.

(51) Int. Cl. A61K 38/08; A61P 35/00; C07K 7/06

(52) U.S. Cl. 514/19.3; 530/328

(57) ABSTRACT

A prophylactic/therapeutic agent for androgen-independent cancer is provided. A prophylactic/therapeutic agent for androgen-independent cancer includes a metastin derivative, and is particularly useful as a prophylactic/therapeutic agent for androgen-independent cancer, in particular, androgen-independent prostate cancer.

### BACKGROUND OF THE INVENTION

1. Field of the Invention

The present invention relates to a prophylactic/therapeutic agent for androgen-independent prostate cancer.

2. Description of the Related Art

Prostate cancer is a type of cancer which occurs primarily in elderly males. Androgens are closely associated with the progression of this disease. It is therefore possible to curb the growth of the tumor by inhibiting the production or function of androgens. Modalities for treating prostate cancer by inhibiting androgen production or function include surgical castration by orchiectomy, chemical castration with GnRH agonists, blocking androgen signals with androgen antagonists and inhibiting androgen production with estrogen agents.

Known therapeutic agents for prostate cancer include diethylstilbestrol, chlormadinone acetate, cyproterone acetate, goserelin acetate, buserelin acetate, leuprorelin acetate, ganirelix, flutamide, bicalutamide, nilutamide, finasteride, dexamethasone, prednisolone, ketoconazole and lyase inhibitors (see, for example, WO 2004/063221). In particular, surgical castration such as orchiectomy, chemical castration with a GnRH agonist, and the blocking of androgen signals with androgen antagonists all have a high rate of efficacy and few side effects, and are thus very useful therapies.

In the cancer treatment setting, when the patient acquires tolerance to a therapeutic drug, the efficacy of the drug weakens, resulting in, for example, recurrence of the cancer or metastasis. Accordingly, there exists a desire for the development of drugs for administration in cancer patients who have developed tolerance to therapeutic agents. Even among prostate cancer patients who have received therapy to suppress the production or function of androgens, there are cases where the tumor once again acquires the ability to grow. Prostate cancer that has reacquired the ability to grow after the tumor growth had been suppressed by the inhibition of androgen production or function using a treatment modality such as orchiectomy or hormone therapy is called androgen-independent prostate cancer (AIPC), hormone-refractory prostate cancer (HRPC) or castration-resistant prostate cancer (CRPC). Conceivable mechanisms for prostate cancer reacquiring the ability to grow include: (1) stimulation of tumor growth by lower androgen levels, (2) a decline in ligand selectivity due to changes in the androgen receptors (see, for example, “Novel mutations of androgen receptor: A possible mechanism of bicalutamide withdrawal syndrome,” T. Hara et al., Cancer Research 63, 149-153 (2003)), and (3) an increase in the expression of enzymes which convert low-activity androgens (e.g., DHEA, DHEA-S) that are produced by the adrenal glands and cannot be suppressed by surgical castration such as orchiectomy, castration with GnRH agonists or the inhibition of androgen production by estrogen agents into high-activity androgens (e.g., testosterone, dihydrotestosterone) (see, for example, “Increased expression of genes converting adrenal androgens to testosterone in androgen-independent prostate cancer,” M. Stanbrough et al., Cancer Research 66, 2815-2825 (2006)). However, drugs which are effective against androgen-independent prostate cancer have yet to be found.

In light of the above, there exists a desire for medications which are effective against androgen-independent prostate cancer in the clinical setting.

Metastin derivatives, which are compounds that have a cancer metastasis-inhibiting activity or a cancer growth-inhibiting activity and are effective, as cancer metastasis inhibitors or cancer growth inhibitors, in the prevention or treatment of cancer, have been disclosed in the art (WO 20004/063221, WO 2006/001499 and WO 2007/072997).

### SUMMARY OF THE INVENTION

It is therefore an object of the invention to provide a prophylactic/therapeutic agent for androgen-independent prostate cancer, which agent is highly effective as a medication.

In the course of extensive investigations aimed at finding good prophylactic/therapeutic agents for androgen-independent prostate cancer, the inventors have discovered that metastin derivative (IV) mentioned hereinafter (referred to below as “the inventive compound”) is useful for preventing and treating androgen-independent prostate cancer. Moreover, the inventors have found that medications obtained by combining the inventive compound with a concomitant drug are useful for preventing and treating prostate cancer or androgen-independent prostate cancer. Furthermore, the inventors have found that medications obtained by combining the inventive compound with a concomitant drug are useful for administration in cancer patients who have developed tolerance to therapeutic agents. The present invention has been accomplished on the basis of the abovementioned discovery.

Accordingly, the present invention provides:
[1] A prophylactic/therapeutic agent for androgen-independent cancer, comprising a metastin derivative (IV) of the following general formula, or a salt or prodrug thereof,

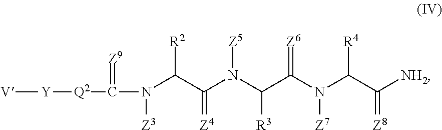

wherein V′ is a group of the formula

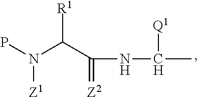

a group of the formula

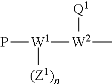

or a group of the formula

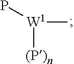

n represents 0 or 1;

W^{l} represents N, CH or O (provided that when W¹ is N or CH, n represents 1 and when W¹ is O, n represents 0);

W² represents N or CH;
Z¹, Z³, Z⁵ and Z⁷ each represents hydrogen atom or a C₁₋₃ alkyl group;
Z², Z⁴, Z⁶ and Z⁸ each represents hydrogen atom, O or S;
R¹ represents (1) a hydrogen atom, (2) a C₁₋₈ alkyl optionally substituted with a substituent selected from the group consisting of an optionally substituted carbamoyl group, an optionally substituted hydroxyl group and an optionally substituted aromatic cyclic group, (3) a cyclic or linear C₁₋₁₀ alkyl group, (4) a C₁₋₁₀ alkyl group consisting of a cyclic alkyl group and a linear alkyl group or (5) an optionally substituted aromatic cyclic group;

R² represents (1) hydrogen atom or (2) a cyclic or linear C₁₋₁₀ alkyl group, (3) a C₁₋₁₀ alkyl group consisting of a cyclic alkyl group and a linear alkyl group, or (4) a C₁₋₈ alkyl group optionally substituted with a substituent selected from the group consisting of an optionally substituted carbamoyl group, an optionally substituted hydroxyl group and an optionally substituted aromatic cyclic group;

R³ represents (1) a C₁₋₈ alkyl group having an optionally substituted basic group and optionally having an additional substituent, (2) an aralkyl group having an optionally substituted basic group and optionally having an additional substituent, (3) a C₁₋₄ alkyl group having a non-aromatic cyclic hydrocarbon group of carbon atoms not greater than 7 having an optionally substituted basic group, and optionally having an additional substituent, or (4) a C₁₋₄ alkyl group having a non-aromatic heterocyclic group of carbon atoms not greater than 7 having an optionally substituted basic group, and optionally having an additional substituent;

R⁴ represents a C₁₋₄ alkyl group, which may optionally be substituted with a substituent selected from the group consisting of (1) an optionally substituted C₆₋₁₂ aromatic hydrocarbon group, (2) an optionally substituted 5- to 14-membered aromatic heterocyclic group consisting of 1 to 7 carbon atoms and hetero atoms selected from the group consisting of nitrogen, oxygen and sulfur atoms, (3) an optionally substituted C₈₋₁₄ aromatic fused-ring group, (4) an optionally substituted 5- to 14-membered aromatic fused heterocyclic group consisting of 3 to 11 carbon atoms and hetero atoms selected from the group consisting of nitrogen, oxygen and sulfur atoms, (5) an optionally substituted non-aromatic cyclic hydrocarbon group having carbon atoms not greater than 7, and (6) an optionally substituted non-aromatic heterocyclic group having carbon atoms not greater than 7;

Q¹ represents a C₁₋₄ alkyl group, which may optionally be substituted with a substituent selected from the group consisting of (1) an optionally substituted C₆₋₁₂ aromatic hydrocarbon group, (2) an optionally substituted 5- to 14-membered aromatic heterocyclic group consisting of 1 to 7 carbon atoms and hetero atoms selected from the group consisting of nitrogen, oxygen and sulfur atoms, (3) an optionally substituted C₈₋₁₄ aromatic fused-ring group, (4) an optionally substituted 5- to 14-membered aromatic fused heterocyclic group consisting of 3 to 11 carbon atoms and hetero atoms selected from the group consisting of nitrogen, oxygen and sulfur atoms, (5) an optionally substituted non-aromatic cyclic hydrocarbon group having carbon atoms not greater than 7, and (6) an optionally substituted non-aromatic heterocyclic group having carbon atoms not greater than 7;

Q² represents (1) CH₂, which may optionally be substituted with an optionally substituted C₁₋₄ alkyl group with a substituent selected from the group consisting of carbamoyl group and hydroxyl group, (2) NH, which may optionally be substituted with an optionally substituted C₁₋₄ alkyl group with a substituent selected from the group consisting of carbamoyl group and hydroxyl group, or (3) O;

Y represents a group represented by formula: —CONH—, —CSNH—, —CH₂NH—, —NHCO—, —CH₂O—, —CH₂S—, —COO—, —CSO—, —CH₂CH₂—, or —CH═CH—, which may optionally be substituted with a C₁₋₆ alkyl group; and,

Z⁹ represents hydrogen atom, O or S; and,

P and P′, which may be the same or different, each may form a ring by combining P and P′ or P and Q¹ together and represents:

(1) hydrogen atom;

(2) an optional amino acid residue continuously or discontinuously bound from the C terminus of the 1-48 amino acid sequence in the amino acid sequence represented by SEQ ID NO: 1;

(3) a group represented by formula:

J¹-J²-C(J³)(Q³)Y¹C(J⁴)(Q⁴)Y²C(J⁵)(Q⁵)Y³C(J⁶)(Q⁶)C(═Z¹⁰)—

(wherein:

J¹ represents (a) hydrogen atom or (b) (i) a C₁₋₁₅ acyl group, (ii) a C₁₋₁₅ alkyl group, (iii) a C₆₋₁₄ aryl group, (iv) carbamoyl group, (v) carboxyl group, (vi) sulfino group, (vii) amidino group, (viii) glyoxyloyl group or (ix) amino group, which groups may optionally be substituted with a substituent containing an optionally substituted cyclic group;

J² represents (1) NH optionally substituted with a C₁₋₆ alkyl group, (2) CH₂ optionally substituted with a C₁₋₆ alkyl group, (3) O or (4) S;

J³ through J⁶ each represents hydrogen atom or a C₁₋₃ alkyl group;

Q³ through Q⁶ each represents a C₁₋₄ alkyl group, which may optionally have a substituent selected from the group consisting of:

(1) an optionally substituted C₆₋₁₂ aromatic hydrocarbon group,

(2) an optionally substituted 5- to 14-membered aromatic heterocyclic group consisting of 1 to 7 carbon atoms and hetero atoms selected from the group consisting of nitrogen, oxygen and sulfur atoms,

(3) an optionally substituted C₈₋₁₄ aromatic fused-ring group,

(4) an optionally substituted 5- to 14-membered aromatic fused heterocyclic group consisting of 3 to 11 carbon atoms and hetero atoms selected from the group consisting of nitrogen, oxygen and sulfur atoms,

(5) an optionally substituted non-aromatic cyclic hydrocarbon group having carbon atoms not greater than 7,

(6) an optionally substituted non-aromatic heterocyclic group having carbon atoms not greater than 7,

(7) an optionally substituted amino group,

(8) an optionally substituted guanidino group,

(9) an optionally substituted hydroxyl group,

(10) an optionally substituted carboxyl group,

(11) an optionally substituted carbamoyl group, and

(12) an optionally substituted sulfhydryl group,

or hydrogen atom;

J³ and Q³, J⁴ and Q⁴, J⁵ and Q⁵ or J⁶ and Q⁶ may be combined together, or, Z¹ and R¹, J² and Q³, Y¹ and Q⁴, Y² and Q⁵, or Y³ and Q⁶ may be combined together, to form a ring;

Y¹ through Y³ each represents a group represented by formula:

—CON(J¹³)-, —CSN(J¹³)-, —C(J¹⁴)N(J¹³)- or —N(J¹³)CO— (wherein J¹³ and J¹⁴ each represents hydrogen atom or a C₁₋₃ alkyl group); and,

Z¹⁰ represents hydrogen atom, O or S);

(4) a group represented by formula:

J¹-J²-C(J⁷)(Q⁷)Y²C(J⁸)(Q⁸)Y³C(J⁹)(Q⁹)C(═Z¹⁰)—

(wherein:

J¹ and J², each has the same significance as defined above;

J⁷ through J⁹ have the same significance as for J³;

Q⁷ through Q⁹ have the same significance as for Q³;

Y² and Y³ each has the same significance as defined above;

Z¹⁰ has the same significance as defined above;

J⁷ and Q⁷, J⁸ and Q⁸ or J⁹ and Q⁹ may be combined together, or, J² and Q⁷, Y² and Q⁸ or Y³ and Q⁹ may be combined together, to form a ring);

(5) a group represented by formula:

J¹-J²-C(J¹⁰(Q¹⁰)Y³C(J¹¹)(Q¹¹)C(═Z¹⁰)—

(wherein:

J¹ and J² have the same significance as defined above represents;

J¹⁰ and J¹¹ have the same significance as for J³;

Q¹⁰ and Q¹¹ have the same significance as for Q³;

Y³ has the same significance as defined above;

Z¹⁰ has the same significance as defined above; and,

J¹⁰ and Q¹⁰ or J¹¹ and Q¹¹ may be combined together, or J² and Q¹⁰ or Y³ and Q¹¹ may be combined together, to form a ring);

(6) a group represented by formula:

J¹-J²-C(J¹²)(Q¹²)C(═Z¹⁰)—

(wherein;

J¹ and J² have the same significance as defined above;

J¹² has the same significance as for J³;

Q¹² has the same significance as for Q³;

Z¹⁰ has the same significance as defined above; and,

J¹² and Q¹² may be combined together, or J² and Q¹² may be combined together, to form a ring); or,

(7) a group represented by formula:

J¹ (where J¹ has the same significance as defined above).
[2] The agent of [1] above, wherein the androgen-independent cancer is androgen-independent prostate cancer;
[3] The agent of [1] above, wherein the metastin derivative (IV) is Ac-D-Tyr-Hyp-Asn-Thr-Phe-AzaGly-Leu-Arg(Me)-Trp-NH₂ (Compound No. 723) or a salt thereof;
[4] The agent of [1] above, wherein the metastin derivative (IV) is Ac-D-Tyr-D-Trp-Asn-Thr-Phe-AzaGly-Leu-Arg(Me)-Trp-NH₂ (Compound No. 550) or a salt thereof;
[5] A prophylactic/therapeutic agent for androgen-independent cancer, comprising; Ac-D-Tyr-D-Trp-Asn-Thr-Phe-AzaGly-Leu-Arg(Me)-Trp-NH₂ (Compound No. 550), or a salt thereof;
[6] The agent of [5] above, wherein the androgen-independent cancer is androgen-independent prostate cancer;
[7] A prophylactic/therapeutic agent for androgen-independent cancer, comprising; Ac-D-Tyr-Hyp-Asn-Thr-Phe-AzaGly-Leu-Arg(Me)-Trp-NH₂ (Compound No. 723), or a salt thereof;
[8] The agent of [7] above, wherein the androgen-independent cancer is androgen-independent prostate cancer;
[9] A prophylactic/therapeutic method for androgen-independent cancer in mammals, the method being comprised of administering an effective dose of a metastin derivative (IV) as defined in [1] above, or a salt or prodrug thereof;
[10] Use of a metastin derivative (IV) as defined in [1] above, or a salt or prodrug thereof for producing prophylactic/therapeutic agent for androgen-independent cancer.

In addition, the present invention also provides, for example:
[11] The prophylactic/therapeutic agent for androgen-independent prostate cancer of [1] above in combination with a concomitant drug;
[12] The agent of [11] above, wherein the concomitant drug is one or more selected from among hormonal agents, alkylating agents, metabolic antagonists, anticancer antibiotics, plant alkaloids, immunotherapeutic agents, and drugs which inhibit the action of cell growth factors and receptors thereof;
[13] The agent of [11] above, wherein the concomitant drug is a LHRH receptor agonist or a LHRH receptor antagonist;
[14] A medication for administration to cancer patients who have developed tolerance (resistance) to a therapeutic agent, the medication being a combination of the metastin derivative (IV) as defined in [1] above, or a salt or prodrug thereof and a concomitant drug;
[15] The medication of [14] above, wherein the therapeutic agent is one or more selected from among hormonal agents, alkylating agents, metabolic antagonists, anticancer antibiotics, plant alkaloids, immunotherapeutic agents, and drugs which inhibit the action of cell growth factors and receptors thereof;
[16] The medication of [14] above, wherein the therapeutic agent is a LHRH receptor agonist or a LHRH receptor antagonist;
[17] The medication of [14] above, wherein the concomitant drug is one or more selected from among hormonal agents, alkylating agents, metabolic antagonists, anticancer antibiotics, plant alkaloids, immunotherapeutic agents, and drugs which inhibit the action of cell growth factors and receptors thereof;
[18] The medication of [14] above, wherein the concomitant drug is a LHRH receptor agonist or a LHRH receptor antagonist.

The prophylactic/therapeutic agents for androgen-independent cancer (especially prostate cancer) of the present invention are useful because they can be administered to patients with androgen-independent cancer (especially prostate cancer), which has posed a challenge in the clinical setting. Moreover, the medication according to the present invention is a combination of the inventive compound and a concomitant drug, and is particularly useful as a prophylactic/therapeutic agent for prostate cancer and androgen-independent prostate cancer. The inventive medication is also useful for administration in cancer patients who have developed tolerance (resistance) to therapeutic agents.

### BRIEF DESCRIPTION OF THE DRAWINGS

FIG. 1 is a graph showing the androgen-independent R3327-G antitumor effects of Compound No. 550 and Compound No. 723. The bars in the graph indicate a mean value+the standard deviation, or a mean value − the standard deviation (solvent group, n=6; Compound No. 550 group, n=10; Compound No. 723 group, n=7).
**: p 0.01 (Dunnett's test, compared with solvent group)

FIG. 2 shows antitumor activity of Compound No. 550 and Compound No. 723 against the DU145 tumor-bearing model (74 days after the transplantation of DU145 cells). In the graph, whisker ends of the box-and-whisker plot indicate the maximum value and the minimum value, the upper base of the box indicates the third quantile, the lower base of the box indicates the first quantile, and indicates the median value.

### DESCRIPTION OF THE PREFERRED EMBODIMENTS

In the formulas described above, n represents 0 or 1; W¹ represents N,CH or O (provided that when W¹ is N or CH, n represents 1 and when W¹ is O, n represents 0); W² represents N or CH; each of Z¹, Z³, Z⁵ and Z⁷ represents hydrogen atom or a C₁₋₃ alkyl group; and each of Z², Z⁴, Z⁶ and Z⁸ represents hydrogen atom, O or S.

Herein, when Z², Z⁴, Z⁶ or Z⁸ represents hydrogen atom, the moiety shown by >C═Z², >C═Z⁴, >C═Z⁶ or >C═Z⁸ each indicates the structure of >CH₂

The C₁₋₃ alkyl group used includes methyl group, ethyl group, propyl group and isopropyl group.

W¹ is preferably N and W² is preferably CH.

Preferred combinations of Z¹ through Z⁸ further include the cases where Z¹ and Z³ are hydrogen atoms and each of Z⁵ and Z⁷ represents hydrogen atom or a C₁₋₃ alkyl group and each of Z², Z⁴, Z⁶ and Z⁸ represents O or S.

More preferably, the combinations of Z¹ to Z⁸ include:

(a) the case where Z¹ is hydrogen atom, Z³ is hydrogen atom, Z⁵ is hydrogen atom, Z⁷ is hydrogen atom, Z² is O, Z⁴ is O, Z⁶ is O and Z⁸ is O;

(b) the case where Z¹ is hydrogen atom, Z³ is hydrogen atom, Z⁵ is hydrogen atom, Z⁷ is hydrogen atom, Z² is O, Z⁴ is O, Z⁶ is O and Z⁸ is S;

(c) the case where Z¹ and Z³ are hydrogen atoms, Z⁵ is hydrogen atom, Z⁷ is methyl group, Z² is O, Z⁴ is O, Z⁶ is O and Z⁸ is O; etc. Inter alia, (a) and (b) are preferred.

R¹ represents (1) hydrogen atom, (2) a C₁₋₈ alkyl group optionally substituted with a substituent selected from the group consisting of an optionally substituted carbamoyl group, an optionally substituted hydroxyl group and an optionally substituted aromatic cyclic group, (3) a cyclic or linear C₁₋₁₀ alkyl group, (4) a C₁₋₁₀ alkyl group consisting of a cyclic alkyl group and a linear alkyl group or (5) an optionally substituted aromatic cyclic group; inter alia, (1) hydrogen atom, or (2) a C₁₋₈ alkyl group optionally substituted with a substituent selected from the group consisting of an optionally substituted carbamoyl group, an optionally substituted hydroxyl group and an optionally substituted aromatic cyclic group; preferably (1) hydrogen atom, or (2) a C₁₋₈ alkyl group substituted with a substituent selected from the group consisting of an optionally substituted carbamoyl group, an optionally substituted hydroxyl group and an optionally substituted aromatic cyclic group.

The “C₁₋₈ alkyl group” used includes, for example, a linear or branched C₁₋₈ alkyl group such as methyl, ethyl, propyl, isopropyl, butyl, isobutyl, sec-butyl, tert-butyl, pentyl, isopentyl, neopentyl, hexyl, heptyl, octyl, etc., a cyclic C₃₋₈ alkyl group such as cyclopropyl, cyclobutyl, cyclopentyl, cyclohexyl, etc. Inter alia, a C₁₋₃ alkyl group such as methyl, ethyl, etc. are particularly preferred.

The “optionally substituted carbamoyl group” used includes, for example, carbamoyl, a mono-C₁₋₆ alkylcarbamoyl group (e.g., methylcarbamoyl, ethylcarbamoyl, etc.), a di-C₁₋₆ alkylcarbamoyl group (e.g., dimethylcarbamoyl, diethylcarbamoyl, ethylmethylcarbamoyl, etc.), a mono- or di-C₆₋₁₄ arylcarbamoyl group (e.g., phenylcarbamoyl, 1-naphthylcarbamoyl, 2-naphthylcarbamoyl, etc.), a mono- or di-5- or 7-membered heterocyclic carbamoyl group containing 1 to 4 hetero atoms of 1 or 2 species selected from nitrogen, sulfur and oxygen atoms in addition to carbon atoms (e.g., 2-pyridylcarbamoyl, 3-pyridylcarbamoyl, 4-pyridylcarbamoyl, 2-thienylcarbamoyl, 3-thienylcarbamoyl, etc.), and the like.

The “optionally substituted hydroxyl group” used includes, for example, hydroxy group, an optionally substituted C₁₋₆ alkoxy group, an optionally substituted C₆₋₁₄ aryloxy group, an optionally substituted C₇₋₁₆ aralkyloxy group, etc. The “optionally substituted C₁₋₆ alkoxy group,” “optionally substituted C₆₋₁₄ aryloxy group” and “optionally substituted C₇₋₁₆ aralkyloxy group” used are those given for the “optionally substituted C₁₋₆ alkoxy group,” “optionally substituted C₆₋₁₄ aryloxy group” and “optionally substituted C₇₋₁₆ aralkyloxy group” in Substituent Group A, which will be later described.

The “aromatic cyclic group” in “optionally substituted aromatic cyclic group” used includes, for example, an aromatic hydrocarbon group, aromatic heterocyclic group, an aromatic fused-ring group, an aromatic fused heterocyclic group, etc.

The “aromatic hydrocarbon group” used includes, for example, a C₆₋₁₄ aryl group such as phenyl, 2-biphenylyl, 3-biphenylyl, 4-biphenylyl, cyclooctatetraenyl, etc.

The “aromatic heterocyclic group” used includes, for example, a 5- to 14-membered, preferably 5- to 10-membered, more preferably 5- or 6-membered aromatic heterocyclic group containing 1 to 4 hetero atoms of 1 or 2 species selected from nitrogen, sulfur and oxygen atoms in addition to carbon atoms. Specific examples are thienyl (e.g., 2-thienyl, 3-thienyl), furyl (e.g., 2-furyl, 3-furyl), pyridyl (e.g., 2-pyridyl, 3-pyridyl, 4-pyridyl), thiazolyl (e.g., 2-thiazolyl, 4-thiazolyl, 5-thiazolyl), oxazolyl (e.g., 2-oxazolyl, 4-oxazolyl), pyrazinyl, pyrimidinyl (e.g., 2-pyrimidinyl, 4-pyrimidinyl), pyrrolyl (e.g., 1-pyrrolyl, 2-pyrrolyl, 3-pyrrolyl), imidazolyl (e.g., 1-imidazolyl, 2-imidazolyl, 4-imidazolyl), pyrazolyl (e.g., 1-pyrazolyl, 3-pyrazolyl, 4-pyrazolyl), pyridazinyl (e.g., 3-pyridazinyl, 4-pyridazinyl), isothiazolyl (e.g., 3-isothiazolyl), isoxazolyl (e.g., 3-isoxazolyl), etc.

The “aromatic fused-ring group” used includes a C₈₋₁₄ aromatic fused-ring group such as naphthyl (e.g., 1-naphthyl, 2-naphthyl), anthryl (e.g., 2-anthryl, 9-anthryl) and the like.

The “aromatic fused heterocyclic group” used includes, for example, a 5- to 14-memberd (preferably 5- to 10-membered) bicyclic or tricyclic aromatic heterocyclic group containing 1 to 4 hetero atoms of 1 or 2 species selected from nitrogen, sulfur and oxygen atoms in addition to 3 to 11 carbon atoms, or a monovalent group formed by removing one optional hydrogen atom from a 7- to 10-membered aromatic bridged-hetero ring in 5- to 14-membered (preferably 5- to 10-membered) ring containing 1 to 4 hetero atoms of 1 or 2 species selected from nitrogen, sulfur and oxygen atoms in addition to carbon atoms. Specific examples of these groups used are quinolyl (e.g., 2-quinolyl, 3-quinolyl, 4-quinolyl, 5-quinolyl, 8-quinolyl), isoquinolyl (e.g., 1-isoquinolyl, 3-isoquinolyl, 4-isoquinolyl, 5-isoquinolyl), indolyl (e.g., 1-indolyl, 2-indolyl, 3-indolyl), 2-benzothiazolyl, benzo[b]thienyl, (e.g., 2-benzo[b]thienyl, 3-benzo[b]thienyl), benzo[b]furanyl (e.g., 2-benzo[b]furanyl, 3-benzo[b]furanyl) and the like.

The “substituent” used in the “aromatic cyclic group” includes a substituent selected from Substituent Group A, which will be later described.

As R¹, there are used hydrogen atom, carbamoylmethyl, 2-carbamoylethyl, hydroxymethyl, 1-hydroxyethyl, benzyl, 4-hydroxybenzyl, 2-pyridylmethyl, 3-pyridylmethyl, 4-pyridylmethyl, 2-thienylmethyl, 3-thienylmethyl, 1-naphthylmethyl, 2-naphthylmethyl, 3-indolemethyl, methyl, ethyl, propyl, isopropyl, butyl, isobutyl, sec-butyl, tert-butyl, cyclohexylmethyl, phenyl, acetoxymethyl, methoxymethyl, etc.; among others, preferred are hydroxymethyl, 1-hydroxyethyl, benzyl, 4-hydroxybenzyl, 3-indolemethyl, methyl, isobutyl, etc., more preferably, hydroxymethyl, 1-hydroxyethyl, etc.

R² represents (1) hydrogen atom, (2) a cyclic or linear C₁₋₁₀ alkyl group, (3) a C₁₋₁₀ alkyl group consisting of a cyclic alkyl group and a linear alkyl group, or (4) a C₁₋₈ alkyl group optionally substituted with a substituent selected from the group consisting of an optionally substituted carbamoyl group, an optionally substituted hydroxyl group and an optionally substituted aromatic cyclic group. Among others, preferred are (1) hydrogen atom, (2) a cyclic or linear C₁₋₁₀ alkyl group, or (3) a C₁₋₁₀ alkyl group consisting of a cyclic alkyl group and a linear alkyl group. In particular, (3) a linear C₁₋₁₀ alkyl group or a C₁₋₁₀ alkyl group consisting of a cyclic alkyl group and a linear alkyl group is preferred.

The cyclic C₁₋₁₀ alkyl group used includes, for example, a C₃₋₈ cycloalkyl group such as cyclopropyl, cyclobutyl, cyclopentyl, cyclohexyl, etc.

Examples of the linear C₁₋₁₀ alkyl group include methyl, ethyl, propyl, isopropyl, butyl, isobutyl, sec-butyl, tert-butyl, pentyl, isopentyl, neopentyl, hexyl, heptyl, octyl, nonanyl, decanyl, etc.

The C₁₋₁₀ alkyl group consisting of a cyclic alkyl group and a linear alkyl group used includes, for example, a C₃₋₇ cycloalkyl-C₁₋₃ alkyl group such as cyclopentylmethyl, cyclohexylmethyl, etc.

Examples of R² include methyl, ethyl, propyl, isopropyl, butyl, isobutyl, sec-butyl, tert-butyl, cyclohexylmethyl, benzyl, hydroxymethyl, 2-carbamoylethyl, tert-pentyl, etc.; among others, preferred are methyl, ethyl, propyl, isopropyl, isobutyl, sec-butyl, tert-butyl, etc., more preferably, propyl, isopropyl, isobutyl, etc.

R³ represents:
(1) a C₁₋₈ alkyl group having an optionally substituted basic group and optionally having an additional substituent,
(2) an aralkyl group having an optionally substituted basic group and optionally having an additional substituent,
(3) a C₁₋₄ alkyl group having a non-aromatic cyclic hydrocarbon group of carbon atoms not greater than 7 having an optionally substituted basic group, and optionally having an additional substituent, or,
(4) a C₁₋₄ alkyl group having a non-aromatic heterocyclic group of carbon atoms not greater than 7 having an optionally substituted basic group, and optionally having an additional substituent; particularly preferably (1) a C₁₋₈ alkyl group having an optionally substituted basic group and optionally having an additional substituent.

The “optionally substituted basic group” used includes, for example, (1) a guanidino group optionally having 1 or 2 substituents from C₁₋₆ alkyl, C₁₋₆ acyl (e.g., methyl, ethyl, propyl, isopropyl, butyl, acetyl, propionyl, etc.), etc., (2) an amino group optionally having 1 to 3 substituents from C₁₋₆ alkyl, C₁₋₆ acyl (e.g., methyl, ethyl, propyl, isopropyl, butyl, acetyl, propionyl, etc.), etc., (3) a C₁₋₆ alkylcarbonylamino group (e.g., acetamido) optionally substituted with a guanidino group optionally having 1 or 2 substituents from C₁₋₆ alkyl, C₁₋₆ acyl (e.g., methyl, ethyl, propyl, isopropyl, butyl, acetyl, propionyl, etc.), etc., (4) a C₁₋₆ alkylcarbonylamino group (e.g., acetamido) optionally substituted with an amino group optionally having 1 to 3 substituents from C₁₋₆ alkyl, C₁₋₆ acyl (e.g., methyl, ethyl, propyl, isopropyl, butyl, acetyl, propionyl, etc.), etc. Among others, preferred are guanidino, N-methylguanidino, N, N-dimethylguanidino, N, N′-dimethylguanidino, N-ethylguanidino, N-acetylguanidino, amino, N-methylamino, N, N-dimethylamino, aminoacetamido, guanidinoacetamido, amidino, and the like.

The “additional substituent” other than the “optionally substituted basic group” used includes a substituent selected from Substituent Group A later described.

Examples of the “C₁₋₈ alkyl group” used are methyl, ethyl, propyl, isopropyl, butyl, isobutyl, sec-butyl, tert-butyl, pentyl, isopentyl, neopentyl, hexyl, heptyl, octyl, etc.

The “aralkyl group” used includes, for example, a C₇₋₁₆ aralkyl group such as benzyl, phenethyl, diphenylmethyl, 1-naphthylmethyl, 2-naphthylmethyl, 2,2-diphenylethyl, 3-phenylpropyl, 4-phenylbutyl, 5-phenylpentyl, 2-biphenylylmethyl, 3-biphenylylmethyl, 4-biphenylylmethyl, etc.

The “non-aromatic cyclic hydrocarbon group of carbon atoms not greater than 7” used includes, for example, a C₃₋₇ cycloalkyl group such as cyclopropyl, cyclobutyl, cyclopentyl, cyclohexyl, etc.

The “non-aromatic heterocyclic group of carbon atoms not greater than 7” used includes, for example, a 5- to 10-membered non-aromatic heterocyclic group containing 1 to 4 hetero atoms of 1 or 2 species selected from nitrogen, sulfur and oxygen atoms, in addition to 1 to 7 carbon atoms, etc. Specific examples used are pyrrolidinyl (e.g., 1-pyrrolidinyl, 2-pyrrolidinyl, 3-pyrrolidinyl), oxazolidinyl (e.g., 2-oxazolidinyl), imidazolinyl (e.g., 1-imidazolinyl, 2-imidazolinyl, 4-imidazolinyl), piperidinyl (e.g., 1-piperidinyl, 2-piperidinyl, 3-piperidinyl, 4-piperidinyl), piperazinyl (e.g., 1-piperazinyl, 2-piperazinyl), morpholino, thiomorpholino, etc.

Examples of the “C₁₋₄ alkyl group” used include methyl, ethyl, propyl, isopropyl, butyl, isobutyl, sec-butyl, tert-butyl, etc.

For R³, there are used, for example, (1) 3-guanidinopropyl, 3-(N-methylguanidino)propyl, 3-(N, N-dimethylguanidino)propyl, 3-(N, N′-dimethylguanidino)propyl, 3-(N-ethylguanidino)propyl, 3-(N-propylguanidino)propyl, 3-(N-acetylguanidino)propyl, 4-guanidinobutyl, 4-(N-methylguanidino)butyl, 2-guanidinoethyl, 2-(N-methylguanidino)ethyl, 4-aminobutyl, 4-(N-methylamino)butyl, 4-(N, N-dimethylamino)butyl, 3-aminopropyl, 2-aminoethyl, aminomethyl, aminoacetamidomethyl, guanidinoacetamidomethyl, 2-(guanidinocarbonyl)ethyl, (2)4-guanidinobenzyl, 4-aminobenzyl, (3)4-guanidinocyclohexylmethyl, 4-aminocyclohexylmethyl, (4)1-amidinopiperidin-4-ylmethyl, 4-pyridylmethyl, etc.; among others, preferred are 3-guanidinopropyl, 3-(N-methylguanidino)propyl, 3-(N, N-dimethylguanidino)propyl, 3-(N, N′-dimethylguanidino)propyl, 3-(N-ethylguanidino)propyl, 3-(N-propylguanidino)propyl, 3-(N-acetylguanidino)propyl, 4-guanidinobutyl, 4-(N-methylguanidino)butyl, 2-guanidinoethyl, 2-(N-methylguanidino)ethyl, 4-aminobutyl, 4-(N-methylamino)butyl, 4-(N, N-dimethylamino)butyl, 3-aminopropyl, 2-aminoethyl, 4-aminobenzyl, aminoacetamidomethyl, guanidinoacetamidomethyl, etc., particularly preferably, 3-guanidinopropyl, 3-(N-methylguanidino)propyl, 3-(N, N-dimethylguanidino)propyl, 3-(N, N′-dimethylguanidino)propyl, 3-(N-ethylguanidino)propyl, 3-(N-acetylguanidino)propyl, 4-guanidinobutyl, 4-(N-methylguanidino)butyl, 2-guanidinoethyl, 4-aminobutyl, etc.

R⁴ represents a C₁₋₄ alkyl group, which may optionally be substituted with a substituent selected from the group consisting of: (1) an optionally substituted C₆₋₁₂ aromatic hydrocarbon group, (2) an optionally substituted 5- to 14-membered aromatic heterocyclic group consisting of 1 to 7 carbon atoms and hetero atoms selected from the group consisting of nitrogen, oxygen and sulfur atoms, (3) an optionally substituted C₈₋₁₄ aromatic fused-ring group, (4) an optionally substituted 5- to 14-membered aromatic fused heterocyclic group consisting of 3 to 11 carbon atoms and hetero atoms selected from the group consisting of nitrogen, oxygen and sulfur atoms, (5) an optionally substituted non-aromatic cyclic hydrocarbon group having carbon atoms not greater than 7, and, (6) an optionally substituted non-aromatic heterocyclic group having carbon atoms not greater than 7; inter alia, preferably C₁₋₄ alkyl group, which is optionally substituted with a substituent selected from the group consisting of: (1) an optionally substituted C₆₋₁₂ aromatic hydrocarbon group, (2) an optionally substituted 5- to 14-membered aromatic heterocyclic group consisting of 1 to 7 carbon atoms and hetero atoms selected from the group consisting of nitrogen, oxygen and sulfur atoms, (3) an optionally substituted C₈₋₁₄ aromatic fused-ring group, (4) an optionally substituted 5- to 14-membered aromatic fused heterocyclic group consisting of 3 to 11 carbon atoms and hetero atoms selected from the group consisting of nitrogen, oxygen and sulfur atoms, (5) an optionally substituted non-aromatic cyclic hydrocarbon group having carbon atoms not greater than 7, and (6) an optionally substituted non-aromatic heterocyclic group having carbon atoms not greater than 7.

The “C₁₋₄ alkyl groups” includes methyl, ethyl, propyl, isopropyl, butyl, isobutyl, sec-butyl and tert-butyl.

The “C₆₋₁₂ aromatic hydrocarbon group” includes monocyclic C₆₋₁₂ aromatic hydrocarbon groups such as phenyl and cyclooctatetraenyl.

The “5- to 14-membered aromatic heterocyclic group consisting of 1 to 7 carbon atoms and heteroatoms selected from the group consisting of nitrogen, oxygen and sulfur atoms” may be a 5- to 14-membered, preferably 5- to 10-membered, and more preferably 5- or 6-membered, monocyclic aromatic heterocyclic group which includes, other than the 1 to 7 carbon atoms, from 1 to 4 heteroatoms of one or two species selected from among nitrogen, sulfur and oxygen atoms. Illustrative examples include thienyl (e.g., 2-thienyl, 3-thienyl), furyl (e.g., 2-furyl, 3-furyl), pyridyl (e.g., 2-pyridyl, 3-pyridyl, 4-pyridyl), thiazolyl (e.g., 2-thiazolyl, 4-thiazolyl, 5-thiazolyl), oxazolyl (e.g., 2-oxazolyl, 4-oxazolyl), pyrazinyl, pyrimidinyl (e.g., 2-pyrimidinyl, 4-pyrimidinyl), pyrrolyl (e.g., 1-pyrrolyl, 2-pyrrolyl, 3-pyrrolyl), imidazolyl (e.g., 1-imidazolyl, 2-imidazolyl, 4-imidazolyl), pyrazolyl (e.g., 1-pyrazolyl, 3-pyrazolyl, 4-pyrazolyl), pyridazinyl (e.g., 3-pyridazinyl, 4-pyridazinyl), isothiazolyl (e.g., 3-isothiazolyl) and isoxazolyl (e.g., 3-isoxazolyl).

The “C₈₋₁₄ aromatic fused-ring groups” include naphthyl (e.g., 1-naphthyl, 2-naphthyl) and anthryl (e.g., (2-anthryl, 9-anthryl).

The “5- to 14-membered aromatic fused heterocyclic group consisting of 3 to 11 carbon atoms and heteroatoms selected from the group consisting of nitrogen, oxygen and sulfur atoms” may be a 5- to 14-membered (preferably 5- to 10-membered) bicyclic or tricyclic aromatic heterocyclic group which includes, other than the 3 to 11 carbon atoms, from 1 to 4 heteroatoms of one or two species selected from among nitrogen, sulfur and oxygen atoms, or may be a monovalent 5- to 14-membered (preferably 5- to 10-membered) group which includes, other than carbon atoms, from 1 to 4 heteroatoms of one or two species selected from among nitrogen, sulfur and oxygen atoms and is obtained by removing any one hydrogen atom from a 7- to 10-membered aromatic heterobridged ring. Illustrative examples include quinolyl (e.g., 2-quinolyl, 3-quinolyl, 4-quinolyl, 5-quinolyl, 8-quinolyl), isoquinolyl (e.g., 1-isoquinolyl, 3-isoquinolyl, 4-isoquinolyl, 5-isoquinolyl), indolyl (e.g., 1-indolyl, 2-indolyl, 3-indolyl), 2-benzothiazolyl, benzo[b]thienyl (e.g., 2-benzo[b]thienyl, 3-benzo[b]thienyl) and benzo[b]furanyl (e.g., 2-benzo[b]furanyl, 3-benzo[b]furanyl).

Radicals that may be used as the “non-aromatic cyclic hydrocarbon groups having carbon atoms not greater than 7” include C₃₋₇ cycloalkyl radicals such as cyclopropyl, cyclobutyl, cyclopentyl, cyclohexyl.

The “non-aromatic heterocyclic group having carbon atoms not greater than 7” used includes, for example, a 5- or 10-membered non-aromatic heterocyclic group containing 1 to 4 hetero atoms of 1 or 2 species selected from nitrogen, sulfur and oxygen atoms, in addition to 1 to 7 carbon atoms, such as pyrrolidinyl (e.g., 1-pyrrolidinyl, 2-pyrrolidinyl, 3-pyrrolidinyl), oxazolidinyl (e.g., 2-oxazolidinyl), imidazolinyl (e.g., 1-imidazolinyl, 2-imidazolinyl, 4-imidazolinyl), piperidinyl (e.g., 1-piperidinyl, 2-piperidinyl, 3-piperidinyl, 4-piperidinyl), piperazinyl (e.g., 1-piperazinyl, 2-piperazinyl), morpholino, thiomorpholino, etc.

The substituents used for these “C₆₋₁₂ aromatic hydrocarbon group,” “5- to 14-membered aromatic heterocyclic group consisting of 1 to 7 carbon atoms and hetero atoms selected from the group consisting of nitrogen, oxygen and sulfur atoms,” “C₈₋₁₄ aromatic fused-ring group,” “5- to 14-membered aromatic fused heterocyclic group consisting of 3 to 11 carbon atoms and hetero atoms selected from the group consisting of nitrogen, oxygen and sulfur atoms,” “non-aromatic cyclic hydrocarbon group having carbon atoms not greater than 7” and “non-aromatic heterocyclic group having carbon atoms not greater than 7” include, for example, substituents selected from oxo, a halogen atom (e.g., fluorine, chlorine, bromine, iodine, etc.), C₁₋₃ alkylenedioxy (e.g., methylenedioxy, ethylenedioxy, etc.), nitro, cyano, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₆₋₁₄ aryl, optionally substituted C₇₋₁₆ aralkyl, optionally substituted C₁₋₆ alkoxy, hydroxy, optionally substituted C₆₋₁₄ aryloxy, optionally substituted C₇₋₁₆ aralkyloxy, mercapto, optionally substituted C₁₋₆ alkylthio, optionally substituted C₆₋₁₄ arylthio, optionally substituted C₇₋₁₆ aralkylthio, optionally substituted amino [e.g., amino, optionally substituted mono- or di-C₁₋₆ alkylamino (e.g., methylamino, dimethylamino, ethylamino, diethylamino, propylamino, isopropylamino, etc.), optionally substituted mono- or di-C₂₋₆ alkenylamino (e.g., vinylamino, propenylamino, isopropenylamino), optionally substituted C₂₋₆ alkynylamino (e.g., 2-butyn-1-yl-amino, 4-pentyn-1-yl-amino, 5-hexyn-1-yl-amino), optionally substituted mono- or di-C₃₋₈ cycloalkylamino (e.g., cyclopropylamino, cyclohexylamino), optionally substituted C₆₋₁₄ aryl-amino (e.g., phenylamino, diphenylamino, naphthylamino), optionally substituted C₁₋₆ alkoxy-amino (e.g., methoxyamino, ethoxyamino, propoxyamino, isopropoxyamino), formylamino, optionally substituted C₁₋₆ alkylcarbonylamino (e.g., acetylamino, propionylamino, pivaloylamino, etc.), optionally substituted C₃₋₈ cycloalkylcarbonylamino (e.g., cyclopropylcarbonylamino, cyclopentylcarbonylamino, cyclohexylcarbonylamino, etc.), optionally substituted C₆₋₁₄ aryl-carbonylamino (e.g., benzoylamino, naphthoylamino, etc.), optionally substituted C₁₋₆ alkoxy-carbonylamino (e.g., methoxycarbonylamino, ethoxycarbonylamino, propoxycarbonylamino, butoxycarbonylamino, etc.), optionally substituted C₁₋₆ alkylsulfonylamino (e.g., methylsulfonylamino, ethylsulfonylamino, etc.), optionally substituted C₆₋₁₄ arylsulfonylamino (e.g., phenylsulfonylamino, 2-naphthylsulfonylamino, 1-naphthylsulfonylamino, etc.)], formyl, carboxy, optionally substituted C₁₋₆ alkylcarbonyl (e.g., acetyl, propionyl, pivaloyl, etc.), optionally substituted C₃₋₈ cycloalkylcarbonyl (e.g., cyclopropylcarbonyl, cyclopentylcarbonyl, cyclohexylcarbonyl, 1-methyl-cyclohexyl-carbonyl, etc.), optionally substituted C₆₋₁₄ aryl-carbonyl (e.g., benzoyl, 1-naphthoyl, 2-naphthoyl, etc.), optionally substituted C₇₋₁₆ aralkyl-carbonyl (e.g., phenylacetyl, 3-phenylpropionyl, etc.), optionally substituted 5- to 7-membered heterocyclic carbonyl containing 1 to 4 hetero atoms of 1 or 2 species selected from nitrogen, sulfur and oxygen atoms in addition to carbon atoms (e.g., nicotinoyl, isonicotinoyl, thenoyl, furoyl, morpholinocarbonyl, thiomorpholinocarbonyl, piperazin-1-ylcarbonyl, pyrrolidin-1-ylcarbonyl, etc.), optionally esterified carboxyl, optionally substituted carbamoyl, optionally substituted C₁₋₆ alkylsulfonyl (e.g., methylsulfonyl, ethylsulfonyl, etc.), optionally substituted C₁₋₆ alkylsulfinyl (e.g., methylsulfinyl, ethylsulfinyl, etc.), optionally substituted C₆₋₁₄ arylsulfonyl (e.g., phenylsulfonyl, 1-naphthylsulfonyl, 2-naphthylsulfonyl, etc.), optionally substituted C₆₋₁₄ arylsulfinyl (e.g., phenylsulfinyl, 1-naphthylsulfinyl, 2-naphthylsulfinyl, etc.), optionally substituted C₁₋₆ alkylcarbonyloxy (e.g., acetoxy, propionyloxy, etc.), optionally substituted C₆₋₁₄ aryl-carbonyloxy (e.g., benzoyloxy, naphthylcarbonyloxy, etc.), optionally substituted C₁₋₆ alkoxy-carbonyloxy (e.g., methoxycarbonyloxy, ethoxycarbonyloxy, propoxycarbonyloxy, butoxycarbonyloxy, etc.), optionally substituted mono-C₁₋₆ alkylcarbamoyloxy (e.g., methylcarbamoyloxy, ethylcarbamoyloxy, etc.), optionally substituted di-C₁₋₆ alkylcarbamoyloxy (e.g., dimethylcarbamoyloxy, diethylcarbamoyloxy, etc.), optionally substituted mono- or di-C₆₋₁₄ arylcarbamoyloxy (e.g., phenylcarbamoyloxy, naphthylcarbamoyloxy, etc.), optionally substituted heterocyclic group, sulfo, sulfamoyl, sulfinamoyl, sulfenamoyl, or a group of 2 or more (e.g., 2 or 3) of these substituents combined, and the like (to be referred as “Substituent Group A” in the present specification). The number of the substituents is not particularly limited but these rings may have 1 to 5, preferably 1 to 3 substituents in substitutable positions, and when there are two or more substituents, each substituent may be the same or different.

The “optionally esterified carboxyl” in Substituent Group A includes, for example, an optionally substituted C₁₋₆ alkoxy-carbonyl (e.g., methoxycarbonyl, ethoxycarbonyl, propoxycarbonyl, tert-butoxycarbonyl, etc.), an optionally substituted C₆₋₁₄ aryloxy-carbonyl (e.g., phenoxycarbonyl, etc.), an optionally substituted C₇₋₁₆ aralkyloxy-carbonyl (e.g., benzyloxycarbonyl, phenethyloxycarbonyl, etc.), and the like.

The “C₁₋₆ alkyl” in the “optionally substituted C₁₋₆ alkyl” in Substituent Group A includes, for example, methyl, ethyl, propyl, isopropyl, butyl, isobutyl, sec-butyl, tert-butyl, pentyl, isopentyl, neopentyl, hexyl, etc.

The “C₂₋₆ alkenyl” in the “optionally substituted C₂₋₆ alkenyl” in Substituent Group A includes, for example, vinyl, propenyl, isopropenyl, 2-buten-1-yl, 4-penten-1-yl, 5-hexen-1-yl, etc.

The “C₂₋₆ alkynyl” in the “optionally substituted C₂₋₆ alkynyl” in Substituent Group A includes, for example, 2-butyn-1-yl, 4-pentyn-1-yl, 5-hexyn-1-yl, etc.

The “C₃₋₈ cycloalkyl” in the “optionally substituted C₃₋₈ cycloalkyl” in Substituent Group A includes, for example, cyclopropyl, cyclobutyl, cyclopentyl, cyclohexyl, etc.

The “C₆₋₁₄ aryl” in the “optionally substituted C₆₋₁₄ aryl” in Substituent Group A includes, for example, phenyl, 1-naphthyl, 2-naphthyl, 2-biphenylyl, 3-biphenylyl, 4-biphenylyl, 2-anthryl, etc.

The “C₇₋₁₆ aralkyl” in the “optionally substituted C₇₋₁₆ aralkyl” in Substituent Group A includes, for example, benzyl, phenethyl, diphenyllmethyl, 1-naphthylmethyl, 2-naphthylmethyl, 2,2-diphenylethyl, 3-phenylpropyl, 4-phenylbutyl, 5-phenylpentyl, 2-biphenylylmethyl, 3-biphenylylmethyl, 4-biphenylylmethyl, etc.

The “C₁₋₆ alkoxy” in the “optionally substituted C₁₋₆ alkoxy” in Substituent Group A includes, for example, methoxy, ethoxy, propoxy, isopropoxy, butoxy, isobutoxy, sec-butoxy, pentyloxy, hexyloxy, etc.

The “C₆₋₁₄ aryloxy” in the “optionally substituted C₆₋₁₄ aryloxy” in Substituent Group A includes, for example, phenyloxy, 1-naphthyloxy, 2-naphthyloxy, etc.

The “C₇₋₁₆ aralkyloxy” in the “optionally substituted C₇₋₁₆ aralkyloxy” in Substituent Group A includes, for example, benzyloxy, phenethyloxy, etc.

The “C₁₋₆ alkylthio” in the “optionally substituted C₁₋₆ alkylthio” in Substituent Group A includes, for example, methylthio, ethylthio, propylthio, isopropylthio, butylthio, sec-butylthio, tert-butylthio, etc.

The “C₆₋₁₄ arylthio” in the “optionally substituted C₆₋₁₄ arylthio” in Substituent Group A includes, for example, phenylthio, 1-naphthylthio, 2-naphthylthio, etc.

The “C₇₋₁₆ aralkylthio” in the “optionally substituted C₇₋₁₆ aralkylthio” in Substituent Group A includes, for example, benzylthio, phenethylthio, etc.

The substituents in these “C₁₋₆ alkoxy-carbonyl,” “C₁₋₆ alkyl”, “C₂₋₆ alkenyl”, “C₂₋₆ alkynyl”, “C₁₋₆ alkoxy”, “C₁₋₆ alkylthio”, “C₁₋₆ alkyl-amino”, “C₂₋₆ alkenyl-amino”, “C₂₋₆ alkynyl-amino”, “C₁₋₆ alkoxy-amino”, “C₁₋₆ alkyl-carbonyl”, “C₁₋₆ alkylsulfonyl”, “C₁₋₆ alkylsulfinyl”, “C₁₋₆ alkyl-carbonylamino”, “C₁₋₆ alkoxy-carbonylamino”, “C₁₋₆ alkylsulfonylamino”, “C₁₋₆ alkyl-carbonyloxy”, “C₁₋₆ alkoxy-carbonyloxy”, “mono-C₁₋₆ alkylcarbamoyloxy” and “di-C₁₋₆ alkylcarbamoyloxy” in Substituent Group A include, for example, 1 to 5 substituents selected from, for example, a halogen atom (e.g., fluorine atom, chlorine atom, bromine atom, iodine atom), carboxy, hydroxy, amino, mono- or di-C₁₋₆ alkylamino, mono- or di-C₆₋₁₄ arylamino, C₃₋₈ cycloalkyl, C₁₋₆ alkoxy, C₁₋₆ alkoxy-carbonyl, C₁₋₆ alkylthio, C₁₋₆ alkylsulfinyl, C₁₋₆ alkylsulfonyl, the optionally esterified carboxyl described above, carbamoyl, thiocarbamoyl, mono-C₁₋₆ alkylcarbamoyl (e.g., methylcarbamoyl, ethylcarbamoyl, etc.), di-C₁₋₆ alkylcarbamoyl (e.g., dimethylcarbamoyl, diethylcarbamoyl, ethylmethylcarbamoyl, etc.), mono- or di-C₆₋₁₄ arylcarbamoyl (e.g., phenylcarbamoyl, 1-naphthylcarbamoyl, 2-naphthylcarbamoyl, etc.), mono- or di-5- to 7-membered heterocyclic carbamoyl containing 1 to 4 hetero atoms of 1 or 2 species selected from nitrogen, sulfur and oxygen atoms in addition to carbon atoms (e.g., 2-pyridylcarbamoyl, 3-pyridylcarbamoyl, 4-pyridylcarbamoyl, 2-thienylcarbamoyl, 3-thienylcarbamoyl, etc.), and the like.

The substituents for the “C₆₋₁₄ aryloxy-carbonyl”, “C₇₋₁₆ aralkyloxy-carbonyl”, “C₃₋₈ cycloalkyl”, “C₆₋₁₄ aryl”, “C₇₋₁₆ aralkyl”, “C₆₋₁₄ aryloxy”, “C₇₋₁₆ aralkyloxy”, “C₆₋₁₄ arylthio”, “C₇₋₁₆ aralkylthio”, “C₃₋₈ cycloalkyl-amino”, “C₆₋₁₄ aryl-amino”, “C₃₋₈ cycloalkyl-carbonyl”, “C₆₋₁₄ aryl-carbonyl”, “C₇₋₁₆ aralkyl-carbonyl”, “5- to 7-membered heterocyclic carbonyl containing 1 to 4 hetero atoms of 1 or 2 species selected from nitrogen, sulfur and oxygen atoms in addition to carbon atoms”, “C₆₋₁₄ arylsulfonyl”, “C₆₋₁₄ arylsulfinyl”, “C₃₋₈ cycloalkyl-carbonylamino”, “C₆₋₁₄ aryl-carbonylamino”, “C₆₋₁₄ arylsulfonylamino”, “C₆₋₁₄ aryl-carbonyloxy” and “mono- or di-C₆₋₁₄ aryl-carbamoyloxy” in Substituent Group A include, for example, 1 to 5 substituents selected from, for example, a halogen atom, hydroxy, carboxy, nitro, cyano, the optionally substituted C₁₋₆ alkyl described above, the optionally substituted C₂₋₆ alkenyl described above, the optionally substituted C₂₋₆ alkynyl described above, the optionally substituted C₃₋₈ cycloalkyl described above, the optionally substituted C₁₋₆ alkoxy described above, the optionally substituted C₁₋₆ alkylthio described above, the optionally substituted C₁₋₆ alkylsulfinyl described above, the optionally substituted C₁₋₆ alkylsulfonyl described above, the optionally esterified carboxyl described above, carbamoyl, thiocarbamoyl, mono-C₁₋₆ alkylcarbamoyl, di-C₁₋₆ alkylcarbamoyl, mono- or di-C₆₋₁₄ arylcarbamoyl, mono- or di-5- to 7-membered heterocyclic carbamoyl containing 1 to 4 hetero atoms of 1 or 2 species selected from nitrogen, sulfur and oxygen atoms in addition to carbon atoms, and the like.

The “optionally substituted heterocyclic group” in Substituent Group A includes, for example, a 5- to 14-membered (monocyclic, bicyclic or tricyclic) heterocyclic group containing 1 to 4 hetero atoms of 1 or 2 species selected from nitrogen, sulfur and oxygen atoms in addition to carbon atoms, which may optionally be substituted with a halogen atom, hydroxy, carboxy, nitro, cyano, the optionally substituted C₁₋₆ alkyl described above, the optionally substituted C₂₋₆ alkenyl described above, the optionally substituted C₂₋₆ alkynyl described above, the optionally substituted C₃₋₈ cycloalkyl described above, the optionally substituted C₆₋₁₄ aryl described above, the optionally substituted C₁₋₆ alkoxy described above, the optionally substituted C₁₋₆ alkylthio described above, the optionally substituted C₆₋₁₄ arylthio described above, the optionally substituted C₇₋₁₆ aralkylthio described above, the optionally substituted C₁₋₆ alkylsulfinyl described above, the optionally substituted C₆₋₁₄ arylsulfinyl described above, the optionally substituted C₁₋₆ alkylsulfonyl described above, the optionally substituted C₆₋₁₄ arylsulfonyl described above, the optionally esterified carboxyl described above, carbamoyl, thiocarbamoyl, mono-C₁₋₆ alkylcarbamoyl, di-lower alkylcarbamoyl, mono- or di-C₆₋₁₄ arylcarbamoyl, mono- or di-5- or 7-membered heterocyclic carbamoyl containing 1 to 4 hetero atoms of 1 or 2 species selected from nitrogen, sulfur and oxygen atoms in addition to carbon atoms, or the like; preferably (i) a 5- to 14-membered (preferably, 5- to 10-membered) aromatic heterocyclic group, (ii) a 5- to 10-membered non-aromatic heterocyclic group or (iii) a monovalent group formed by removing one optional hydrogen atom from 7- to 10-membered bridged-hetero ring, and the like, are employed; among others, preferably used is a 5-membered aromatic heterocyclic group. Specifically used are an aromatic heterocyclic group such as thienyl (e.g., 2-thienyl, 3-thienyl), furyl (e.g., 2-furyl, 3-furyl), pyridyl (e.g., 2-pyridyl, 3-pyridyl, 4-pyridyl), thiazolyl (e.g., 2-thiazolyl, 4-thiazolyl, 5-thiazolyl), oxazolyl (e.g., 2-oxazolyl, 4-oxazolyl), quinolyl (e.g., 2-quinolyl, 3-quinolyl, 4-quinolyl, 5-quinolyl, 8-quinolyl), isoquinolyl (e.g., 1-isoquinolyl, 3-isoquinolyl, 4-isoquinolyl, 5-isoquinolyl), pyrazinyl, pyrimidinyl (e.g., 2-pyrimidinyl, 4-pyrimidinyl), pyrrolyl (e.g., 1-pyrrolyl, 2-pyrrolyl, 3-pyrrolyl), imidazolyl (e.g., 1-imidazolyl, 2-imidazolyl, 4-imidazolyl), pyrazolyl (e.g., 1-pyrazolyl, 3-pyrazolyl, 4-pyrazolyl), pyridazinyl (e.g., 3-pyridazinyl, 4-pyridazinyl), isothiazolyl (e.g., 3-isothiazolyl), isoxazolyl (e.g., 3-isoxazolyl), indolyl (e.g., 1-indolyl, 2-indolyl, 3-indolyl), 2-benzothiazolyl, benzo[b]thienyl, (e.g., 2-benzo[b]thienyl, 3-benzo[b]thienyl), benzo[b]furanyl (e.g., 2-benzo[b]furanyl, 3-benzo[b]furanyl), etc., a non-aromatic heterocyclic group such as pyrrolidinyl (e.g., 1-pyrrolidinyl, 2-pyrrolidinyl, 3-pyrrolidinyl), oxazolidinyl (e.g., 2-oxazolidinyl), imidazolinyl (e.g., 1-imidazolinyl, 2-imidazolinyl, 4-imidazolinyl), piperidinyl (e.g., 1-piperidinyl, 2-piperidinyl, 3-piperidinyl, 4-piperidinyl), piperazinyl (e.g., 1-piperazinyl, 2-piperazinyl), morpholino, thiomorpholino, etc.

The “optionally substituted carbamoyl” in Substituent Group A includes a carbamoyl group, which may optionally be substituted with the optionally substituted C₁₋₆ alkyl, optionally substituted C₂₋₆ alkenyl, an optionally substituted C₂₋₆ alkynyl, an optionally substituted C₃₋₈ cycloalkyl, an optionally substituted C₆₋₁₄ aryl, an optionally substituted heterocyclic group described above, etc., and specific examples are carbamoyl, thiocarbamoyl, mono-C₁₋₆ alkylcarbamoyl (e.g., methylcarbamoyl, ethylcarbamoyl, etc.), di-C₁₋₆ alkylcarbamoyl (e.g., dimethylcarbamoyl, diethylcarbamoyl, ethylmethylcarbamoyl, etc.), C₁₋₆ alkyl (C₁₋₆ alkoxy)carbamoyl (e.g., methyl(methoxy)carbamoyl, ethyl(methoxy)carbamoyl), mono- or di-C₆₋₁₄ arylcarbamoyl (e.g., phenylcarbamoyl, 1-naphthylcarbamoyl, 2-naphthylcarbamoyl, etc.), mono- or di-5- to 7-membered heterocyclic carbamoyl containing 1 to 4 hetero atoms of 1 or 2 species selected from nitrogen, sulfur and oxygen atoms in addition to carbon atoms (e.g., 2-pyridylcarbamoyl, 3-pyridylcarbamoyl, 4-pyridylcarbamoyl, 2-thienylcarbamoyl, 3-thienylcarbamoyl, etc.), 5- to 7-membered cyclic carbamoyl (e.g., 1-pyrrolidinylcarbonyl, 1-piperidinylcarbonyl, hexamethyleneiminocarbonyl), and the like.

The “optionally substituted amino” in Substituent Group A includes an amino, which may optionally be substituted with 1 or 2 groups selected from the optionally substituted C₁₋₆ alkyl described above, the optionally substituted C₂₋₆ alkenyl described above, the optionally substituted C₂₋₆ alkynyl described above, the optionally substituted C₃₋₈ cycloalkyl described above, the optionally substituted C₆₋₁₄ aryl described above, the optionally substituted C₁₋₆ alkoxy described above, formyl, the optionally substituted C₁₋₆ alkyl-carbonyl described above, the optionally substituted C₃₋₈ cycloalkyl-carbonyl described above, the optionally substituted C₆₋₁₄ aryl-carbonyl described above, the optionally substituted C₁₋₆ alkoxy-carbonyl described above, the optionally substituted C₁₋₆ alkylsulfonyl described above, the optionally substituted C₆₋₁₄ arylsulfonyl, and the like.

More preferably, the substituents used for these “C₆₋₁₂ aromatic hydrocarbon group,” “5- to 14-membered aromatic heterocyclic group consisting of 1 to 7 carbon atoms and hetero atoms selected from the group consisting of nitrogen, oxygen and sulfur atoms,” “C₈₋₁₄ aromatic fused-ring group,” “5- to 14-membered aromatic fused heterocyclic group consisting of 3 to 11 carbon atoms and hetero atoms selected from the group consisting of nitrogen, oxygen and sulfur atoms,” “non-aromatic cyclic hydrocarbon group having carbon atoms not greater than 7” and “non-aromatic heterocyclic group having carbon atoms not greater than 7” are a halogen atom, hydroxy, C₁₋₆ alkoxy, an optionally halogenated C₁₋₆ alkyl, an optionally halogenated C₁₋₆ alkoxy, amino, nitro, cyano, etc.

Examples of R⁴ used include:
(1) “a C₁₋₄ alkyl group having an optionally substituted C₆₋₁₂ aromatic hydrocarbon group” such as benzyl, 2-fluorobenzyl, 3-fluorobenzyl, 4-fluorobenzyl, 4-chlorobenzyl, 3, 4-difluorobenzyl, 3, 4-dichlorobenzyl, pentafluorobenzyl, 4-hydroxybenzyl, 4-methoxybenzyl, 3-trifluoromethylbenzyl, 4-aminobenzyl, 4-nitrobenzyl, 4-cyanobenzyl, phenethyl, etc.;
(2) “a C₁₋₄ alkyl group having an optionally substituted 5- to 14-membered aromatic heterocyclic group consisting of 1 to 7 carbon atoms and hetero atoms selected from the group consisting of nitrogen, oxygen and sulfur atoms” such as 2-pyridylmethyl, 3-pyridylmethyl, 4-pyridylmethyl, 2-thienylmethyl, 3-thienylmethyl, 4-thiazolylmethyl, etc.;
(3) “a C₁₋₄ alkyl group having an optionally substituted C₈₋₁₄ aromatic fused-ring group” such as 1-naphthylmethyl, 2-naphthylmethyl, inden-2-ylmethyl, etc.;
(4) “a C₁₋₄ alkyl group having an optionally substituted 5- to 14-membered aromatic fused heterocyclic group consisting of 3 to 11 carbon atoms and hetero atoms selected from the group consisting of nitrogen, oxygen and sulfur atoms” such as 3-indolemethyl, 1-formylindol-3-ylmethyl, 3-benzo[b]thienylmethyl, 2-quinolylmethyl, etc.;
(5) “a C₁₋₄ alkyl group having an optionally substituted non-aromatic cyclic hydrocarbon group having carbon atoms not greater than 7” such as cyclohexylmethyl, cyclopentylmethyl, indan-2-ylmethyl, etc.;
(6) “a C₁₋₄ alkyl group having an optionally substituted non-aromatic heterocyclic group having carbon atoms not greater than 7” such as 4-piperidinylmethyl, tetrahydrofurfuryl, tetrahydrofuran-2-yl, tetrahydropyran-3-yl, indolin-3-yl, etc.; among others, preferred are benzyl, 2-fluorobenzyl, 3-fluorobenzyl, 4-fluorobenzyl, 4-hydroxybenzyl, 4-aminobenzyl, 4-nitrobenzyl, 4-chlorobenzyl, 4-methoxybenzyl, 4-cyanobenzyl, 3-trifluoromethylbenzyl, 3, 4-dichlorobenzyl, 3, 4-difluorobenzyl, pentafluorobenzyl, 3-pyridylmethyl, 4-pyridylmethyl, 3-indolemethyl, 1-formylindol-3-ylmethyl, 3-benzo[b]thienylmethyl, 2-quinolylmethyl, 1-naphthylmethyl, 2-naphthylmethyl, cyclohexylmethyl, phenethyl, etc. are preferred, especially benzyl, 2-fluorobenzyl, 3-fluorobenzyl, 4-fluorobenzyl, 4-hydroxybenzyl, 4-aminobenzyl, 4-nitrobenzyl, 4-chlorobenzyl, 4-methoxybenzyl, 4-cyanobenzyl, 3-trifluoromethylbenzyl, 3, 4-dichlorobenzyl, 3, 4-difluorobenzyl, pentafluorobenzyl, 3-pyridylmethyl, 4-pyridylmethyl, 3-indolemethyl, 3-benzo[b]thienylmethyl, 1-naphthylmethyl, 2-naphthylmethyl, cyclohexylmethyl, etc.

Q¹, which may be the same as R⁴, represents a C₁₋₄ alkyl group which may be optionally substituted with a substituent selected from the group consisting of:
(1) an optionally substituted C₆₋₁₂ aromatic hydrocarbon group;
(2) an optionally substituted 5- to 14-membered aromatic heterocyclic group consisting of 1 to 7 carbon atoms and heteroatoms selected from the group consisting of nitrogen, oxygen and sulfur atoms;
(3) an optionally substituted C₈₋₁₄ aromatic fused-ring group;
(4) an optionally substituted 5- to 14-membered aromatic fused heterocyclic group consisting of 3 to 11 carbon atoms and heteroatoms selected from the group consisting of nitrogen, oxygen and sulfur atoms;
(5) an optionally substituted non-aromatic cyclic hydrocarbon group having carbon atoms not greater than 7; and
(6) an optionally substituted non-aromatic heterocyclic group having carbon atoms not greater than 7.

Illustrative examples of Q¹ include:
(1) C₁₋₄ alkyl groups having an optionally substituted C₆₋₁₂ aromatic hydrocarbon group, such as benzyl, 2-fluorobenzyl, 3-fluorobenzyl, 4-fluorobenzyl, 4-chlorobenzyl, 3,4-difluorobenzyl, 3-4,dichlorobenzyl, pentafluorobenzyl, 4-hydroxybenzyl, 4-methoxybenzyl, 4-trifluoromethylbenzyl, 4-aminobenzyl, 4-nitrobenzyl, 4-cyanobenzyl and phenethyl;
(2) C₁₋₄ alkyl groups having an optionally substituted 5- to 14-membered aromatic heterocyclic group consisting of 1 to 7 carbon atoms and heteroatoms selected from the group consisting of nitrogen, oxygen and sulfur atoms, such as 2-pyridylmethyl, 3-pyridylmethyl, 4-pyridylmethyl, 2-thienylmethyl, 3-thienylmethyl and 4-thiazolylmethyl;
(3) C₁₋₄ alkyl groups having an optionally substituted C₈₋₁₄ aromatic fused-ring group, such as 1-naphthylmethyl, 2-naphthylmethyl and inden-2-ylmethyl;
(4) C₁₋₄ alkyl groups having an optionally substituted 5- to 14-membered aromatic fused-heterocyclic group which consists of 3 to 11 carbon atoms and heteroatoms selected from the group consisting of nitrogen, oxygen and sulfur atoms, such as 3-indolemethyl, 1-formylindol-3-ylmethyl, 3-benzo[b]thienylmethyl and 2-quinolylmethyl;
(5) C₁₋₄ alkyl groups having an optionally substituted non-aromatic cyclic hydrocarbon group of up to 7 carbon atoms, such as cyclohexylmethyl, cyclopentylmethyl and indan-2-ylmethyl; and
(6) C₁₋₄ alkyl groups having an optionally substituted non-aromatic heterocyclic group of up to 7 carbon atoms, such as 4-piperidinylmethyl, tetrahydrofurfuryl, tetrahydrofuran-2-yl, tetrahydropyran-3-yl and indolin-3-yl. Of these, cyclohexylmethyl, benzyl, 4-fluorobenzyl, 4-hydroxybenzyl, 4-methoxybenzyl, pentafluorobenzyl, 2-pyridylmethyl, 4-pyridylmethyl, 1-naphthylmethyl, 2-naphthylmethyl, 3-indolemethyl and 2-thienylmethyl are preferred. Benzyl, 4-fluorobenzyl and cyclohexylmethyl are especially preferred.

Q² represents (1) CH₂ which may optionally be substituted with an optionally substituted C₁₋₄ alkyl group with a substituent selected from the group consisting of carbamoyl group and hydroxyl group, (2) NH which may optionally be substituted with an optionally substituted C₁₋₄ alkyl group with a substituent selected from the group consisting of carbamoyl group and hydroxyl group, or (3) an oxygen atom (O).

Examples of the “C₁₋₄ alkyl group” used include methyl, ethyl, propyl, isopropyl, butyl, isobutyl, sec-butyl and tert-butyl.

Preferred examples of Q² include CH₂, CH(CH₃), CH(CH₂OH) and NH.

Y represents a group represented by the formula: —CONH—, —CSNH—, —CH₂NH—, —NHCO—, —CH₂O—, —CH₂S—, —COO—, —CSO—, —CH₂CH₂— or —CH═CH—, which may optionally be substituted with a C₁₋₆ alkyl group.

Examples of the “C₁₋₆ alkyl group” used include methyl, ethyl, propyl, isopropyl, butyl, isobutyl, sec-butyl, tert-butyl, pentyl, isopentyl, neopentyl and hexyl.

Preferred examples of Y include groups of the formula: —CONH—, —CSNH—, —NHCO—, —CH₂NH—, —CH₂O—, —COO— and —CSO—. Of these, groups of the formulas: —CONH—, —CSNH—, —NHCO— and —CH₂NH— are especially preferred.

Z⁹ represents a hydrogen atom, oxygen (O) or sulfur (S), and preferably oxygen or sulfur.

Here, when Z⁹ is a hydrogen atom, the moiety represented by the formula >C═Z⁹ has the structure >CH_{2.}

P and P′, which may be the same or different, each may form a ring by combining P and P′ or P and Q¹ together and represents:
(1) hydrogen atom,
(2) an optional amino acid residue continuously or discontinuously bound from the C-terminal end of the 1-48 amino acid sequence in the amino acid sequence represented by SEQ ID NO: 1 (54 amino acid residues of human metastin); (3) a group represented by formula: J¹-J²-C(J³)(Q³)Y¹C(J⁴)(Q⁴)Y²C(J⁵)(Q⁵)Y³C(J⁶)(Q⁶)C(═Z¹⁰)— (wherein each symbol has the same significance as described above),
(4) a group represented by formula:

-J¹-J²-C(J⁷)(Q⁷)Y²C(J⁸)(Q⁸)Y³C(J⁹)(Q⁹)C(═Z¹⁰)—

(wherein each symbol has the same significance as described above),
(5) a group represented by formula:

J¹-J²-C(J¹⁰)(Q¹⁰)Y³C(J¹¹)(Q¹¹)C(═Z¹⁰)—

(wherein each symbol has the same significance as described above),
(6) a group represented by formula:

J¹-J²-C(J¹²)(Q¹²)C(═Z¹⁰)—

(wherein each symbol has the same significance as described above), or,
(7) a group represented by formula: J¹-
   (wherein J¹ has the same significance as described above).

Specific examples of the “optional amino acid residue continuously or discontinuously bound from the C-terminal end of the 1-48 amino acid sequence in the amino acid sequence represented by SEQ ID NO: 1,” which are employed, include:

```
  (1)Asn-
  (2)Trp Asn-,
  (3)Asn Trp Asn-,
  (4)Tyr Asn Trp Asn-,
  (5)Asn Tyr Asn Trp Asn-,
  (6)Pro Asn Tyr Asn Trp Asn-,
  (7)Leu Pro Asn Tyr Asn Trp Asn-,
  (8)Asp Leu Pro Asn Tyr Asn Trp Asn-,
  (9)Lys Asp Leu Pro Asn Tyr Asn Trp Asn-,
(10)Glu Lys Asp Leu Pro Asn Tyr Asn Trp Asn-,
(11)Arg Glu Lys Asp Leu Pro Asn Tyr Asn Trp Asn-,
(12)Gln Arg Glu Lys Asp Leu Pro Asn Tyr Asn Trp Asn-,
(13)Val Gln Arg Glu Lys Asp Leu Pro Asn Tyr Asn Trp Asn-,
(14)Leu Val Gln Arg Glu Lys Asp Leu Pro Asn Tyr Asn Trp Asn-,
(15)Val Leu Val Gln Arg Glu Lys Asp Leu Pro Asn Tyr Asn Trp Asn-,
(16)Ala Val Leu Val Gln Arg Glu Lys Asp Leu Pro Asn Tyr Asn Trp Asn-,
(17)Gly Ala Val Leu Val Gln Arg Glu Lys Asp Leu Pro Asn Tyr Asn Trp Asn-,
(18)Gln Gly Ala Val Leu Val Gln Arg Glu Lys Asp Leu Pro Asn Tyr Asn Trp Asn-,
(19)Pro Gln Gly Ala Val Leu Val Gln Arg Glu Lys Asp Leu Pro Asn Tyr Asn Trp Asn-,
(20)Ala Pro Gln Gly Ala Val Leu Val Gln Arg Glu Lys Asp Leu Pro Asn Tyr Asn Trp Asn-,
(21)Pro Ala Pro Gln Gly Ala Val Leu Val Gln Arg Glu Lys Asp Leu Pro Asn Tyr Asn Trp Asn-,
(22)Ile Pro Ala Pro Gln Gly Ala Val Leu Val Gln Arg Glu Lys Asp Leu Pro Asn Tyr Asn Trp
Asn-,
(23)Gln Ile Pro Ala Pro Gln Gly Ala Val Leu Val Gln Arg Glu Lys Asp Leu Pro Asn Tyr Asn
Trp Asn-,
(24)Arg Gln Ile Pro Ala Pro Gln Gly Ala Val Leu Val Gln Arg Glu Lys Asp Leu Pro Asn Tyr
Asn Trp Asn-,
(25)Ser Arg Gln Ile Pro Ala Pro Gln Gly Ala Val Leu Val Gln Arg Glu Lys Asp Leu Pro Asn
Tyr Asn Trp Asn-,
(26)His Ser Arg Gln Ile Pro Ala Pro Gln Gly Ala Val Leu Val Gln Arg Glu Lys Asp Leu Pro
Asn Tyr Asn Trp Asn-,
(27)Pro His Ser Arg Gln Ile Pro Ala Pro Gln Gly Ala Val Leu Val Gln Arg Glu Lys Asp Leu Pro
Asn Tyr Asn Trp Asn-,
(28)Ala Pro His Ser Arg Gln Ile Pro Ala Pro Gln Gly Ala Val Leu Val Gln Arg Glu Lys Asp Leu
Pro Asn Tyr Asn Trp Asn-,
(29)Ser Ala Pro His Ser Arg Gln Ile Pro Ala Pro Gln Gly Ala Val Leu Val Gln Arg Glu Lys Asp
Leu Pro Asn Tyr Asn Trp Asn-,
(30)Leu Ser Ala Pro His Ser Arg Gln Ile Pro Ala Pro Gln Gly Ala Val Leu Val Gln Arg Glu Lys
Asp Leu Pro Asn Tyr Asn Trp Asn-,
(31)Gly Leu Ser Ala Pro His Ser Arg Gln Ile Pro Ala Pro Gln Gly Ala Val Leu Val Gln Arg Glu
Lys Asp Leu Pro Asn Tyr Asn Trp Asn-,
(32)Pro Gly Leu Ser Ala Pro His Ser Arg Gln Ile Pro Ala Pro Gln Gly Ala Val Leu Val Gln Arg
Glu Lys Asp Leu Pro Asn Tyr Asn Trp Asn-,
(33)Gln Pro Gly Leu Ser Ala Pro His Ser Arg Gln Ile Pro Ala Pro Gln Gly Ala Val Leu Val Gln
Arg Glu Lys Asp Leu Pro Asn Tyr Asn Trp Asn-,
(34)Gln Gln Pro Gly Leu Ser Ala Pro His Ser Arg Gln Ile Pro Ala Pro Gln Gly Ala Val Leu Val
Gln Arg Glu Lys Asp Leu Pro Asn Tyr Asn Trp Asn-,
(35)Arg Gln Gln Pro Gly Leu Ser Ala Pro His Ser Arg Gln Ile Pro Ala Pro Gln Gly Ala Val Leu
Val Gln Arg Glu Lys Asp Leu Pro Asn Tyr Asn Trp Asn-,
(36)Ser Arg Gln Gln Pro Gly Leu Ser Ala Pro His Ser Arg Gln Ile Pro Ala Pro Gln Gly Ala Val
Leu Val Gln Arg Glu Lys Asp Leu Pro Asn Tyr Asn Trp Asn-,
(37)Gly Ser Arg Gln Gln Pro Gly Leu Ser Ala Pro His Ser Arg Gln Ile Pro Ala Pro Gln Gly Ala
Val Leu Val Gln Arg Glu Lys Asp Leu Pro Asn Tyr Asn Trp Asn-,
(38)Ser Gly Ser Arg Gln Gln Pro Gly Leu Ser Ala Pro His Ser Arg Gln Ile Pro Ala Pro Gln Gly
Ala Val Leu Val Gln Arg Glu Lys Asp Leu Pro Asn Tyr Asn Trp Asn-,
(39)Ser Ser Gly Ser Arg Gln Gln Pro Gly Leu Ser Ala Pro His Ser Arg Gln Ile Pro Ala Pro Gln
Gly Ala Val Leu Val Gln Arg Glu Lys Asp Leu Pro Asn Tyr Asn Trp Asn-,
(40)Glu Ser Ser Gly Ser Arg Gln Gln Pro Gly Leu Ser Ala Pro His Ser Arg Gln Ile Pro Ala Pro
Gln Gly Ala Val Leu Val Gln Arg Glu Lys Asp Leu Pro Asn Tyr Asn Trp Asn-,
(41)Pro Glu Ser Ser Gly Ser Arg Gln Gln Pro Gly Leu Ser Ala Pro His Ser Arg Gln Ile Pro Ala
Pro Gln Gly Ala Val Leu Val Gln Arg Glu Lys Asp Leu Pro Asn Tyr Asn Trp Asn-,
(42)Pro Pro Glu Ser Ser Gly Ser Arg Gln Gln Pro Gly Leu Ser Ala Pro His Ser Arg Gln Ile Pro
Ala Pro Gln Gly Ala Val Leu Val Gln Arg Glu Lys Asp Leu Pro Asn Tyr Asn Trp Asn-,
(43)Pro Pro Pro Glu Ser Ser Gly Ser Arg Gln Gln Pro Gly Leu Ser Ala Pro His Ser Arg Gln Ile
Pro Ala Pro Gln Gly Ala Val Leu Val Gln Arg Glu Lys Asp Leu Pro Asn Tyr Asn Trp Asn-,
(44)Ser Pro Pro Pro Glu Ser Ser Gly Ser Arg Gln Gln Pro Gly Leu Ser Ala Pro His Ser Arg Gln
Ile Pro Ala Pro Gln Gly Ala Val Leu Val Gln Arg Glu Lys Asp Leu Pro Asn Tyr Asn Trp Asn-,
(45)Leu Ser Pro Pro Pro Glu Ser Ser Gly Ser Arg Gln Gln Pro Gly Leu Ser Ala Pro His Ser Arg
Gln Ile Pro Ala Pro Gln Gly Ala Val Leu Val Gln Arg Glu Lys Asp Leu Pro Asn Tyr Asn Trp
Asn-,
(46)Ser Leu Ser Pro Pro Pro Glu Ser Ser Gly Ser Arg Gln Gln Pro Gly Leu Ser Ala Pro His Ser
Arg Gln Ile Pro Ala Pro Gln Gly Ala Val Leu Val Gln Arg Glu Lys Asp Leu Pro Asn Tyr Asn
Trp Asn-,
(47)Thr Ser Leu Ser Pro Pro Pro Glu Ser Ser Gly Ser Arg Gln Gln Pro Gly Leu Ser Ala Pro His
Ser Arg Gln Ile Pro Ala Pro Gln Gly Ala Val Leu Val Gln Arg Glu Lys Asp Leu Pro Asn Tyr
Asn Trp Asn-,
(48)Gly Thr Ser Leu Ser Pro Pro Pro Glu Ser Ser Gly Ser Arg Gln Gln Pro Gly Leu Ser Ala Pro
His Ser Arg Gln Ile Pro Ala Pro Gln Gly Ala Val Leu Val Gln Arg Glu Lys Asp Leu Pro Asn
Tyr Asn Trp Asn-
```

J¹ represents (a) hydrogen atom or (b) (i) a C₁₋₁₅ acyl group, (ii) a C₁₋₁₅ alkyl group, (iii) a C₆₋₁₄ aryl group, (iv) carbamoyl group, (v) carboxyl group, (vi) sulfino group or (vii) amidino group, (viii) glyoxyloyl group or (ix) amino group, which groups may optionally be substituted with a substituent containing an optionally substituted cyclic group;

The “cyclic group” used includes, for example, “an optionally substituted aromatic hydrocarbon group,” “an optionally substituted aromatic heterocyclic group,” “an optionally substituted aromatic fused-ring group,” “an optionally substituted aromatic fused heterocyclic group,” “an optionally substituted non-aromatic cyclic hydrocarbon group,” “an optionally substituted non-aromatic heterocyclic group,” etc., and examples of the “aromatic hydrocarbon group,” “aromatic heterocyclic group,” “aromatic fused-ring group” and “aromatic fused heterocyclic group” used are the same as those given above.

The “non-aromatic cyclic hydrocarbon group” used includes a C₃₋₈ cycloalkyl group such as cyclopropyl, cyclobutyl, cyclopentyl, cyclohexyl, etc.

The “non-aromatic heterocyclic group” used includes a 5- to 10-membered non-aromatic heterocyclic group containing 1 to 4 hetero atoms of 1 or 2 species selected from nitrogen, sulfur and oxygen atoms in addition to 1 to 7 carbon atoms such as pyrrolidinyl (e.g., 1-pyrrolidinyl, 2-pyrrolidinyl, 3-pyrrolidinyl), oxazolidinyl (e.g., 2-oxazolidinyl), imidazolinyl (e.g., 1-imidazolinyl, 2-imidazolinyl, 4-imidazolinyl), piperidinyl (e.g., 1-piperidinyl, 2-piperidinyl, 3-piperidinyl, 4-piperidinyl), piperazinyl (e.g., 1-piperazinyl, 2-piperazinyl), morpholino, thiomorpholino, etc.

The substituent optionally present on the “cyclic group” includes the same substituents as Substituent Group A described above.

The “C₁₋₁₅ acyl group” used includes, for example, formyl, C₁₋₁₄ alkyl-carbonyl (e.g., C₁₋₆ alkyl-carbonyl such as acetyl, propionyl, pivaloyl, etc.) and the like.

The “C₁₋₁₅ alkyl group” used include, for example, methyl, ethyl, propyl, isopropyl, butyl, isobutyl, sec-butyl, tert-butyl, pentyl, isopentyl, neopentyl, hexyl, heptyl, octyl, nonanyl, decanyl, etc.

The “C₆₋₁₄ aryl group” used includes, for example, phenyl, 1-naphthyl, 2-naphthyl, biphenyl, etc.

(1) The C₁₋₁₅ acyl group, which may optionally be substituted with a substituent containing a cyclic group, includes (i) formyl, (ii) C₁₋₁₄ alkyl-carbonyl (e.g., C₁₋₆ alkyl-carbonyl such as acetyl, propionyl, pivaloyl, etc.), (iii) C₃₋₈ cycloalkyl-carbonyl (e.g., cyclopropylcarbonyl, cyclopentylcarbonyl, cyclohexylcarbonyl, 1-methylcyclohexylcarbonyl, etc.), (iv) C₃₋₈ cycloalkyl-C₁₋₆ alkyl-carbonyl (e.g., cyclopropylacetyl, cyclopentylacetyl, cyclohexylacetyl, etc.), (v) C₆₋₁₄ aryl-carbonyl (e.g., benzoyl, 1-naphthoyl, 2-naphthoyl, etc.), C₆₋₁₄ aralkyl-carbonyl (e.g., phenylacetyl, 3-phenylpropionyl, etc.), (vi) 5- to 7-membered monocyclic heterocyclic carbonyl containing 1 to 4 hetero atoms of 1 or 2 species selected from nitrogen, sulfur and oxygen atoms in addition to carbon atoms (e.g., nicotinoyl, isonicotinoyl, thenoyl, furoyl, morpholinocarbonyl, thiomorpholinocarbonyl, piperazin-1-ylcarbonyl, pyrrolidin-1-ylcarbonyl, etc.), (vii) 5- to 7-membered monocyclic heterocycle-C₁₋₆ alkylcarbonyl, which contains 1 to 4 hetero atoms of 1 or 2 species selected from nitrogen, sulfur and oxygen atoms in addition to carbon atoms (e.g., 3-pyridylacetyl, 4-pyridylacetyl, 2-thienylacetyl, 2-furylacetyl, morpholinoacetyl, thiomorpholinoacetyl, piperidin-2-acetyl, pyrrolidine-2-ylacetyl, etc.), (viii) 5- to 14-membered (preferably, 5- to 10-membered) bicyclic or tricyclic aromatic heterocyclic carbonyl containing 1 to 4 hetero atoms of 1 or 2 species selected from nitrogen, sulfur and oxygen atoms in addition to 3 to 11 carbon atoms (e.g., 2-indolecarbonyl, 3-indolecarbonyl, 2-quinolylcarbonyl, 1-isoquinolylcarbonyl, 2-benzo[b]thienylcarbonyl, 2-benzo[b]furanylcarbonyl, etc.), (ix) 5- to 14-membered (preferably 5- to 10-membered) bicyclic or tricyclic aromatic heterocycle-C₁₋₆ alkylcarbonyl, which contains 1 to 4 hetero atoms of 1 or 2 species selected from nitrogen, sulfur and oxygen atoms in addition to 3 to 11 carbon atoms (e.g., 2-indoleacetyl, 3-indoleacetyl, 2-quinolylacetyl, 1-isoquinolylacetyl, 2-benzo[b]thienylacetyl, 2-benzo[b]furanylacetyl, etc.), etc., among others, preferably used are acetyl, 2-indolecarbonyl, 3-indolecarbonyl, 3-indoleacetyl, 3-indolepropionyl, 2-indolinecarbonyl, 3-phenylpropionyl, diphenylacetyl, 2-pyridinecarbonyl, 3-pyridinecarbonyl, 4-pyridinecarbonyl, 1-pyridinioacetyl, 2-pyridineacetyl, 3-pyridineacetyl, 4-pyridineacetyl, 3-(1-pyridinio)propionyl, 3-(pyridin-2-yl)propionyl, 3-(pyridin-3-yl)propionyl, 3-(pyridin-4-yl)propionyl, 4-imidazoleacetyl, cyclohexanecarbonyl, 1-piperidineacetyl, 1-methyl-1-piperidinioacetyl, 4-piperidinecarbonyl, 2-pyrimidinecarbonyl, 4-pyrimidinecarbonyl, 5-pyrimidinecarbonyl, 2-pyrimidineacetyl, 4-pyrimidineacetyl, 5-pyrimidineacetyl, 3-(pyrimidin-2-yl)propionyl, 3-(pyrimidin-4-yl)propionyl, 3-(pyrimidin-5-yl)propionyl, butanoyl, hexanoyl, octanoyl, D-glucuronyl, amino-(4-hydroxyphenyl)acetyl, etc.

(2) The C₁₋₁₅ alkyl group, which may optionally be substituted with a substituent containing a cyclic group, includes, for example, (i) mono- or di-C₁₋₁₅ alkyl (e.g., methyl, ethyl, propyl, isopropyl, butyl, isobutyl, sec-butyl, tert-butyl, pentyl, isopentyl, neopentyl, hexyl, heptyl, octyl, nonanyl, decanyl), (ii) mono- or di-C₃₋₈ cycloalkyl (e.g., cyclopropyl, cyclopentyl, etc.), (iii) mono- or di-C₃₋₈ cycloalkyl-C₁₋₇ alkyl (e.g., cyclopropylmethyl, cyclopentylmethyl, cyclohexylethyl, etc.), (iv) mono- or di-C₇₋₂₀ (preferably, C₇₋₁₇, more preferably C₇₋₁₅) aralkyl (e.g., benzyl, phenethyl, etc.), (v) mono- or di-5- to 7-membered monocyclic heterocycle-C₁₋₆ alkyl group, which contains 1 to 4 hetero atoms of 1 or 2 species selected from nitrogen, sulfur and oxygen atoms in addition to carbon atoms (e.g., 3-pyridylmethyl, 4-pyridylmethyl, 2-thienylmethyl, furfuryl, etc.), (vi) mono- or di-5- to 14-membered (preferably, 5- to 10-membered) bicyclic or tricyclic aromatic heterocycle-C₁₋₆ alkyl, which contains 1 to 4 hetero atoms of 1 or 2 species selected from nitrogen, sulfur and oxygen atoms in addition to 3 to 11 carbon atoms (e.g., 2-indolemethyl, 3-indolemethyl, 3-(indol-3-yl)propyl, 2-quinolylmethyl, 1-isoquinolylmethyl, 2-benzo[b]thienylmethyl, 2-benzo[b]furanylmethyl, etc.), etc.; among others, methyl, ethyl, benzyl, 3-(indol-3-yl)propyl, etc. are preferably used.

(3) The C₆₋₁₄ aryl group, which may optionally be substituted with a substituent containing a cyclic group, includes, for example, a C₆₋₁₄ aryl group (e.g., phenyl, naphthyl, biphenyl), which may optionally be substituted with (i) a C₆₋₁₄ carbocyclic group (e.g., cycloalkyl, phenyl, 1-naphthyl, 2-naphthyl, etc.), (ii) a 5- to 7-membered monocyclic heterocyclic group containing 1 to 4 hetero atoms of 1 or 2 species selected from nitrogen, sulfur and oxygen atoms in addition to carbon atoms (e.g., 3-pyridyl, 2-thienyl, etc.), (iii) a 5- to 14-membered (preferably, 5- to 10-membered) bicyclic or tricyclic aromatic heterocyclic group containing 1 to 4 hetero atoms of 1 or 2 species selected from nitrogen, sulfur and oxygen atoms in addition to 3 to 11 carbon atoms (e.g., 2-indolyl, 3-indolyl, 2-quinolyl, 1-isoquinolyl, 2-benzo[b]thienyl, 2-benzo[b]furanyl, etc.), etc.

(4) The carbamoyl group, which may optionally be substituted with a substituent containing a cyclic group, includes (i) carbamoyl, (ii) mono- or di-C₁₋₁₅ alkylcarbamoyl (e.g., methylcarbamoyl, ethylcarbamoyl), (iii) mono- or di-C₃₋₈ cycloalkylcarbamoyl (e.g., cyclopropylcarbamoyl, cyclopentylcarbamoyl, cyclohexylcarbamoyl, etc.), (iv) mono- or di-C₃₋₈ cycloalkyl-C₁₋₆ alkyl-carbamoyl (e.g., cyclopropylmethylcarbamoyl, cyclopentylmethylcarbamoyl, 2-cyclohexylethylcarbamoyl, etc.), (v) mono- or di-C₆₋₁₄ aryl-carbamoyl (e.g., phenylcarbamoyl, etc.), a mono- or di-C₆₋₁₄ aralkyl-carbamoyl (e.g., benzylcarbamoyl, phenethylcarbamoyl, etc.), (vi) mono- or di-5- to 7-membered monocyclic heterocyclic carbamoyl containing 1 to 4 hetero atoms of 1 or 2 species selected from nitrogen, sulfur and oxygen atoms in addition to carbon atoms (e.g., 3-pyridinecarbamoyl, 2-thiophenecarbamoyl, piperidin-3-ylcarbamoyl, etc.), (vii) mono- or di-5- to 7-membered monocyclic heterocycle-C₁₋₆ alkylcarbamoyl, which contains 1 to 4 hetero atoms of 1 or 2 species selected from nitrogen, sulfur and oxygen atoms in addition to carbon atoms (e.g., 3-pyridylmethylcarbamoyl, 2-(pyridin-2-yl)ethylcarbamoyl, 2-(piperidin-1-yl)ethylcarbamoyl, etc.), (viii) mono- or di-5- to 14-membered (preferably, 5- to 10-membered) bicyclic or tricyclic aromatic heterocyclic carbamoyl containing 1 to 4 hetero atoms of 1 or 2 species selected from nitrogen, sulfur and oxygen atoms in addition to 3 to 11 carbon atoms (e.g., 4-indolecarbamoyl, 5-indolecarbamoyl, 3-quinolylcarbamoyl, 5-quinolylcarbamoyl, etc.), (ix) mono- or di-5- to 14-membered (preferably, 5- to 10-membered) bicyclic or tricyclic aromatic heterocyclic-C₁₋₆ alkylcarbonyl containing 1 to 4 hetero atoms of 1 or 2 species selected from nitrogen, sulfur and oxygen atoms in addition to 3 to 11 carbon atoms (e.g., benzimidazol-2-ylmethylcarbamoyl, 2-(indol-3-yl)ethylcarbamoyl, etc.), (x) 5- to 7-membered cyclic carbamoyl (e.g., 1-pyrrolidinylcarbonyl, 1-piperidinylcarbonyl, hexamethyleneiminocarbonyl, etc.), (xi) C₁₋₁₅ acylcarbamoyl (C₁₋₁₅ acyl as used herein has the same significance as the “C₁₋₁₅ acyl group” in the “C₁₋₁₅ acyl group, which may optionally be substituted with a substituent containing a cyclic group”), (xii) C₁₋₁₅ alkylaminocarbamoyl (C₁₋₁₅ alkyl as used herein has the same significance as the “C₁₋₁₅ alkyl group” in the “C₁₋₁₅ alkyl group, which may optionally be substituted with a substituent containing a cyclic group”), (xiii) C₆₋₁₄ arylaminocarbamoyl (C₆₋₁₄ aryl as used herein has the same significance as the “C₆₋₁₄ aryl group” in the “C₆₋₁₄ aryl group, which may optionally be substituted with a substituent containing a cyclic group”), etc.; among others, 2-(indol-3-yl)ethylcarbamoyl, etc. are preferably used.

(5) The carboxyl group, which may optionally be substituted with a substituent containing a cyclic group, includes (i) C₁₋₁₅ alkyloxycarbonyl (C₁₋₁₅ alkyl herein has the same significance as the “C₁₋₁₅ alkyl group” in the “C₁₋₁₅ alkyl group, which may optionally be substituted with a substituent containing a cyclic group,” e.g., tert-butyloxycarbonyl, benzyloxycarbonyl, 9-fluorenylmethoxycarbonyl), (ii) C₆₋₁₄ aryloxycarbonyl (C₆₋₁₄ aryl herein has the same significance as the “C₆₋₁₄ aryl group” in the “C₆₋₁₄ aryl group, which may optionally be substituted with a substituent containing a cyclic group,” e.g., phenoxycarbonyl), etc.

(6) The sulfino group, which may optionally be substituted with a substituent containing a cyclic group, includes (i) C₁₋₁₅ alkylsulfonyl (C₁₋₁₅ alkyl as used herein has the same significance as the “C₁₋₁₅ alkyl group” in the “C₁₋₁₅ alkyl group, which may optionally be substituted with a substituent containing a cyclic group,” e.g., benzylsulfonyl), (ii) C₆₋₁₄ arylsulfonyl (C₆₋₁₄ aryl as used herein has the same significance as the “C₆₋₁₄ aryl group” in the “C₆₋₁₄ aryl group, which may optionally be substituted with a substituent containing a cyclic group,” e.g., tosyl), etc.

(7) The amidino group, which may optionally be substituted with a substituent containing a cyclic group, includes (i) amidino, (ii) C₁₋₁₅ alkylamidino (C₁₋₁₅ alkyl as used herein has the same significance as the “C₁₋₁₅ alkyl group” in the “C₁₋₁₅ alkyl group, which may optionally be substituted with a substituent containing a cyclic group,” e.g., N-methylamidino), (iii) C₁₋₁₅ acylamidino (C₁₋₁₅ acyl as used herein has the same significance as the “C₁₋₁₅ acyl group” in the “C₁₋₁₅ acyl group, which may optionally be substituted with a substituent containing a cyclic group,” e.g., N-acetylamidino), etc.

(8) The glyoxyloyl group, which may optionally be substituted with a substituent containing a cyclic group, includes (i) C₁₋₁₅ alkyloxalyl (C₁₋₁₅ alkyl as used herein has the same significance as the “C₁₋₁₅ alkyl group” in the “C₁₋₁₅ alkyl group, which may optionally be substituted with a substituent containing a cyclic group,” e.g., ethyloxalyl), (ii) C₆₋₁₄ aryloxalyl (C₆₋₁₄ aryl as used herein has the same significance as the “C₆₋₁₄ aryl group” in the “C₆₋₁₄ aryl group, which may optionally be substituted with a substituent containing a cyclic group,” e.g., phenyloxalyl), etc.

(9) The use of the amino group, which may optionally be substituted with a substituent containing a cyclic group, includes (i) C₁₋₁₅ alkylamino (C₁₋₁₅ alkyl as used herein has the same significance as the “C₁₋₁₅ alkyl group” in the “C₁₋₁₅ alkyl group, which may optionally be substituted with a substituent containing a cyclic group”).

Among those described above, preferred examples of J¹ used include hydrogen atom, formyl, acetyl, 3-indolecarbonyl, 3-(indol-3-yl)propionyl, 3-phenylpropionyl, diphenylacetyl, 3-(pyridin-3-yl)propionyl, 4-imidazoleacetyl, cyclohexanecarbonyl, 1-piperidineacetyl, 1-methyl-1-piperidinioacetyl, 4-piperidinecarbonyl, hexanoyl, amino-(4-hydroxyphenyl)acetyl, D-glucuronyl, 2-(indol-3-yl)ethylcarbamoyl, tert-butyloxycarbonyl, 9-fluorenylmethoxycarbonyl, amidino, 4-guanidomethylbenzoyl, benzoyl, 3-indoleacetyl, benzyloxycarbonyl, tosyl, phenyl, benzyl, phenethyl, 3-pyridinecarbonyl, 2-pyridinecarbonyl, 4-pyridinecarbonyl, propionyl, isobutyryl, phenylacetyl, 2-methylnicotinoyl, 5-methylnicotinoyl, 6-methylnicotinoyl, pyrazinecarbonyl, cyclopropanecarbonyl, trifluoroacetyl, (R)-3-hydroxy-2-methylpropionyl, 2-hydroxyisobutyryl, 3-furancarbonyl, pyrrole-2-carbonyl, 4-imidazolecarbonyl, 6-hydroxynicotinoyl, 6-chloronicotinoyl, 6-(trifluoromethyl)nicotinoyl, dimethylcarbamoyl, 1-azetidinecarbonyl, 2-azetidinecarbonyl, 4-aminobenzoyl, 4-aminomethylbenzoyl, pyrrole-3-carbonyl, pyrimidine-4-carbonyl, pyrimidine-2-carbonyl, pyridazine-4-carbonyl, 6-aminocaproyl, glycyl, glycylglycyl, glycylglycylglycyl, alanylalanylalanyl, alanylalanylalanylalanyl, acetylglycyl, acetylglycylglycyl, acetylglycylglycylglycyl, acetylalanylalanylalanyl, acetylalanylalanylalanylalanyl, D-arginylglycyl, D-arginylglycylglycyl, D-arginylglycylglycylglycyl, D-arginylalanylalanylalanyl, D-arginylalanylalanylalanylalanyl, acetyl-D-arginylglycyl, acetyl-D-arginylglycylglycyl, acetyl-D-arginylglycylglycylglycyl, acetyl-D-arginylalanylalanylalanyl, acetyl-D-arginylalanylalanylalanylalanyl, cyclopropanecarbonyl, cyclopentanecarbonyl, cyclobutanecarbonyl, cyclohexanecarbonyl, 1-naphthoyl, 2-naphthoyl, arginyl, arginylarginyl, 6-(arginylamino)caproyl, 6-(D-arginylamino)caproyl, 6-(D-arginyl-D-arginylamino)caproyl, 6-(acetyl-D-arginylamino)caproyl, 6-((R)-2, 3-diaminopropionylamino)caproyl, 6-(D-norleucylamino)caproyl, 3-(D-arginylamino)propionyl, 4-(D-arginylamino)butyryl, 4-(D-arginyl-D-arginylamino)butyryl, 4-(D-arginyl-D-arginyl-D-arginylamino)butyryl, 3-(4-hydroxyphenyl)propionyl, butyryl, methyl, adipoyl, pyroglutamyl, glycoloyl, etc.; among others, preferred are hydrogen atom, formyl, acetyl, propionyl, 3-indolecarbonyl, 3-(indol-3-yl)propionyl, 3-phenylpropionyl, 3-(pyridin-3-yl)propionyl, 4-imidazoleacetyl, cyclohexanecarbonyl, hexanoyl, amino-(4-hydroxyphenyl)acetyl, 2-(indol-3-yl)ethylcarbamoyl, 9-fluorenylmethoxycarbonyl, amidino, 4-guanidomethylbenzoyl, benzoyl, 3-indoleacetyl, benzyl, phenethyl, 3-pyridinecarbonyl, 2-pyridinecarbonyl, 4-pyridinecarbonyl, isobutyryl, phenylacetyl, 6-methylnicotinoyl, pyrazinecarbonyl, cyclopropanecarbonyl, trifluoroacetyl, (R)-3-hydroxy-2-methylpropionyl, 2-hydroxyisobutyryl, 3-furancarbonyl, pyrrole-2-carbonyl, 4-imidazolecarbonyl, 6-hydroxynicotinoyl, 6-chloronicotinoyl, 6-(trifluoromethyl)nicotinoyl, dimethylcarbamoyl, 1-azetidinecarbonyl, 4-aminobenzoyl, 4-aminomethylbenzoyl, pyrrole-3-carbonyl, pyrimidine-4-carbonyl, pyrimidine-2-carbonyl, pyridazine-4-carbonyl, 6-aminocaproyl, cyclopropanecarbonyl, 2-naphthoyl, arginyl, 6-(arginylamino)caproyl, 6-(D-arginylamino)caproyl, 6-(D-arginyl-D-arginylamino)caproyl, 6-(acetyl-D-arginylamino)caproyl, 6-((R)-2, 3-diaminopropionylamino)caproyl, 6-(D-norleucylamino)caproyl, 3-(D-arginylamino)propionyl, 4-(D-arginylamino)butyryl, 4-(D-arginyl-D-arginylamino)butyryl, 4-(D-arginyl-D-arginyl-D-arginylamino)butyryl, 3-(4-hydroxyphenyl)propionyl, butyryl, adipoyl, pyroglutamyl, etc.

J² represents (1) NH optionally substituted with a C₁₋₆ alkyl group, (2) CH₂ optionally substituted with a C₁₋₆ alkyl group, (3) O or (4) S.

The “C₁₋₆ alkyl group” used includes methyl, ethyl, propyl, isopropyl, butyl, isobutyl, sec-butyl, tert-butyl, pentyl, isopentyl, neopentyl, hexyl, etc.

Preferably, J² is NH.

Each of J³ through J¹² represents hydrogen atom or a C₁₋₃ alkyl group.

The “C₁₋₃ alkyl group” used includes methyl, ethyl, propyl, isopropyl, etc.

Preferably, J³ is hydrogen atom.

Preferably, J⁴ is hydrogen atom.

Preferably, J⁵ is hydrogen atom.

Preferably, J⁶ is hydrogen atom.

Preferably, J⁷ is hydrogen atom.

Preferably, J⁸ is hydrogen atom.

Preferably, J⁹ is hydrogen atom.

Preferably, J¹⁰ is hydrogen atom.

Preferably, J¹¹ is hydrogen atom.

Preferably, J¹² is hydrogen atom.

Each of Q³ through Q¹² represents a C₁₋₄ alkyl group, which may optionally have a substituent selected from the group consisting of:

(1) an optionally substituted C₆₋₁₂ aromatic hydrocarbon group,

(2) an optionally substituted 5- to 14-membered aromatic heterocyclic group consisting of 1 to 7 carbon atoms and hetero atoms selected from the group consisting of nitrogen, oxygen and sulfur atoms,

(3) an optionally substituted C₈₋₁₄ aromatic fused-ring group,

(4) an optionally substituted 5- to 14-membered aromatic fused heterocyclic group consisting of 3 to 11 carbon atoms and hetero atoms selected from the group consisting of nitrogen, oxygen and sulfur atoms,

(5) an optionally substituted non-aromatic cyclic hydrocarbon group having carbon atoms not greater than 7,

(6) an optionally substituted non-aromatic heterocyclic group having carbon atoms not greater than 7,

(7) an optionally substituted amino group;

(8) an optionally substituted guanidino group;

(9) an optionally substituted hydroxyl group;

(10) an optionally substituted carboxyl group;

(11) an optionally substituted carbamoyl group; and,

(12) an optionally substituted sulfhydryl group;

or hydrogen atom.

Particularly preferred Q³ to Q⁹ are a C₁₋₄ alkyl group having a substituent selected from the group consisting of:

(1) an optionally substituted C₆₋₁₂ aromatic hydrocarbon group,

(2) an optionally substituted 5- to 14-membered aromatic heterocyclic group consisting of 1 to 7 carbon atoms and hetero atoms selected from the group consisting of nitrogen, oxygen and sulfur atoms,

(3) an optionally substituted C₈₋₁₄ aromatic fused-ring group,

(4) an optionally substituted 5- to 14-membered aromatic fused heterocyclic group consisting of 3 to 11 carbon atoms and hetero atoms selected from the group consisting of nitrogen, oxygen and sulfur atoms,

(5) an optionally substituted non-aromatic cyclic hydrocarbon group having carbon atoms not greater than 7,

(6) an optionally substituted non-aromatic heterocyclic group having carbon atoms not greater than 7,

(7) an optionally substituted amino group;

(8) an optionally substituted guanidino group;

(9) an optionally substituted hydroxyl group;

(10) an optionally substituted carboxyl group;

(11) an optionally substituted carbamoyl group; and,

(12) an optionally substituted sulfhydryl group,

or hydrogen atom.

The “optionally substituted C₆₋₁₂ aromatic hydrocarbon group,” “optionally substituted 5- to 14-membered aromatic heterocyclic group consisting of 1 to 7 carbon atoms and hetero atoms selected from the group consisting of nitrogen, oxygen and sulfur atoms,” “optionally substituted C₈₋₁₄ aromatic fused-ring group,” “optionally substituted 5- to 14-membered aromatic fused heterocyclic group consisting of 3 to 11 carbon atoms and hetero atoms selected from the group consisting of nitrogen, oxygen and sulfur atoms,” “optionally substituted non-aromatic cyclic hydrocarbon group having carbon atoms not greater than 7” and “optionally substituted non-aromatic heterocyclic group having carbon atoms not greater than 7” used are the same as those given above.

(1) As the C₁₋₄ alkyl group having an optionally substituted C₆₋₁₂ aromatic hydrocarbon group, there are used, for example, benzyl, 4-hydroxybenzyl, 2-chlorobenzyl, 3-chlorobenzyl, 4-chlorobenzyl, 4-aminobenzyl, etc.

(2) As the C₁₋₄ alkyl group having an optionally substituted 5- to 14-membered aromatic heterocyclic group consisting of 1 to 7 carbon atoms and hetero atoms selected from the group consisting of nitrogen, oxygen and sulfur atoms, there are used, for example, 2-pyridylmethyl, 3-pyridylmethyl, 4-pyridylmethyl, 4-imidazolemethyl, etc.

(3) As the C₁₋₄ alkyl group having an optionally substituted C₈₋₁₄ aromatic fused-ring group, there are used, for example, 1-naphthylmethyl, 2-naphthylmethyl, etc.

(4) As the C₁₋₄ alkyl group having an optionally substituted 5- to 14-membered aromatic fused heterocyclic group consisting of 3 to 11 carbon atoms and hetero atoms selected from the group consisting of nitrogen, oxygen and sulfur atoms, there are used, for example, 3-indolemethyl, 1-formylindol-3-ylmethyl, 2-quinolylmethyl, etc.

(5) As the C₁₋₄ alkyl group having an optionally substituted non-aromatic cyclic hydrocarbon group having carbon atoms not greater than 7, there are used, for example, cyclohexylmethyl, etc.

(6) As the C₁₋₄ alkyl group having an optionally substituted non-aromatic heterocyclic group having carbon atoms not greater than 7, there are used, for example, piperidin-1-ylmethyl, etc.

(7) As the C₁₋₄ alkyl group having an optionally substituted amino group, there are used, for example, 2-aminoethyl, 3-aminopropyl, 4-aminobutyl, 4-acetamidobutyl, etc.

(8) As the C₁₋₄ alkyl group having an optionally substituted guanidino group, there are used, for example, 3-guanidinopropyl, 3-(N-tosyl)guanidinopropyl, etc.

(9) As the C₁₋₄ alkyl group having an optionally substituted hydroxyl group, there are used, for example, hydroxymethyl, 1-hydroxyethyl, benzyloxymethyl, etc.

(10) As the C₁₋₄ alkyl group having an optionally substituted carboxyl group, there are used, for example, carboxylmethyl, 2-carboxylethyl, benzyloxycarbonylmethyl, etc.

(11) As the C₁₋₄ alkyl group having an optionally substituted carbamoyl group, there are used, for example, carbamoylmethyl, 2-carbamoylethyl, xanthylcarbamoyl, etc.

(12) As the C₁₋₄ alkyl group having an optionally substituted sulfhydryl group, there are used, for example, sulfhydrylmethyl, 2-(methylsulfhydryl)ethyl, etc.

(13) As the unsubstituted C₁₋₄ alkyl group, there are used, for example, methyl, ethyl, propyl, isopropyl, butyl, isobutyl, sec-butyl, tert-butyl, etc.

Preferred examples of Q³ used include hydrogen atom, 4-hydroxybenzyl, 3-pyridylmethyl, 4-pyridylmethyl, methyl, isobutyl, hydroxymethyl, carboxymethyl, 4-aminobutyl, etc., particularly preferably, 4-hydroxybenzyl, 3-pyridylmethyl, 4-pyridylmethyl, etc.

Preferred examples of Q⁴ used include carbamoylmethyl, 2-carbamoylethyl, 4-hydroxybenzyl, 4-imidazolemethyl, isobutyl, hydroxymethyl, 1-hydroxyethyl, carboxymethyl, 4-aminobutyl, etc., particularly preferably, carbamoylmethyl, 2-carbamoylethyl, 4-hydroxybenzyl, etc.

Preferred examples of Q⁵ used include benzyl, 2-chlorobenzyl, 3-chlorobenzyl, 4-chlorobenzyl, 4-aminobenzyl, 2-pyridylmethyl, 3-pyridylmethyl, 4-pyridylmethyl, 1-naphthylmethyl, 2-naphthylmethyl, 3-indolemethyl, 1-formylindol-3-ylmethyl, 2-quinolylmethyl, cyclohexylmethyl, hydroxymethyl, 1-hydroxyethyl, methyl, isopropyl, isobutyl, sec-butyl, carboxymethyl, 4-aminobutyl, etc., particularly preferably, benzyl, 2-chlorobenzyl, 3-chlorobenzyl, 4-chlorobenzyl, 4-aminobenzyl, 2-pyridylmethyl, 3-pyridylmethyl, 4-pyridylmethyl, 1-naphthylmethyl, 2-naphthylmethyl, 3-indolemethyl, 2-quinolylmethyl, cyclohexylmethyl, 1-hydroxyethyl, isopropyl, isobutyl, sec-butyl, etc.

Preferred examples of Q⁶ used are methyl, hydroxymethyl, 1-hydroxyethyl, carbamoylmethyl, 2-carbamoylethyl, etc., particularly preferably, carbamoylmethyl, etc.

Preferred examples of Q⁷ used are 4-hydroxybenzyl, carbamoylmethyl, 3-pyridylmethyl, methyl, isobutyl, benzyl, 4-aminobutyl, 3-indolemethyl, etc., particularly preferably, 4-hydroxybenzyl, etc.

Preferred examples of Q⁸ used include benzyl, 2-pyridylmethyl, 3-pyridylmethyl, 4-pyridylmethyl, 2-naphthylmethyl, 3-indolemethyl, hydroxymethyl, cyclohexylmethyl, sec-butyl, 1-hydroxyethyl, methyl, isobutyl, 4-aminobutyl, 3-carboxylpropyl, etc., more preferably, 4-pyridylmethyl, 3-indolemethyl, 2-carboxyethyl, and sec-butyl.

Preferred examples of Q⁹ used include hydrogen atom, methyl, ethyl, hydroxymethyl, 1-hydroxyethyl, carbamoylmethyl, 2-carbamoylethyl, ureidomethyl, acetamidomethyl, diethyl, formamidemethyl, methylcarbamoylmethyl, dimethylcarbamoylmethyl, etc., particularly preferably, carbamoylmethyl, ureidomethyl, etc.

Preferred examples of Q¹⁰ used include 4-hydroxybenzyl, 3-indolemethyl, methyl, 1-hydroxyethyl, 3-guanidinopropyl, etc., particularly preferably, 3-indolemethyl, etc.

Preferred examples of Q¹¹ used include carbamoylmethyl, etc.

Preferred examples of Q¹² used include methyl, carbamoylmethyl, etc., particularly preferably, carbamoylmethyl, etc.

Each of Y¹ through Y³ represents a group represented by formula: —CON(J¹³)—, —CSN(J¹³)—, —C(J¹⁴)N(¹³)— or —N(J¹³)CO— (wherein each of J¹³ and J¹⁴ represents hydrogen atom or a C₁₋₃ alkyl group).

As the C₁₋₃ alkyl group shown by J¹³ and J¹⁴, methyl, ethyl, propyl or isopropyl is used.

J¹³ is preferably hydrogen atom.

J¹⁴ is preferably hydrogen atom.

Y¹ is preferably a group shown by formula: —CONH— or —CH₂NH—, etc.

Y² is preferably a group shown by formula: —CONH— or —CH₂NH— , etc.

Y³ is preferably a group shown by formula: —CONH—, etc.

J³ and Q³, J⁴ and Q⁴, J⁵ and Q⁵, J⁶ and Q⁶, J⁷ and Q⁷, J⁸ and Q⁸, J⁹ and Q⁹, J¹⁰ and Q¹⁰, J¹¹ and Q¹¹, or J¹² and Q¹² may be combined together to form a ring. In this case, C(J³)(Q³), C(J⁴)(Q⁴), C(J⁵)(Q⁵), C(J⁶)(Q⁶), C(J⁷)(Q⁷), C(J⁸)(Q⁸), C(J⁹)(Q⁹), C(J¹⁰)(Q¹⁰), C(J¹¹)(Q¹¹) or C(J¹²)(Q¹²) may form, for example, cyclopentane, cyclohexane, piperidine, etc.

Z¹ and R¹, J² and Q³, Y¹ and Q⁴, Y² and Q⁵, Y³ and Q⁶, J² and Q⁷, Y² and Q⁸, Y³ and Q⁹, J² and Q¹⁰, Y³ and Q¹¹, or J² and Q¹² (preferably, J² and Q³, Y¹ and Q⁴, Y² and Q⁵, Y³ and Q⁶, J² and Q⁷, Y² and Q⁸, Y³ and Q⁹, J² and Q¹⁰, Y³ and Q¹¹, or J² and Q¹²) may be combined together to form a ring. The ring that has been formed may be substituted, and a fused ring may be formed.

Ring formation by the bonding of Z¹ with R¹, J² with Q³, J² with Q⁷, J² with Q¹⁰ or J² with Q¹² results in the formation of a compound such as azetidine, pyrrolidine, piperidine or thiazolidine of the formula Z¹—N—CH—R¹, J²-C(J³)(Q³), J²-C(J⁷)(Q⁷), J²-C(J¹⁰)(Q¹⁰) or J²-C(J¹²)(Q¹²), respectively. The ring that has formed may be substituted; also a fused ring may be formed. Preferred examples of Z¹—N—CH—R¹ include azetidine, pyrrolidine, 4-hydroxypyrrolidine and piperidine.

Ring formation by the bonding of Y¹ with Q⁴, Y² with Q⁵, Y³ with Q⁶, Y² with Q⁸, Y³ with Q⁹ or Y³ with Q¹¹ results in the formation of a radical such as pyrrolidin-2-carbonyl, piperidin-2-carbonyl or thiazolidin-4-carbonyl of the formula Y¹C(J⁴)(Q⁴), Y²C(J⁵)(Q⁵), Y³C(J⁶)(Q⁶), Y²C(J⁸)(Q⁸), Y³C(J⁹)(Q⁹) or Y³C(J¹¹)(Q¹¹), respectively. The ring that has formed may be substituted may also be substituted, and a fused ring may be formed.

Preferred groups represented by the formula: J¹-J²-C(J³)(Q³)Y¹C(J⁴)(Q⁴)Y²C(J⁵)(Q⁵)Y³C(J⁶)(Q⁶)C(═Z¹⁰)— include:
Tyr Asn Trp Asn-,
Tyr Asn Trp D-Asn-,
Tyr Asn D-Trp Asn-,
Tyr D-Asn Trp Asn-,
D-Tyr Asn Trp Asn-,
Tyr Lys Trp Asn-,
Tyr Asp Trp Asn-,
Tyr Tyr Trp Asn-,
Tyr Leu Trp Asn-,
Tyr Asn Ala Asn-,
Tyr Asn Leu Asn-,
Tyr Asn Ser Asn-,
Tyr Asn Asp Asn-,
Tyr Asn Lys Asn-,
Ala Asn Trp Asn-,
Leu Asn Trp Asn-,
Ser Asn Trp Asn-,
Asp Asn Trp Asn-,
Lys Asn Trp Asn-,
Tyr Asn Trp(For) Asn-,
D-Tyr Asn D-Trp Asn-,
D-Tyr Asn Ala Asn-,
D-Tyr Asn Ser Asn-,
D-Tyr Asn Cha Asn-,
D-Tyr Asn Thr Asn-,
D-Tyr Asn Ile Asn-,
D-Tyr Gln Trp Asn-,
D-Tyr Thr Trp Asn-,
D-Tyr Asn Val Asn-,
D-Tyr D-Asn Trp Asn-,
D-Tyr D-Asn D-Trp Asn-,
D-Tyr Asn Phe Asn-,
D-Tyr Asn Nal(1) Asn-,
D-Tyr Asn Nal(2) Asn-,
D-Tyr Asn Phe(2Cl) Asn-,
D-Tyr Asn Phe(3Cl) Asn-,
D-Tyr Asn Phe(4Cl) Asn-,
D-Tyr Asn Phe(4NH₂) Asn-,
D-Tyr Asn Pya(3) Asn-,
D-Tyr D-Asn Phe Asn-,
D-Tyr D-Asn Cha Asn-,
D-Tyr D-Asn Thr Asn-,
D-Tyr Asn Pya(2) Asn-,
D-Tyr Asn Pya(4) Asn-,
D-Tyr D-Ser Trp Asn-,
D-Tyr D-His Trp Asn-,
D-Pya(3) D-Asn Cha Asn-,
D-Pya(3) D-Tyr Cha Asn-,
TyrΨ(CH₂NH)Asn Trp Asn-,
D-Tyr AsnΨ(CH₂NH)Trp Asn-,
TyrΨ(CH₂NH)Asn D-Trp Asn-,
D-Tyr Asn Ala(2-Qui) Asn-,
D-Tyr Asn D-Pya(4) Asn-,
D-Tyr D-Asn Pya(4) Asn-,
Tyr D-Asn Cha Asn-,
Dap D-Tyr Asn Trp Asn-,
Arg D-Tyr D-Pya(4) Asn-,
Arg Arg D-Tyr D-Pya(4) Asn-,
Arg Acp D-Tyr D-Pya(4) Asn-,
D-Arg Acp D-Tyr D-Trp Asn-,
D-Arg D-Arg Acp D-Tyr D-Trp Asn-,
Ac D-Arg Acp D-Tyr D-Trp Asn-,
D-Dap Acp D-Tyr D-Trp Asn-,
D-Nle Acp D-Tyr D-Trp Asn-,
D-Arg β-Ala D-Tyr D-Trp Asn-,
D-Arg γ-Abu D-Tyr D-Trp Asn-,
D-Arg D-Arg γ-Abu D-Tyr D-Trp Asn-,
D-Arg D-Arg D-Arg γ-Abu D-Tyr D-Trp Asn-,
Gly D-Tyr D-Trp Asn-,
Ac Gly D-Tyr D-Trp Asn-,
D-Tyr D-Tyr D-Trp Asn-,
Ac D-Tyr D-Tyr D-Trp Asn-,
pGlu D-Tyr D-Trp Asn-,
Tyr D-Tyr D-Trp Asn-, and
Ac Tyr D-Tyr D-Trp Asn.

Prefered groups represented by the formula: J¹-J²-C(J⁷)(Q⁷)Y²C(J⁸)(Q⁸)Y³C(J⁹)(Q⁹)C(═Z¹⁰)— include:
Fmoc Asn Trp Asn-,
D-Asn Trp Asn-,
D-Tyr Trp Asn-,
D-Tyr D-Trp Asn-,
D-Tyr Ser Asn-,
D-Tyr Thr Asn-,
D-Tyr Ile Asn-,
D-Tyr Phe Asn-,
D-Tyr Nal(2) Asn-,
D-Pya(3) Phe Asn-,
D-Pya(3) Trp Asn-,
D-Tyr D-Pya(4) Asn-,
D-Asn Cha Asn-,
D-Tyr D-Pya(4) Ala-,
D-Tyr D-Pya(4) Thr-,
D-Tyr Pya(4) Ala-,
D-Tyr D-Trp Ala-,
D-Tyr D-Trp Abu-,
D-Tyr D-Phe Ala-6-Aminocaproyl-,
D-Tyr D-Pya(4) Asn-,
Ac D-Tyr D-Pya(4) Asn-,
Benzoyl D-Tyr D-Trp Asn-,
Cyclopropanecarbonyl D-Tyr D-Trp Asn-,
Butyryl D-Tyr D-Trp Asn-,
Me D-Tyr D-Trp Asn-,
Ac D-Tyr D-Trp Gln-,
Ac D-Tyr D-Trp Ser-,
Ac D-Tyr D-Trp Thr-,
Ac D-Tyr D-Trp Alb-,
Ac D-Tyr D-Trp Dap(Ac)-,
Ac D-Tyr D-Trp Dap(For)-,
Ac D-Tyr Trp Asn-,
Ac D-NMeTyr D-Trp Asn-,
For D-Tyr D-Trp Asn-,
Propionyl D-Tyr D-Trp Asn-,
Amidino D-Tyr D-Trp Asn-,
Ac D-Ala D-Trp Asn-,
Ac D-Leu D-Trp Asn-,
Ac D-Phe D-Trp Asn-,
Ac D-Nal(1) D-Trp Asn-,
Ac D-Nal(2) D-Trp Asn-,
Ac D-Lys D-Trp Asn-,
Ac D-Glu D-Trp Asn-,
Ac D-Tyr D-Ala Asn-,
Ac D-Tyr D-Leu Asn-,
Ac D-Tyr D-Phe Asn-,
Ac D-Tyr D-Thr Asn-,
Ac D-Tyr D-Lys Asn-,
Ac D-Tyr D-Glu Asn-,
Ac D-Tyr D-Trp Asp-,
Ac D-Tyr D-Trp D-Asn-,
Ac D-Tyr D-Trp NMeAsn-,
Ac D-Tyr Pro Asn-,
Ac D-Tyr D-Pya(2) Asn-,
Ac D-Tyr D-Pya(3) Asn-,
Ac D-Tyr D-Pro Asn-,
Ac D-Tyr Tic Asn-,
Ac Tyr Trp Asn-,
Ac D-Tyr NMMeTrp Asn-,
Glycoloyl D-Tyr D-Trp Asn-,
Ac D-Tyr D-Trp Gly-,
Ac D-Tyr D-Trp Dap-,
Ac D-Tyr D-Trp Asp(NHMe)-, and
Ac D-Tyr D-Trp Asp(NMe2)-.

Preferred groups represented by the formula: J¹-J²C(J¹⁰)(Q¹⁰)Y³C(J¹¹)(Q¹¹)C(═Z¹⁰)— include:
Fmoc Trp Asn-,
Boc Tyr Asn-,
Tyr Asn-,
D-Trp Asn-,
Ac Trp Asn-,
Amidino Trp Asn-,
Ac Ala Asn-,
Ac Arg Asn-,
Ac Thr Asn-,
D-Tyr D-Pya(4)-,
3-(4-Hydroxyphenyl)propionyl D-Trp Asn-,
D-Trp Asn-,
Ac D-Trp Asn-,
Hexanoyl D-Trp Asn-,
Cyclohexanecarbonyl D-Trp Asn-,
Benzoyl D-Trp Asn-,
3-Pyridinepropionyl D-Trp Asn-,
Adipoyl D-Trp Asn-,
6-Aminocaproyl D-Trp Asn-,
Amidino D-Trp Asn-, and
Glycoloyl D-Trp Asn-.

Preferred groups represented by the formula: J¹-J²-C(J¹²)(Q¹²)C(═Z¹⁰)— include, for example:
Fmoc Asn-,
3-(Indol-3-yl)propionyl Asn-,
3-Indolecarbonyl Asn-,
3-Indoleacetyl Asn-,
4-(Indol-3-yl)butyryl Asn-,
Diphenylacetyl Asn-,
Hexanoyl Asn-,
Cyclohexanecabonyl Asn-,
2-(Indol-3-yl)ethylcabamoyl Asn-,
3-(3-Pyridyl)propionyl Asn-,
4-Imidzoleacetyl Asn-,
Piperidinecarbonyl Asn-,
1-Piperidineacetyl Asn-,
1-Methyl-1-piperidinioacetyl Asn-,
1-Pyridinioacetyl Asn-,
D-Glucronyl Asn-,
3-Phenylpropionyl Asn-,
3-Phenylpropionyl Ala-,
Benzoyl Asn-,
Ac Asn-,
Cyclopropanecarbonyl Asn-, and
2-Naphthoyl Asn-.

Preferred groups represented by the formula: J¹- include, for example:
hydrogen,
GuAmb-,
3-(3-Indolyl)propionyl-,
3-(3-Pyridyl)propionyl-,
Benzoyl-,
Indole-3-carbonyl-,
Indole-3-acetyl-,
Ac—,
Hexanoyl-,
Z—,
Tos-,
3-Phenylpropionyl-,
2-(Indol-3-yl)ethylcarbamoyl-,
Benzyl-,
Phenethyl-,
2-Pyridinecarbonyl-,
4-Pyridinecarbonyl-,
Propionyl-,
Isobutyryl-,
Cyclohexanecarbonyl-,
Phenylacetyl-,
2-Methylnicotinoyl-,
5-Methylnicotinoyl-,
6-Methylnicotinoyl-,
Pyrazinecarbonyl-,
Cyclopropanecarbonyl-,
Trifluoroacetyl-,
(R)-3-hydroxy-2-methylpropionyl,
2-Hydroxyisobutyryl-,
3-Furancarbonyl-,
Pyrrole-2-carbonyl-,
4-Imidazolecarbonyl-,
6-Hydroxynicotinoyl-,
6-Chloronicotinoyl-,
6-(Trifluoromethyl)nicotinoyl-,
Dimethylcarbamoyl-,
1-Azetidinecarbonyl-,
2-Azetidinecarbonyl-,
4-Aminobenzoyl-,
4-Aminomethylbenzoyl-,
Pyrrole-3-carbonyl-,
Pyrimidine-4-carbonyl-,
Pyrimidine-2-carbonyl-, and
Pyridazine-4-carbonyl-.

The metastin derivative (IV) of the present invention is the group of compounds disclosed as metastin derivative (III) in WO 2006/001499.

In the metastin derivative (IV) of the present invention, the metastin derivative (I) of the present invention wherein V′ is a group represented by the formula

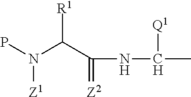

(each symbol in the formula having the same meaning as indicated above) is the group of compounds disclosed in WO 2004/063221 or the group of compounds disclosed as metastin derivative (I) in WO 2006/001499, and the metastin derivative (II) of the present invention wherein V′ is a group represented by the formula

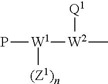

(each symbol in the formula having the same meaning as indicated above) or a group represented by the formula

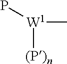

(each symbol in the formula having the same meaning as indicated above) is the group of compounds disclosed as metastin derivative (II) in WO 2006/001499.

All compounds in which radicals of the various symbols mentioned above are combined in any way may be advantageously used as the metastin derivative (IV) of the invention, although preferred use may be made of Compounds 1 to 703 mentioned in WO 2006/001499. Of these, the compounds having the following compound numbers are especially preferred.

MS10: Tyr-Asn-Trp-Asn-Ser-Phe-Gly-Leu-Arg-Phe-NH₂
1 2 3 4 5 6 7 8 9 10
Compound No. 17: [Pya(4)10]MS10
Tyr-Asn-Trp-Asn-Ser-Phe-Gly-Leu-Arg-Pya(4)-NH₂
Compound No. 18: [Tyr(Me)10]MS10
Tyr-Asn-Trp-Asn-Ser-Phe-Gly-Leu-Arg-Tyr(Me)-NH₂
Compound No. 19: [Phe(2F)10]MS10
Tyr-Asn-Trp-Asn-Ser-Phe-Gly-Leu-Arg-Phe(2F)-NH₂
Compound No. 23: [Tyr5]MS10
Tyr-Asn-Trp-Asn-Tyr-Phe-Gly-Leu-Arg-Phe-NH₂
Compound No. 24: [Leu5]MS10
Tyr-Asn-Trp-Asn-Leu-Phe-Gly-Leu-Arg-Phe-NH₂
Compound No. 30: Acetyl-MS10
Acetyl-Tyr-Asn-Trp-Asn-Ser-Phe-Gly-Leu-Arg-Phe-NH₂
Compound No. 31: Fmoc-MS10
Fmoc-Tyr-Asn-Trp-Asn-Ser-Phe-Gly-Leu-Arg-Phe-NH₂
Compound No. 38: [D-Ser5]MS10
Tyr-Asn-Trp-Asn-D-Ser-Phe-Gly-Leu-Arg-Phe-NH₂
Compound No. 39: [D-Asn4]MS10
Tyr-Asn-Trp-D-Asn-Ser-Phe-Gly-Leu-Arg-Phe-NH₂
Compound No. 40: [D-Trp3]MS10
Tyr-Asn-D-Trp-Asn-Ser-Phe-Gly-Leu-Arg-Phe-NH₂
Compound No. 41: [D-Asn2]MS10
Tyr-D-Asn-Trp-Asn-Ser-Phe-Gly-Leu-Arg-Phe-NH₂
Compound No. 42: [D-Tyr1]MS10
D-Tyr-Asn-Trp-Asn-Ser-Phe-Gly-Leu-Arg-Phe-NH₂
Compound No. 44: [Lys9]MS10
Tyr-Asn-Trp-Asn-Ser-Phe-Gly-Leu-Lys-Phe-NH₂
Compound No. 45: [Ala8]MS10
Tyr-Asn-Trp-Asn-Ser-Phe-Gly-Ala-Arg-Phe-NH₂
Compound No. 50: [Ala7]MS10
Tyr-Asn-Trp-Asn-Ser-Phe-Ala-Leu-Arg-Phe-NH₂
Compound No. 51: [NMePhe10]MS10
Tyr-Asn-Trp-Asn-Ser-Phe-Gly-Leu-Arg-NMePhe-NH₂
Compound No. 53: des(1-3)-Fmoc-MS10
Fmoc-Asn-Ser-Phe-Gly-Leu-Arg-Phe-NH₂
Compound No. 54: des(1-2)-Fmoc-MS10
Fmoc-Trp-Asn-Ser-Phe-Gly-Leu-Arg-Phe-NH₂
Compound No. 55: des(1)-Fmoc-MS10
Fmoc-Asn-Trp-Asn-Ser-Phe-Gly-Leu-Arg-Phe-NH₂
Compound No. 56: [Lys2]MS10
Tyr-Lys-Trp-Asn-Ser-Phe-Gly-Leu-Arg-Phe-NH₂
Compound No. 57: [Asp2]MS10
Tyr-Asp-Trp-Asn-Ser-Phe-Gly-Leu-Arg-Phe-NH₂
Compound No. 58: [Tyr2]MS10
Tyr-Tyr-Trp-Asn-Ser-Phe-Gly-Leu-Arg-Phe-NH₂
Compound No. 59: [Leu2]MS10
Tyr-Leu-Trp-Asn-Ser-Phe-Gly-Leu-Arg-Phe-NH₂
Compound No. 60: [Pya(3)10]MS10
Tyr-Asn-Trp-Asn-Ser-Phe-Gly-Leu-Arg-Pya(3)-NH₂
Compound No. 61: [Phe(4F)10]MS10
Tyr-Asn-Trp-Asn-Ser-Phe-Gly-Leu-Arg-Phe(4F)-NH₂
Compound No. 67: [Ala3]MS10
Tyr-Asn-Ala-Asn-Ser-Phe-Gly-Leu-Arg-Phe-NH₂
Compound No. 68: [Leu3]MS10
Tyr-Asn-Leu-Asn-Ser-Phe-Gly-Leu-Arg-Phe-NH₂
Compound No. 69: [Ser3]MS10
Tyr-Asn-Ser-Asn-Ser-Phe-Gly-Leu-Arg-Phe-NH₂
Compound No. 70: [Asp3]MS10
Tyr-Asn-Asp-Asn-Ser-Phe-Gly-Leu-Arg-Phe-NH₂
Compound No. 71: [Lys3]MS10
Tyr-Asn-Lys-Asn-Ser-Phe-Gly-Leu-Arg-Phe-NH₂
Compound No. 72: [Ala1]MS10
Ala-Asn-Trp-Asn-Ser-Phe-Gly-Leu-Arg-Phe-NH₂
Compound No. 73: [Leu1]MS10
Leu-Asn-Trp-Asn-Ser-Phe-Gly-Leu-Arg-Phe-NH₂
Compound No. 74: [Ser1]MS10
Ser-Asn-Trp-Asn-Ser-Phe-Gly-Leu-Arg-Phe-NH₂
Compound No. 75: [Asp1]MS10
Asp-Asn-Trp-Asn-Ser-Phe-Gly-Leu-Arg-Phe-NH₂
Compound No. 76: [Lys1]MS10
Lys-Asn-Trp-Asn-Ser-Phe-Gly-Leu-Arg-Phe-NH₂
Compound No. 77: [Phe(4CN)10]MS10
Tyr-Asn-Trp-Asn-Ser-Phe-Gly-Leu-Arg-Phe(4CN)-NH₂
Compound No. 78: [Trp(For)3,Phe(4CN)10]MS10
Tyr-Asn-Trp(For)-Asn-Ser-Phe-Gly-Leu-Arg-Phe(4CN)-
NH₂
Compound No. 79: [Hph10]MS10
Tyr-Asn-Trp-Asn-Ser-Phe-Gly-Leu-Arg-Hph-NH₂
Compound No. 81: [NMeArg9]MS10
Tyr-Asn-Trp-Asn-Ser-Phe-Gly-Leu-NMeArg-Phe-NH₂
Compound No. 82: [Arg(Me)9]MS10
Tyr-Asn-Trp-Asn-Ser-Phe-Gly-Leu-Arg(Me)-Phe-NH₂
Compound No. 83: [Arg(asyMe₂)9]MS10
Tyr-Asn-Trp-Asn-Ser-Phe-Gly-Leu-Arg(asyMe₂)-Phe-NH₂
Compound No. 87: des(4-5)-Boc-MS10
Boc-Tyr-Asn-Trp-Phe-Gly-Leu-Arg-Phe-NH₂
Compound No. 88: des(4-5)-MS10
Tyr-Asn-Trp-Phe-Gly-Leu-Arg-Phe-NH₂
Compound No. 90: [Lys9,9Ψ10,CH₂NH]MS10
Tyr-Asn-Trp-Asn-Ser-Phe-Gly-Leu-LysΨ(CH₂NH)Phe-NH₂
Compound No. 91: [8Ψ9,CH₂NH]MS10
Tyr-Asn-Trp-Asn-Ser-Phe-Gly-LeuΨ(CH₂NH)Arg-Phe-NH₂
Compound No. 97: [Har9]MS10
Tyr-Asn-Trp-Asn-Ser-Phe-Gly-Leu-Har-Phe-NH₂
Compound No. 98: [Lys(Me₂)9]MS10
Tyr-Asn-Trp-Asn-Ser-Phe-Gly-Leu-Lys(Me₂)-Phe-NH₂
Compound No. 101: [Ser7]MS10
Tyr-Asn-Trp-Asn-Ser-Phe-Ser-Leu-Arg-Phe-NH₂
Compound No. 105: [Nle8]MS10
Tyr-Asn-Trp-Asn-Ser-Phe-Gly-Nle-Arg-Phe-NH₂
Compound No. 107: [Val8]MS10
Tyr-Asn-Trp-Asn-Ser-Phe-Gly-Val-Arg-Phe-NH₂
Compound No. 109: [Tyr10]MS10
Tyr-Asn-Trp-Asn-Ser-Phe-Gly-Leu-Arg-Tyr-NH₂
Compound No. 110: [Nal(2)10]MS10
Tyr-Asn-Trp-Asn-Ser-Phe-Gly-Leu-Arg-Nal(2)-NH₂
Compound No. 111: [Phe(F5)10]MS10
Tyr-Asn-Trp-Asn-Ser-Phe-Gly-Leu-Arg-Phe(F5)-NH₂
Compound No. 112: [Cha10]MS10
Tyr-Asn-Trp-Asn-Ser-Phe-Gly-Leu-Arg-Cha-NH₂
Compound No. 114: des(1-3)-3-(3-Indolyl)propionyl-
MS10
3-(3-Indolyl)propionyl-Asn-Ser-Phe-Gly-Leu-Arg-
Phe-NH₂
Compound No. 121: des(1-4)-[Trp5]MS10
Trp-Phe-Gly-Leu-Arg-Phe-NH₂
Compound No. 123: [NMeLeu8]MS10
Tyr-Asn-Trp-Asn-Ser-Phe-Gly-NMeLeu-Arg-Phe-NH₂
Compound No. 126: [NMeSer5]MS10
Tyr-Asn-Trp-Asn-NMeSer-Phe-Gly-Leu-Arg-Phe-NH₂
Compound No. 127: [D-Asn4,NMePhe6]MS10
Tyr-Asn-Trp-D-Asn-Ser-NMePhe-Gly-Leu-Arg-Phe-NH₂
Compound No. 128: [10Ψ,CSNH]MS10
Tyr-Asn-Trp-Asn-Ser-Phe-Gly-Leu-Arg-PheΨ(CSNH)NH₂
Compound No. 129: [Arg(symMe₂)9]MS10
Tyr-Asn-Trp-Asn-Ser-Phe-Gly-Leu-Arg(symMe₂)-Phe-NH₂
Compound No. 130: [Phe(4Cl)10]MS10
Tyr-Asn-Trp-Asn-Ser-Phe-Gly-Leu-Arg-Phe(4Cl)-NH₂
Compound No. 131: [Phe(4NH₂)10]MS10
Tyr-Asn-Trp-Asn-Ser-Phe-Gly-Leu-Arg-Phe(4NH₂)-NH₂
Compound No. 132: [Phe(4NO₂)10]MS10
Tyr-Asn-Trp-Asn-Ser-Phe-Gly-Leu-Arg-Phe(4NO₂)-NH₂
Compound No. 133: [Nal(1)10]MS10
Tyr-Asn-Trp-Asn-Ser-Phe-Gly-Leu-Arg-Nal(1)-NH₂
Compound No. 134: [Trp10]MS10
Tyr-Asn-Trp-Asn-Ser-Phe-Gly-Leu-Arg-Trp-NH₂
Compound No. 137: [Nle9]MS10
Tyr-Asn-Trp-Asn-Ser-Phe-Gly-Leu-Nle-Phe-NH₂
Compound No. 138: [Cit9]MS10
Tyr-Asn-Trp-Asn-Ser-Phe-Gly-Leu-Cit-Phe-NH₂
Compound No. 140: [Arg(Me)9,NMePhe10]MS10
Tyr-Asn-Trp-Asn-Ser-Phe-Gly-Leu-Arg(Me)-NMePhe-NH₂
Compound No. 141: [D-Tyr1,Arg(Me)9]MS10
D-Tyr-Asn-Trp-Asn-Ser-Phe-Gly-Leu-Arg(Me)-Phe-NH₂
Compound No. 142: [D-Tyr1,D-Trp3,Arg(Me)9]MS10
D-Tyr-Asn-D-Trp-Asn-Ser-Phe-Gly-Leu-Arg(Me)-Phe-
NH₂
Compound No. 143: [D-Trp3,Arg(Me)9]MS10
Tyr-Asn-D-Trp-Asn-Ser-Phe-Gly-Leu-Arg(Me)-Phe-NH₂
Compound No. 144: des(1-3)-Fmoc-[Arg(Me)9]MS10
Fmoc-Asn-Ser-Phe-Gly-Leu-Arg(Me)-Phe-NH₂
Compound No. 145: des(1-2)-Fmoc-[Arg(Me)9]MS10
Fmoc-Trp-Asn-Ser-Phe-Gly-Leu-Arg(Me)-Phe-NH₂
Compound No. 146: [10Ψ,CSNH,D-Tyr1]MS10
D-Tyr-Asn-Trp-Asn-Ser-Phe-Gly-Leu-Arg-PheΨ(CSNH)
NH₂
Compound No. 150: [Tyr6]MS10
Tyr-Asn-Trp-Asn-Ser-Tyr-Gly-Leu-Arg-Phe-NH₂
Compound No. 151: [Nal(1)6]MS10
Tyr-Asn-Trp-Asn-Ser-Nal(1)-Gly-Leu-Arg-Phe-NH₂
Compound No. 152: [Nal(2)6]MS10
Tyr-Asn-Trp-Asn-Ser-Nal(2)-Gly-Leu-Arg-Phe-NH₂
Compound No. 153: [Phe(F5)6]MS10
Tyr-Asn-Trp-Asn-Ser-Phe(F5)-Gly-Leu-Arg-Phe-NH₂
Compound No. 154: [Phe(4F)6]MS10
Tyr-Asn-Trp-Asn-Ser-Phe(4F)-Gly-Leu-Arg-Phe-NH₂
Compound No. 156: [Cha6]MS10
Tyr-Asn-Trp-Asn-Ser-Cha-Gly-Leu-Arg-Phe-NH₂
Compound No. 163: [6Ψ7,CH₂NH]MS10
Tyr-Asn-Trp-Asn-Ser-PheΨ(CH₂NH)Gly-Leu-Arg-Phe-
NH₂
Compound No. 165: [Dap(Gly)9]-MS10
Tyr-Asn-Trp-Asn-Ser-Phe-Gly-Leu-Dap(Gly)-Phe-NH₂
Compound No. 166: [6Ψ7,CSNH]MS10
Tyr-Asn-Trp-Asn-Ser-PheΨ(CSNH)Gly-Leu-Arg-Phe-NH₂
Compound No. 169: [D-Tyr1,Ala3,Arg(Me)9]MS10
D-Tyr-Asn-Ala-Asn-Ser-Phe-Gly-Leu-Arg(Me)-Phe-NH₂
Compound No. 170: [D-Tyr1,Ser3,Arg(Me)9]MS10
D-Tyr-Asn-Ser-Asn-Ser-Phe-Gly-Leu-Arg(Me)-Phe-NH₂
Compound No. 171: [D-Tyr1,Cha3,Arg(Me)9]MS10
D-Tyr-Asn-Cha-Asn-Ser-Phe-Gly-Leu-Arg(Me)-Phe-NH₂
Compound No. 172: [D-Tyr1,Cha6,Arg(Me)9]MS10
D-Tyr-Asn-Trp-Asn-Ser-Cha-Gly-Leu-Arg(Me)-Phe-NH₂
Compound No. 173: [D-Tyr1,Ala7,Arg(Me)9]MS10
D-Tyr-Asn-Trp-Asn-Ser-Phe-Ala-Leu-Arg(Me)-Phe-NH₂
Compound No. 174: [D-Tyr1,Arg(Me)9,Trp10]MS10
D-Tyr-Asn-Trp-Asn-Ser-Phe-Gly-Leu-Arg(Me)-Trp-NH₂
Compound No. 176: [AzaGly7]MS10
Tyr-Asn-Trp-Asn-Ser-Phe-AzaGly-Leu-Arg-Phe-NH₂
Compound No. 181: [D-Tyr1,Cha3,6,Arg(Me)9]MS10
D-Tyr-Asn-Cha-Asn-Ser-Cha-Gly-Leu-Arg(Me)-Phe-NH₂
Compound No. 182: [D-Tyr1,Cha3,6,Arg(Me)9,Trp10]
MS10
D-Tyr-Asn-Cha-Asn-Ser-Cha-Gly-Leu-Arg(Me)-Trp-NH₂
Compound No. 183: [Phe(4NH₂)9]MS10
Tyr-Asn-Trp-Asn-Ser-Phe-Gly-Leu-Phe(4NH₂)-Phe-NH₂
Compound No. 184: [Phe(4-Guanidino)9]MS10
Tyr-Asn-Trp-Asn-Ser-Phe-Gly-Leu-Phe(4-Guanidino)-
Phe-NH₂
Compound No. 185: [Dap(GnGly)9]MS10
Tyr-Asn-Trp-Asn-Ser-Phe-Gly-Leu-Dap(GnGly)-Phe-NH₂
Compound No. 186: [Trp(For)10]MS10
Tyr-Asn-Trp-Asn-Ser-Phe-Gly-Leu-Arg-Trp(For)-NH₂
Compound No. 187: [Abu8]MS10
Tyr-Asn-Trp-Asn-Ser-Phe-Gly-Abu-Arg-Phe-NH₂
Compound No. 189: [Ala(3-Bzt)10]MS10
Tyr-Asn-Trp-Asn-Ser-Phe-Gly-Leu-Arg-Ala(3-Bzt)-NH₂
Compound No. 190: [D-Tyr1,Cha3,AzaGly7,Arg(Me)9]
MS10
D-Tyr-Asn-Cha-Asn-Ser-Phe-AzaGly-Leu-Arg(Me)-Phe-
NH₂
Compound No. 191: [D-Tyr1,Ser3,AzaGly7,Arg(Me)9]
MS10
D-Tyr-Asn-Ser-Asn-Ser-Phe-AzaGly-Leu-Arg(Me)-Phe-
NH₂
Compound No. 192: [D-Tyr1,Arg(Et)9]MS10
D-Tyr-Asn-Trp-Asn-Ser-Phe-Gly-Leu-Arg(Et)-Phe-NH₂
Compound No. 193: [D-Tyr1,Arg(n-Pr)9]MS10
D-Tyr-Asn-Trp-Asn-Ser-Phe-Gly-Leu-Arg(n-Pr)-Phe-
NH₂
Compound No. 194: [D-Tyr1,Arg(Ac)9]MS10
D-Tyr-Asn-Trp-Asn-Ser-Phe-Gly-Leu-Arg(Ac)-Phe-NH₂
Compound No. 197: [Phe(3F)10]MS10
Tyr-Asn-Trp-Asn-Ser-Phe-Gly-Leu-Arg-Phe(3F)-NH₂
Compound No. 198: [Phe(3,4F₂)10]MS10
Tyr-Asn-Trp-Asn-Ser-Phe-Gly-Leu-Arg-Phe(3,4F₂)-
NH₂
Compound No. 199: [Phe(3,4Cl₂)10]MS10
Tyr-Asn-Trp-Asn-Ser-Phe-Gly-Leu-Arg-Phe(3,4Cl₂)-
NH₂
Compound No. 200: [Phe(3CF₃)10]MS10
Tyr-Asn-Trp-Asn-Ser-Phe-Gly-Leu-Arg-Phe(3CF₃)-NH₂
Compound No. 201: [Ala(2-Qui)10]MS10
Tyr-Asn-Trp-Asn-Ser-Phe-Gly-Leu-Arg-Ala(2-Qui)-NH₂
Compound No. 203: [D-Tyr1,Cha6,Arg(Me)9]MS10
D-Tyr-Asn-Trp-Asn-Ser-Cha-Gly-Leu-Arg(Me)-Phe-NH₂
Compound No. 204: [D-Tyr1,A1a7,Arg(Me)9]MS10
D-Tyr-Asn-Trp-Asn-Ser-Phe-Ala-Leu-Arg(Me)-Phe-NH₂
Compound No. 205: [D-Tyr1,Thr3,Arg(Me)9]MS10
D-Tyr-Asn-Thr-Asn-Ser-Phe-Gly-Leu-Arg(Me)-Phe-NH₂
Compound No. 206: [D-Tyr1,Ile3,Arg(Me)9]MS10
D-Tyr-Asn-Ile-Asn-Ser-Phe-Gly-Leu-Arg(Me)-Phe-NH₂
Compound No. 207: [D-Tyr1,Ser4,Arg(Me)9]MS10
D-Tyr-Asn-Trp-Ser-Ser-Phe-Gly-Leu-Arg(Me)-Phe-NH₂
Compound No. 208: [D-Tyr1,Thr4,Arg(Me)9]MS10
D-Tyr-Asn-Trp-Thr-Ser-Phe-Gly-Leu-Arg(Me)-Phe-NH₂
Compound No. 209: [D-Tyr1,Gln4,Arg(Me)9]MS10
D-Tyr-Asn-Trp-Gln-Ser-Phe-Gly-Leu-Arg(Me)-Phe-NH₂
Compound No. 210: [D-Tyr1,Ala4,Arg(Me)9]MS10
D-Tyr-Asn-Trp-Ala-Ser-Phe-Gly-Leu-Arg(Me)-Phe-NH₂
Compound No. 211: [D-Tyr1,Thr5,Arg(Me)9]MS10
D-Tyr-Asn-Trp-Asn-Thr-Phe-Gly-Leu-Arg(Me)-Phe-NH₂
Compound No. 212: [D-Tyr1,Ala5,Arg(Me)9]MS10
D-Tyr-Asn-Trp-Asn-Ala-Phe-Gly-Leu-Arg(Me)-Phe-NH₂
Compound No. 213: [D-Tyr1,Val8,Arg(Me)9]MS10
D-Tyr-Asn-Trp-Asn-Ser-Phe-Gly-Val-Arg(Me)-Phe-NH₂
Compound No. 214: [D-Tyr1,Gln2,Arg(Me)9]MS10
D-Tyr-Gln-Trp-Asn-Ser-Phe-Gly-Leu-Arg(Me)-Phe-NH₂
Compound No. 215: [D-Tyr1,Thr2,Arg(Me)9]MS10
D-Tyr-Thr-Trp-Asn-Ser-Phe-Gly-Leu-Arg(Me)-Phe-NH₂
Compound No. 216: des(1)-[D-Asn2,Arg(Me)9]MS10
D-Asn-Trp-Asn-Ser-Phe-Gly-Leu-Arg(Me)-Phe-NH₂
Compound No. 217: des(1)-[D-Tyr2,Arg(Me)9]MS10
D-Tyr-Trp-Asn-Ser-Phe-Gly-Leu-Arg(Me)-Phe-NH₂
Compound No. 218: [N((CH₂)₃Gn)]Gly9]MS10
Tyr-Asn-Trp-Asn-Ser-Phe-Gly-Leu-N((CH₂)₃Gn)Gly-Phe-
NH₂
Compound No. 220: [Arg(Et)9]MS10
Tyr-Asn-Trp-Asn-Ser-Phe-Gly-Leu-Arg(Et)-Phe-NH₂
Compound No. 221: [D-Tyr1,Thr3,AzaGly7,Arg(Me)9]
MS10
D-Tyr-Asn-Thr-Asn-Ser-Phe-AzaGly-Leu-Arg(Me)-Phe-
NH₂
Compound No. 222: des(1)-[D-Tyr2,AzaGly7,Arg(Me)9]
MS10
D-Tyr-Trp-Asn-Ser-Phe-AzaGly-Leu-Arg(Me)-Phe-NH₂
Compound No. 223: des(1-2)-[D-Trp3,Arg(Me)9]MS10
D-Trp-Asn-Ser-Phe-Gly-Leu-Arg(Me)-Phe-NH₂
Compound No. 224: des(1)-[D-Tyr2,D-Trp3,Arg(Me)9]
MS10
D-Tyr-D-Trp-Asn-Ser-Phe-Gly-Leu-Arg(Me)-Phe-NH₂
Compound No. 225: des(1)-[D-Asn2,D-Trp3,Arg(Me)9]
MS10
D-Asn-D-Trp-Asn-Ser-Phe-Gly-Leu-Arg(Me)-Phe-NH₂
Compound No. 226: des(1)-[D-Tyr2,Ser3,Arg(Me)9]MS10
D-Tyr-Ser-Asn-Ser-Phe-Gly-Leu-Arg(Me)-Phe-NH₂
Compound No. 227: des(1)-[D-Tyr2,Thr3,Arg(Me)9]MS10
D-Tyr-Thr-Asn-Ser-Phe-Gly-Leu-Arg(Me)-Phe-NH₂
Compound No. 228: des(1)-[D-Tyr2,Ile3,Arg(Me)9]MS10
D-Tyr-Ile-Asn-Ser-Phe-Gly-Leu-Arg(Me)-Phe-NH₂
Compound No. 229: [D-Tyr1,Val3,Arg(Me)9]MS10
D-Tyr-Asn-Val-Asn-Ser-Phe-Gly-Leu-Arg(Me)-Phe-NH₂
Compound No. 230: [D-Tyr1,D-Asn2,Arg(Me)9]MS10
D-Tyr-D-Asn-Trp-Asn-Ser-Phe-Gly-Leu-Arg(Me)-Phe-
NH₂
Compound No. 231: [D-Tyr1,D-Asn2,D-Trp3,Arg(Me)9]
MS10
D-Tyr-D-Asn-D-Trp-Asn-Ser-Phe-Gly-Leu-Arg(Me)-Phe-
NH₂
Compound No. 232: [D-Tyr1,AzaGly7,Arg(Me)9]MS10
D-Tyr-Asn-Trp-Asn-Ser-Phe-AzaGly-Leu-Arg(Me)-Phe-
NH₂
Compound No. 233: [D-Tyr1,Ile3,AzaGly7,Arg(Me)9]
MS10
D-Tyr-Asn-Ile-Asn-Ser-Phe-AzaGly-Leu-Arg(Me)-Phe-
NH₂
Compound No. 234: [D-Tyr1,Val3,AzaGly7,Arg(Me)9]
MS10
D-Tyr-Asn-Val-Asn-Ser-Phe-AzaGly-Leu-Arg(Me)-Phe-
NH₂
Compound No. 235: [D-Tyr1,Ala3,AzaGly7,Arg(Me)9]
MS10
D-Tyr-Asn-Ala-Asn-Ser-Phe-AzaGly-Leu-Arg(Me)-Phe-
NH₂
Compound No. 236: [D-Tyr1,D-Trp3,AzaGly7,Arg(Me)9]
MS10
D-Tyr-Asn-D-Trp-Asn-Ser-Phe-AzaGly-Leu-Arg(Me)-
Phe-NH₂
Compound No. 237: [D-Tyr1,D-Asn2,AzaGly7,Arg(Me)9]
MS10
D-Tyr-D-Asn-Trp-Asn-Ser-Phe-AzaGly-Leu-Arg(Me)-
Phe-NH₂
Compound No. 238: [D-Tyr1,D-Asn2,D-Trp3,AzaGly7,
Arg(Me)9]MS10
D-Tyr-D-Asn-D-Trp-Asn-Ser-Phe-AzaGly-Leu-Arg(Me)-
Phe-NH₂
Compound No. 239: des(1)-[D-Tyr2,Ser3,AzaGly7,Arg
(Me)9]MS10
D-Tyr-Ser-Asn-Ser-Phe-AzaGly-Leu-Arg(Me)-Phe-NH₂
Compound No. 240: des(1)-[D-Tyr2,Ile3,AzaGly7,Arg
(Me)9]MS10
D-Tyr-Ile-Asn-Ser-Phe-AzaGly-Leu-Arg(Me)-Phe-NH₂
Compound No. 241: des(1)-[D-Tyr2,Thr3,AzaGly7,Arg
(Me)9]MS10
D-Tyr-Thr-Asn-Ser-Phe-AzaGly-Leu-Arg(Me)-Phe-NH₂
Compound No. 242: des(1)-[D-Tyr2,D-Trp3,AzaGly7,
Arg(Me)9]MS10
D-Tyr-D-Trp-Asn-Ser-Phe-AzaGly-Leu-Arg(Me)-Phe-
NH₂
Compound No. 244: [D-Tyr1,Phe3,AzaGly7,Arg(Me)9]
MS10
D-Tyr-Asn-Phe-Asn-Ser-Phe-AzaGly-Leu-Arg(Me)-Phe-
NH₂
Compound No. 245: [D-Tyr1,Nal(1)3,AzaGly7,Arg
(Me)9]MS10
D-Tyr-Asn-Nal(1)-Asn-Ser-Phe-AzaGly-Leu-Arg(Me)-
Phe-NH₂
Compound No. 246: [D-Tyr1,Nal(2)3,AzaGly7,Arg
(Me)9]MS10
D-Tyr-Asn-Nal(2)-Asn-Ser-Phe-AzaGly-Leu-Arg(Me)-
Phe-NH₂
Compound No. 247: [D-Tyr1,Phe(2Cl)3,AzaGly7,Arg
(Me)9]MS10
D-Tyr-Asn-Phe(2Cl)-Asn-Ser-Phe-AzaGly-Leu-Arg(Me)-
Phe-NH₂
Compound No. 248: [D-Tyr1,Phe(3Cl)3,AzaGly7,Arg
(Me)9]MS10
D-Tyr-Asn-Phe(3Cl)-Asn-Ser-Phe-AzaGly-Leu-Arg
(Me)-Phe-NH₂
Compound No. 249: [D-Tyr1,Phe(4Cl)3,AzaGly7,Arg
(Me)9]MS10
D-Tyr-Asn-Phe(4Cl)-Asn-Ser-Phe-AzaGly-Leu-Arg(Me)-
Phe-NH₂
Compound No. 250: [D-Tyr1,Phe(4NH2)3,AzaGly7,Arg
(Me)9]MS10
D-Tyr-Asn-Phe(4NH2)-Asn-Ser-Phe-AzaGly-Leu-Arg
(Me)-Phe-NH₂
Compound No. 251: [D-Tyr1,Pya(3)3,AzaGly7,Arg
(Me)9]MS10
D-Tyr-Asn-Pya(3)-Asn-Ser-Phe-AzaGly-Leu-Arg(Me)-
Phe-NH₂
Compound No. 252: [D-Tyr1,D-Ala3,AzaGly7,Arg(Me)9]
MS10
D-Tyr-Asn-D-Ala-Asn-Ser-Phe-AzaGly-Leu-Arg(Me)-
Phe-NH₂
Compound No. 253: [D-Tyr1,Pro3,AzaGly7,Arg(Me)9]
MS10
D-Tyr-Asn-Pro-Asn-Ser-Phe-AzaGly-Leu-Arg(Me)-Phe-
NH₂
Compound No. 254: des(1)-[D-Tyr2,Phe3,AzaGly7,Arg
(Me)9]MS10
D-Tyr-Phe-Asn-Ser-Phe-AzaGly-Leu-Arg(Me)-Phe-NH₂
Compound No. 255: des(1)-[D-Tyr2,Nal(2)3,AzaGly7,
Arg(Me)9]MS10
D-Tyr-Nal(2)-Asn-Ser-Phe-AzaGly-Leu-Arg(Me)-Phe-
NH₂
Compound No. 256: des(1)-[D-Pya(3)2,Phe3,AzaGly7,
Arg(Me)9]MS10
D-Pya(3)-Phe-Asn-Ser-Phe-AzaGly-Leu-Arg(Me)-Phe-NH₂
Compound No. 257: [D-Tyr1,D-Asn2,Phe3,AzaGly7,Arg
(Me)9]MS10
D-Tyr-D-Asn-Phe-Asn-Ser-Phe-AzaGly-Leu-Arg(Me)-Phe-
NH₂
Compound No. 258: [D-Pya(3)1,AzaGly7,Arg(Me)9]MS10
D-Pya(3)-Asn-Trp-Asn-Ser-Phe-AzaGly-Leu-Arg(Me)-
Phe-NH₂
Compound No. 259: [D-Ala1,AzaGly7,Arg(Me)9]MS10
D-Ala-Asn-Trp-Asn-Ser-Phe-AzaGly-Leu-Arg(Me)-Phe-
NH₂
Compound No. 260: des(1-3)-3-(3-Indolyl)propionyl-
[AzaGly7,Arg(Me)9]MS10
3-(3-Indolyl)propionyl-Asn-Ser-Phe-AzaGly-Leu-Arg
(Me)-Phe-NH₂
Compound No. 261: [7Ψ8,CH₂NH]MS10
Tyr-Asn-Trp-Asn-Ser-Phe-GlyΨ(CH₂NH)Leu-Arg-Phe-NH₂
Compound No. 265: des(1-3)-Indole-3-carbonyl-
[AzaGly7,Arg(Me)9]MS10
Indole-3-carbonyl-Asn-Ser-Phe-AzaGly-Leu-Arg(Me)-
Phe-NH₂
Compound No. 266: des(1-3)-Indole-3-acetyl-[Aza
Gly7,Arg(Me)9]MS10
Indol-3-acetyl-Asn-Ser-Phe-AzaGly-Leu-Arg(Me)-Phe-
NH₂
Compound No. 267: des(1-3)-4-(3-Indolyl)butyryl-
[AzaGly7,Arg(Me)9]MS10
4-(3-Indolyl)butyryl-Asn-Ser-Phe-AzaGly-Leu-Arg
(Me)-Phe-NH₂
Compound No. 268: des(1-3)-Diphenylacetyl-[Aza
Gly7,Arg(Me)9]MS10
Diphenylacetyl-Asn-Ser-Phe-AzaGly-Leu-Arg(Me)-
Phe-NH₂
Compound No. 269: des(1-3)-3-Phenylpropionyl-[Aza
Gly7,Arg(Me)9]MS10
3-Phenylpropionyl-Asn-Ser-Phe-AzaGly-Leu-Arg(Me)-
Phe-NH₂
Compound No. 270: [D-Tyr1,Phe3,Ser-Phe5,AzaGly7,
Arg(Me)9]MS10
D-Tyr-Asn-Phe-Asn-Ser-Phe-Phe-AzaGly-Leu-Arg(Me)-
Phe-NH₂
Compound No. 271: des(1-2)-[AzaGly7,Arg(Me)9]MS10
Trp-Asn-Ser-Phe-AzaGly-Leu-Arg(Me)-Phe-NH₂
Compound No. 272: des(1-2)-Acetyl-[AzaGly7,Arg
(Me)9]MS10
Acetyl-Trp-Asn-Ser-Phe-AzaGly-Leu-Arg(Me)-Phe-NH₂
Compound No. 273: des(1-2)-Amidino-[AzaGly7,Arg
(Me)9]MS10
Amidino-Trp-Asn-Ser-Phe-AzaGly-Leu-Arg(Me)-Phe-NH₂
Compound No. 274: des(1-2)-Acetyl-[Ala3,AzaGly7,
Arg(Me)9]MS10
Acetyl-Ala-Asn-Ser-Phe-AzaGly-Leu-Arg(Me)-Phe-NH₂
Compound No. 275: des(1-2)-Acetyl-[Arg3,AzaGly7,
Arg(Me)9]MS10
Acetyl-Arg-Asn-Ser-Phe-AzaGly-Leu-Arg(Me)-Phe-NH₂
Compound No. 276: des(1-2)-Acetyl-[Thr3,AzaGly7,
Arg(Me)9]MS10
Acetyl-Thr-Asn-Ser-Phe-AzaGly-Leu-Arg(Me)-Phe-NH₂
Compound No. 277: des(1-3)-n-Hexanoyl-[AzaGly7,
Arg(Me)9]MS10
n-Hexanoyl-Asn-Ser-Phe-AzaGly-Leu-Arg(Me)-Phe-NH₂
Compound No. 278: des(1-3)-Cyclohexanecarbonyl-
[AzaGly7,Arg(Me)9]MS10
Cyclohexanecarbonyl-Asn-Ser-Phe-AzaGly-Leu-Arg
(Me)-Phe-NH₂
Compound No. 279: des(1-3)-2-(Indol-3-yl)ethyl
carbamoyl-[AzaGly7,Arg(Me)9]MS10
2-(indol-3-yl)ethylcarbamoyl-Asn-Ser-Phe-AzaGly-
Leu-Arg(Me)-Phe-NH₂
Compound No. 281: [D-Tyr1,Pya(2)6,Arg(Me)9]MS10
D-Tyr-Asn-Trp-Asn-Ser-Pya(2)-Gly-Leu-Arg(Me)-Phe-
NH₂
Compound No. 282: [D-Tyr1,Pya(4)6,Arg(Me)9]MS10
D-Tyr-Asn-Trp-Asn-Ser-Pya(4)-Gly-Leu-Arg(Me)-Phe-
NH₂
Compound No. 283: [D-Tyr1,D-Asn2,Cha3,AzaGly7,
Arg(Me)9]MS10
D-Tyr-D-Asn-Cha-Asn-Ser-Phe-AzaGly-Leu-Arg(Me)-
Phe-NH₂
Compound No. 284: [D-Tyr1,D-Asn2,Thr3,AzaGly7,Arg
(Me)9]MS10
D-Tyr-D-Asn-Thr-Asn-Ser-Phe-AzaGly-Leu-Arg(Me)-
Phe-NH₂
Compound No. 285: [D-Tyr1,Pya(2)3,AzaGly7,Arg
(Me)9]MS10
D-Tyr-Asn-Pya(2)-Asn-Ser-Phe-AzaGly-Leu-Arg(Me)-
Phe-NH₂
Compound No. 286: [D-Tyr1,Pya(4)3,AzaGly7,Arg
(Me)9]MS10
D-Tyr-Asn-Pya(4)-Asn-Ser-Phe-AzaGly-Leu-Arg(Me)-
Phe-NH₂
Compound No. 287: [D-Tyr1,D-Ser2,AzaGly7,Arg
(Me)9]MS10
D-Tyr-D-Ser-Trp-Asn-Ser-Phe-AzaGly-Leu-Arg(Me)-
Phe-NH₂
Compound No. 288: [D-Tyr1,D-His2,AzaGly7,Arg
(Me)9]MS10
D-Tyr-D-His-Trp-Asn-Ser-Phe-AzaGly-Leu-Arg(Me)-
Phe-NH₂
Compound No. 289: des(1)-[D-Pya(3)2,AzaGly7,Arg
(Me)9]MS10
D-Pya(3)-Trp-Asn-Ser-Phe-AzaGly-Leu-Arg(Me)-Phe-
NH₂
Compound No. 290: [D-Pya(3)1,D-Asn2,Cha3,AzaGly7,
Arg(Me)9]MS10
D-Pya(3)-D-Asn-Cha-Asn-Ser-Phe-AzaGly-Leu-Arg
(Me)-Phe-NH₂
Compound No. 291: [D-Pya(3)1,D-Tyr2,Cha3,AzaGly7,
Arg(Me)9]MS10
D-Pya(3)-D-Tyr-Cha-Asn-Ser-Phe-AzaGly-Leu-Arg
(Me)-Phe-NH₂
Compound No. 293: [4Ψ5,CH₂NH]MS10
Tyr-Asn-Trp-AsnΨ(CH₂NH)Ser-Phe-Gly-Leu-Arg-Phe-
NH₂
Compound No. 294: [1Ψ2,CH₂NH]MS10
TyrΨ(CH₂NH)Asn-Trp-Asn-Ser-Phe-Gly-Leu-Arg-Phe-
NH₂
Compound No. 295: [2Ψ3,CH₂NH]MS10
Tyr-AsnΨ(CH₂NH)Trp-Asn-Ser-Phe-Gly-Leu-Arg-Phe-
NH₂
Compound No. 296: [6Ψ7,CSNH,D-Tyr1,Arg(Me)9]MS10
D-Tyr-Asn-Trp-Asn-Ser-PheΨ(CSNH)Gly-Leu-Arg(Me)-
Phe-NH₂
Compound No. 297: [D-Tyr1,Thr5,AzaGly7,Arg(Me)9]
MS10
D-Tyr-Asn-Trp-Asn-Thr-Phe-AzaGly-Leu-Arg(Me)-Phe-
NH₂
Compound No. 298: [D-Tyr1,D-Asn2,Thr5,AzaGly7,Arg
(Me)9]MS10
D-Tyr-D-Asn-Trp-Asn-Thr-Phe-AzaGly-Leu-Arg(Me)-
Phe-NH₂
Compound No. 299: [1Ψ2,CH₂NH,AzaGly7,Arg(Me)9]-
MS10
TyrΨ(CH₂NH)Asn-Trp-Asn-Ser-Phe-AzaGly-Leu-Arg(Me)-
Phe-NH₂
Compound No. 300: [1Ψ2,CH₂NH,D-Trp3,AzaGly7,Arg
(Me)9]-MS10
TyrΨ(CH₂NH)Asn-D-Trp-Asn-Ser-Phe-AzaGly-Leu-Arg
(Me)-Phe-NH₂
Compound No. 301: [D-Tyr1,Ala(2-Qui)3,AzaGly7,Arg
(Me)9]MS10
D-Tyr-Asn-Ala(2-Qui)-Asn-Ser-Phe-AzaGly-Leu-Arg
(Me)-Phe-NH₂
Compound No. 302: [D-Tyr1,D-Pya(4)3,AzaGly7,Arg
(Me)9]MS10
D-Tyr-Asn-D-Pya(4)-Asn-Ser-Phe-AzaGly-Leu-Arg
(Me)-Phe-NH₂
Compound No. 303: [D-Tyr1,D-Asn2,Pya(4)3,AzaGly7,
Arg(Me)9]MS10
D-Tyr-D-Asn-Pya(4)-Asn-Ser-Phe-AzaGly-Leu-Arg
(Me)-Phe-NH₂
Compound No. 304: [D-Asn2,Pya(4)3,AzaGly7,Arg
(Me)9]MS10
Tyr-D-Asn-Pya(4)-Asn-Ser-Phe-AzaGly-Leu-Arg(Me)-
Phe-NH₂
Compound No. 305: des(1)-[D-Tyr2,D-Pya(4)3,Aza
Gly7,Arg(Me)9]MS10
D-Tyr-D-Pya(4)-Asn-Ser-Phe-AzaGly-Leu-Arg(Me)-Phe-
NH₂
Compound No. 306: [D-Pya(4)1,D-Asn2,Cha3,AzaGly7,
Arg(Me)9]MS10
D-Pya(4)-D-Asn-Cha-Asn-Ser-Phe-AzaGly-Leu-Arg(Me)-
Phe-NH₂
Compound No. 307: [7Ψ8,CH₂NH,D-Tyr1,Arg(Me)9]MS10
D-Tyr-Asn-Trp-Asn-Ser-Phe-GlyΨ(CH₂NH)Leu-Arg(Me)-
Phe-NH₂
Compound No. 308: [6Ψ7,CH₂NH,D-Tyr1,Arg(Me)9]MS10
D-Tyr-Asn-Trp-Asn-Ser-PheΨ(CH₂NH)Gly-Leu-Arg(Me)-
Phe-NH₂
Compound No. 310: [Nar9]MS10
Tyr-Asn-Trp-Asn-Ser-Phe-Gly-Leu-Nar-Phe-NH₂
Compound No. 311: [Nar(Me)9]MS10
Tyr-Asn-Trp-Asn-Ser-Phe-Gly-Leu-Nar(Me)-Phe-NH₂
Compound No. 312: [Har(Me)9]MS10
Tyr-Asn-Trp-Asn-Ser-Phe-Gly-Leu-Har(Me)-Phe-NH₂
Compound No. 313: [Dab9]MS10
Tyr-Asn-Trp-Asn-Ser-Phe-Gly-Leu-Dab-Phe-NH₂
Compound No. 314: [Orn9]MS10
Tyr-Asn-Trp-Asn-Ser-Phe-Gly-Leu-Orn-Phe-NH₂
Compound No. 315: des(1)-[D-Asn2,Cha3,AzaGly7,Arg
(Me)9]MS10
D-Asn-Cha-Asn-Ser-Phe-AzaGly-Leu-Arg(Me)-Phe-NH₂
Compound No. 316: [D-Tyr1,D-Asn2,Thr3,AzaGly7,Arg
(Me)9,Phe(4F)10]MS10
D-Tyr-D-Asn-Thr-Asn-Ser-Phe-AzaGly-Leu-Arg(Me)-
Phe(4F)-NH₂
Compound No. 317: [D-Tyr1,D-Asn2,Pya(4)3,AzaGly7,
Arg(Me)9,Phe(4F)10]MS10
D-Tyr-D-Asn-Pya(4)-Asn-Ser-Phe-AzaGly-Leu-Arg(Me)-
Phe(4F)-NH₂
Compound No. 318: [D-Tyr1,AzaGly7,Arg(Me)9,Phe
(4F)10]MS10
D-Tyr-Asn-Trp-Asn-Ser-Phe-AzaGly-Leu-Arg(Me)-Phe
(4F)-NH₂
Compound No. 319: [6Ψ7,NHCO,D-Tyr1,Arg(Me)9]MS10
D-Tyr-Asn-Trp-Asn-Ser-PheΨ(NHCO)Gly-Leu-Arg(Me)-
Phe-NH₂
Compound No. 322: des(1-3)-3-(3-Pyridyl)propionyl-
[AzaGly7,Arg(Me)9]MS10
3-(3-Pyridyl)propionyl-Asn-Ser-Phe-AzaGly-Leu-Arg
(Me)-Phe-NH₂
Compound No. 323: des(1-3)-4-Imidazoleacetyl-[Aza
Gly7,Arg(Me)9]MS10
4-Imidazoleacetyl-Asn-Ser-Phe-AzaGly-Leu-Arg(Me)-
Phe-NH₂
Compound No. 324: des(1-3)-4-Piperidinecarbonyl-
[AzaGly7,Arg(Me)9]MS10
Piperidinecarbonyl-Asn-Ser-Phe-AzaGly-Leu-Arg(Me)-
Phe-NH₂
Compound No. 325: des(1-3)-1-Piperidineacetyl-[Aza
Gly7,Arg(Me)9]MS10
1-Piperidineacetyl-Asn-Ser-Phe-AzaGly-Leu-Arg(Me)-
Phe-NH₂
Compound No. 326:des(1-3)-1-Methylpiperidinio-l-
acetyl-[AzaGly7,Arg(Me)9]MS10
1-Methylpiperidino-1-acetyl-Asn-Ser-Phe-AzaGly-
Leu-Arg(Me)-Phe-NH₂
Compound No. 327: des(1-3)-1-Pyridinioacetyl-[Aza
Gly7,Arg(Me)9]MS10
1-Pyridinoacetyl-Asn-Ser-Phe-AzaGly-Leu-Arg(Me)-
Phe-NH₂
Compound No. 328: des(1-3)-D-Glucronyl-[AzaGly7,
Arg(Me)9]MS10
D-Glucronyl-Asn-Ser-Phe-AzaGly-Leu-Arg(Me)-Phe-NH₂
Compound No. 375: 2-Aminoethyl-Gly-[D-Tyr1,Arg
(Me)9]MS10
2-Aminoethyl-Gly-D-Tyr-Asn-Trp-Asn-Ser-Phe-Gly-
Leu-Arg(Me)-Phe-NH₂
Compound No. 385: des(1)-[D-Tyr2,D-Pya(4)3,AzaGly7,
Arg(Me)9,Trp10]MS10
D-Tyr-D-Pya(4)-Asn-Ser-Phe-AzaGly-Leu-Arg(Me)-Trp-
NH₂
Compound No. 386: des(1-3)-3-(3-Pyridyl)propionyl-
[AzaGly7,Arg(Me)9,Trp10]MS10
3-(3-Pyridyl)propionyl-Asn-Ser-Phe-AzaGly-Leu-Arg
(Me)-Trp-NH₂
Compound No. 387: Dap-[D-Tyr1,Arg(Me)9]MS10
Dap-D-Tyr-Asn-Trp-Asn-Ser-Phe-Gly-Leu-Arg(Me)-Phe-
NH₂
Compound No. 397: Methylthiocarbamoyl-Sar-[D-Tyr1,
Arg(Me)9]MS10
Methylthiocarbamoyl-Sar-D-Tyr-Asn-Trp-Asn-Ser-Phe-
Gly-Leu-Arg(Me)-Phe-NH₂
Compound No. 400: (S)-1-(Quinolin-8-yl-carbamoyl)-
4-thiapentylcarbamoy1-[D-Tyr1,Arg(Me)9]MS10
(S)-1-(Quinolin-8-yl-carbamoy1)-4-thiapentylcar-
bamoyl-D-Tyr-Asn-Trp-Asn-Ser-Phe-Gly-Leu-Arg(Me)-
Phe-NH₂
Compound No. 481: des(1)-[D-Tyr2,D-Pya(4)3,AzaGly7,
Har9,Trp10]MS10
D-Tyr-D-Pya(4)-Asn-Ser-Phe-AzaGly-Leu-Har-Trp-NH₂
Compound No. 486: des(1)-[D-Tyr2,D-Pya(4)3,AzaGly7,
Orn9]MS10
D-Tyr-D-Pya(4)-Asn-Ser-Phe-AzaGly-Leu-Orn-Phe-NH₂
Compound No. 487: des(1)-[D-Tyr2,D-Pya(4)3,AzaGly7,
Lys9]MS10
D-Tyr-D-Pya(4)-Asn-Ser-Phe-AzaGly-Leu-Lys-Phe-NH₂
Compound No. 488: des(1)-[D-Tyr2,D-Pya(4)3,AzaGly7,
Har9]MS10
D-Tyr-D-Pya(4)-Asn-Ser-Phe-AzaGly-Leu-Har-Phe-NH₂
Compound No. 489: des(1)-[D-Tyr2,D-Pya(4)3,AzaGly7,
Har(Me)9]MS10
D-Tyr-D-Pya(4)-Asn-Ser-Phe-AzaGly-Leu-Har(Me)-Phe-
NH₂
Compound No. 490: des(1)-[D-Tyr2,Pya(4)3,AzaGly7,Arg
(Me)9]MS10
D-Tyr-Pya(4)-Asn-Ser-Phe-AzaGly-Leu-Arg(Me)-Phe-
NH₂
Compound No. 491: des(1)-[D-Tyr2,D-Pya(4)3,Trp5,
AzaGly7,Arg(Me)9,Trp10]MS10
D-Tyr-D-Pya(4)-Asn-Trp-Phe-AzaGly-Leu-Arg(Me)-Trp-
NH₂
Compound No. 492: des(1)-[D-Tyr2,D-Pya(4)3,Ala4,
AzaGly7,Arg(Me)9,Trp10]MS10
D-Tyr-D-Pya(4)-Ala-Ser-Phe-AzaGly-Leu-Arg(Me)-Trp-
NH₂
Compound No. 493: des(1)-[D-Tyr2,D-Pya(4)3,Thr4,
AzaGly7,Arg(Me)9,Trp10]MS10
D-Tyr-D-Pya(4)-Thr-Ser-Phe-AzaGly-Leu-Arg(Me)-Trp-
NH₂
Compound No. 494: des(1,4)-[D-Tyr2,D-Pya(4)3,Aza
Gly7,Arg(Me)9,Trp10]MS10
D-Tyr-D-Pya(4)-Ser-Phe-AzaGly-Leu-Arg(Me)-Trp-NH₂
Compound No. 495: des(1-3)-[D-Tyr4,Pya(4)5,Aza
Gly7,Arg(Me)9,Trp10]MS10
D-Tyr-Pya(4)-Phe-AzaGly-Leu-Arg(Me)-Trp-NH₂
Compound No. 496: des(1)-[D-Tyr2,D-Pya(4)3,Cha6,
Arg(Me)9,Trp10]MS10
D-Tyr-D-Pya(4)-Asn-Ser-Cha-Gly-Leu-Arg(Me)-Trp-NH₂
Compound No. 497: des(1)-[D-Tyr2,D-Pya(4)3,Cha6,
Ala7,Arg(Me)9,Trp10]MS10
D-Tyr-D-Pya(4)-Asn-Ser-Cha-Ala-Leu-Arg(Me)-Trp-NH₂
Compound No. 498: des(1)-[D-Tyr2,D-Pya(4)3,Ile5,
AzaGly7,Arg(Me)9,Trp10]MS10
D-Tyr-D-Pya(4)-Asn-Ile-Phe-AzaGly-Leu-Arg(Me)-Trp-
NH₂
Compound No. 499: des(1-3)-3-Phenylpropionyl-[Aza
Gly7,Arg(Me)9,Trp10]MS10
3-Phenylpropionyl-Asn-Ser-Phe-AzaGly-Leu-Arg(Me)-
Trp-NH₂
Compound No. 500: des(1-3)-3-Phenylpropionyl-
[Ala4,AzaGly7,Arg(Me)9,Trp10]MS10
3-Phenylpropionyl-Ala-Ser-Phe-AzaGly-Leu-Arg(Me)-
Trp-NH₂
Compound No. 501: des(1)-[D-Tyr2,D-Pya(4)3,Thr5,Aza
Gly7,Arg(Me)9,Trp10]MS10
D-Tyr-D-Pya(4)-Asn-Thr-Phe-AzaGly-Leu-Arg(Me)-Trp-
NH₂
Compound No. 502: des(1)-[D-Tyr2,Pya(4)3,Ala4,Aza
Gly7,Arg(Me)9,Trp10]MS10
D-Tyr-Pya(4)-Ala-Ser-Phe-AzaGly-Leu-Arg(Me)-Trp-
NH₂
Compound No. 503: des(1)-[D-Tyr2,D-Trp3,Ala4,Aza
Gly7,Arg(Me)9,Trp10]MS10
D-Tyr-D-Trp-Ala-Ser-Phe-AzaGly-Leu-Arg(Me)-Trp-NH₂
Compound No. 504: [Acp1,D-Tyr2,D-Pya(4)3,AzaGly7,
Arg(Me)9]MS10
Acp-D-Tyr-D-Pya(4)-Asn-Ser-Phe-AzaGly-Leu-Arg(Me)-
Phe-NH₂
Compound No. 505: des(1-3)-3-Phenylpropionyl-
[Thr5,AzaGly7,Arg(Me)9,Trp10]MS10
3-Phenylpropionyl-Asn-Thr-Phe-AzaGly-Leu-Arg(Me)-
Trp-NH₂
Compound No. 506: des(1-3)-3-Phenylpropionyl-
[Ile5,AzaGly7,Arg(Me)9,Trp10]MS10
3-Phenylpropionyl-Asn-Ile-Phe-AzaGly-Leu-Arg(Me)-
Trp-NH₂
Compound No. 507: des(1-3)-3-Phenylpropionyl-
[Trp6,AzaGly7,Arg(Me)9,Trp10]MS10
3-Phenylpropionyl-Asn-Ser-Trp-AzaGly-Leu-Arg(Me)-
Trp-NH₂
Compound No. 508: des(1-3)-3-Phenylpropionyl-[Phe
(4F)6,AzaGly7,Arg(Me)9,Trp10]MS10
3-Phenylpropionyl-Asn-Ser-Phe(4F)-AzaGly-Leu-Arg
(Me)-Trp-NH₂
Compound No. 509: des(1-3)-Benzoyl-[AzaGly7,Arg
(Me)9,Trp10]MS10
Benzoyl-Asn-Ser-Phe-AzaGly-Leu-Arg(Me)-Trp-NH₂
Compound No. 510: des(1-3)-Ac-[AzaGly7,Arg(Me)9,
Trp10]MS10
Ac-Asn-Ser-Phe-AzaGly-Leu-Arg(Me)-Trp-NH₂
Compound No. 511: des(1)-[D-Tyr2,D-Trp3,Ala4,Thr5,
AzaGly7,Arg(Me)9,Trp10]MS10
D-Tyr-D-Trp-Ala-Thr-Phe-AzaGly-Leu-Arg(Me)-Trp-NH₂
Compound No. 512: des(1)-[D-Tyr2,D-Trp3,Thr5,Aza
Gly7,Arg(Me)9,Trp10]MS10
D-Tyr-D-Trp-Asn-Thr-Phe-AzaGly-Leu-Arg(Me)-Trp-NH₂
Compound No. 513: des(1)-[D-Tyr2,D-Trp3,Abu4,Aza
Gly7,Arg(Me)9,Trp10]MS10
D-Tyr-D-Trp-Abu-Ser-Phe-AzaGly-Leu-Arg(Me)-Trp-NH₂
Compound No. 514: des(1)-[D-Tyr2,D-Phe3,Ala4,Aza
Gly7,Arg(Me)9,Trp10]MS10
D-Tyr-D-Phe-Ala-Ser-Phe-AzaGly-Leu-Arg(Me)-Trp-NH₂
Compound No. 515: des(1)-[D-Tyr2,D-Pya(4)3,Val5,
AzaGly7,Arg(Me)9,Trp10]MS10
D-Tyr-D-Pya(4)-Asn-Val-Phe-AzaGly-Leu-Arg(Me)-Trp-
NH₂
Compound No. 516: des(1)-Ac-[D-Tyr2,D-Pya(4)3,Aza
Gly7,Arg(Me)9]MS10
Ac-D-Tyr-D-Pya(4)-Asn-Ser-Phe-AzaGly-Leu-Arg(Me)-
Phe-NH₂
Compound No. 517: des(1-3)-3-Phenylpropionyl-
[Hyp5,AzaGly7,Arg(Me)9,Trp10]MS10
3-Phenylpropionyl-Asn-Hyp-Phe-AzaGly-Leu-Arg(Me)-
Trp-NH₂
Compound No. 518: des(1-3)-3-Phenylpropionyl-
[Cha6,Arg(Me)9,Trp10]MS10
3-Phenylpropionyl-Asn-Ser-Cha-Gly-Leu-Arg(Me)-Trp-
NH₂
Compound No. 519: des(1-3)-Phenylacetyl-[AzaGly7,
Arg(Me)9,Trp10]MS10
Phenylacetyl-Asn-Ser-Phe-AzaGly-Leu-Arg(Me)-Trp-
NH₂
Compound No. 521: des(1)-[D-Tyr2,D-Pya(4)3,AzaGly7]
MS10
D-Tyr-D-Pya(4)-Asn-Ser-Phe-AzaGly-Leu-Arg-Phe-NH₂
Compound No. 522: des(1-3)-Benzoy1-[Thr5,AzaGly7,
Arg(Me)9,Trp10]MS10
Benzoyl-Asn-Thr-Phe-AzaGly-Leu-Arg(Me)-Trp-NH₂
Compound No. 523: des(1-3)-Benzoyl-[Thr5,Phe(4F)6,
AzaGly7,Arg(Me)9,Trp10]MS10
Benzoyl-Asn-Thr-Phe(4F)-AzaGly-Leu-Arg(Me)-Trp-NH₂
Compound No. 524: des(1-3)-3-Phenylpropionyl-
[Pro5,AzaGly7,Arg(Me)9,Trp10]MS10
3-Phenylpropionyl-Asn-Pro-Phe-AzaGly-Leu-Arg(Me)-
Trp-NH₂
Compound No. 527: des(1)-[D-Tyr2,D-Pya(4)3,Hyp5,
AzaGly7,Arg(Me)9,Trp10]MS10
D-Tyr-D-Pya(4)-Asn-Hyp-Phe-AzaGly-Leu-Arg(Me)-Trp-
NH₂
Compound No. 528: des(1)-[D-Tyr2,D-Pya(4)3,Pro5,
AzaGly7,Arg(Me)9,Trp10]MS10
D-Tyr-D-Pya(4)-Asn-Pro-Phe-AzaGly-Leu-Arg(Me)-Trp-
NH₂
Compound No. 529: des(1)-[D-Tyr2,D-Pya(4)3,Tle5,
AzaGly7,Arg(Me)9,Trp10]MS10
D-Tyr-D-Pya(4)-Asn-Tle-Phe-AzaGly-Leu-Arg(Me)-Trp-
NH₂
Compound No. 530: des(1)-[D-Tyr2,D-Pya(4)3,Phg5,
AzaGly7,Arg(Me)9,Trp10]MS10
D-Tyr-D-Pya(4)-Asn-Phg-Phe-AzaGly-Leu-Arg(Me)-Trp-
NH₂
Compound No. 531: des(1-3)-3-Phenylpropionyl-
[Pic(2)5,AzaGly7,Arg(Me)9,Trp10]MS10
3-Phenylpropionyl-Asn-Pic(2)-Phe-AzaGly-Leu-Arg
(Me)-Trp-NH₂
Compound No. 532: des(1-3)-3-Phenylpropionyl-
[Aze(2)5,AzaGly7,Arg(Me)9,Trp10]MS10
3-Phenylpropionyl-Asn-Aze(2)-Phe-AzaGly-Leu-Arg
(Me)-Trp-NH₂
Compound No. 533: des(1-3)-3-Phenylpropionyl-[D-
Pro5,AzaGly7,Arg(Me)9,Trp10]MS10
3-Phenylpropionyl-Asn-D-Pro-Phe-AzaGly-Leu-Arg
(Me)-Trp-NH₂
Compound No. 534: des(1-3)-Cyclopropanecarbonyl-
[AzaGly7,Arg(Me)9,Trp10]MS10
Cyclopropanecarbonyl-Asn-Ser-Phe-AzaGly-Leu-Arg
(Me)-Trp-NH₂
Compound No. 535: des(1-3)-2-Naphthoyl-[AzaGly7,
Arg(Me)9,Trp10]MS10
2-Naphthoyl-Asn-Ser-Phe-AzaGly-Leu-Arg(Me)-Trp-NH₂
Compound No. 536: [Arg1,D-Tyr2,D-Pya(4)3,AzaGly7,
Arg(Me)9,Trp10]MS10
Arg-D-Tyr-D-Pya(4)-Asn-Ser-Phe-AzaGly-Leu-Arg(Me)-
Trp-NH₂
Compound No. 537: Arg-[Arg1,D-Tyr2,D-Pya(4)3,Aza
Gly7,Arg(Me)9,Trp10]MS10
Arg-Arg-D-Tyr-D-Pya(4)-Asn-Ser-Phe-AzaGly-Leu-Arg
(Me)-Trp-NH₂
Compound No. 538: Arg-[Acp1,D-Tyr2,D-Pya(4)3,Aza
Gly7,Arg(Me)9,Trp10]MS10
Arg-Acp-D-Tyr-D-Pya(4)-Asn-Ser-Phe-AzaGly-Leu-Arg
(Me)-Trp-NH₂
Compound No. 539: des(1)-[D-Tyr2,D-Trp3,Val5,Aza
Gly7,Arg(Me)9,Trp10]MS10
D-Tyr-D-Trp-Asn-Val-Phe-AzaGly-Leu-Arg(Me)-Trp-NH₂
Compound No. 540: des(1)-[D-Tyr2,D-Trp3,AzaGly7,
Arg(Me)9,Trp10]MS10
D-Tyr-D-Trp-Asn-Ser-Phe-AzaGly-Leu-Arg(Me)-Trp-NH₂
Compound No. 541: D-Arg-[Acp1,D-Tyr2,D-Trp3,Thr5,
AzaGly7,Arg(Me)9,Trp10]MS10
D-Arg-Acp-D-Tyr-D-Trp-Asn-Thr-Phe-AzaGly-Leu-Arg
(Me)-Trp-NH₂
Compound No. 542: D-Arg-D-Arg-[Acp1,D-Tyr2,D-Trp3,
Thr5,AzaGly7,Arg(Me)9,Trp10]MS10
D-Arg-D-Arg-Acp-D-Tyr-D-Trp-Asn-Thr-Phe-AzaGly-
Leu-Arg(Me)-Trp-NH₂
Compound No. 545: des(1-3)-Benzoyl-[Phe(4F)6,Aza
Gly7,Arg(Me)9,Trp10]MS10
Benzoyl-Asn-Ser-Phe(4F)-AzaGly-Leu-Arg(Me)-Trp-NH₂
Compound No. 546: des(1-3)-3-Phenylpropionyl-[Ser
(Ac)5,AzaGly7,Arg(Me)9,Trp10]MS10
3-Phenylpropionyl-Asn-Ser(Ac)-Phe-AzaGly-Leu-Arg
(Me)-Trp-NH₂
Compound No. 547: des(1)-[D-Tyr2,D-Pya(4)3,Ser
(Ac)5,AzaGly7,Arg(Me)9,Trp10]MS10
D-Tyr-D-Pya(4)-Asn-Ser(Ac)-Phe-AzaGly-Leu-Arg(Me)-
Trp-NH₂
Compound No. 548: des(1)-[D-Tyr2,D-Pya(4)3,Aza
Gly7,Arg(Me)9,10Ψ,CSNH]MS10
D-Tyr-D-Pya(4)-Asn-Ser-Phe-AzaGly-Leu-Arg(Me)-PheΨ
(CSNH)NH₂
Compound No. 550: des(1)-Ac-[D-Tyr2,D-Trp3,Thr5,Aza
Gly7,Arg(Me)9,Trp10]MS10
Ac-D-Tyr-D-Trp-Asn-Thr-Phe-AzaGly-Leu-Arg(Me)-Trp-
NH₂
Compound No. 551: Ac-D-Arg-[Acp1,D-Tyr2,D-Trp3,
Thr5,AzaGly7,Arg(Me)9,Trp10]MS10
Ac-D-Arg-Acp-D-Tyr-D-Trp-Asn-Thr-Phe-AzaGly-Leu-
Arg(Me)-Trp-NH₂
Compound No. 552: D-Dap-[Acp1,D-Tyr2,D-Trp3,Thr5,
AzaGly7,Arg(Me)9,Trp10]MS10
D-Dap-Acp-D-Tyr-D-Trp-Asn-Thr-Phe-AzaGly-Leu-Arg
(Me)-Trp-NH₂
Compound No. 553: D-Nle-[Acp1,D-Tyr2,D-Trp3,Thr5,
AzaGly7,Arg(Me)9,Trp10]MS10
D-Nle-Acp-D-Tyr-D-Trp-Asn-Thr-Phe-AzaGly-Leu-Arg
(Me)-Trp-NH₂
Compound No. 554: D-Arg-[β-Ala1,D-Tyr2,D-Trp3,
Thr5,AzaGly7,Arg(Me)9,Trp10]MS10
D-Arg-β-Ala-D-Tyr-D-Trp-Asn-Thr-Phe-AzaGly-Leu-
Arg(Me)-Trp-NH₂
Compound No. 555: D-Arg-[γ-Abu1,D-Tyr2,D-Trp3,
Thr5,AzaGly7,Arg(Me)9,Trp10]MS10
D-Arg-γ-Abu-D-Tyr-D-Trp-Asn-Thr-Phe-AzaGly-Leu-
Arg(Me)-Trp-NH₂
Compound No. 556: D-Arg-D-Arg-[γ-Abu1,D-Tyr2,D-
Trp3,Thr5,AzaGly7,Arg(Me)9,Trp10]MS10
D-Arg-D-Arg-γ-Abu-D-Tyr-D-Trp-Asn-Thr-Phe-AzaGly-
Leu-Arg(Me)-Trp-NH₂
Compound No. 557: D-Arg-D-Arg-D-Arg-[γ-Abu1,D-
Tyr2,D-Trp3,Thr5,AzaGly7,Arg(Me)9,Trp10]MS10
D-Arg-D-Arg-D-Arg-γ-Abu-D-Tyr-D-Trp-Asn-Thr-Phe-
AzaGly-Leu-Arg(Me)-Trp-NH₂
Compound No. 558: des(1)-Ac[D-Tyr2,D-Trp3,AzaGly7,
Arg(Me)9,Trp10]MS10
D-Tyr-D-Trp-Asn-Ser-Phe-AzaGly-Leu-Arg(Me)-Trp-NH₂
Compound No. 559: des(1-2)-3-(4-Hydroxyphenyl)
propionyl-[D-Trp3,Thr5,AzaGly7,Arg(Me)9,Trp10]MS10
3-(4-Hydroxyphenyl)propionyl-D-Trp-Asn-Thr-Phe-Aza
Gly-Leu-Arg(Me)-Trp-NH₂
Compound No. 561: D-Arg-[Acp1,D-Tyr2,D-Trp3,Abu4,
AzaGly7,Arg(Me)9,Trp10]MS10
D-Arg-Acp-D-Tyr-D-Trp-Abu-Ser-Phe-AzaGly-Leu-Arg
(Me)-Trp-NH₂
Compound No. 562: des(1)-Ac-[D-Tyr2,D-Pya(4)3,
Thr5,AzaGly7,Arg(Me)9,Trp10]MS10
Ac-D-Tyr-D-Pya(4)-Asn-Thr-Phe-AzaGly-Leu-Arg(Me)-
Trp-NH₂
Compound No. 563: des(1)-Ac-[D-Tyr2,D-Trp3,Aze
(2)5,AzaGly7,Arg(Me)9,Trp10]MS10
Ac-D-Tyr-D-Trp-Asn-Aze(2)-Phe-AzaGly-Leu-Arg(Me)-
Trp-NH₂
Compound No. 564: des(1)-Ac-[D-Tyr2,D-Trp3,Val5,
AzaGly7,Arg(Me)9,Trp10]MS10
Ac-D-Tyr-D-Trp-Asn-Val-Phe-AzaGly-Leu-Arg(Me)-Trp-
NH₂
Compound No. 565: des(1)-Benzoyl-[D-Tyr2,D-Trp3,
Thr5,AzaGly7,Arg(Me)9,Trp10]MS10
Benzoyl-D-Tyr-D-Trp-Asn-Thr-Phe-AzaGly-Leu-Arg
(Me)-Trp-NH₂
Compound No. 566: des(1)-Cyclopropanecarbonyl-[D-
Tyr2,D-Trp3,Thr5,AzaGly7,Arg(Me)9,Trp10]MS10
Cyclopropanecarbonyl-D-Tyr-D-Trp-Asn-Thr-Phe-Aza
Gly-Leu-Arg(Me)-Trp-NH₂
Compound No. 567: des(1)-Butyryl-[D-Tyr2,D-Trp3,
Thr5,AzaGly7,Arg(Me)9,Trp10]MS10
Butyryl-D-Tyr-D-Trp-Asn-Thr-Phe-AzaGly-Leu-Arg
(Me)-Trp-NH₂
Compound No. 568: Ac-[D-Arg1,D-Tyr2,D-Trp3,Thr5,
AzaGly7,Arg(Me)9,Trp10]MS10
Ac-D-Arg-D-Tyr-D-Trp-Asn-Thr-Phe-AzaGly-Leu-Arg
(Me)-Trp-NH₂
Compound No. 569: des(1)-Ac-[D-Tyr2,D-Trp3,Thr5,
6Ψ7,CH₂NH,Arg(Me)9,Trp10]MS10
Ac-D-Tyr-D-Trp-Asn-Thr-PheΨ(CH₂NH)Gly-Leu-Arg(Me)-
Trp-NH₂
Compound No. 570: des(1)-Me-[D-Tyr2,D-Trp3,Thr5,
AzaGly7,Arg(Me)9,Trp10]MS10
Me-D-Tyr-D-Trp-Asn-Thr-Phe-AzaGly-Leu-Arg(Me)-Trp-
NH₂
Compound No. 571: des(1)-Ac-[D-Tyr2,D-Trp3,Thr5,
AzaGly7,Arg(Me)9]MS10
Ac-D-Tyr-D-Trp-Asn-Thr-Phe-AzaGly-Leu-Arg(Me)-Phe-
NH₂
Compound No. 572: des(1)-[D-Trp2,D-Pya(4)3,Aza
Gly7,Arg(Me)9,Trp10]MS10
D-Trp-D-Pya(4)-Asn-Ser-Phe-AzaGly-Leu-Arg(Me)-Trp-
NH₂
Compound No. 573: des(1)-Ac-[D-Tyr2,D-Trp3,Abu4,
AzaGly7,Arg(Me)9,Trp10]MS10
Ac-D-Tyr-D-Trp-Abu-Ser-Phe-AzaGly-Leu-Arg(Me)-Trp-
NH₂
Compound No. 576: des(1)-Ac-[D-Tyr2,D-Trp3,Gln4,
AzaGly7,Arg(Me)9,Trp10]MS10
Ac-D-Tyr-D-Trp-Gln-Ser-Phe-AzaGly-Leu-Arg(Me)-Trp-
NH₂
Compound No. 577: des(1)-Ac-[D-Tyr2,D-Trp3,Ser4,
AzaGly7,Arg(Me)9,Trp10]MS10
Ac-D-Tyr-D-Trp-Ser-Ser-Phe-AzaGly-Leu-Arg(Me)-Trp-
NH₂
Compound No. 578: des(1)-Ac-[D-Tyr2,D-Trp3,Thr4,
AzaGly7,Arg(Me)9,Trp10]MS10
Ac-D-Tyr-D-Trp-Thr-Ser-Phe-AzaGly-Leu-Arg(Me)-Trp-
NH₂
Compound No. 579: des(1)-Ac-[D-Tyr2,D-Trp3,Alb4,
AzaGly7,Arg(Me)9,Trp10]MS10
Ac-D-Tyr-D-Trp-Alb-Ser-Phe-AzaGly-Leu-Arg(Me)-Trp-
NH₂
Compound No. 580: des(1)-Ac-[D-Tyr2,D-Trp3,Ser
(Me)5,AzaGly7,Arg(Me)9,Trp10]MS10
Ac-D-Tyr-D-Trp-Asn-Ser(Me)-Phe-AzaGly-Leu-Arg(Me)-
Trp-NH₂
Compound No. 584: des(1)-Ac-[D-Tyr2,D-Trp3,Dap
(Ac)4,AzaGly7,Arg(Me)9,Trp10]MS10
Ac-D-Tyr-D-Trp-Dap(Ac)-Ser-Phe-AzaGly-Leu-Arg(Me)-
Trp-NH₂
Compound No. 585: des(1)-Ac-[D-Tyr2,D-Trp3,Dap
(For)4,AzaGly7,Arg(Me)9,Trp10]MS10
Ac-D-Tyr-D-Trp-Dap(For)-Ser-Phe-AzaGly-Leu-Arg
(Me)-Trp-NH₂
Compound No. 586: des(1)-Ac-[D-Tyr2,Thr5,D-Phe6,
AzaGly7,Arg(Me)9,Trp10]MS10
Ac-D-Tyr-Trp-Asn-Thr-D-Phe-AzaGly-Leu-Arg(Me)-Trp-
NH₂
Compound No. 589: des(1)-Ac-[D-Tyr2,D-Trp3,Thr5,
AzaGly7,Arg(Me)9,Nal(2)10]MS10
Ac-D-Tyr-D-Trp-Asn-Thr-Phe-AzaGly-Leu-Arg(Me)-Nal
(2)-NH₂
Compound No. 590: des(1)-Ac-[D-Tyr2,D-Trp3,Thr5,
AzaGly7,Arg(Me)9,Thi10]MS10
Ac-D-Tyr-D-Trp-Asn-Thr-Phe-AzaGly-Leu-Arg(Me)-Thi-
NH₂
Compound No. 591: des(1)-Ac-[D-Tyr2,D-Trp3,Thr5,
AzaGly7,Arg(Me)9,Tyr10]MS10
Ac-D-Tyr-D-Trp-Asn-Thr-Phe-AzaGly-Leu-Arg(Me)-Tyr-
NH₂
Compound No. 592: des(1)-Ac-[D-Tyr2,D-Trp3,Thr5,
AzaGly7,Arg(Me)9,Phe(4F)10]MS10
Ac-D-Tyr-D-Trp-Asn-Thr-Phe-AzaGly-Leu-Arg(Me)-
Phe(4F)-NH₂
Compound No. 594: des(1)-Ac-[D-Tyr2,D-Trp3,Thr5,
AzaGly7,Arg(Me)9,Hph10]MS10
Ac-D-Tyr-D-Trp-Asn-Thr-Phe-AzaGly-Leu-Arg(Me)-Hph-
NH₂
Compound No. 595: des(1)-Ac-[D-Tyr2,D-Trp3,Thr5,
AzaGly7,Arg(Me)9,Cha10]MS10
Ac-D-Tyr-D-Trp-Asn-Thr-Phe-AzaGly-Leu-Arg(Me)-Cha-
NH₂
Compound No. 596: des(1)-Ac-[D-Tyr2,D-Trp3,Thr5,
AzaGly7,Arg(Me)9,Leu10]MS10
Ac-D-Tyr-D-Trp-Asn-Thr-Phe-AzaGly-Leu-Arg(Me)-Leu-
NH₂
Compound No. 597: des(1)-Ac-[D-Tyr2,D-Trp3,Thr5,D-
Phe6,AzaGly7,Arg(Me)9,Trp10]MS10
Ac-D-Tyr-D-Trp-Asn-Thr-D-Phe-AzaGly-Leu-Arg(Me)-
Trp-NH₂
Compound No. 598: des(1)-Ac-[D-Tyr2,D-Trp3,Thr5,
Arg(Me)9,Trp10]MS10
Ac-D-Tyr-D-Trp-Asn-Thr-Phe-Gly-Leu-Arg(Me)-Trp-
NH₂
Compound No. 599: des(1)-Ac-[D-Tyr2,D-Trp3,Thr5,
AzaGly7,Orn9,Trp10]MS10
Ac-D-Tyr-D-Trp-Asn-Thr-Phe-AzaGly-Leu-Orn-Trp-NH₂
Compound No. 600: des(1)-Ac-[D-Tyr2,D-Trp3,Thr5,
AzaGly7,Trp10]MS10
Ac-D-Tyr-D-Trp-Asn-Thr-Phe-AzaGly-Leu-Arg-Trp-NH₂
Compound No. 601: des(1)-Ac-[D-Tyr2,D-Trp3,Thr5,
D-Phe6,Arg(Me)9,Trp10]MS10
Ac-D-Tyr-D-Trp-Asn-Thr-D-Phe-Gly-Leu-Arg(Me)-Trp-
NH₂
Compound No. 602: des(1)-Ac-[D-NMeTyr2,D-Trp3,
Thr5,AzaGly7,Arg(Me)9,Trp10]MS10
Ac-D-NMeTyr-D-Trp-Asn-Thr-Phe-AzaGly-Leu-Arg(Me)-
Trp-NH₂
Compound No. 603: des(1)-Ac-[D-Tyr2,D-Pya(4)3,
Thr5,D-Phe6,AzaGly7,Arg(Me)9,Trp10]MS10
Ac-D-Tyr-D-Pya(4)-Asn-Thr-D-Phe-AzaGly-Leu-Arg
(Me)-Trp-NH₂
Compound No. 604: des(1)-Ac-[D-Tyr2,D-Trp3,Thr5,
AzaGly7,Arg(Tos)9,Trp10]MS10
Ac-D-Tyr-D-Trp-Asn-Thr-Phe-AzaGly-Leu-Arg(Tos)-
Trp-NH₂
Compound No. 605: des(1)-Ac-[D-Tyr2,D-Trp3,Thr5,
AzaGly7,Arg(NO2)9,Trp10]MS10
Ac-D-Tyr-D-Trp-Asn-Thr-Phe-AzaGly-Leu-Arg(NO2)-
Trp-NH₂
Compound No. 607: des(1)-Ac-[D-Tyr2,D-Trp3,Thr5,
AzaGly7,Arg(Me2)asym9,Trp10]MS10
Ac-D-Tyr-D-Trp-Asn-Thr-Phe-AzaGly-Leu-Arg(Me2)
asym-Trp-NH₂
Compound No. 608: des(1)-Ac-[D-Tyr2,D-Trp3,Thr5,
AzaGly7,Arg(Me2)sym9,Trp10]MS10
Ac-D-Tyr-D-Trp-Asn-Thr-Phe-AzaGly-Leu-Arg(Me2)sym-
Trp-NH₂
Compound No. 609: des(1)-Ac-[D-Tyr2,D-Trp3,Thr5,
AzaGly7,Arg(Et)9,Trp10]MS10
Ac-D-Tyr-D-Trp-Asn-Thr-Phe-AzaGly-Leu-Arg(Et)-Trp-
NH₂
Compound No. 610: des(1)-Ac-[D-Tyr2,D-Trp3,Thr5,
AzaGly7,Lys(Me2)9,Trp10]MS10
Ac-D-Tyr-D-Trp-Asn-Thr-Phe-AzaGly-Leu-Lys(Me2)-
Trp-NH₂
Compound No. 611: des(1)-Ac-[Tyr2,D-Pya(4)3,Thr5,
AzaGly7,Arg(Me)9,Trp10]MS10
Ac-Tyr-D-Pya(4)-Asn-Thr-Phe-AzaGly-Leu-Arg(Me)-
Trp-NH₂
Compound No. 612: des(1)-For-[D-Tyr2,D-Trp3,Thr5,
AzaGly7,Arg(Me)9,Trp10]MS10
For-D-Tyr-D-Trp-Asn-Thr-Phe-AzaGly-Leu-Arg(Me)-
Trp-NH₂
Compound No. 613: des(1)-Propionyl-[D-Tyr2,D-Trp3,
Thr5,AzaGly7,Arg(Me)9,Trp10]MS10
Propionyl-D-Tyr-D-Trp-Asn-Thr-Phe-AzaGly-Leu-Arg
(Me)-Trp-NH₂
Compound No. 614: des(1)-Amidino-[D-Tyr2,D-Trp3,
Thr5,AzaGly7,Arg(Me)9,Trp10]MS10
Amidino-D-Tyr-D-Trp-Asn-Thr-Phe-AzaGly-Leu-Arg(Me)-
Trp-NH₂
Compound No. 615: des(1)-Ac-[Tyr2,D-Pya(4)3,Thr5,
AzaGly7,Arg(Me)9,Trp10]MS10
Ac-Tyr-D-Pya(4)-Asn-Thr-Phe-AzaGly-Leu-Arg(Me)-Trp-
NH₂
Compound No. 616: des(1)-Ac-[D-Ala2,D-Trp3,Thr5,Aza
Gly7,Arg(Me)9,Trp10]MS10
Ac-D-Ala-D-Trp-Asn-Thr-Phe-AzaGly-Leu-Arg(Me)-Trp-
NH₂
Compound No. 617: des(1)-Ac-[D-Leu2,D-Trp3,Thr5,
AzaGly7,Arg(Me)9,Trp10]MS10
Ac-D-Leu-D-Trp-Asn-Thr-Phe-AzaGly-Leu-Arg(Me)-Trp-
NH₂
Compound No. 618: des(1)-Ac-[D-Phe2,D-Trp3,Thr5,Aza
Gly7,Arg(Me)9,Trp10]MS10
Ac-D-Phe-D-Trp-Asn-Thr-Phe-AzaGly-Leu-Arg(Me)-Trp-
NH₂
Compound No. 619: des(1)-Ac-[D-Nal(1)2,D-Trp3,
Thr5,AzaGly7,Arg(Me)9,Trp10]MS10
Ac-D-Nal(1)-D-Trp-Asn-Thr-Phe-AzaGly-Leu-Arg(Me)-
Trp-NH₂
Compound No. 620: des(1)-Ac-[D-Nal(2)2,D-Trp3,
Thr5,AzaGly7,Arg(Me)9,Trp10]MS10
Ac-D-Nal(2)-D-Trp-Asn-Thr-Phe-AzaGly-Leu-Arg(Me)-
Trp-NH₂
Compound No. 621: des(1)-Ac-[D-Lys2,D-Trp3,Thr5,
AzaGly7,Arg(Me)9,Trp10]MS10
Ac-D-Lys-D-Trp-Asn-Thr-Phe-AzaGly-Leu-Arg(Me)-Trp-
NH₂
Compound No. 622: des(1)-Ac-[D-Glu2,D-Trp3,Thr5,
AzaGly7,Arg(Me)9,Trp10]MS10
Ac-D-Glu-D-Trp-Asn-Thr-Phe-AzaGly-Leu-Arg(Me)-Trp-
NH₂
Compound No. 623: des(1)-Ac-[D-Tyr2,Thr5,AzaGly7,
Arg(Me)9,Trp10]MS10
Ac-D-Tyr-Trp-Asn-Thr-Phe-AzaGly-Leu-Arg(Me)-Trp-
NH₂
Compound No. 624: des(1)-Ac-[D-Tyr2,Pya(4)3,Thr5,
AzaGly7,Arg(Me)9,Trp10]MS10
Ac-D-Tyr-Pya(4)-Asn-Thr-Phe-AzaGly-Leu-Arg(Me)-
Trp-NH₂
Compound No. 625: des(1)-Ac-[D-Tyr2,D-Ala3,Thr5,
AzaGly7,Arg(Me)9,Trp10]MS10
Ac-D-Tyr-D-Ala-Asn-Thr-Phe-AzaGly-Leu-Arg(Me)-Trp-
NH₂
Compound No. 626: des(1)-Ac-[D-Tyr2,D-Leu3,Thr5,
AzaGly7,Arg(Me)9,Trp10]MS10
Ac-D-Tyr-D-Leu-Asn-Thr-Phe-AzaGly-Leu-Arg(Me)-Trp-
NH₂
Compound No. 627: des(1)-Ac-[D-Tyr2,D-Phe3,Thr5,
AzaGly7,Arg(Me)9,Trp10]MS10
Ac-D-Tyr-D-Phe-Asn-Thr-Phe-AzaGly-Leu-Arg(Me)-
Trp-NH₂
Compound No. 628: des(1)-Ac-[D-Tyr2,D-Thr3,Thr5,
AzaGly7,Arg(Me)9,Trp10]MS10
Ac-D-Tyr-D-Thr-Asn-Thr-Phe-AzaGly-Leu-Arg(Me)-Trp-
NH₂
Compound No. 629: des(1)-Ac-[D-Tyr2,D-Lys3,Thr5,
AzaGly7,Arg(Me)9,Trp10]MS10
Ac-D-Tyr-D-Lys-Asn-Thr-Phe-AzaGly-Leu-Arg(Me)-Trp-
NH₂
Compound No. 630: des(1)-Ac-[D-Tyr2,D-Glu3,Thr5,
AzaGly7,Arg(Me)9,Trp10]MS10
Ac-D-Tyr-D-Glu-Asn-Thr-Phe-AzaGly-Leu-Arg(Me)-Trp-
NH₂
Compound No. 631: des(1)-Ac-[D-Tyr2,D-Trp3,Thr5,
Ala6,AzaGly7,Arg(Me)9,Trp10]MS10
Ac-D-Tyr-D-Trp-Asn-Thr-Ala-AzaGly-Leu-Arg(Me)-Trp-
NH₂
Compound No. 632: des(1)-Ac-[D-Tyr2,D-Trp3,Thr5,
Leu6,AzaGly7,Arg(Me)9,Trp10]MS10
Ac-D-Tyr-D-Trp-Asn-Thr-Leu-AzaGly-Leu-Arg(Me)-Trp-
NH₂
Compound No. 633: des(1)-Ac-[D-Tyr2,D-Trp3,Thr5,
Lys6,AzaGly7,Arg(Me)9,Trp10]MS10
Ac-D-Tyr-D-Trp-Asn-Thr-Lys-AzaGly-Leu-Arg(Me)-Trp-
NH₂
Compound No. 634: des(1)-Ac-[D-Tyr2,D-Trp3,Thr5,
Glu6,AzaGly7,Arg(Me)9,Trp10]MS10
Ac-D-Tyr-D-Trp-Asn-Thr-Glu-AzaGly-Leu-Arg(Me)-Trp-
NH₂
Compound No. 635: des(1)-Ac-[D-Tyr2,D-Trp3,Thr5,
Pya(4)6,AzaGly7,Arg(Me)9,Trp10]MS10
Ac-D-Tyr-D-Trp-Asn-Thr-Pya(4)-AzaGly-Leu-Arg(Me)-
Trp-NH₂
Compound No. 636: des(1)-Ac-[D-Tyr2,D-Trp3,Thr5,
NMePhe6,AzaGly7,Arg(Me)9,Trp10]MS10
Ac-D-Tyr-D-Trp-Asn-Thr-NMePhe-AzaGly-Leu-Arg(Me)-
Trp-NH₂
Compound No. 637: des(1)-Ac-[D-Tyr2,D-Trp3,Thr5,
Phe(4F)6,AzaGly7,Arg(Me)9,Trp10]MS10
Ac-D-Tyr-D-Trp-Asn-Thr-Phe(4F)-AzaGly-Leu-Arg(Me)-
Trp-NH₂
Compound No. 638: des(1)-Ac-[D-Tyr2,D-Pya(4)3,
Thr5,Phe(4F)6,AzaGly7,Arg(Me)9,Trp10]MS10
Ac-D-Tyr-D-Pya(4)-Asn-Thr-Phe(4F)-AzaGly-Leu-Arg
(Me)-Trp-NH₂
Compound No. 639: des(1)-Ac-[D-Tyr2,D-Trp3,Thr5,
AzaGly7,Lys9,Trp10]MS10
Ac-D-Tyr-D-Trp-Asn-Thr-Phe-AzaGly-Leu-Lys-Trp-NH₂
Compound No. 641: des(1)-Ac-[D-Tyr2,D-Trp3,Thr5,
AzaGly7,A1a8,Arg(Me)9,Trp10]MS10
Ac-D-Tyr-D-Trp-Asn-Thr-Phe-AzaGly-Ala-Arg(Me)-Trp-
NH₂
Compound No. 642: des(1)-Ac-[D-Tyr2,D-Trp3,Thr5,
AzaGly7,Val8,Arg(Me)9,Trp10]MS10
Ac-D-Tyr-D-Trp-Asn-Thr-Phe-AzaGly-Val-Arg(Me)-Trp-
NH₂
Compound No. 643: des(1)-Ac-[D-Tyr2,D-Trp3,Thr5,
AzaGly7,Phe8,Arg(Me)9,Trp10]MS10
Ac-D-Tyr-D-Trp-Asn-Thr-Phe-AzaGly-Phe-Arg(Me)-Trp-
NH₂
Compound No. 644: des(1)-Ac-[D-Tyr2,D-Trp3,Thr5,
AzaGly7,Ser8,Arg(Me)9,Trp10]MS10
Ac-D-Tyr-D-Trp-Asn-Thr-Phe-AzaGly-Ser-Arg(Me)-Trp-
NH₂
Compound No. 645: des(1)-Ac-[D-Tyr2,D-Trp3,Thr5,
AzaGly7,Har9,Trp10]MS10
Ac-D-Tyr-D-Trp-Asn-Thr-Phe-AzaGly-Leu-Har-Trp-NH₂
Compound No. 646: des(1)-Ac-[D-Tyr2,D-Trp3,Thr5,
AzaGly7,Har(Me)9,Trp10]MS10
Ac-D-Tyr-D-Trp-Asn-Thr-Phe-AzaGly-Leu-Har(Me)-Trp-
NH₂
Compound No. 647: des(1)-Ac-[D-Tyr2,D-Trp3,Asp4,
Thr5,AzaGly7,Arg(Me)9,Trp10]MS10
Ac-D-Tyr-D-Trp-Asp-Thr-Phe-AzaGly-Leu-Arg(Me)-Trp-
NH₂
Compound No. 648: [Gly1,D-Tyr2,D-Trp3,Thr5,Aza
Gly7,Arg(Me)9,Trp10]MS10
Gly-D-Tyr-D-Trp-Asn-Thr-Phe-AzaGly-Leu-Arg(Me)-
Trp-NH₂
Compound No. 649: Ac-[Gly1,D-Tyr2,D-Trp3,Thr5,Aza
Gly7,Arg(Me)9,Trp10]MS10
Ac-Gly-D-Tyr-D-Trp-Asn-Thr-Phe-AzaGly-Leu-Arg(Me)-
Trp-NH₂
Compound No. 650: [D-Tyr1,D-Tyr2,D-Trp3,Thr5,Aza
Gly7,Arg(Me)9,Trp10]MS10
D-Tyr-D-Tyr-D-Trp-Asn-Thr-Phe-AzaGly-Leu-Arg(Me)-
Trp-NH₂
Compound No. 651: Ac-[D-Tyr1,D-Tyr2,D-Trp3,Thr5,
AzaGly7,Arg(Me)9,Trp10]MS10
Ac-D-Tyr-D-Tyr-D-Trp-Asn-Thr-Phe-AzaGly-Leu-Arg
(Me)-Trp-NH₂
Compound No. 652: des(1)-pGlu-[D-Tyr2,D-Trp3,Thr5,
AzaGly7,Arg(Me)9,Trp10]MS10
pGlu-D-Tyr-D-Trp-Asn-Thr-Phe-AzaGly-Leu-Arg(Me)-
Trp-NH₂
Compound No. 653: des(1)-Ac-[D-Tyr2,D-Trp3,D-Asn4,
Thr5,AzaGly7,Arg(Me)9,Trp10]MS10
Ac-D-Tyr-D-Trp-D-Asn-Thr-Phe-AzaGly-Leu-Arg(Me)-
Trp-NH₂
Compound No. 654: des(1)-Ac-[D-Tyr2,D-Trp3,D-Thr5,
AzaGly7,Arg(Me)9,Trp10]MS10
Ac-D-Tyr-D-Trp-Asn-D-Thr-Phe-AzaGly-Leu-Arg(Me)-
Trp-NH₂
Compound No. 655: des(1)-Ac-[D-Tyr2,D-Trp3,NMeAsn4,
Thr5,AzaGly7,Arg(Me)9,Trp10]MS10
Ac-D-Tyr-D-Trp-NMeAsn-Thr-Phe-AzaGly-Leu-Arg(Me)-
Trp-NH₂
Compound No. 656: des(1)-Ac-[D-Tyr2,D-Trp3,NMe
Ser5,AzaGly7,Arg(Me)9,Trp10]MS10
Ac-D-Tyr-D-Trp-Asn-NMeSer-Phe-AzaGly-Leu-Arg(Me)-
Trp-NH₂
Compound No. 657: des(1)-Ac-[D-Tyr2,Pro3,Thr5,Aza
Gly7,Arg(Me)9,Trp10]MS10
Ac-D-Tyr-Pro-Asn-Thr-Phe-AzaGly-Leu-Arg(Me)-Trp-
NH₂
Compound No. 658: des(1)-Ac-[D-Tyr2,D-Pya(2)3,
Thr5,AzaGly7,Arg(Me)9,Trp10]MS10
Ac-D-Tyr-D-Pya(2)-Asn-Thr-Phe-AzaGly-Leu-Arg(Me)-
Trp-NH₂
Compound No. 659: des(1)-Ac-[D-Tyr2,D-Trp3,allo-
Thr5,AzaGly7,Arg(Me)9,Trp10]MS10
Ac-D-Tyr-D-Trp-Asn-allo-Thr-Phe-AzaGly-Leu-Arg
(Me)-Trp-NH₂
Compound No. 660: des(1)-Ac-[D-Tyr2,D-Pya(3)3,
Thr5,AzaGly7,Arg(Me)9,Trp10]MS10
Ac-D-Tyr-D-Pya(3)-Asn-Thr-Phe-AzaGly-Leu-Arg(Me)-
Trp-NH₂
Compound No. 661: des(1)-Ac-[D-Tyr2,D-Pro3,Thr5,
AzaGly7,Arg(Me)9,Trp10]MS10
Ac-D-Tyr-D-Pro-Asn-Thr-Phe-AzaGly-Leu-Arg(Me)-Trp-
NH₂
Compound No. 662: des(1)-Ac-[D-Tyr2,Tic3,Thr5,
AzaGly7,Arg(Me)9,Trp10]MS10
Ac-D-Tyr-Tic-Asn-Thr-Phe-AzaGly-Leu-Arg(Me)-Trp-
NH₂
Compound No. 663: des(1)-Ac-[D-Trp2,D-Trp3,Thr5,
AzaGly7,Arg(Me)9,Trp10]MS10
Ac-D-Trp-D-Trp-Asn-Thr-Phe-AzaGly-Leu-Arg(Me)-Trp-
NH₂
Compound No. 664: des(1)-Ac-[Tyr2,Thr5,AzaGly7,
Arg(Me)9,Trp10]MS10
Ac-Tyr-Trp-Asn-Thr-Phe-AzaGly-Leu-Arg(Me)-Trp-NH₂
Compound No. 665: des(1-2)-[D-Trp3,Thr5,AzaGly7,
Arg(Me)9,Trp10]MS10
D-Trp-Asn-Thr-Phe-AzaGly-Leu-Arg(Me)-Trp-NH₂
Compound No. 666: des(1-2)-Ac-[D-Trp3,Thr5,Aza
Gly7,Arg(Me)9,Trp10]MS10
Ac-D-Trp-Asn-Thr-Phe-AzaGly-Leu-Arg(Me)-Trp-NH₂
Compound No. 667: des(1-2)-Hexanoyl-[D-Trp3,Thr5,
AzaGly7,Arg(Me)9,Trp10]MS10
Hexanoyl-D-Trp-Asn-Thr-Phe-AzaGly-Leu-Arg(Me)-Trp-
NH₂
Compound No. 668: des(1-2)-Cyclohexanecarbonyl-[D-
Trp3,Thr5,AzaGly7,Arg(Me)9,Trp10]MS10
Cyclohexanecarbonyl-D-Trp-Asn-Thr-Phe-AzaGly-Leu-
Arg(Me)-Trp-NH₂
Compound No. 669: des(1-2)-Benzoyl-[D-Trp3,Thr5,
AzaGly7,Arg(Me)9,Trp10]MS10
Benzoyl-D-Trp-Asn-Thr-Phe-AzaGly-Leu-Arg(Me)-Trp-NH₂
Compound No. 670: des(1-2)-3-Pyridinepropionyl-[D-
Trp3,Thr5,AzaGly7,Arg(Me)9,Trp10]MS10
3-Pyridinepropionyl-D-Trp-Asn-Thr-Phe-AzaGly-Leu-
Arg(Me)-Trp-NH₂
Compound No. 671: des(1-2)-Adipoyl-[D-Trp3,Thr5,Aza
Gly7,Arg(Me)9,Trp10]MS10
Adipoyl-D-Trp-Asn-Thr-Phe-AzaGly-Leu-Arg(Me)-Trp-NH₂
Compound No. 672: des(1)-Ac-[D-Tyr2,NMeTrp3,Thr5,
AzaGly7,Arg(Me)9,Trp10]MS10
Ac-D-Tyr-NMeTrp-Asn-Thr-Phe-AzaGly-Leu-Arg(Me)-Trp-
NH₂
Compound No. 674: des(1-2)-6-Aminocaproyl-[D-Trp3,
Thr5,AzaGly7,Arg(Me)9,Trp10]MS10
6-Aminocaproyl-D-Trp-Asn-Thr-Phe-AzaGly-Leu-Arg
(Me)-Trp-NH₂
Compound No. 675: [D-Tyr2,D-Trp3,Thr5,AzaGly7,Arg
(Me)9,Trp10]MS10
Tyr-D-Tyr-D-Trp-Asn-Thr-Phe-AzaGly-Leu-Arg(Me)-
Trp-NH₂
Compound No. 676: Ac-[D-Tyr2,D-Trp3,Thr5,AzaGly7,
Arg(Me)9,Trp10]MS10
Ac-Tyr-D-Tyr-D-Trp-Asn-Thr-Phe-AzaGly-Leu-Arg(Me)-
Trp-NH₂
Compound No. 677: Ac-des(1)-[D-Tyr2,D-Trp3,Thr5,
AzaGly7,Nva8,Arg(Me)9,Trp10]MS10
Ac-D-Tyr-D-Trp-Asn-Thr-Phe-AzaGly-Nva-Arg(Me)-Trp-
NH₂
Compound No. 678: Ac-des(1)-[D-Tyr2,D-Trp3,Thr5,
AzaGly7,Ile8,Arg(Me)9,Trp10]MS10
Ac-D-Tyr-D-Trp-Asn-Thr-Phe-AzaGly-Ile-Arg(Me)-Trp-
NH₂
Compound No. 679: des(1-2)-Amidino-[D-Trp3,Thr5,
AzaGly7,Arg(Me)9,Trp10]MS10
Amidino-D-Trp-Asn-Thr-Phe-AzaGly-Leu-Arg(Me)-Trp-
NH₂
Compound No. 680: des(1-2)-Glycoloyl-[D-Trp3,Thr5,
AzaGly7,Arg(Me)9,Trp10]MS10
Glycoloyl-D-Trp-Asn-Thr-Phe-AzaGly-Leu-Arg(Me)-
Trp-NH₂
Compound No. 681: des(1)-Glycoloyl-[D-Tyr2,D-Trp3,
Thr5,AzaGly7,Arg(Me)9,Trp10]MS10
Glycoloyl-D-Tyr-D-Trp-Asn-Thr-Phe-AzaGly-Leu-Arg
(Me)-Trp-NH₂
Compound No. 682: des(1)-Ac-[D-Tyr2,D-Trp3,Thr5,
AzaGly7,G1n8,Arg(Me)9,Trp10]MS10
Ac-D-Tyr-D-Trp-Asn-Thr-Phe-AzaGly-Gln-Arg(Me)-Trp-
NH₂
Compound No. 685: des(1)-Ac-[D-Tyr2,D-Pya(4)3,Thr5,
AzaGly7,Arg(Me)9]MS10
Ac-D-Tyr-D-Pya(4)-Asn-Thr-Phe-AzaGly-Leu-Arg(Me)-
Phe-NH₂
Compound No. 686: des(1)-Ac-[D-Tyr2,D-Trp3,Gly4,Aza
Gly7,Arg(Me)9,Trp10]MS10
Ac-D-Tyr-D-Trp-Gly-Ser-Phe-AzaGly-Leu-Arg(Me)-Trp-
NH₂
Compound No. 688: des(1)-Ac-[D-Tyr2,D-Trp3,Thr5,
AzaGly7,Pya(4)9,Trp10]MS10
Ac-D-Tyr-D-Trp-Asn-Thr-Phe-AzaGly-Leu-Pya(4)-Trp-
NH₂
Compound No. 689: des(1)-Ac-[D-Tyr2,D-Trp3,Thr5,Aza
Gly7,Arg(Me)9,D-Trp10]MS10
Ac-D-Tyr-D-Trp-Asn-Thr-Phe-AzaGly-Leu-Arg(Me)-D-
Trp-NH₂
Compound No. 691: des(1)-Ac-[D-Tyr2,D-Trp3,Thr5,
Tyr6,AzaGly7,Arg(Me)9,Trp10]MS10
Ac-D-Tyr-D-Trp-Asn-Thr-Tyr-AzaGly-Leu-Arg(Me)-Trp-
NH₂
Compound No. 692: des(1)-Ac-[D-Tyr2,D-Trp3,Thr5,
Trp6,AzaGly7,Arg(Me)9,Trp10]MS10
Ac-D-Tyr-D-Trp-Asn-Thr-Trp-AzaGly-Leu-Arg(Me)-Trp-
NH₂
Compound No. 693: des(1)-Ac-[D-Tyr2,D-
Trp3,Thr5,Tyr(Me)6,AzaGly7,Arg(Me)9,Trp10]MS10
Ac-D-Tyr-D-Trp-Asn-Thr-Tyr(Me)-AzaGly-Leu-Arg
(Me)-Trp-NH₂
Compound No. 694: des(1)-Ac-[D-Tyr2,D-Trp3,Thr5,
Nal(2)6,AzaGly7,Arg(Me)9,Trp10]MS10
Ac-D-Tyr-D-Trp-Asn-Thr-Nal(2)-AzaGly-Leu-Arg(Me)-
Trp-NH₂
Compound No. 695: des(1)-Ac-[D-Tyr2,D-Trp3,Thr5,
Thi6,AzaGly7,Arg(Me)9,Trp10]MS10
Ac-D-Tyr-D-Trp-Asn-Thr-Thi-AzaGly-Leu-Arg(Me)-Trp-
NH₂
Compound No. 696: des(1)-Ac-[D-Tyr2,D-Trp3,Thr5,
Cha6,AzaGly7,Arg(Me)9,Trp10]MS10
Ac-D-Tyr-D-Trp-Asn-Thr-Cha-AzaGly-Leu-Arg(Me)-Trp-
NH₂
Compound No. 698: des(1)-Ac-[D-Tyr2,D-Trp3,Thr5,
AzaGly7,Abu8,Arg(Me)9,Trp10]MS10
Ac-D-Tyr-D-Trp-Asn-Thr-Phe-AzaGly-Abu-Arg(Me)-
Trp-NH₂
Compound No. 699: des(1)-Ac-[D-Tyr2,D-Trp3,Thr5,
AzaGly7,γMeLeu8,Arg(Me)9,Trp10]MS10
Ac-D-Tyr-D-Trp-Asn-Thr-Phe-AzaGly-γMeLeu-Arg(Me)-
Trp-NH₂
Compound No. 700: des(1)-Ac-[D-Tyr2,D-Trp3,Thr5,
Aib8,Arg(Me)9,Trp10]MS10
Ac-D-Tyr-D-Trp-Asn-Thr-Phe-Gly-Aib-Arg(Me)-Trp-
NH₂
Compound No. 701: des(1)-Ac-[D-Tyr2,D-Trp3,Dap4,
AzaGly7,Arg(Me)9,Trp10]MS10
Ac-D-Tyr-D-Trp-Dap-Ser-Phe-AzaGly-Leu-Arg(Me)-
Trp-NH₂
Compound No. 702: des(1)-Ac-[D-Tyr2,D-
Trp3,Asp(NHMe)4,Thr5,AzaGly7,Arg(Me)9,Trp10]MS10
Ac-D-Tyr-D-Trp-Asp(NHMe)-Thr-Phe-AzaGly-Leu-
Arg(Me)-Trp-NH₂
Compound No. 703: des(1)-Ac-[D-Tyr2,D-
Trp3,Asp(NMe2)4,Thr5,AzaGly7,Arg(Me)9,Trp10]MS10
Ac-D-Tyr-D-Trp-Asp(NMe2)-Thr-Phe-AzaGly-Leu-
Arg(Me)-Trp-NH₂

It is preferable that the metastin derivative (II) of the invention not include a peptide (natural human metastin or partial peptide thereof) composed of any of the following amino acid sequences shown in SEQ ID NO: 1: amino acids 1 to 54 (Compound No. 1), amino acids 2 to 54, amino acids 3 to 54, amino acids 4 to 54, amino acids 5 to 54, amino acids 6 to 54, amino acids 7 to 54, amino acids 8 to 54, amino acids 9 to 54, amino acids 10 to 54, amino acids 11 to 54, amino acids 12 to 54, amino acids 13 to 54, amino acids 14 to 54, amino acids 15 to 54, amino acids 16 to 54, amino acids 17 to 54, amino acids 18 to 54, amino acids 19 to 54, amino acids 20 to 54, amino acids 21 to 54, amino acids 22 to 54, amino acids 23 to 54, amino acids 24 to 54, amino acids 25 to 54, amino acids 26 to 54, amino acids 27 to 54, amino acids 28 to 54, amino acids 29 to 54, amino acids 30 to 54, amino acids 31 to 54, amino acids 32 to 54, amino acids 33 to 54, amino acids 34 to 54, amino acids 35 to 54, amino acids 36 to 54, amino acids 37 to 54, amino acids 38 to 54, amino acids 39 to 54, amino acids 40 to 54 (Compound No. 2), amino acids 41 to 54, amino acids 42 to 54 (Compound No. 32), amino acids 43 to 54, amino acids 44 to 54, amino acids 45 to 54 (Compound 3), amino acids 46 to 54 (Compound No. 4), amino acids 47 to 54, amino acids 48 to 54 or amino acids 49 to 54.

All compounds in which any of the groups of the various symbols mentioned above have been combined may be advantageously used as the metastin derivative (II), although the use of compounds indicated by the following compound numbers is especially preferred.

Compound No. 332:
des(1-5)-GuAmb-[AzaGly7,Arg(Me)9]MS10
GuAmb-Phe-AzaGly-Leu-Arg(Me)-Phe-NH₂
Compound No. 333:
des(1-5)-GuAmb-[Arg(Me)9]MS10
GuAmb-Phe-Gly-Leu-Arg(Me)-Phe-NH₂
Compound No. 334:
des(1-5)-GuAmb-[AzaGly7,Arg(Me)9,Trp10]MS10
GuAmb-Phe-AzaGly-Leu-Arg(Me)-Trp-NH₂
Compound No. 339:
des(1-5)-3-(3-Indolyl)propionyl-[AzaGly7,
Arg(Me)9]MS10
3-(3-Indolyl)propionyl-Phe-AzaGly-Leu-Arg(Me)-
Phe-NH₂
Compound No. 340:
des(1-5)-3-(3-Pyridyl)propionyl-[AzaGly7,
Arg(Me)9]MS10
3-(3-Pyridyl)propionyl-Phe-AzaGly-Leu-Arg(Me)-
Phe-NH₂
Compound No. 341:
des(1-5)-Benzoyl-[AzaGly7,Arg(Me)9]MS10
Benzoyl-Phe-AzaGly-Leu-Arg(Me)-Phe-NH₂
Compound No. 344:
des(1-5)-Indole-3-carbonyl-[AzaGly7,
Arg(Me)9]MS10
Indole-3-carbonyl-Phe-AzaGly-Leu-Arg(Me)-Phe-NH₂
Compound No. 345:
des(1-5)-Indole-3-acetyl-[AzaGly7,Arg(Me)9]MS10
Indole-3-acetyl-Phe-AzaGly-Leu-Arg(Me)-Phe-NH₂
Compound No. 346:
des(1-5)-Ac-[AzaGly7,Arg(Me)9]MS10
Ac-Phe-AzaGly-Leu-Arg(Me)-Phe-NH₂
Compound No. 347:
des(1-5)-n-Hexanoyl-[AzaGly7,Arg(Me)9]MS10
n-Hexanoyl-Phe-AzaGly-Leu-Arg(Me)-Phe-NH₂
Compound No. 348:
des(1-5)-Z-[AzaGly7,Arg(Me)9]MS10
Z-Phe-AzaGly-Leu-Arg(Me)-Phe-NH₂
Compound No. 349:
des(1-5)-Tos-[AzaGly7,Arg(Me)9]MS10
Tos-Phe-AzaGly-Leu-Arg(Me)-Phe-NH₂
Compound No. 351: des(1-5)-Benzoyl-MS10
Benzoyl-Phe-Gly-Leu-Arg-Phe-NH₂
Compound No. 352:
des(1-5)-3-(3-Indolyl)propionyl-MS10
3-(3-Indolyl)propionyl-Phe-Gly-Leu-Arg-Phe-NH₂
Compound No. 353:
des(1-5)-Benzoyl-[AzaGly7,Arg(Me)9,Trp10]MS10
Benzoyl-Phe-AzaGly-Leu-Arg(Me)-Trp-NH₂
Compound No. 354:
des(1-5)-3-(3-Indolyl)propionyl-[AzaGly7,
Arg(Me)9,Trp10]MS10
3-(3-Indolyl)propionyl-Phe-AzaGly-Leu-Arg(Me)-
Trp-NH₂
Compound No. 358:
des(1-5)-Ac-[AzaGly7,Arg(Me)9,Trp10]MS10
Ac-Phe-AzaGly-Leu-Arg(Me)-Trp-NH₂
Compound No. 362:
des(1-6)-3-Phenylpropionyl-[AzaGly7,
Arg(Me)9]MS10
3-Phenylpropionyl-AzaGly-Leu-Arg(Me)-Phe-NH₂
Compound No. 364:
des(1-5)-2-(Indol-3-yl)ethylcarbamoyl-[AzaGly7,
Arg(Me)9]MS10
2-(Indol-3-yl)ethylcarbamoyl-Phe-AzaGly-Leu-
Arg(Me)-Phe-NH₂
Compound No. 366:
des(1-5)-n-Hexanoyl-[AzaGly7,Arg(Me)9,
Trp10]MS10
n-Hexanoyl-Phe-AzaGly-Leu-Arg(Me)-Trp-NH₂
Compound No. 367:
des(1-5)-Z-[AzaGly7,Arg(Me)9,Trp10]MS10
Z-Phe-AzaGly-Leu-Arg(Me)-Trp-NH₂
Compound No. 368:
des(1-5)-Tos-[AzaGly7,Arg(Me)9,Trp10]MS10
Tos-Phe-AzaGly-Leu-Arg(Me)-Trp-NH₂
Compound No. 369:
des(1-5)-2-(Indol-3-yl)ethylcarbamoyl-[AzaGly7,
Arg(Me)9,Trp10]MS10
2-(Indol-3-yl)ethylcarbamoyl-Phe-AzaGly-Leu-
Arg(Me)-Trp-NH₂
Compound No. 373:
des(1-6)-(2S)-2-acetoxy-3-phenylpropionyl-
[AzaGly7,Arg(Me)9,Trp10]MS10
(25)-2-acetoxy-3-phenylpropionyl-AzaGly-Leu-
Arg(Me)-Trp-NH₂
Compound No. 374:
des(1-6)-Z-[AzaGly7,Arg(Me)9,Trp10]MS10
Z-AzaGly-Leu-Arg(Me)-Trp-NH₂
Compound No. 378:
des(1-6)-Diphenylacetyl-[AzaGly7,Arg(Me)9,
Trp10]MS10
Diphenylacetyl-AzaGly-Leu-Arg(Me)-Trp-NH₂
Compound No. 379:
des(1-6)-(2S)-2-(3-Indolylprpionyloxy)-3-
phenylpropionyl-[AzaGly7,Arg(Me)9,Trp10]MS10
(2S)-2-(3-Indolylprpionyloxy)-3-phenylpropionyl-
AzaGly-Leu-Arg(Me)-Trp-NH₂
Compound No. 380:
des(1-6)-(2S)-2-Benzoyloxy-3-phenylpropionyl-
[AzaGly7,Arg(Me)9,Trp10]MS10
(2S)-2-Benzoyloxy-3-phenylpropionyl-AzaGly-Leu-
Arg(Me)-Trp-NH₂
Compound No. 392:
des(1-5)-Benzoyl-[Ala6,AzaGly7,Arg(Me)9,
Trp10]MS10
Benzoyl-Ala-AzaGly-Leu-Arg(Me)-Trp-NH₂
Compound No. 393:
des(1-6)-Dibenzylcarbamoyl-[AzaGly7,Arg(Me)9,
Trp10]MS10
Dibenzylcarbamoyl-AzaGly-Leu-Arg(Me)-Trp-NH₂
Compound No. 408:
des(1-6)-1-Oxo-isochroman-3-carbonyl-[AzaGly7,
Arg(Me)9,Trp10]MS10
1-Oxo-isochroman-3-carbonyl-AzaGly-Leu-Arg(Me)-
Trp-NH₂
Compound No. 412:
des(1-6)-(2R)-2-Benzoyloxy-3-phenylpropionyl-
[AzaGly7,Arg(Me)9,Trp10]MS10
(2R)-2-Benzoyloxy-3-phenylpropionyl-AzaGly-Leu-
Arg(Me)-Trp-NH₂
Compound No. 417:
des(1-6)-Benzylphenethylcarbamoyl-[AzaGly7,
Arg(Me)9,Trp10]MS10
Benzylphenethylcarbamoyl-AzaGly-Leu-Arg(Me)-
Trp-NH₂
Compound No. 421:
des(1-5)-Benzoyl-[6Ψ7,CH2O,Arg(Me)9,
Trp10]MS10
Benzoyl-PheΨ(CH2O)Gly-Leu-Arg(Me)-Trp-NH₂
Compound No. 423:
des(1-5)-Benzoyl-[6Ψ7,NHCO,Arg(Me)9,
Trp10]MS10
Benzoyl-PheΨ(NHCO)Gly-Leu-Arg(Me)-Trp-NH₂
Compound No. 428:
des(1-6)-Dibenzylaminocarbamoyl-[AzaGly7,
Arg(Me)9,Trp10]MS10
Dibenzylaminocarbamoyl-AzaGly-Leu-Arg(Me)-Trp-NH₂
Compound No. 431:
des(1-5)-Benzoyl-[AzaPhe6,AzaGly7,Arg(Me)9,
Trp10]MS10
Benzoyl-AzaPhe-AzaGly-Leu-Arg(Me)-Trp-NH₂
Compound No. 432:
des(1-5)-3-Pyridinecarbonyl-[AzaGly7,Arg(Me)9,
Trp10]MS10
3-Pyridinecarbonyl-Phe-AzaGly-Leu-Arg(Me)-Trp-NH₂
Compound No. 434:
des(1-7)-Dibenzylaminocarbamoylacetyl-
[Arg(Me)9,Trp10]MS10
Dibenzylaminocarbamoylacetyl-Leu-Arg(Me)-Trp-NH₂
Compound No. 435:
des(1-5)-2-Pyridinecarbonyl-[AzaGly7,Arg(Me)9,
Trp10]MS10
2-Pyridinecarbonyl-Phe-AzaGly-Leu-Arg(Me)-Trp-NH₂
Compound No. 436:
des(1-5)-4-Pyridinecarbonyl-[AzaGly7,Arg(Me)9,
Trp10]MS10
4-Pyridinecarbonyl-Phe-AzaGly-Leu-Arg(Me)-Trp-NH₂
Compound No. 437:
des(1-5)-Propionyl-[AzaGly7,Arg(Me)9,
Trp10]MS10
Propionyl-Phe-AzaGly-Leu-Arg(Me)-Trp-NH₂
Compound No. 438:
des(1-5)-Isobutyryl-[AzaGly7,Arg(Me)9,
Trp10]MS10
Isobutyryl-Phe-AzaGly-Leu-Arg(Me)-Trp-NH₂
Compound No. 439:
des(1-5)-Cyclohexanecarbonyl-[AzaGly7,
Arg(Me)9,Trp10]MS10
Cyclohexanecarbonyl-Phe-AzaGly-Leu-Arg(Me)-
Trp-NH₂
Compound No. 440:
des(1-5)-Phenylacetyl-[AzaGly7,Arg(Me)9,
Trp10]MS10
Phenylacetyl-Phe-AzaGly-Leu-Arg(Me)-Trp-NH₂
Compound No. 441:
des(1-5)-Benzoyl-[Pya(2)6,AzaGly7,Arg(Me)9,
Trp10]MS10
Benzoyl-Pya(2)-AzaGly-Leu-Arg(Me)-Trp-NH₂
Compound No. 442:
des(1-5)-Benzoyl-[Pya(4)6,AzaGly7,Arg(Me)9,
Trp10]MS10
Benzoyl-Pya(4)-AzaGly-Leu-Arg(Me)-Trp-NH₂
Compound No. 443:
des(1-5)-2-Methylnicotinoyl-[AzaGly7,
Arg(Me)9,Trp10]MS10
2-Methylnicotinoyl-Phe-AzaGly-Leu-Arg(Me)-Trp-NH₂
Compound No. 444:
des(1-5)-5-Methylnicotinoyl-[AzaGly7,Arg(Me)9,
Trp10]MS10
5-Methylnicotinoyl-Phe-AzaGly-Leu-Arg(Me)-Trp-NH₂
Compound No. 445:
des(1-5)-6-Methylnicotinoyl-[AzaGly7,Arg(Me)9,
Trp10]MS10
6-Methylnicotinoyl-Phe-AzaGly-Leu-Arg(Me)-Trp-NH₂
Compound No. 446:
des(1-5)-Pyrazinecarbonyl-[AzaGly7,Arg(Me)9,
Trp10]MS10
Pyrazinecarbonyl-Phe-AzaGly-Leu-Arg(Me)-Trp-NH₂
Compound No. 447:
des(1-5)-Cyclopropanecarbonyl-[AzaGly7,Arg(Me)9,
Trp10]MS10
Cyclopropanecarbonyl-Phe-AzaGly-Leu-Arg(Me)-Trp-
NH₂
Compound No. 448:
des(1-5)-Trifluoroacetyl-[AzaGly7,Arg(Me)9,
Trp10]MS10
Trifluoroacetyl-Phe-AzaGly-Leu-Arg(Me)-Trp-NH₂
Compound No. 449:
des(1-5)-Benzoyl-[Cha6,AzaGly7,Arg(Me)9,
Trp10]MS10
Benzoyl-Cha-AzaGly-Leu-Arg(Me)-Trp-NH₂
Compound No. 450:
des(1-5)-Benzyl-[AzaGly7,Arg(Me)9,Trp10]MS10
Benzyl-Phe-AzaGly-Leu-Arg(Me)-Trp-NH₂
Compound No. 451:
des(1-5)-Cyclopropanecarbonyl-[Cha6,AzaGly7,
Arg(Me)9,Trp10]MS10
Cyclopropanecarbonyl-Cha-AzaGly-Leu-Arg(Me)-Trp-
NH₂
Compound No. 452:
des(1-5)-(R)-3-hydroxy-2-methylpropionyl-
[AzaGly7,Arg(Me)9,Trp10]MS10
(R)-3-hydroxy-2-methylpropionyl-Phe-AzaGly-Leu-
Arg(Me)-Trp-NH₂
Compound No. 453:
des(1-5)-2-Hydroxyisobutyryl-[AzaGly7,Arg(Me)9,
Trp10]MS10
2-Hydroxyisobutyryl-Phe-AzaGly-Leu-Arg(Me)-Trp-NH₂
Compound No. 454:
des(1-5)-3-Furancarbonyl-[AzaGly7,Arg(Me)9,
Trp10]MS10
3-Furancarbonyl-Phe-AzaGly-Leu-Arg(Me)-Trp-NH₂
Compound No. 455:
des(1-5)-Pyrrole-2-carbonyl-[AzaGly7,Arg(Me)9,
Trp10]MS10
Pyrrole-2-carbonyl-Phe-AzaGly-Leu-Arg(Me)-Trp-NH₂
Compound No. 459:
des(1-5)-4-Imidazolecarbonyl-[AzaGly7,Arg(Me)9,
Trp10]MS10
4-Imidazolecarbonyl-Phe-AzaGly-Leu-Arg(Me)-Trp-NH₂
Compound No. 460:
des(1-5)-4-Pyridinecarbonyl-[AzaGly7,Val8,
Arg(Me)9,Trp10]MS10
4-Pyridinecarbonyl-Phe-AzaGly-Val-Arg(Me)-Trp-NH₂
Compound No. 461:
des(1-5)-4-Pyridinecarbonyl-[AzaGly7,Arg(Me)9,
Nal(2)10]MS10
4-Pyridinecarbonyl-Phe-AzaGly-Leu-Arg(Me)-Nal(2)-
NH₂
Compound No. 462:
des(1-5)-6-Hydroxynicotinoyl-[AzaGly7,Arg(Me)9,
Trp10]MS10
6-Hydroxynicotinoyl-Phe-AzaGly-Leu-Arg(Me)-Trp-NH₂
Compound No. 463:
des(1-5)-6-Chloronicotinoyl-[AzaGly7,Arg(Me)9,
Trp10]MS10
6-Chloronicotinoyl-Phe-AzaGly-Leu-Arg(Me)-Trp-NH₂
Compound No. 464:
des(1-5)-6-(Trifluoromethyl)nicotinoyl-[AzaGly7,
Arg(Me)9,Trp10]MS10
6-(Trifluoromethyl)nicotinoyl-Phe-AzaGly-Leu-
Arg(Me)-Trp-NH₂
Compound No. 466:
des(1-5)-2-Azetidinecarbonyl-[AzaGly7,Arg(Me)9,
Trp10]MS10
2-Azetidinecarbonyl-Phe-AzaGly-Leu-Arg(Me)-Trp-NH₂
Compound No. 467:
des(1-5)-Dimethylcarbamoyl-[AzaGly7,Arg(Me)9,
Trp10]MS10
Dimethylcarbamoyl-Phe-AzaGly-Leu-Arg(Me)-Trp-NH₂
Compound No. 468:
des(1-5)-1-Azetidinecarbonyl-[AzaGly7,Arg(Me)9,
Trp10]MS10
1-Azetidinecarbonyl-Phe-AzaGly-Leu-Arg(Me)-Trp-NH₂
Compound No. 471:
des(1-5)-4-Pyridinecarbonyl-[AzaGly7,
Arg(Me)9]MS10
4-Pyridinecarbonyl-Phe-AzaGly-Leu-Arg(Me)-Phe-NH₂
Compound No. 472:
des(1-5)-4-Aminobenzoyl-[AzaGly7,Arg(Me)9,
Trp10]MS10
4-Aminobenzoyl-Phe-AzaGly-Leu-Arg(Me)-Trp-NH₂
Compound No. 473:
des(1-5)-4-Aminomethylbenzoyl-[AzaGly7,Arg(Me)9,
Trp10]MS10
4-Aminomethylbenzoyl-Phe-AzaGly-Leu-Arg(Me)-Trp-
NH₂
Compound No. 474:
des(1-5)-Pyrrole-3-carbonyl-[AzaGly7,Arg(Me)9,
Trp10]MS10
Pyrrole-3-carbonyl-Phe-AzaGly-Leu-Arg(Me)-Trp-NH₂
Compound No. 475:
des(1-5)-Pyrimidine-4-carbonyl-[AzaGly7,
Arg(Me)9,Trp10]MS10
Pyrimidine-4-carbonyl-Phe-AzaGly-Leu-Arg(Me)-Trp-
NH₂
Compound No. 477:
des(1-5)-4-Pyridinecarbonyl-[AzaGly7,Orn9,
Trp10]MS10
4-Pyridinecarbonyl-Phe-AzaGly-Leu-Orn-Trp-NH₂
Compound No. 478:
des(1-5)-4-Pyridinecarbonyl-[AzaGly7,Har9,
Trp10]MS10
4-Pyridinecarbonyl-Phe-AzaGly-Leu-Har-Trp-NH₂
Compound No. 479:
des(1-5)-Pyrimidine-2-carbonyl-[AzaGly7,
Arg(Me)9,Trp10]MS10
Pyrimidine-2-carbonyl-Phe-AzaGly-Leu-Arg(Me)-
Trp-NH₂
Compound No. 480:
des(1-5)-Pyridazine-4-carbonyl-[AzaGly7,
Arg(Me)9,Trp10]MS10
Pyridazine-4-carbonyl-Phe-AzaGly-Leu-Arg(Me)-
Trp-NH₂
Compound No. 481:
des(1)-[D-Tyr2,D-Pya(4)3,AzaGly7,Har9,
Trp10]MS10
D-Tyr-D-Pya(4)-Asn-Ser-Phe-AzaGly-Leu-Har-Trp-NH₂
Compound No. 486:
des(1)-[D-Tyr2,D-Pya(4)3,AzaGly7,Orn9]MS10
D-Tyr-D-Pya(4)-Asn-Ser-Phe-AzaGly-Leu-Orn-Phe-NH₂
Compound No. 487:
des(1)-[D-Tyr2,D-Pya(4)3,AzaGly7,Lys9]MS10
D-Tyr-D-Pya(4)-Asn-Ser-Phe-AzaGly-Leu-Lys-Phe-NH₂
Compound No. 488:
des(1)-[D-Tyr2,D-Pya(4)3,AzaGly7,Har9]MS10
D-Tyr-D-Pya(4)-Asn-Ser-Phe-AzaGly-Leu-Har-Phe-NH₂
Compound No. 489:
des(1)-[D-Tyr2,D-Pya(4)3,AzaGly7,Har(Me)9]MS10
D-Tyr-D-Pya(4)-Asn-Ser-Phe-AzaGly-Leu-Har(Me)-Phe-
NH₂
Compound No. 490:
des(1)-[D-Tyr2,Pya(4)3,AzaGly7,Arg(Me)9]MS10
D-Tyr-Pya(4)-Asn-Ser-Phe-AzaGly-Leu-Arg(Me)-Phe-NH₂
Compound No. 491:
des(1)-[D-Tyr2,D-Pya(4)3,Trp5,AzaGly7,Arg(Me)9,
Trp10]MS10
D-Tyr-D-Pya(4)-Asn-Trp-Phe-AzaGly-Leu-Arg(Me)-Trp-
NH₂
Compound No. 492:
des(1)-[D-Tyr2,D-Pya(4)3,Ala4,AzaGly7,Arg(Me)9,
Trp10]MS10
D-Tyr-D-Pya(4)-Ala-Ser-Phe-AzaGly-Leu-Arg(Me)-Trp-
NH₂
Compound No. 493:
des(1)-[D-Tyr2,D-Pya(4)3,Thr4,AzaGly7,Arg(Me)9,
Trp10]MS10
D-Tyr-D-Pya(4)-Thr-Ser-Phe-AzaGly-Leu-Arg(Me)-Trp-
NH₂
Compound No. 494:
des(1,4)-[D-Tyr2,D-Pya(4)3,AzaGly7,Arg(Me)9,
Trp10]MS10
D-Tyr-D-Pya(4)-Ser-Phe-AzaGly-Leu-Arg(Me)-Trp-NH₂
Compound No. 495:
des(1-3)-[D-Tyr4,Pya(4)5,AzaGly7,Arg(Me)9,
Trp10]MS10
D-Tyr-Pya(4)-Phe-AzaGly-Leu-Arg(Me)-Trp-NH₂
Compound No. 496:
des(1)-[D-Tyr2,D-Pya(4)3,Cha6,Arg(Me)9,Trp10]MS10
D-Tyr-D-Pya(4)-Asn-Ser-Cha-Gly-Leu-Arg(Me)-Trp-NH₂
Compound No. 497:
des(1)-[D-Tyr2,D-Pya(4)3,Cha6,Ala7,Arg(Me)9,
Trp10]MS10
D-Tyr-D-Pya(4)-Asn-Ser-Cha-Ala-Leu-Arg(Me)-Trp-NH₂
Compound No. 498:
des(1)-[D-Tyr2,D-Pya(4)3,Ile5,AzaGly7,Arg(Me)9,
Trp10]MS10
D-Tyr-D-Pya(4)-Asn-Ile-Phe-AzaGly-Leu-Arg(Me)-
Trp-NH₂
Compound No. 499:
des(1-3)-3-Phenylpropionyl-[AzaGly7,Arg(Me)9,
Trp10]MS10
3-Phenylpropionyl-Asn-Ser-Phe-AzaGly-Leu-Arg(Me)-
Trp-NH₂
Compound No. 500:
des(1-3)-3-Phenylpropionyl-[Ala4,AzaGly7,Arg(Me)9,
Trp10]MS10
3-Phenylpropionyl-Ala-Ser-Phe-AzaGly-Leu-Arg(Me)-
Trp-NH₂
Compound No. 501:
des(1)-[D-Tyr2,D-Pya(4)3,Thr5,AzaGly7,Arg(Me)9,
Trp10]MS10
D-Tyr-D-Pya(4)-Asn-Thr-Phe-AzaGly-Leu-Arg(Me)-Trp-
NH₂
Compound No. 502:
des(1)-[D-Tyr2,Pya(4)3,Ala4,AzaGly7,Arg(Me)9,
Trp10]MS10
D-Tyr-Pya(4)-Ala-Ser-Phe-AzaGly-Leu-Arg(Me)-Trp-
NH₂
Compound No. 503:
des(1)-[D-Tyr2,D-Trp3,Ala4,AzaGly7,Arg(Me)9,
Trp10]MS10
D-Tyr-D-Trp-Ala-Ser-Phe-AzaGly-Leu-Arg(Me)-Trp-NH₂
Compound No. 504:
[Acp1,D-Tyr2,D-Pya(4)3,AzaGly7,Arg(Me)9]MS10
Acp-D-Tyr-D-Pya(4)-Asn-Ser-Phe-AzaGly-Leu-Arg(Me)-
Phe-NH₂
Compound No. 505:
des(1-3)-3-Phenylpropionyl-[Thr5,AzaGly7,Arg(Me)9,
Trp10]MS10
3-Phenylpropionyl-Asn-Thr-Phe-AzaGly-Leu-Arg(Me)-
Trp-NH₂
Compound No. 506:
des(1-3)-3-Phenylpropionyl-[AzaGly7,Arg(Me)9,
Trp10]MS10
3-Phenylpropionyl-Asn-Ile-Phe-AzaGly-Leu-Arg(Me)-
Trp-NH₂
Compound No. 507:
des(1-3)-3-Phenylpropionyl-[Trp6,AzaGly7,Arg(Me)9,
Trp10]MS10
3-Phenylpropionyl-Asn-Ser-Trp-AzaGly-Leu-Arg(Me)-
Trp-NH₂
Compound No. 508:
des(1-3)-3-Phenylpropionyl-[Phe(4F)6,AzaGly7,
Arg(Me)9,Trp10]MS10
3-Phenylpropionyl-Asn-Ser-Phe(4F)-AzaGly-Leu-
Arg(Me)-Trp-NH₂
Compound No. 509:
des(1-3)-Benzoyl-[AzaGly7,Arg(Me)9,Trp10]MS10
Benzoyl-Asn-Ser-Phe-AzaGly-Leu-Arg(Me)-Trp-NH₂
Compound No. 510:
des(1-3)-Ac-[AzaGly7,Arg(Me)9,Trp10]MS10
Ac-Asn-Ser-Phe-AzaGly-Leu-Arg(Me)-Trp-NH₂
Compound No. 511:
des(1)-[D-Tyr2,D-Trp3,Ala4,Thr5,AzaGly7,Arg(Me)9,
Trp10]MS10
D-Tyr-D-Trp-Ala-Thr-Phe-AzaGly-Leu-Arg(Me)-Trp-NH₂
Compound No. 512:
des(1)-[D-Tyr2,D-Trp3,Thr5,AzaGly7,Arg(Me)9,
Trp10]MS10
D-Tyr-D-Trp-Asn-Thr-Phe-AzaGly-Leu-Arg(Me)-Trp-NH₂
Compound No. 513:
des(1)-[D-Tyr2,D-Trp3,Abu4,AzaGly7,Arg(Me)9,
Trp10]MS10
D-Tyr-D-Trp-Abu-Ser-Phe-AzaGly-Leu-Arg(Me)-Trp-NH₂
Compound No. 514:
des(1)-[D-Tyr2,D-Phe3,Ala4,AzaGly7,Arg(Me)9,
Trp10]MS10
D-Tyr-D-Phe-Ala-Ser-Phe-AzaGly-Leu-Arg(Me)-Trp-NH₂
Compound No. 515:
des(1)-[D-Tyr2,D-Pya(4)3,Val5,AzaGly7,Arg(Me)9,
Trp10]MS10
D-Tyr-D-Pya(4)-Asn-Val-Phe-AzaGly-Leu-Arg(Me)-
Trp-NH₂
Compound No. 516:
des(1)-Ac-[D-Tyr2,D-Pya(4)3,AzaGly7,Arg(Me)9]MS10
Ac-D-Tyr-D-Pya(4)-Asn-Ser-Phe-AzaGly-Leu-Arg(Me)-
Phe-NH₂
Compound No. 517:
des(1-3)-3-Phenylpropionyl-[Hyp5,AzaGly7,Arg(Me)9,
Trp10]MS10
3-Phenylpropionyl-Asn-Hyp-Phe-AzaGly-Leu-Arg(Me)-
Trp-NH₂
Compound No. 518:
des(1-3)-3-Phenylpropionyl-[Cha6,Arg(Me)9,
Trp10]MS10
3-Phenylpropionyl-Asn-Ser-Cha-Gly-Leu-Arg(Me)-Trp-
NH₂
Compound No. 519:
des(1-3)-Phenylacetyl-[AzaGly7,Arg(Me)9,Trp10]MS10
Phenylacetyl-Asn-Ser-Phe-AzaGly-Leu-Arg(Me)-Trp-
NH₂
Compound No. 521:
des(1)-[D-Tyr2,D-Pya(4)3,AzaGly7]MS10
D-Tyr-D-Pya(4)-Asn-Ser-Phe-AzaGly-Leu-Arg-Phe-NH₂
Compound No. 522:
des(1-3)-Benzoyl-[Thr5,AzaGly7,Arg(Me)9,Trp10]MS10
Benzoyl-Asn-Thr-Phe-AzaGly-Leu-Arg(Me)-Trp-NH₂
Compound No. 523:
des(1-3)-Benzoyl-[Thr5,Phe(4F)6,AzaGly7,Arg(Me)9,
Trp10]MS10
Benzoyl-Asn-Thr-Phe(4F)-AzaGly-Leu-Arg(Me)-Trp-NH₂
Compound No. 524:
des(1-3)-3-Phenylpropionyl-[Pro5,AzaGly7,Arg(Me)9,
Trp10]MS10
3-Phenylpropionyl-Asn-Pro-Phe-AzaGly-Leu-Arg(Me)-
Trp-NH₂
Compound No. 527:
des(1)-[D-Tyr2,D-Pya(4)3,Hyp5,AzaGly7,Arg(Me)9,
Trp10]MS10
D-Tyr-D-Pya(4)-Asn-Hyp-Phe-AzaGly-Leu-Arg(Me)-Trp-
NH₂
Compound No. 528:
des(1)-[D-Tyr2,D-Pya(4)3,Pro5,AzaGly7,Arg(Me)9,
Trp10]MS10
D-Tyr-D-Pya(4)-Asn-Pro-Phe-AzaGly-Leu-Arg(Me)-Trp-
NH₂
Compound No. 529:
des(1)-[D-Tyr2,D-Pya(4)3,Tle5,AzaGly7,Arg(Me)9,
Trp10]MS10
D-Tyr-D-Pya(4)-Asn-Tle-Phe-AzaGly-Leu-Arg(Me)-Trp-
NH₂
Compound No. 530:
des(1)-[D-Tyr2,D-Pya(4)3,Phg5,AzaGly7,Arg(Me)9,
Trp10]MS10
D-Tyr-D-Pya(4)-Asn-Phg-Phe-AzaGly-Leu-Arg(Me)-Trp-
NH₂
Compound No. 531:
des(1-3)-3-Phenylpropionyl-[Pic(2)5,AzaGly7,
Arg(Me)9,Trp10]MS10
3-Phenylpropionyl-Asn-Pic(2)-Phe-AzaGly-Leu-
Arg(Me)-Trp-NH₂
Compound No. 532:
des(1-3)-3-Phenylpropionyl-[Aze(2)5,AzaGly7,
Arg(Me)9,Trp10]MS10
3-Phenylpropionyl-Asn-Aze(2)-Phe-AzaGly-Leu-
Arg(Me)-Trp-NH₂
Compound No. 533:
des(1-3)-3-Phenylpropionyl-[D-Pro5,AzaGly7,
Arg(Me)9,Trp10]MS10
3-Phenylpropionyl-Asn-D-Pro-Phe-AzaGly-Leu-
Arg(Me)-Trp-NH₂
Compound No. 534:
des(1-3)-Cyclopropanecarbonyl-[AzaGly7,
Arg(Me)9,Trp10]MS10
Cyclopropanecarbonyl-Asn-Ser-Phe-AzaGly-Leu-
Arg(Me)-Trp-NH₂
Compound No. 535:
des(1-3)-2-Naphthoyl-[AzaGly7,Arg(Me)9,Trp10]MS10
2-Naphthoyl-Asn-Ser-Phe-AzaGly-Leu-Arg(Me)-Trp-NH₂
Compound No. 536:
[Arg1,D-Tyr2,D-Pya(4)3,AzaGly7,Arg(Me)9,Trp10]MS10
Arg-D-Tyr-D-Pya(4)-Asn-Ser-Phe-AzaGly-Leu-Arg(Me)-
Trp-NH₂
Compound No. 537:
Arg-[Arg1,D-Tyr2,D-Pya(4)3,AzaGly7,Arg(Me)9,
Trp10]MS10
Arg-Arg-D-Tyr-D-Pya(4)-Asn-Ser-Phe-AzaGly-Leu-
Arg(Me)-Trp-NH₂
Compound No. 538:
Arg-[Acp1,D-Tyr2,D-Pya(4)3,AzaGly7,Arg(Me)9,
Trp10]MS10
Arg-Acp-D-Tyr-D-Pya(4)-Asn-Ser-Phe-AzaGly-Leu-
Arg(Me)-Trp-NH₂
Compound No. 539:
des(1)-[D-Tyr2,D-Trp3,Val5,AzaGly7,Arg(Me)9,
Trp10]MS10
D-Tyr-D-Trp-Asn-Val-Phe-AzaGly-Leu-Arg(Me)-Trp-NH₂
Compound No. 540:
des(1)-[D-Tyr2,D-Trp3,AzaGly7,Arg(Me)9,Trp10]MS10
D-Tyr-D-Trp-Asn-Ser-Phe-AzaGly-Leu-Arg(Me)-Trp-NH₂
Compound No. 541:
D-Arg-[Acp1,D-Tyr2,D-Trp3,Thr5,AzaGly7,Arg(Me)9,
Trp10]MS10
D-Arg-Acp-D-Tyr-D-Trp-Asn-Thr-Phe-AzaGly-Leu-
Arg(Me)-Trp-NH₂
Compound No. 542:
D-Arg-D-Arg-[Acp1,D-Tyr2,D-Trp3,Thr5,AzaGly7,
Arg(Me)9,Trp10]MS10
D-Arg-D-Arg-Acp-D-Tyr-D-Trp-Asn-Thr-Phe-AzaGly-
Leu-Arg(Me)-Trp-NH₂
Compound No. 545:
des(1-3)-Benzoyl-[Phe(4F)6,AzaGly7,Arg(Me)9,
Trp10]MS10
Benzoyl-Asn-Ser-Phe(4F)-AzaGly-Leu-Arg(Me)-Trp-NH₂
Compound No. 546:
des(1-3)-3-Phenylpropionyl-[Ser(Ac)5,AzaGly7,
Arg(Me)9,Trp10]MS10
3-Phenylpropionyl-Asn-Ser(Ac)-Phe-AzaGly-Leu-
Arg(Me)-Trp-NH₂
Compound No. 547:
des(1)-[D-Tyr2,D-Pya(4)3,Ser(Ac)5,AzaGly7,Arg(Me)9,
Trp10]MS10
D-Tyr-D-Pya(4)-Asn-Ser(Ac)-Phe-AzaGly-Leu-Arg(Me)-
Trp-NH₂
Compound No. 548:
des(1)-[D-Tyr2,D-Pya(4)3,AzaGly7,Arg(Me)9,10Ψ,
CSNH]MS10
D-Tyr-D-Pya(4)-Asn-Ser-Phe-AzaGly-Leu-Arg(Me)-PheΨ,
(CSNH)NH₂
Compound No. 550:
des(1)-Ac-[D-Tyr2,D-Trp3,Thr5,AzaGly7,Arg(Me)9,
Trp10]MS10
Ac-D-Tyr-D-Trp-Asn-Thr-Phe-AzaGly-Leu-Arg(Me)-Trp-
NH₂
Compound No. 551:
Ac-D-Arg-[Acp1,D-Tyr2,D-Trp3,Thr5,AzaGly7,
Arg(Me)9,Trp10]MS10
Ac-D-Arg-Acp-D-Tyr-D-Trp-Asn-Thr-Phe-AzaGly-Leu-
Arg(Me)-Trp-NH₂
Compound No. 552:
D-Dap-[Acp1,D-Tyr2,D-Trp3,Thr5,AzaGly7,Arg(Me)9,
Trp10]MS10
D-Dap-Acp-D-Tyr-D-Trp-Asn-Thr-Phe-AzaGly-Leu-
Arg(Me)-Trp-NH₂
Compound No. 553:
D-Nle-[Acp1,D-Tyr2,D-Trp3,Thr5,AzaGly7,Arg(Me)9,
Trp10]MS10
D-Nle-Acp-D-Tyr-D-Trp-Asn-Thr-Phe-AzaGly-Leu-
Arg(Me)-Trp-NH₂
Compound No. 554:
D-Arg-[β-Ala1,D-Tyr2,D-Trp3,Thr5,AzaGly7,Arg(Me)9,
Trp10]MS10
D-Arg-β-Ala-D-Tyr-D-Trp-Asn-Thr-Phe-AzaGly-Leu-
Arg(Me)-Trp-NH₂
Compound No. 555:
D-Arg-[γ-Abu1,D-Tyr2,D-Trp3,Thr5,AzaGly7,Arg(Me)9,
Trp10]MS10
D-Arg-γ-Abu-D-Tyr-D-Trp-Asn-Thr-Phe-AzaGly-Leu-
Arg(Me)-Trp-NH₂
Compound No. 556:
D-Arg-D-Arg-[γ-Abu1,D-Tyr2,D-
Trp3,Thr5,AzaGly7,Arg(Me)9,Trp10]MS10
D-Arg-D-Arg-γ-Abu-D-Tyr-D-Trp-Asn-Thr-Phe-AzaGly-
Leu-Arg(Me)-Trp-NH₂
Compound No. 557:
D-Arg-D-Arg-D-Arg-[γ-Abu1,D-Tyr2,D-
Trp3,Thr5,AzaGly7,Arg(Me)9,Trp10]MS10
D-Arg-D-Arg-D-Arg-γ-Abu-D-Tyr-D-Trp-Asn-Thr-Phe-
AzaGly-Leu-Arg(Me)-Trp-NH₂
Compound No. 558:
des(1)-Ac-[D-Tyr2,D-Trp3,AzaGly7,Arg(Me)9,
Trp10]MS10
D-Tyr-D-Trp-Asn-Ser-Phe-AzaGly-Leu-Arg(Me)-Trp-NH₂
Compound No. 559:
des(1-2)-3-(4-Hydroxyphenyl)propionyl-[D-
Trp3,Thr5,AzaGly7,Arg(Me)9,Trp10]MS10
3-(4-Hydroxyphenyl)propionyl-D-Trp-Asn-Thr-Phe-
AzaGly-Leu-Arg(Me)-Trp-NH₂
Compound No. 561:
D-Arg-[Acp1,D-Tyr2,D-Trp3,Abu4,AzaGly7,Arg(Me)9,
Trp10]MS10
D-Arg-Acp-D-Tyr-D-Trp-Abu-Ser-Phe-AzaGly-Leu-
Arg(Me)-Trp-NH₂
Compound No. 562:
des(1)-Ac-[D-Tyr2,D-Pya(4)3,Thr5,AzaGly7,Arg(Me)9,
Trp10]MS10
Ac-D-Tyr-D-Pya(4)-Asn-Thr-Phe-AzaGly-Leu-Arg(Me)-
Trp-NH₂
Compound No. 563:
des(1)-Ac-[D-Tyr2,D-Trp3,Aze(2)5,AzaGly7,Arg(Me)9,
Trp10]MS10
Ac-D-Tyr-D-Trp-Asn-Aze(2)-Phe-AzaGly-Leu-Arg(Me)-
Trp-NH₂
Compound No. 564:
des(1)-Ac-[D-Tyr2,D-Trp3,Val5,AzaGly7,Arg(Me)9,
Trp10]MS10
Ac-D-Tyr-D-Trp-Asn-Val-Phe-AzaGly-Leu-Arg(Me)-Trp-
NH₂
Compound No. 565:
des(1)-Benzoyl-[D-Tyr2,D-Trp3,Thr5,AzaGly7,
Arg(Me)9,Trp10]MS10
Benzoyl-D-Tyr-D-Trp-Asn-Thr-Phe-AzaGly-Leu-
Arg(Me)-Trp-NH₂
Compound No. 566:
des(1)-Cyclopropanecarbonyl-[D-Tyr2,D-
Trp3,Thr5,AzaGly7,Arg(Me)9,Trp10]MS10
Cyclopropanecarbonyl-D-Tyr-D-Trp-Asn-Thr-Phe-
AzaGly-Leu-Arg(Me)-Trp-NH₂
Compound No. 567:
des(1)-Butyryl-[D-Tyr2,D-Trp3,Thr5,AzaGly7,
Arg(Me)9,Trp10]MS10
Butyryl-D-Tyr-D-Trp-Asn-Thr-Phe-AzaGly-Leu-
Arg(Me)-Trp-NH₂
Compound No. 568:
Ac-[D-Arg1,D-Tyr2,D-Trp3,Thr5,AzaGly7,Arg(Me)9,
Trp10]MS10
Ac-D-Arg-D-Tyr-D-Trp-Asn-Thr-Phe-AzaGly-Leu-
Arg(Me)-Trp-NH₂
Compound No. 569:
des(1)-Ac-[D-Tyr2,D-Trp3,Thr5,6Ψ7,CH2NH,
Arg(Me)9,Trp10]MS10
Ac-D-Tyr-D-Trp-Asn-Thr-PheΨ(CH₂NH)Gly-Leu-Arg(Me)-
Trp-NH₂
Compound No. 570:
des(1)-Me-[D-Tyr2,D-Trp3,Thr5,AzaGly7,Arg(Me)9,
Trp10]MS10
Me-D-Tyr-D-Trp-Asn-Thr-Phe-AzaGly-Leu-Arg(Me)-Trp-
NH₂
Compound No. 571:
des(1)-Ac-[D-Tyr2,D-Trp3,Thr5,AzaGly7,
Arg(Me)9]MS10
Ac-D-Tyr-D-Trp-Asn-Thr-Phe-AzaGly-Leu-Arg(Me)-Phe-
NH₂
Compound No. 572:
des(1)-[D-Trp2,D-Pya(4)3,AzaGly7,Arg(Me)9,
Trp10]MS10
D-Trp-D-Pya(4)-Asn-Ser-Phe-AzaGly-Leu-Arg(Me)-Trp-
NH₂
Compound No. 573:
des(1)-Ac-[D-Tyr2,D-Trp3,Abu4,AzaGly7,Arg(Me)9,
Trp10]MS10
Ac-D-Tyr-D-Trp-Abu-Ser-Phe-AzaGly-Leu-Arg(Me)-Trp-
NH₂
Compound No. 576:
des(1)-Ac-[D-Tyr2,D-Trp3,Gln4,AzaGly7,Arg(Me)9,
Trp10]MS10
Ac-D-Tyr-D-Trp-Gln-Ser-Phe-AzaGly-Leu-Arg(Me)-Trp-
NH₂
Compound No. 577:
des(1)-Ac-[D-Tyr2,D-Trp3,Ser4,AzaGly7,Arg(Me)9,
Trp10]MS10
Ac-D-Tyr-D-Trp-Ser-Ser-Phe-AzaGly-Leu-Arg(Me)-Trp-
NH₂
Compound No. 578:
des(1)-Ac-[D-Tyr2,D-Trp3,Thr4,AzaGly7,Arg(Me)9,
Trp10]MS10
Ac-D-Tyr-D-Trp-Thr-Ser-Phe-AzaGly-Leu-Arg(Me)-Trp-
NH₂
Compound No. 579:
des(1)-Ac-[D-Tyr2,D-Trp3,Alb4,AzaGly7,Arg(Me)9,
Trp10]MS10
Ac-D-Tyr-D-Trp-Alb-Ser-Phe-AzaGly-Leu-Arg(Me)-Trp-
NH₂
Compound No. 580:
des(1)-Ac-[D-Tyr2,D-Trp3,Ser(Me)5,AzaGly7,
Arg(Me)9,Trp10]MS10
Ac-D-Tyr-D-Trp-Asn-Ser(Me)-Phe-AzaGly-Leu-Arg(Me)-
Trp-NH₂
Compound No. 584:
des(1)-Ac-[D-Tyr2,D-Trp3,Dap(Ac)4,AzaGly7,
Arg(Me)9,Trp10]MS10
Ac-D-Tyr-D-Trp-Dap(Ac)-Ser-Phe-AzaGly-Leu-Arg(Me)-
Trp-NH₂
Compound No. 585:
des(1)-Ac-[D-Tyr2,D-Trp3,Dap(For)4,AzaGly7,
Arg(Me)9,Trp10]MS10
Ac-D-Tyr-D-Trp-Dap(For)-Ser-Phe-AzaGly-Leu-
Arg(Me)-Trp-NH₂
Compound No. 586:
des(1)-Ac-[D-Tyr2,Thr5,D-Phe6,AzaGly7,Arg(Me)9,
Trp10]MS10
Ac-D-Tyr-Trp-Asn-Thr-D-Phe-AzaGly-Leu-Arg(Me)-Trp-
NH₂
Compound No. 589:
des(1)-Ac-[D-Tyr2,D-Trp3,Thr5,AzaGly7,Arg(Me)9,
Nal(2)10]MS10
Ac-D-Tyr-D-Trp-Asn-Thr-Phe-AzaGly-Leu-Arg(Me)-
Nal(2)-NH₂
Compound No. 590:
des(1)-Ac-[D-Tyr2,D-Trp3,Thr5,AzaGly7,Arg(Me)9,
Thi(2)10]MS10
Ac-D-Tyr-D-Trp-Asn-Thr-Phe-AzaGly-Leu-Arg(Me)-
Thi-NH₂
Compound No. 591:
des(1)-Ac-[D-Tyr2,D-Trp3,Thr5,AzaGly7,Arg(Me)9,
Tyr10]MS10
Ac-D-Tyr-D-Trp-Asn-Thr-Phe-AzaGly-Leu-Arg(Me)-Tyr-
NH₂
Compound No. 592:
des(1)-Ac-[D-Tyr2,D-Trp3,Thr5,AzaGly7,Arg(Me)9,
Phe(4F)10]MS10
Ac-D-Tyr-D-Trp-Asn-Thr-Phe-AzaGly-Leu-Arg(Me)-
Phe(4F)-NH₂
Compound No. 594:
des(1)-Ac-[D-Tyr2,D-Trp3,Thr5,AzaGly7,Arg(Me)9,
Hph10]MS10
Ac-D-Tyr-D-Trp-Asn-Thr-Phe-AzaGly-Leu-Arg(Me)-Hph-
NH₂
Compound No. 595:
des(1)-Ac-[D-Tyr2,D-Trp3,Thr5,AzaGly7,Arg(Me)9,
Cha10]MS10
Ac-D-Tyr-D-Trp-Asn-Thr-Phe-AzaGly-Leu-Arg(Me)-Cha-
NH₂
Compound No. 596:
des(1)-Ac-[D-Tyr2,D-Trp3,Thr5,AzaGly7,Arg(Me)9,
Leu10]MS10
Ac-D-Tyr-D-Trp-Asn-Thr-Phe-AzaGly-Leu-Arg(Me)-Leu-
NH₂
Compound No. 597:
des(1)-Ac-[D-Tyr2,D-Trp3,Thr5,D-Phe6,AzaGly7,
Arg(Me)9,Trp10]MS10
Ac-D-Tyr-D-Trp-Asn-Thr-D-Phe-AzaGly-Leu-Arg(Me)-
Trp-NH₂
Compound No. 598:
des(1)-Ac-[D-Tyr2,D-Trp3,Thr5,Arg(Me)9,Trp10]MS10
Ac-D-Tyr-D-Trp-Asn-Thr-Phe-Gly-Leu-Arg(Me)-Trp-NH₂
Compound No. 599:
des(1)-Ac-[D-Tyr2,D-Trp3,Thr5,AzaGly7,Orn9,
Trp10]MS10
Ac-D-Tyr-D-Trp-Asn-Thr-Phe-AzaGly-Leu-Orn-Trp-NH₂
Compound No. 600:
des(1)-Ac-[D-Tyr2,D-Trp3,Thr5,AzaGly7,Trp10]MS10
Ac-D-Tyr-D-Trp-Asn-Thr-Phe-AzaGly-Leu-Arg-Trp-NH₂
Compound No. 601:
des(1)-Ac-[D-Tyr2,D-Trp3,Thr5,D-Phe6,Arg(Me)9,
Trp10]MS10
Ac-D-Tyr-D-Trp-Asn-Thr-D-Phe-Gly-Leu-Arg(Me)-Trp-
NH₂
Compound No. 602:
des(1)-Ac-[D-NMeTyr2,D-Trp3,Thr5,AzaGly7,Arg(Me)9,
Trp10]MS10
Ac-D-NMeTyr-D-Trp-Asn-Thr-Phe-AzaGly-Leu-Arg(Me)-
Trp-NH₂
Compound No. 603:
des(1)-Ac-[D-Tyr2,D-Pya(4)3,Thr5,D-
Phe6,AzaGly7,Arg(Me)9,Trp10]MS10
Ac-D-Tyr-D-Pya(4)-Asn-Thr-D-Phe-AzaGly-Leu-
Arg(Me)-Trp-NH₂
Compound No. 604:
des(1)-Ac-[D-Tyr2,D-Trp3,Thr5,AzaGly7,Arg(Tos)9,
Trp10]MS10
Ac-D-Tyr-D-Trp-Asn-Thr-Phe-AzaGly-Leu-Arg(Tos)-
Trp-NH₂
Compound No. 605:
des(1)-Ac-[D-Tyr2,D-Trp3,Thr5,AzaGly7,Arg(NO2)9,
Trp10]MS10
Ac-D-Tyr-D-Trp-Asn-Thr-Phe-AzaGly-Leu-Arg(NO2)-
Trp-NH₂
Compound No. 607:
des(1)-Ac-[D-Tyr2,D-Trp3,Thr5,AzaGly7,
Arg(Me2)asym9,Trp10]MS10
Ac-D-Tyr-D-Trp-Asn-Thr-Phe-AzaGly-Leu-
Arg(Me2)asym-Trp-NH₂
Compound No. 608:
des(1)-Ac-[D-Tyr2,D-Trp3,Thr5,AzaGly7,
Arg(Me2)sym9,Trp10]MS10
Ac-D-Tyr-D-Trp-Asn-Thr-Phe-AzaGly-Leu-
Arg(Me2)sym-Trp-NH₂
Compound No. 609:
des(1)-Ac-[D-Tyr2,D-Trp3,Thr5,AzaGly7,Arg(Et)9,
Trp10]MS10
Ac-D-Tyr-D-Trp-Asn-Thr-Phe-AzaGly-Leu-Arg(Et)-
Trp-NH₂
Compound No. 610:
des(1)-Ac-[D-Tyr2,D-Trp3,Thr5,AzaGly7,Lys(Me2)9,
Trp10]MS10
Ac-D-Tyr-D-Trp-Asn-Thr-Phe-AzaGly-Leu-Lys(Me2)-
Trp-NH₂
Compound No. 611:
des(1)-Ac-[Tyr2,D-Pya(4)3,Thr5,AzaGly7,Arg(Me)9,
Trp10]MS10
Ac-Tyr-D-Pya(4)-Asn-Thr-Phe-AzaGly-Leu-Arg(Me)-
Trp-NH₂
Compound No. 612:
des(1)-For-[D-Tyr2,D-Trp3,Thr5,AzaGly7,Arg(Me)9,
Trp10]MS10
For-D-Tyr-D-Trp-Asn-Thr-Phe-AzaGly-Leu-Arg(Me)-
Trp-NH₂
Compound No. 613:
des(1)-Propionyl-[D-Tyr2,D-Trp3,Thr5,AzaGly7,
Arg(Me)9,Trp10]MS10
Propionyl-D-Tyr-D-Trp-Asn-Thr-Phe-AzaGly-Leu-
Arg(Me)-Trp-NH₂
Compound No. 614:
des(1)-Amidino-[D-Tyr2,D-Trp3,Thr5,AzaGly7,
Arg(Me)9,Trp10]MS10
Amidino-D-Tyr-D-Trp-Asn-Thr-Phe-AzaGly-Leu-
Arg(Me)-Trp-NH₂
Compound No. 615:
des(1)-Ac-[Tyr2,D-Pya(4)3,Thr5,AzaGly7,Arg(Me)9,
Trp10]MS10
Ac-Tyr-D-Pya(4)-Asn-Thr-Phe-AzaGly-Leu-Arg(Me)-
Trp-NH₂
Compound No. 616:
des(1)-Ac-[D-Ala2,D-Trp3,Thr5,AzaGly7,Arg(Me)9,
Trp10]MS10
Ac-D-Ala-D-Trp-Asn-Thr-Phe-AzaGly-Leu-Arg(Me)-
Trp-NH₂
Compound No. 617:
des(1)-Ac -[D-Leu2,D-Trp3,Thr5,AzaGly7,Arg(Me)9,
Trp10]MS10
Ac-D-Leu-D-Trp-Asn-Thr-Phe-AzaGly-Leu-Arg(Me)-
Trp-NH₂
Compound No. 618:
des(1)-Ac-[D-Phe2,D-Trp3,Thr5,AzaGly7,Arg(Me)9,
Trp10]MS10
Ac-D-Phe-D-Trp-Asn-Thr-Phe-AzaGly-Leu-Arg(Me)-
Trp-NH₂
Compound No. 619:
des(1)-Ac-[D-Nal(1)2,D-Trp3,Thr5,AzaGly7,
Arg(Me)9,Trp10]MS10
Ac-D-Nal(1)-D-Trp-Asn-Thr-Phe-AzaGly-Leu-
Arg(Me)-Trp-NH₂
Compound No. 620:
des(1)-Ac-[D-Nal(2)2,D-Trp3,Thr5,AzaGly7,
Arg(Me)9,Trp10]MS10
Ac-D-Nal(2)-D-Trp-Asn-Thr-Phe-AzaGly-Leu-
Arg(Me)-Trp-NH₂
Compound No. 621:
des(1)-Ac-[D-Lys2,D-Trp3,Thr5,AzaGly7,Arg(Me)9,
Trp10]MS10
Ac-D-Lys-D-Trp-Asn-Thr-Phe-AzaGly-Leu-Arg(Me)-
Trp-NH₂
Compound No. 622:
des(1)-Ac-[D-Glu2,D-Trp3,Thr5,AzaGly7,Arg(Me)9,
Trp10]MS10
Ac-D-Glu-D-Trp-Asn-Thr-Phe-AzaGly-Leu-Arg(Me)-
Trp-NH₂
Compound No. 623:
des(1)-Ac-[D-Tyr2,Thr5,AzaGly7,Arg(Me)9,
Trp10]MS10
Ac-D-Tyr-Trp-Asn-Thr-Phe-AzaGly-Leu-Arg(Me)-
Trp-NH₂
Compound No. 624:
des(1)-Ac-[D-Tyr2,Pya(4)3,Thr5,AzaGly7,Arg(Me)9,
Trp10]MS10
Ac-D-Tyr-Pya(4)-Asn-Thr-Phe-AzaGly-Leu-Arg(Me)-
Trp-NH₂
Compound No. 625:
des(1)-Ac-[D-Tyr2,D-Ala3,Thr5,AzaGly7,Arg(Me)9,
Trp10]MS10
Ac-D-Tyr-D-Ala-Asn-Thr-Phe-AzaGly-Leu-Arg(Me)-
Trp-NH₂
Compound No. 626:
des(1)-Ac-[D-Tyr2,D-Leu3,Thr5,AzaGly7,Arg(Me)9,
Trp10]MS10
Ac-D-Tyr-D-Leu-Asn-Thr-Phe-AzaGly-Leu-Arg(Me)-
Trp-NH₂
Compound No. 627:
des(1)-Ac-[D-Tyr2,D-Phe3,Thr5,AzaGly7,Arg(Me)9,
Trp10]MS10
Ac-D-Tyr-D-Phe-Asn-Thr-Phe-AzaGly-Leu-Arg(Me)-
Trp-NH₂
Compound No. 628:
des(1)-Ac-[D-Tyr2,D-Thr3,Thr5,AzaGly7,Arg(Me)9,
Trp10]MS10
Ac-D-Tyr-D-Thr-Asn-Thr-Phe-AzaGly-Leu-Arg(Me)-
Trp-NH₂
Compound No. 629:
des(1)-Ac-[D-Tyr2,D-Lys3,Thr5,AzaGly7,Arg(Me)9,
Trp10]MS10
Ac-D-Tyr-D-Lys-Asn-Thr-Phe-AzaGly-Leu-Arg(Me)-
Trp-NH₂
Compound No. 630:
des(1)-Ac-[D-Tyr2,D-Glu3,Thr5,AzaGly7,Arg(Me)9,
Trp10]MS10
Ac-D-Tyr-D-Glu-Asn-Thr-Phe-AzaGly-Leu-Arg(Me)-
Trp-NH₂
Compound No. 631:
des(1)-Ac-[D-Tyr2,D-Trp3,Thr5,Ala6,AzaGly7,
Arg(Me)9,Trp10]MS10
Ac-D-Tyr-D-Trp-Asn-Thr-Ala-AzaGly-Leu-Arg(Me)-
Trp-NH₂
Compound No. 632:
des(1)-Ac-[D-Tyr2,D-Trp3,Thr5,Leu6,AzaGly7,
Arg(Me)9,Trp10]MS10
Ac-D-Tyr-D-Trp-Asn-Thr-Leu-AzaGly-Leu-Arg(Me)-
Trp-NH₂
Compound No. 633:
des(1)-Ac-[D-Tyr2,D-Trp3,Thr5,Lys6,AzaGly7,
Arg(Me)9,Trp10]MS10
Ac-D-Tyr-D-Trp-Asn-Thr-Lys-AzaGly-Leu-Arg(Me)-
Trp-NH₂
Compound No. 634:
des(1)-Ac-[D-Tyr2,D-Trp3,Thr5,Glu6,AzaGly7,
Arg(Me)9,Trp10]MS10
Ac-D-Tyr-D-Trp-Asn-Thr-Glu-AzaGly-Leu-Arg(Me)-
Trp-NH₂
Compound No. 635:
des(1)-Ac-[D-Tyr2,D-Trp3,Thr5,Pya(4)6,AzaGly7,
Arg(Me)9,Trp10]MS10
Ac-D-Tyr-D-Trp-Asn-Thr-Pya(4)-AzaGly-Leu-
Arg(Me)-Trp-NH₂
Compound No. 636:
des(1)-Ac-[D-Tyr2,D-Trp3,Thr5,NMePhe6,AzaGly7,
Arg(Me)9,Trp10]MS10
Ac-D-Tyr-D-Trp-Asn-Thr-NMePhe-AzaGly-Leu-
Arg(Me)-Trp-NH₂
Compound No. 637:
des(1)-Ac-[D-Tyr2,D-Trp3,Thr5,Phe(4F)6,AzaGly7,
Arg(Me)9,Trp10]MS10
Ac-D-Tyr-D-Trp-Asn-Thr-Phe(4F)-AzaGly-Leu-
Arg(Me)-Trp-NH₂
Compound No. 638:
des(1)-Ac-[D-Tyr2,D-Pya(4)3,Thr5,Phe(4F)6,
AzaGly7,Arg(Me)9,Trp10]MS10
Ac-D-Tyr-D-Pya(4)-Asn-Thr-Phe(4F)-AzaGly-Leu-
Arg(Me)-Trp-NH₂
Compound No. 639:
des(1)-Ac-[D-Tyr2,D-Trp3,Thr5,AzaGly7,Lys9,
Trp10]MS10
Ac-D-Tyr-D-Trp-Asn-Thr-Phe-AzaGly-Leu-Lys-Trp-
NH₂
Compound No. 641:
des(1)-Ac-[D-Tyr2,D-Trp3,Thr5,AzaGly7,Ala8,
Arg(Me)9,Trp10]MS10
Ac-D-Tyr-D-Trp-Asn-Thr-Phe-AzaGly-Ala-Arg(Me)-
Trp-NH₂
Compound No. 642:
des(1)-Ac-[D-Tyr2,D-Trp3,Thr5,AzaGly7,Val8,
Arg(Me)9,Trp10]MS10
Ac-D-Tyr-D-Trp-Asn-Thr-Phe-AzaGly-Val-Arg(Me)-
Trp-NH₂
Compound No. 643:
des(1)-Ac-[D-Tyr2,D-Trp3,Thr5,AzaGly7,Phe8,
Arg(Me)9,Trp10]MS10
Ac-D-Tyr-D-Trp-Asn-Thr-Phe-AzaGly-Phe-Arg(Me)-
Trp-NH₂
Compound No. 644:
des(1)-Ac-[D-Tyr2,D-Trp3,Thr5,AzaGly7,Ser8,
Arg(Me)9,Trp10]MS10
Ac-D-Tyr-D-Trp-Asn-Thr-Phe-AzaGly-Ser-Arg(Me)-
Trp-NH₂
Compound No. 645:
des(1)-Ac-[D-Tyr2,D-Trp3,Thr5,AzaGly7,Har9,
Trp10]MS10
Ac-D-Tyr-D-Trp-Asn-Thr-Phe-AzaGly-Leu-Har-Trp-
NH₂
Compound No. 646:
des(1)-Ac-[D-Tyr2,D-Trp3,Thr5,AzaGly7,Har(Me)9,
Trp10]MS10
Ac-D-Tyr-D-Trp-Asn-Thr-Phe-AzaGly-Leu-Har(Me)-
Trp-NH₂
Compound No. 647:
des(1)-Ac-[D-Tyr2,D-Trp3,Asp4,Thr5,AzaGly7,
Arg(Me)9,Trp10]MS10
Ac-D-Tyr-D-Trp-Asp-Thr-Phe-AzaGly-Leu-Arg(Me)-
Trp-NH₂
Compound No. 648:
[Gly1,D-Tyr2,D-Trp3,Thr5,AzaGly7,Arg(Me)9,
Trp10]MS10
Gly-D-Tyr-D-Trp-Asn-Thr-Phe-AzaGly-Leu-Arg(Me)-
Trp-NH₂
Compound No. 649:
Ac-[Gly1,D-Tyr2,D-Trp3,Thr5,AzaGly7,Arg(Me)9,
Trp10]MS10
Ac-Gly-D-Tyr-D-Trp-Asn-Thr-Phe-AzaGly-Leu-
Arg(Me)-Trp-NH₂
Compound No. 650:
[D-Tyr1,D-Tyr2,D-Trp3,Thr5,AzaGly7,Arg(Me)9,
Trp10]MS10
D-Tyr-D-Tyr-D-Trp-Asn-Thr-Phe-AzaGly-Leu-
Arg(Me)-Trp-NH₂
Compound No. 651:
Ac-[D-Tyr1,D-Tyr2,D-Trp3,Thr5,AzaGly7,Arg(Me)9,
Trp10]MS10
Ac-D-Tyr-D-Tyr-D-Trp-Asn-Thr-Phe-AzaGly-Leu-
Arg(Me)-Trp-NH₂
Compound No. 652:
pGlu-des(1)-[D-Tyr2,D-Trp3,Thr5,AzaGly7,
Arg(Me)9,Trp10]MS10
pGlu-D-Tyr-D-Trp-Asn-Thr-Phe-AzaGly-Leu-
Arg(Me)-Trp-NH₂
Compound No. 653:
des(1)-Ac-[D-Tyr2,D-Trp3,D-Asn4,Thr5,AzaGly7,
Arg(Me)9,Trp10]MS10
Ac-D-Tyr-D-Trp-D-Asn-Thr-Phe-AzaGly-Leu-
Arg(Me)-Trp-NH₂
Compound No. 654:
des(1)-Ac-[D-Tyr2,D-Trp3,D-Thr5,AzaGly7,
Arg(Me)9,Trp10]MS10
Ac-D-Tyr-D-Trp-Asn-D-Thr-Phe-AzaGly-Leu-
Arg(Me)-Trp-NH₂
Compound No. 655:
des(1)-Ac-[D-Tyr2,D-Trp3,NMeAsn4,Thr5,AzaGly7,
Arg(Me)9,Trp10]MS10
Ac-D-Tyr-D-Trp-NMeAsn-Thr-Phe-AzaGly-Leu-
Arg(Me)-Trp-NH₂
Compound No. 656:
des(1)-Ac-[D-Tyr2,D-Trp3,NMeSer5,AzaGly7,
Arg(Me)9,Trp10]MS10
Ac-D-Tyr-D-Trp-Asn-NMeSer-Phe-AzaGly-Leu-
Arg(Me)-Trp-NH₂
Compound No. 657:
des(1)-Ac-[D-Tyr2,Pro3,Thr5,AzaGly7,Arg(Me)9,
Trp10]MS10
Ac-D-Tyr-Pro-Asn-Thr-Phe-AzaGly-Leu-Arg(Me)-
Trp-NH₂
Compound No. 658:
des(1)-Ac-[D-Tyr2,D-Pya(2)3,Thr5,AzaGly7,
Arg(Me)9,Trp10]MS10
Ac-D-Tyr-D-Pya(2)-Asn-Thr-Phe-AzaGly-Leu-
Arg(Me)-Trp-NH₂
Compound No. 659:
des(1)-Ac-[D-Tyr2,D-Trp3,allo-Thr5,AzaGly7,
Arg(Me)9,Trp10]MS10
Ac-D-Tyr-D-Trp-Asn-allo-Thr-Phe-AzaGly-Leu-
Arg(Me)-Trp-NH₂
Compound No. 660:
des(1)-Ac-[D-Tyr2,D-Pya(3)3,Thr5,AzaGly7,
Arg(Me)9,Trp10]MS10
Ac-D-Tyr-D-Pya(3)-Asn-Thr-Phe-AzaGly-Leu-
Arg(Me)-Trp-NH₂
Compound No. 661:
des(1)-Ac-[D-Tyr2,D-Pro3,Thr5,AzaGly7,Arg(Me)9,
Trp10]MS10
Ac-D-Tyr-D-Pro-Asn-Thr-Phe-AzaGly-Leu-Arg(Me)-
Trp-NH₂
Compound No. 662:
des(1)-Ac-[D-Tyr2,Tic3,Thr5,AzaGly7,Arg(Me)9,
Trp10]MS10
Ac-D-Tyr-Tic-Asn-Thr-Phe-AzaGly-Leu-Arg(Me)-Trp-
NH₂
Compound No. 663:
des(1)-Ac-[D-Trp2,D-Trp3,Thr5,AzaGly7,Arg(Me)9,
Trp10]MS10
Ac-D-Trp-D-Trp-Asn-Thr-Phe-AzaGly-Leu-Arg(Me)-
Trp-NH₂
Compound No. 664:
des(1)-Ac-[Tyr2,Thr5,AzaGly7,Arg(Me)9,Trp10]MS10
Ac-Tyr-Trp-Asn-Thr-Phe-AzaGly-Leu-Arg(Me)-Trp-NH₂
Compound No. 665:
des(1-2)-[D-Trp3,Thr5,AzaGly7,Arg(Me)9,Trp10]MS10
D-Trp-Asn-Thr-Phe-AzaGly-Leu-Arg(Me)-Trp-NH₂
Compound No. 666:
des(1-2)-Ac-[D-Trp3,Thr5,AzaGly7,Arg(Me)9,
Trp10]MS10
Ac-D-Trp-Asn-Thr-Phe-AzaGly-Leu-Arg(Me)-Trp-NH₂
Compound No. 667:
des(1-2)-Hexanoyl-[D-Trp3,Thr5,AzaGly7,Arg(Me)9,
Trp10]MS10
Hexanoyl-D-Trp-Asn-Thr-Phe-AzaGly-Leu-Arg(Me)-
Trp-NH₂
Compound No. 668:
des(1-2)-Cyclohexanecarbonyl-[D-Trp3,Thr5,
AzaGly7,Arg(Me)9,Trp10]MS10
Cyclohexanecarbonyl-D-Trp-Asn-Thr-Phe-AzaGly-
Leu-Arg(Me)-Trp-NH₂
Compound No. 669:
des(1-2)-Benzoyl-[D-Trp3,Thr5,AzaGly7,Arg(Me)9,
Trp10]MS10
Benzoyl-D-Trp-Asn-Thr-Phe-AzaGly-Leu-Arg(Me)-
Trp-NH₂
Compound No. 670:
des(1-2)-3-Pyridinepropionyl-[D-Trp3,Thr5,
AzaGly7,Arg(Me)9,Trp10]MS10
3-Pyridinepropionyl-D-Trp-Asn-Thr-Phe-AzaGly-
Leu-Arg(Me)-Trp-NH₂
Compound No. 671:
des(1-2)-Adipoyl-[D-Trp3,Thr5,AzaGly7,Arg(Me)9,
Trp10]MS10
Adipoyl-D-Trp-Asn-Thr-Phe-AzaGly-Leu-Arg(Me)-
Trp-NH₂
Compound No. 672:
des(1)-Ac-[D-Tyr2,NMeTrp3,Thr5,AzaGly7,Arg(Me)9,
Trp10]MS10
Ac-D-Tyr-NMeTrp-Asn-Thr-Phe-AzaGly-Leu-Arg(Me)-
Trp-NH₂
Compound No. 674:
des(1-2)-6-Aminocaproyl-[D-Trp3,Thr5,AzaGly7,
Arg(Me)9,Trp10]MS10
6-Aminocaproyl-D-Trp-Asn-Thr-Phe-AzaGly-Leu-
Arg(Me)-Trp-NH₂
Compound No. 675:
[D-Tyr2,D-Trp3,Thr5,AzaGly7,Arg(Me)9,Trp10]MS10
Tyr-D-Tyr-D-Trp-Asn-Thr-Phe-AzaGly-Leu-Arg(Me)-
Trp-NH₂
Compound No. 676:
Ac-[D-Tyr2,D-Trp3,Thr5,AzaGly7,Arg(Me)9,
Trp10]MS10
Ac-Tyr-D-Tyr-D-Trp-Asn-Thr-Phe-AzaGly-Leu-
Arg(Me)-Trp-NH₂
Compound No. 677:
Ac-des(1)-[D-Tyr2,D-Trp3,Thr5,AzaGly7,Nva8,
Arg(Me)9,Trp10]MS10
Ac-D-Tyr-D-Trp-Asn-Thr-Phe-AzaGly-Nva-Arg(Me)-
Trp-NH₂
Compound No. 678:
Ac-des(1)-[D-Tyr2,D-Trp3,Thr5,AzaGly7,Ile8,
Arg(Me)9,Trp10]MS10
Ac-D-Tyr-D-Trp-Asn-Thr-Phe-AzaGly-Ile-Arg(Me)-
Trp-NH₂
Compound No. 679:
des(1-2)-Amidino-[D-Trp3,Thr5,AzaGly7,Arg(Me)9,
Trp10]MS10
Amidino-D-Trp-Asn-Thr-Phe-AzaGly-Leu-Arg(Me)-
Trp-NH₂
Compound No. 680:
des(1-2)-Glycoloyl-[D-Trp3,Thr5,AzaGly7,
Arg(Me)9,Trp10]MS10
Glycoloyl-D-Trp-Asn-Thr-Phe-AzaGly-Leu-Arg(Me)-
Trp-NH₂
Compound No. 681:
des(1)-Glycoloyl-[D-Tyr2,D-Trp3,Thr5,AzaGly7,
Arg(Me)9,Trp10]MS10
Glycoloyl-D-Tyr-D-Trp-Asn-Thr-Phe-AzaGly-Leu-
Arg(Me)-Trp-NH₂
Compound No. 682:
des(1)-Ac-[D-Tyr2,D-Trp3,Thr5,AzaGly7,Gln8,
Arg(Me)9,Trp10]MS10
Ac-D-Tyr-D-Trp-Asn-Thr-Phe-AzaGly-Gln-Arg(Me)-
Trp-NH₂
Compound No. 685:
des(1)-Ac-[D-Tyr2,D-Pya(4)3,Thr5,AzaGly7,
Arg(Me)9]MS10
Ac-D-Tyr-D-Pya(4)-Asn-Thr-Phe-AzaGly-Leu-
Arg(Me)-Phe-NH₂
Compound No. 686:
des(1)-Ac-[D-Tyr2,D-Trp3,Gly4,AzaGly7,Arg(Me)9,
Trp10]MS10
Ac-D-Tyr-D-Trp-Gly-Ser-Phe-AzaGly-Leu-Arg(Me)-
Trp-NH₂
Compound No. 688:
des(1)-Ac-[D-Tyr2,D-Trp3,Thr5,AzaGly7,Pya(4)9,
Trp10]MS10
Ac-D-Tyr-D-Trp-Asn-Thr-Phe-AzaGly-Leu-Pya(4)-
Trp-NH₂
Compound No. 689:
des(1)-Ac-[D-Tyr2,D-Trp3,Thr5,AzaGly7,Arg(Me)9,
D-Trp10]MS10
Ac-D-Tyr-D-Trp-Asn-Thr-Phe-AzaGly-Leu-Arg(Me)-
D-Trp-NH₂
Compound No. 691:
des(1)-Ac-[D-Tyr2,D-Trp3,Thr5,Tyr6,AzaGly7,
Arg(Me)9,Trp10]MS10
Ac-D-Tyr-D-Trp-Asn-Thr-Tyr-AzaGly-Leu-Arg(Me)-
Trp-NH₂
Compound No. 692:
des(1)-Ac-[D-Tyr2,D-Trp3,Thr5,Trp6,AzaGly7,
Arg(Me)9,Trp10]MS10
Ac-D-Tyr-D-Trp-Asn-Thr-Trp-AzaGly-Leu-Arg(Me)-
Trp-NH₂
Compound No. 693:
des(1)-Ac-[D-Tyr2,D-Trp3,Thr5,Tyr(Me)6,AzaGly7,
Arg(Me)9,Trp10]MS10
Ac-D-Tyr-D-Trp-Asn-Thr-Tyr(Me)-AzaGly-Leu-
Arg(Me)-Trp-NH₂
Compound No. 694:
des(1)-Ac-[D-Tyr2,D-Trp3,Thr5,Nal(2)6,AzaGly7,
Arg(Me)9,Trp10]MS10
Ac-D-Tyr-D-Trp-Asn-Thr-Nal(2)-AzaGly-Leu-
Arg(Me)-Trp-NH₂
Compound No. 695:
des(1)-Ac-[D-Tyr2,D-Trp3,Thr5,Thi6,AzaGly7,
Arg(Me)9,Trp10]MS10
Ac-D-Tyr-D-Trp-Asn-Thr-Thi-AzaGly-Leu-Arg(Me)-
Trp-NH₂
Compound No. 696:
des(1)-Ac-[D-Tyr2,D-Trp3,Thr5,Cha6,AzaGly7,
Arg(Me)9,Trp10]MS10
Ac-D-Tyr-D-Trp-Asn-Thr-Cha-AzaGly-Leu-Arg(Me)-
Trp-NH₂
Compound No. 698:
des(1)-Ac-[D-Tyr2,D-Trp3,Thr5,AzaGly7,Abu8,
Arg(Me)9,Trp10]MS10
Ac-D-Tyr-D-Trp-Asn-Thr-Phe-AzaGly-Abu-Arg(Me)-
Trp-NH₂
Compound No. 699:
des(1)-Ac-[D-Tyr2,D-Trp3,Thr5,AzaGly7,γMeLeu8,
Arg(Me)9,Trp10]MS10
Ac-D-Tyr-D-Trp-Asn-Thr-Phe-AzaGly-γMeLeu-
Arg(Me)-Trp-NH₂
Compound No. 700:
des(1)-Ac-[D-Tyr2,D-Trp3,Thr5,Aib8,Arg(Me)9,
Trp10]MS10
Ac-D-Tyr-D-Trp-Asn-Thr-Phe-Gly-Aib-Arg(Me)-
Trp-NH₂
Compound No. 701:
des(1)-Ac-[D-Tyr2,D-Trp3,Dap4,AzaGly7,Arg(Me)9,
Trp10]MS10
Ac-D-Tyr-D-Trp-Dap-Ser-Phe-AzaGly-Leu-Arg(Me)-
Trp-NH₂
Compound No. 702:
des(1)-Ac-[D-Tyr2,D-Trp3,Asp(NHMe)4,Thr5,
AzaGly7,Arg(Me)9,Trp10]MS10
Ac-D-Tyr-D-Trp-Asp(NHMe)-Thr-Phe-AzaGly-Leu-
Arg(Me)-Trp-NH₂
Compound No. 703:
des(1)-Ac-[D-Tyr2,D-Trp3,Asp(NMe2)4,Thr5,
AzaGly7,Arg(Me)9,Trp10]MS10
Ac-D-Tyr-D-Trp-Asp(NMe2)-Thr-Phe-AzaGly-Leu-
Arg(Me)-Trp-NH₂

The metastin derivative (II) and/or (IV) is preferably a metastin derivative of the formula

XX0-XX2-XX3-XX4-XX5-XX6-AzaGly-XX8-XX9-XX10-NH₂ (II′),

or a salt thereof. In the above formula:

XX0 is formyl, C₁₋₆ alkanoyl (e.g., acetyl, propionyl, butyrl, hexanoyl; preferably acetyl, propionyl, butyryl; and more preferably acetyl), cyclopropancarbonyl, 6-(acetyl-D-arginylamino)caproyl, 6-((R)-2,3-diaminopropionylamino)caproyl, 6-(D-norleucylamino)caproyl, 4-(D-arginylamino)butyryl or 3-(4-hydroxyphenyl)propionyl, glycyl, tyrosyl, acetylglycyl, acetyltyrosyl, D-tyrosyl, acetyl-D-tyrosyl, pyroglutamyl, 3-(pyridin-3-yl)propionyl, adipoyl or 6-aminocaproyl (preferably acetyl);

XX2 is Tyr, D-Tyr, D-Ala, D-Leu, D-Phe, D-Lys, D-Trp or a valence bond (preferably D-Tyr or a valence bond; and more preferably D-Tyr);

XX3 is Trp, Pro, 4-pyridylalanine, Tic, D-Trp, D-Ala, D-Leu, D-Phe, D-Lys, D-Glu, D-2-pyridylalanine, D-3-pyridylalanine or D-4-pyridylalanine (preferably D-Trp or D-4-pyridylalanine);

XX4 is Asn, 2-amino-3-ureidopropionic acid, N^{β}-formyldiaminopropionic acid or N^{β}-acetyldiaminopropionic acid (preferably Asn);

XX5 is Ser, Thr or Val (preferably Ser or Thr);

XX6 is Phe, Tyr, Trp, Tyr(Me), Thi, Nal(2), Cha, 4-pyridylalanine or 4-fluorophenylalanine (preferably Phe or 4-fluorophenylalanine);

AzaGly is azaglycine;

XX8 is Leu, Nva or Val (preferably Leu);

XX9 is Arg, Orn, Arg(Me) or Arg(symMe2) (preferably Arg(Me)); and

XX10 is Phe, Trp, 2-naphthylalanine, 2-thienylalanine, tyrosine or 4-fluorophenylalanine (preferably Phe or Trp).

The compound represented by the following compound number is also suitable. Ac-D-Tyr-D-Trp-Asn-Thr-Phe-AzaGly-Leu-Arg(Me)-Trp-NH₂ (Compound No. 550), and a salt thereof.

In the metastin derivative (IV) of the present invention, the metastin derivative (III) of the present invention having the formula

XX00-XX02-XX03-XX04-XX05-XX06-AzaGly-XX08-XX09-XX010-NH₂ (III),

wherein:

XX00 is formyl, C₁₋₂₀ alkanoyl, cyclopropanecarbonyl, 6-(acetyl-D-arginylamino)caproyl, 6-((R)-2,3-diaminopropionylamino)caproyl, 6-(D-norleucylamino)caproyl), 4-(D-arginylamino)butyryl, 3-(4-hydroxyphenyl)propionyl, glycyl, tyrosyl, acetylglycyl, acetyltyrosyl, D-tyrosyl, acetyl-D-tyrosyl, pyroglutamyl, 3-(pyridin-3-yl)propionyl, adipoyl, glycoloyl, 6-aminocaproyl, 6-acetylaminocaproyl, 4-[bis-(2-pyridylmethyl)aminomethyl]benzoyl or 4-ureidobenzoyl;

XX02 is Tyr, D-Tyr, D-Ala, D-Leu, D-Phe, D-Lys, D-Trp or a valence bond;

XX03 is
i) an amino acid selected from among Ala, Arg, Asn, Asp, Cys, Gln, Glu, Gly, His, Ile, Leu, Lys, Met, Phe, Ser, Thr, Trp, Tyr and Val which may have a methylated α-amino group,
ii) a cyclic amino acid selected from among Pro, Aze(2), Aze(3), Pic(2), Pic(3), Hyp, Thz, Abz(2), Abz(3), Pzc(3), Pro(4NH₂), Hyp(Bzl), cisHyp, Pro(4F) and lzc,
iii) an amino acid selected from among D-Dap, D-Pya(4), DL-Ala(Pip), Orn, Aib and Tyr(PO₃H₂), or
iv) a valence bond;

XX04 is Asn, 2-amino-3-ureidopropionic acid, N^{β}-formyl-β-diaminopropionic acid, N^{β}-acetyl-β-diaminopropionic acid, N^{ω}-pentylasparagine, N^{ω}-cyclopropylasparagine, N^{ω}-benzylasparagine, 2,4-diaminobutanoic acid, 2,3-diaminopropionic acid, His, Gln, Gly, Arg, Cit, Nva, D-Asn or a valence bond;

XX05 is Ser, Thr, Val, NMeSer, Gly, Ala, Hyp, D-Ala, D-Thr, D-Pro or a valence bond;

XX06 is Phe, Tyr, Trp, Tyr(Me), Thi, Nal(2), Cha, Pya(4), threo-Ser(3Phenyl), erythro-Ser(3Phenyl) or phenylalanine which may be substituted;

AzaGly is azaglycine;

XX08 is Leu, Nva, Val or Ala(cPr);

XX09 is arginine which may be substituted, lysine which may be substituted or ornithine which may be substituted; and

XX010 is 2-naphthylalanine, 2-thienylalanine, tyrosine, phenylalanine which may be substituted or tryptophan which may be substituted, is the group of compounds mentioned in WO 2007/072997.

The metastin derivative (III) of the present invention also is preferred as the metastin derivative (IV) of the invention.

In the above formula, XX00 represents an amino-terminal modifying group, and XX02, XX03, XX04, XX05, XX06, XX08, XX09 and XX10 correspond respectively to the 2 position, 3 position, 4 position, 5 position, 6 position, 8 position, 9 position and 10 position of MS10 above.

The valence bond “—” between XX00, XX02, XX03, XX04, XX05, XX06, AzaGly, XX08, XX09, XX10 and NH₂ in the formula XX00-XX02-XX03-XX04-XX05-XX06-AzaGly-XX08-XX09-XX010-NH₂ has the following meanings.

The valence bond “—” in the formula “XX00-XX02” indicates a bond between the group represented by XX00 and the amino group included in XX02 (amino group at the α position). Specifically, “XX00-XX02” indicates that the hydrogen atom in the amino group (NH₂) included in XX02 has been substituted with the group represented by XX00.

The valence bond “—” in the formula “XX02-XX03” indicates that the carboxyl group included in XX02 (carboxyl group at the a position) and the amino group in XX03 (amino group at α position) are amide bonded. The valence bonds “—” in “XX03-XX04”, “XX04-XX05”, XX05-XX06”, XX08-XX09” and XX09-XX010” also have meanings similar to the above.

The valence bond “—” in the formula “XX06-AzaGly” indicates that the carboxyl group included in XX06 (carboxyl group at the a position) and the amino group in AzaGly (azaglycine) are amide bonded.

The valence bond “—” in the formula “AzaGly-XX08” indicates that the carboxyl group in AzaGly and the amino group in XX08 (amino group at a position) are amide bonded.

The valence bond “—” in the formula “XX010-NH₂” indicates a bond between the carboxyl group included in XX010 (carboxyl group at a position) and NH₂. More specifically, “XX010-NH₂” indicates that —OH in the carboxyl group (—COOH) included in XX010 has been substituted with —NH₂.

When XX02, XX03, XX04 and/or XX05 indicate a valence bond “—”, these valence bonds “—” have meanings similar to those described above.

In the above formula, XX00 is formyl, a C₁₋₂₀ alkanoyl (e.g., acetyl, propionyl, butyryl, hexanoyl, decanoyl; preferably a C₁₋₆ alkanoyl such as acetyl, propionyl or butyryl; and more preferably acetyl), cyclopropanecarbonyl, 6-(acetyl-D-alginylamino)caproyl, 6-((R)-2,3-diaminopropionylamino)caproyl, 6-(D-norleucylamino)caproyl, 4-(D-arginylamino)butyryl, 3-(4-hydroxyphenyl)propionyl, glycyl, tyrosyl, acetylglycyl, acetyltyrosyl, D-tyrosyl, acetyl-D-tyrosyl, pyroglutamyl, 3-(pyridin-3-yl)propionyl, adipoyl, glycoloyl, 6-aminocaproyl, 6-acetylaminocaproyl, 4-[bis-(2-pyridylmethyl)aminomethyl]benzoyl or 4-ureidobenzoyl; preferably a C₁₋₁₂ alkanoyl, 6-aminocaproyl, 6-acetylaminocaproyl, glycoloyl, 4-[bis-(2-pyridylmethyl)aminomethyl]benzoyl, 4-ureidobenzoyl, 3-(4-hydroxyphenyl)propionyl or pyroglutamyl; more preferably formyl, a C₁₋₆ alkanoyl or glycoloyl; even more preferably a C₁₋₆ alkanoyl or glycoloyl; and most preferably acetyl or glycoloyl. The following are also preferred as XX00: formyl, a C₁₋₂₀ alkanoyl, cyclopropanecarbonyl, 6-(acetyl-D-alginylamino)caproyl, 6-((R)-2,3-diaminopropionylamino)caproyl, 6-(D-norleucylamino)caproyl, 4-(D-arginylamino)butyryl, 3-(4-hydroxyphenyl)propionyl, glycyl, tyrosyl, acetylglycyl, acetyltyrosyl, D-tyrosyl, acetyl-D-tyrosyl, pyroglutamyl, 3-(pyridin-3-yl)propionyl, adipoyl, glycoloyl or 6-aminocaproyl; or formyl, a C₁₋₁₂ alkanoyl, cyclopropanecarbonyl, 6-(acetyl-D-arginylamino)caproyl, 6-((R)-2,3-diaminopropionylamino)caproyl, 6-(D-norleucylamino)caproyl, 4-(D-arginylamino)butyryl, 3-(4-hydroxyphenyl)propionyl, glycyl, tyrosyl, acetylglycyl, acetyltyrosyl, D-tyrosyl, acetyl-D-tyrosyl, pyroglutamyl, 3-(pyridin-3-yl)propionyl, adipoyl, glycoloyl or 6-aminocaproyl.

In the above formula, XX02 represents Tyr, D-Tyr, D-Ala, D-Leu, D-Phe, D-Lys, D-Trp or a valence bond; preferably D-Tyr, Tyr or a valence bond; more preferably D-Tyr or a valence bond; and even more preferably D-Tyr.

In the above formula, XX03 represents (i) an amino acid in which the α-amino group may be methylated (an amino acid selected from the group consisting of Ala (alanine), Arg (arginine), Asn (asparagine), Asp (aspartic acid), Cys (cysteine), Gln (glutamine), Glu (glutamic acid), Gly (glycine), His (histidine), Ile (isoleucine), Leu (leucine), Lys (lysine), Met (methionine), Phe (phenylalanine), Ser (serine), Thr (threonine), Trp (tryptophan), Tyr (tyrosine) and Val (valine); (ii) a cyclic amino acid (a cyclic amino acid selected from among Pro (proline), Aze(2), Aze(3), Pic(2), Pic(3), Hyp, Thz, Abz(2), Abz(3), Pzc(2), Pro(4NH₂), Hyp(Bzl), cisHyp, Pro(4F) and lzc); (iii) an amino acid selected from among D-Dap, D-Pya(4), DL-Ala(Pip), Orn, Aib and Tyr(PO₃H₂); or (iv) a valence bond.

Here, Aze(2) represents azetidine-2-carboxylic acid, Aze(3) represents azetidine-3-carboxylic acid, Pic(2) represents pipecolic acid, Pic(3) represents 3-piperidinecarboxylic acid, D-Dap represents D-2,3-diaminopropionic acid, D-Pya(4) represents 4-pyridyl-D-alanine, Hyp represents trans-4-hydroxyproline, Thz represents thioproline, Aib represents α-aminoisobutanoic acid, Abz(2) represents 2-aminobenzoic acid, Abz(3) represents 3-aminobenzoic acid, Izc represents imidazolidine-2-carboxylic acid, DL-Ala(Pip) represents DL-(4-piperidin-1-yl)alanine, Pzc(2) represents piperazine-2-carboxylic acid, Orn represents ornithine, Tyr(PO₃H₂) represents O-phosphotyrosine, Pro(4NH₂) represents 4-aminoproline, Hyp(Bzl) represents trans-4-benzyloxyproline, cisHyp represents cis-4-hydroxyproline, and Pro(4F) represents trans-4-fluoroproline.

Also, in the present specification, unless noted otherwise, an amino acid may be either the L-amino acid or the D-amino acid. Alanine may be either α-alanine or β-alanine. XX03 represents preferably D-Asp, D-Dap (D-2,3-diaminopropionic acid), D-Ser, D-Gln, D-His, D-Trp, D-Tyr, D-Pya(4), D-NMeAla (D-N^{α}-methylalanine), D-NMePhe (D-N^{α}-methylphenylalanine), Aze(2), Aze(3) (azetidine-3-carboxylic acid), Pic(2), Pic(3), Hyp, Thz, NMeAla, Gly, Aib, Abz(2), Abz(3), Sar, Izc, Leu, Lys, Glu, Thr, Trp, Ser, Ala, NMeAla, β-alanine, Pzc(2), Orn, His(3Me) (3-methylhistidine), Tyr(PO₃H₂), Pro(4NH₂), Hyp(Bzl), cisHyp, Pro(4F) or a valence bond; more preferably D-Asp, D-Dap, D-Ser, D-Gln, D-His, D-Trp, D-Tyr, D-Pya(4), D-NMeAla, D-NMePhe, Aze(2), Aze(3), Pic(2), Pic(3), Hyp, Thz, Gly, Aib, Abz(2), Sar, Izc, Leu, Lys, Glu, Thr, Trp, Ser, Ala, NMeAla, β-alanine, DL-Ala(Pip), Pzc(2), Orn, His(3Me), Tyr(PO₃H₂), Pro(4NH₂), Hyp(Bzl), cisHyp, Pro(4F) or a valence bond; even more preferably D-Gln, D-His, Aze(2), Pic(2), Hyp, Thz, Gly, Aib, D-NMeAla, Leu, Lys, Glu, Orn, His(3Me), Tyr(PO₃H₂), Pro(4NH₂), D-NMePhe, Hyp(Bzl), cisHyp or Pro(4F); still more preferably Aze(2), Hyp, Gly, Aib, Leu, Lys, Glu, His(3Me), Tyr(PO₃H₂), Hyp(Bzl), cisHyp or Pro(4F); and most preferably Hyp, Glu, Hyp(Bzl) or Pro(4F). The following are also preferred as XX03: D-Asp, D-Dap, D-Ser, D-Gln, D-His, D-NMeAla, D-NMePhe, Aze(2), Pic(2), Pic(3), Hyp, Thz, NMeAla, Gly, Aib, Abz(2), Abz(3), Sar, Leu, Lys, Glu, β-alanine, Pzc(2), Orn, His(3Me), Tyr(PO₃H₂), Pro(4NH₂) or Hyp(Bzl).

In the above formula, XX04 represents Asn, 2-amino-3-ureidopropionic acid, N^{β}-formyl-β-diaminopropionic acid, N^{β}-acetyl-β-diaminopropionic acid, N^{ω}-pentylasparagine, N^{ω}-cyclopropylasparagine, N^{ω}-benzylasparagine, 2,4-diaminobutanoic acid, 2,3-diaminopropionic acid, His, Gln, Gly, Arg, Cit, Nva, D-Asn or a valence bond; preferably Asn, 2-amino-3-ureidopropionic acid, N^{ω}-pentylasparagine, N^{ω}-cyclopropylasparagine, N^{ω}-benzylasparagine, 2,4-diaminobutanoic acid, 2,3-diaminopropionic acid, His, Gln, Gly, Arg, Cit, Nva, D-Asn or a valence bond; and more preferably Asn or 2-amino-3-ureidopropionic acid. The following are also preferred as XX04: Asn, 2-amino-3-ureidopropionic acid, N^{β}-formyl-β-diaminopropionic acid, N^{β}-acetyl-β-diaminopropionic acid, N^{ω}-pentylasparagine, N^{ω}-cyclopropylasparagine, N^{ω}-benzylasparagine, 2,4-diaminobutanoic acid, His, Gln, Cit or D-Asn; or Asn, 2-amino-3-ureidopropionic acid, N^{β}-formyldiaminopropionic acid, N^{β}-acetyldiaminopropionic acid, N^{ω}-pentylasparagine, N^{ω}-cyclopropylasparagine, N^{ω}-benzylasparagine or 2,4-diaminobutanoic acid.

In the above formula, XX05 represents Ser, Thr, Val, NMeSer, Gly, Ala, Hyp, D-Ala, D-Thr, D-Pro or a valence bond; preferably Thr, NMeSer, Gly, Ala, Hyp, D-Ala, D-Thr, D-Pro or a valence bond; more preferably Ser, Thr or Ala; and even more preferably Thr. The following are also preferred as XX05: Ser, Thr, Val, NMeSer, Gly, Ala, Hyp, D-Ala or D-Thr; or Ser, Thr or Val.

In the above formula, XX06 represents Phe, Tyr, Trp, Tyr(Me), Thi, Nal(2), Cha, Pya(4), threo-Ser(3Phenyl), erythro-Ser(3Phenyl), or phenylalanine which may be substituted. Examples of substituents that may be used here in the phenylalanine which may be substituted include substituents selected from among the following (referred to collectively as “Substituent Group B”): oxo, halogen atoms (e.g., fluorine, chlorine, bromine, iodine), C₁₋₃ alkylenedioxy (e.g., methylenedioxy, ethylenedioxy), nitro, cyano, C₁₋₆ alkyl which may be substituted, C₂₋₆ alkenyl which may be substituted, C₂₋₆ alkynyl which may be substituted, C₃₋₈ cycloalkyl which may be substituted, C₆₋₁₄ aryl which may be substituted, C₇₋₁₆ aralkyl which may be substituted, C₁₋₆ alkoxy which may be substituted, hydroxy, C₆₋₁₄ aryloxy which may be substituted, C₇₋₁₆ aralkyloxy which may be substituted, mercapto, C₁₋₆ alkylthio which may be substituted, C₆₋₁₄ arylthio which may be substituted, C₇₋₁₆ aralkylthio which may be substituted, amino which may be substituted [e.g., amino, mono- or di-C₁₋₆ alkylamino which may be substituted (e.g., methylamino, dimethylamino, ethylamino, diethylamino, propylamino, isopropylamino), mono- or di-C₂₋₆ alkenylamino which may be substituted (e.g., vinylamino, propenylamino, isopropenylamino), C₂₋₆ alkynylamino which may be substituted (e.g., 2-butyn-1-ylamino, 4-pentyn-1-ylamino, 5-hexyn-1-ylamino), mono- or di-C₃₋₈ cycloalkylamino which may be substituted (e.g., cyclopropylamino, cyclohexylamino), C₆₋₁₄ arylamino which may be substituted (e.g., phenylamino, diphenylamino, naphthylamino), C₁₋₆ alkoxyamino which may be substituted (e.g., methoxyamino, ethoxyamino, propoxyamino, isopropoxyamino), formylamino, C₁₋₆ alkylcarbonylamino which may be substituted (e.g., acetylamino, propionylamino, pivaroylamino), C₃₋₈ cycloalkylcarbonylamino which may be substituted (e.g., cyclopropylcarbonylamino, cyclopentylcarbonylamino, cyclohexylcarbonylamino), C₆₋₁₄ arylcarbonylamino which may be substituted (e.g., benzoylamino, naphthoylamino), C₁₋₆ alkoxycarbonylamino which may be substituted (e.g., methoxycarbonylamino, ethoxycarbonylamino, propoxycarbonylamino, butoxycarbonylamino), C₁₋₆ allkylsulfonylamino which may be substituted (e.g., methylsulfonylamino, ethylsulfonylamino), C₆₋₁₄ arylsulfonylamino which may be substituted (e.g., phenylsulfonylamino, 2-naphthylsulfonylamino, 1-naphthylsulfonylamino)], formyl, carboxy, C₁₋₆ alkylcarbonyl which may be substituted (e.g., acetyl, propionyl, pivaroyl), C₃₋₈ cycloalkylcarbonyl which may be substituted (e.g., cyclopropylcarbonyl, cyclopentylcarbonyl, cyclohexylcarbonyl, 1-methylcyclohexylcarbonyl), C₆-₁₄ arylcarbonyl which may be substituted (e.g., benzoyl, 1-napthoyl, 2-naphthoyl), C₇₋₁₆ aralkylcarbonyl which may be substituted (e.g., phenylacetyl, 3-phenylpropionyl), optinally substituted 5- to 7-membered heterocyclic carbonyl including, other than carbon atoms, from 1 to 4 heteroatoms of one or two species selected from among nitrogen, sulfur and oxygen (e.g., nicotinoyl, isonicotinoyl, thenoyl, furoyl, morpholinocarbonyl, thiomorpholinocarbonyl, piperazin-1-ylcarbonyl, pyrrolidin-1-ylcarbonyl), carboxyl which may be esterified, carbamoyl which may be substituted, C₁₋₆ alkylsulfonyl which may be substituted (e.g., methylsulfonyl, ethylsulfonyl), C₁₋₆ alkylsulfinyl which may be substituted (e.g., methylsulfinyl, ethylsulfinyl), C₆₋₁₄ arylsulfonyl which may be substituted (e.g., phenylsulfonyl, 1-naphthylsulfonyl, 2-naphthylsulfonyl), C₆₋₁₄ arylsulfinyl which may be substituted (e.g., phenylsulfinyl, 1-naphthylsulfinyl, 2-naphthylsulfinyl), C₁₋₆ alkylcarbonyloxy which may be substituted (e.g., acetoxy, propionyloxy), C₆₋₁₄ arylcarbonyloxy which may be substituted (e.g., benzoyloxy, naphthylcarbonyloxy), C₁₋₆ alkoxycarbonyloxy which may be substituted (e.g., methoxycarbonyloxy, ethoxycarbonyloxy, propoxycarbonyloxy, butoxycarbonyloxy), mono-C₁₋₆ alkylcarbamoyloxy which may be substituted (e.g., methylcarbamoyloxy, ethylcarbamoyloxy), di-C₁₋₆ alkylcarbamoyloxy which may be substituted (e.g., dimethylcarbamoyloxy, diethylcarbamoyloxy), mono- or di-C₆₋₁₄ arylcarbamoyloxy which may be substituted (e.g., phenylcarbamoyloxy, naphthylcarbamoyloxy), heterocyclic groups which may be substituted, sulfo, sulfamoyl, sulfinamoyl, sulfenamoyl, and radicals to which two or more (e.g., 2 or 3) of these substituents are bonded. The number of substituents is not subject to any particular limitation, although there may be from one to five, and preferably from one to three, at substitutable positions. When the number of substituents is two or more, the respective substituents may be the same or different.

In Substituent Group B, the “carboxyl which may be esterified” is exemplified by C₁₋₆ alkoxycarbonyl which may be substituted (e.g., methoxycarbonyl, ethoxycarbonyl, propoxycarbonyl, tert-butyoxycarbonyl), C₆₋₁₄ aryloxycarbonyl which may be substituted (e.g., phenoxycarbonyl), and C₇₋₁₆ aralkyloxycarbonyl which may be substituted (e.g., benzyloxycarbonyl, phenethyloxycarbonyl).

In Substituent Group B, the “C₁₋₆ alkyl” in the “C₁₋₆ alkyl which may be substituted” is exemplified by methyl, ethyl, propyl, isopropyl, butyl, isobutyl, sec-butyl, tert-butyl, pentyl, isopentyl, neopentyl and hexyl.

In Substituent Group B, the “C₂₋₆ alkenyl” in the “C₂₋₆ alkenyl which may be substituted” is exemplified by vinyl, propenyl, isopropenyl, 2-buten-1-yl, 4-penten-1-yl and 5-hexen-1-yl.

In Substituent Group B, the “C₂₋₆ alkynyl” in the “C₂₋₆ alkynyl which may be substituted” is exemplified by 2-butyn-1-yl, 4-pentyn-1-yl and 5-hexyn-1-yl.

In Substituent Group B, the “C₃₋₈ cycloalkyl” in the “C₃₋₈ cycloalkyl which may be substituted” is exemplified by cyclopropyl, cyclobutyl, cyclopentyl and cyclohexyl.

In Substituent Group B, the “C₆₋₁₄ aryl” in the “C₆₋₁₄ aryl which may be substituted” is exemplified by phenyl, 1-naphthyl, 2-naphthyl, 2-biphenylyl, 3-biphenylyl, 4-biphenylyl and 2-anthryl.

In Substituent Group B, the “C₇₋₁₆ aralkyl” in the “C₇₋₁₆ aralkyl which may be substituted” is exemplified by benzyl, phenethyl, diphenylmethyl, 1-naphthylmethyl, 2-naphthylmethyl, 2,2-diphenylethyl, 3-phenylpropyl, 4-phenylbutyl, 5-phenylpentyl, 2-biphenylylmethyl, 3-biphenylylmethyl and 4-biphenylylmethyl.

In Substituent Group B, the “C₁₋₆ alkoxy” in the “C₁₋₆ alkoxy which may be substituted” is exemplified by methoxy, propoxy, isoporpoxy, butoxy, isobutoxy, sec-butoxy, pentyloxy and hexyloxy.

In Substituent Group B, the “C₆₋₁₄ aryloxy” in the “C₆₋₁₄ aryloxy which may be substituted” is exemplified by phenyloxy, 1-naphthyloxy and 2-naphthyloxy.

In Substituent Group B, the “C₇₋₁₆ aralkyloxy” in the “C₇₋₁₆ aralkyloxy which may be substituted” is exemplified by benzyloxy and phenethyloxy.

In Substituent Group B, the “C₁₋₆ alkylthio” in the “C₁₋₆ alkylthio which may be substituted” is exemplified by methylthio, ethylthio, propylthio, isopropylthio, butylthio, sec-butylthio and tert-butylthio.

In Substituent Group B, the “C₆₋₁₄ arylthio” in the “C₆₋₁₄ arylthio which may be substituted” is exemplified by phenylthio, 1-naphthylthio and 2-naphthylthio.

In Substituent Group B, the “C₇₋₁₆ aralkylthio” in the “C₇₋₁₆ aralkylthio which may be substituted” is exemplified by benzylthio and phenethylthio.

Substituents on these “C₁₋₆ alkoxycarbonyl,” “C₁₋₆ alkyl,” C₂₋₆ alkenyl,” “C₂₋₆ alkynyl,” “C₁₋₆ alkoxy,” “C₁₋₆ alkylthio,” “C₁₋₆ alkylamino,” “C₂₋₆ alkenylamino,” “C₂₋₆ alkynylamino,” “C₁₋₆ alkoxyamino,” “C₁₋₆ alkylcarbonyl,” “C₁₋₆ alkylsulfonyl,” “C₁₋₆ alkylsulfinyl,” “C₁₋₆ alkylcarbonylamino,” “C₁₋₆ alkoxycarbonylamino,” “C₁-₆ alkylsulfonylamino,” “C₁₋₆ alkylcarbonyloxy,” “C₁₋₆ alkoxycarbonyloxy,” “mono-C₁-₆ alkylcarbamoyloxy” and “di-C₁₋₆ alkylcarbamoyloxy” substituents are exemplified by from one to five substituents selected from among halogen atoms (e.g., fluorine, chlorine, bromine, iodine), carboxy, hydroxy, amino, mono- or di-C₁₋₆ alkylamino, mono- or di-C₆₋₁₄ arylamino, C₃₋₈ cycloalkyl, C₁₋₆ alkoxy, C₁₋₆ alkoxycarbonyl, C₁₋₆ alkylthio, C₁₋₆ alkylsulfinyl, C₁₋₆ alkylsulfonyl, the above-mentioned carboxyl which may be esterified, carbamoyl, thiocarbamoyl, mono-C₁₋₆ alkylcarbamoyl (e.g., methylcarbamoyl, ethylcarbamoyl), di-C₁₋₆ alkylcarbamoyl (e.g., dimethylcarbamoyl, diethylcarbamoyl, ethylmethylcarbamoyl), mono- or di-C₆₋₁₄ arylcarbamoyl (e.g., phenylcarbamoyl, 1-naphthylcarbamoyl, 2-naphthylcarbamoyl) and mono- or di- 5- to 7-membered heterocyclic carbamoyl including, other than carbon atoms, from one to four heteroatoms of one or two species selected from among nitrogen, sulfur and oxygen (e.g., 2-pyridylcarbamoyl, 3-pyridylcarbamoyl, 4-pyridylcarbamoyl, 2-thienylcarbamoyl, 3-thienylcarbamoyl).

In Substituent Group B, substituents on “C₆₋₁₄ aryloxycarbonyl,” “C₇₋₁₆ aralkyloxycarbonyl,” “C₃₋₈ cycloalkyl,” “C₆₋₁₄ aryl,” “C₇₋₁₆ aralkyl,” “C₆₋₁₄ aryloxy,” “C₇₋₁₆ aralkyoxy,” “C₆₋₁₄ arylthio,” “C₇₋₁₆ aralkylthio,” “C₃₋₈ cycloalkylamino,” “C₆₋₁₄ arylamino,” “C₃₋₈ cycloalkylcarbonyl,” “C₆₋₁₄ arylcarbonyl,” “C₇₋₁₆ aralkylcarbonyl,” “5- to 7-membered heterocyclic carbonyl including, other than carbon atoms, from 1 to 4 hetero atoms of one or two species selected from among nitrogen, sulfur and oxygen,” “C₆₋₁₄ arylsulfonyl,” “C₆₋₁₄ arylsulfinyl,” “C₃₋₈ cycloalkylcarbonylamino,” “C₆₋₁₄ arylcarbonylamino,” “C₆₋₁₄ arylsulfonylamino,” “C₆₋₁₄ arylcarbonyloxy” and “mono- or di-C₆₋₁₄ arylcarbamoyloxy” are exemplified by from one to five substituents selected from among halogen atoms, hydroxy, carboxy, nitro, cyano, the above-mentioned C₁₋₆ alkyl which may be substituted, the above-mentioned C₂₋₆ alkenyl which may be substituted, the above-mentioned C₂₋₆ alkynyl which may be substituted, the above-mentioned C₃₋₈ cycloalkyl which may be substituted, the above-mentioned C₁₋₆ alkoxy which may be substituted, the above-mentioned C₁₋₆ alkylthio which may be substituted, the above-mentioned C₁₋₆ alkylsulfinyl which may be substituted, the above-mentioned C₁₋₆ alkylsulfonyl which may be substituted, the above-mentioned carboxyl which may be esterified, carbamoyl, thiocarbamoyl, mono-C₁₋₆ alkylcarbamoyl, di-C₁₋₆ alkylcarbamoyl, mono- or di-C₆₋₁₄ arylcarbamoyl and mono- or di- 5- to 7-membered heterocyclic carbamoyl including, other than carbon atoms, from one to four heteroatoms of one or two species selected from among nitrogen, sulfur and oxygen.

In Substituent Group B, examples of “heterocyclic groups which may be substituted” include 5- to 14-membered (monocyclic, bicyclic, or tricyclic) heterocyclic groups including, other than carbon atoms, from one to four heteroatoms of one or two species selected from among nitrogen, sulfur and oxygen, which groups may be substituted with a substituent, including a halogen atom, hydroxy, carboxy, nitro, cyano, the above-mentioned C₁₋₆ alkyl which may be substituted, the above-mentioned C₂₋₆ alkenyl which may be substituted, the above-mentioned C₂₋₆ alkynyl which may be substituted, the above-mentioned C₃₋₈ cycloalkyl which may be substituted, the above-mentioned C₆₋₁₄ aryl which may be substituted, the above-mentioned C₁₋₆ alkoxy which may be substituted, the above-mentioned C₁₋₆ alkylthio which may be substituted, the above-mentioned C₆₋₁₄ arylthio which may be substituted, the above-mentioned C₇₋₁₆ aralkylthio which may be substituted, the above-mentioned C₁₋₆ alkylsulfinyl which may be substituted, the above-mentioned C₆₋₁₄ arylsulfinyl which may be substituted, the above-mentioned C₁₋₆ alkylsulfonyl which may be substituted, the above-mentioned C₆₋₁₄ arylsulfonyl which may be substituted, the above-mentioned carboxyl which may be esterified, carbamoyl, thiocarbamoyl, mono-C₁₋₆ alkylcarbamoyl, di-lower alkylcarbamoyl, mono- or di-C₆₋₁₄ arylcarbamoyl, and mono- or di- 5- to 7-membered heterocyclic carbamoyl including, other than carbon atoms, from one to four heteroatoms of one or two species selected from among nitrogen, sulfur and oxygen. Preferred examples include (i) 5- to 14-membered (preferably 5- to 10-member) aromatic heterocyclic groups, (ii) 5- to 10-membered non-aromatic heterocyclic groups, and (iii) monovalent groups obtained by removing any one hydrogen atom from a 7- to 10-membered heterobridged ring. Of these, a 5-membered aromatic heterocyclic group is especially preferred. Illustrative examples include aromatic heterocyclic groups such as thienyl (e.g., 2-thienyl, 3-thienyl), furyl (e.g., 2-furyl, 3-furyl), pyridyl (e.g., 2-pyridyl, 3-pyridyl, 4-pyridyl), thiazolyl (e.g., 2-thiazolyl, 4-thiazolyl, 5-thiazolyl), oxazolyl (e.g., 2-oxazolyl, 4-oxazolyl), quinolyl (e.g., 2-quinolyl, 3-quinolyl, 4-quinolyl, 5-quinolyl, 8-quinolyl), isoquinolyl (e.g., 1-isoquinolyl, 3-isoquinolyl, 4-isoquinolyl, 5-isoquinolyl), pyrazinyl, pyrimidinyl (e.g., 2-pyrimidinyl, 4-pyrimidinyl), pyrrolyl (e.g., 1-pyrrolyl, 2-pyrrolyl, 3-pyrrolyl), imidazolyl (e.g., 1-imidazolyl, 2-imidazolyl, 4-imidazolyl), pyrazolyl (e.g., 1-pyrazolyl, 3-pyrazolyl, 4-pyrazolyl), pyridazinyl (e.g., 3-pyridazinyl, 4-pyridazinyl), isothiazolyl (e.g., 3-isothiazolyl), isoxazolyl (e.g., 3-isoxazolyl), indolyl (e.g., 1-indolyl, 2-indolyl, 3-indolyl), 2-benzothiazolyl, benzo[b]thienyl (e.g., 2-benzo[b]thienyl, 3-benzo[b]thienyl), benzo[b]furanyl (e.g., 2-benzo[b]furanyl, 3-benzo[b]furanyl); and non-aromatic heterocyclic radicals such as pyrrolidinyl (e.g., 1-pyrrolidinyl, 2-pyrrolidinyl, 3-pyrrolidinyl), oxazolidinyl (e.g., 2-oxazolidinyl), imidazolinyl (e.g., 1-imidazolinyl, 2-imidazolinyl, 4-imidazolinyl), piperidinyl (e.g., 1-piperidinyl, 2-piperidinyl, 3-piperidinyl, 4-piperidinyl), piperazinyl (e.g., 1-piperazinyl, 2-piperazinyl), morpholino and thiomorpholino.

In Substituent Group B, examples of “carbamoyl which may be substituted” include C₁₋₆ alkyl which may be substituted, C₂₋₆ alkenyl which may be substituted, C₂₋₆ alkynyl which may be substituted, C₃₋₈ cycloalkyl which may be substituted, C₆₋₁₄ aryl which may be substituted, and carbamoyl which may be substituted with heterocyclic group that may be substituted. Illustrative examples include carbamoyl, thiocarbamoyl, mono-C₁₋₆ alkylcarbamoyl (e.g., methylcarbamoyl, ethylcarbamoyl), di-C₁₋₆ alkylcarbamoyl (e.g., dimethylcarbamoyl, diethylcarbamoyl, ethylmethylcarbamoyl), C₁₋₆ alkyl (C₁₋₆ alkoxy) carbamoyl (e.g., methyl(methoxy)carbamoyl, ethyl(methoxy)carbamoyl), mono- or di-C₆₋₁₄ arylcarbamoyl (e.g., phenylcarbamoyl, 1-naphthylcarbamoyl, 2-naphthylcarbamoyl), mono- or di- 5- to 7-membered heterocyclic carbamoyl including, other than carbon atoms, one to four heteroatoms of one or two species selected from among nitrogen, sulfur and oxygen atoms (e.g., 2-pyridylcarbamoyl, 3-pyridylcarbamoyl, 4-pyridylcarbamoyl, 2-thienylcarbamoyl, 3-thienylcarbamoyl), and 5- to 7-membered cyclic carbamoyl (e.g., 1-pyrrolidinylcarbonyl, 1-piperidinylcarbonyl, hexamethyleneiminocarbonyl).

In Substituent Group B, examples of “amino which may be substituted” include aminos which may be substituted with one or two groups such as the above-mentioned C₁₋₆ alkyl which may be substituted, the above- mentioned C₂₋₆ alkenyl which may be substituted, the above-mentioned C₂₋₆ alkynyl which may be substituted, the above-mentioned C₃₋₈ cycloalkyl which may be substituted, the above-mentioned C₆₋₁₄ aryl which may be substituted, the C₁₋₆ alkoxy which may be substituted, formyl, the above-mentioned C₁₋₆ alkylcarbonyl which may be substituted, the above-mentioned C₃₋₈ cycloalkylcarbonyl which may be substituted, the above-mentioned C₆₋₁₄ arylcarbonyl which may be substituted, the above-mentioned C₁₋₆ alkoxycarbonyl which may be substituted, the above-mentioned C₁₋₆ alkylsulfonyl which may be substituted and C₆₋₁₄ arylsulfonyl which may be substituted.

Preferred substituents include halogen atoms, hydroxy, C₁₋₆ alkoxy, C₁₋₆ alkyl which may be halogenated, C₁₋₆ alkoxy which may be halogenated, amino, nitro and cyano.

In the above formula, XX06 represents preferably Phe, Tyr, Trp, Tyr(Me) (O-methyltyrosine), Thi (2-thienylalanine), Nal(2) (2-naphthylalanine), Cha (cyclohexylalanine), Pya(4) (4-pyridylalanine), Phe(2F) (2-fluorophenylalanine), Phe(3F) (3-fluorophenylalanine), Phe(4F) (4-fluorophenylalanine), Phe(4Cl) (4-chlorophenylalanine), cLMePhe (α-methylphenylalanine), Phe(2Me), Phe(3Me), Phe(4Me), threo-Ser(3Phenyl), erythro-Ser(3Phenyl) or D-Phe; more preferably Phe, Cha, Phe(2F), Phe(3F), Phe(4F), Phe(4Cl), αMePhe, Phe(2Me), Phe(3Me), Phe(4Me), threo-Ser(3Phenyl), erythro-Ser(3Phenyl) or D-Phe; even more preferably Phe, Phe(2F), Phe(3F), Phe(4F), Phe(4C1), αMePhe, Phe(2Me), Phe(3Me), Phe(4Me), threo-Ser(3Phenyl), erythro-Ser(3Phenyl) or D-Phe; still more preferably Phe, Cha, Phe(2F), Phe (3F), Phe(4F), Phe(4C1), Phe(2Me), Phe(3Me), Phe(4Me), threo-Ser(3Phenyl) or erythro-Ser(3Phenyl); and most preferably Phe, Cha, Phe(3F) or Phe(4F). The following are also preferred as XX06: Phe, Tyr, Trp, Tyr(Me), Thi, Nal(2), Cha, Pya(4), Phe(2F), Phe(3F), Phe(4F), Phe(4C1) or D-Phe; or Phe, Tyr, Trp, Tyr(me), Thi, Nal(2), Cha, Pya(4), Phe(2F), Phe(3F), Phe(4F) or Phe(4Cl).

In the above formula, AzaGly represents azaglycine.

In the above formula, XX08 represents Leu, Nva (norvaline), Val or Ala(cPr) (cyclopropylalanine), and preferably represents Leu or Ala(cPr). The following is also preferred as XX08: Leu, Nva or Val.

In the above formula, XX09 represents arginine which may be substituted, lysine which may be substituted or ornithine which may be substituted. Here, the substituent in the arginine which may be substituted, lysine which may be substituted or ornithine which may be substituted is one or a substitutable number of C₁₋₆ alkyl groups (e.g., methyl, ethyl, propyl, isopropyl, butyl) or C₁₋₆ acyl groups (e.g., acetyl, propionyl). XX09 represents preferably Arg, Orn (ornithine), Arg(Me) (N^{ω}-methylarginine), D-Arg or Arg(asymMe₂) (asymmetric N^{ω,ω}-dimethylarginine); more preferably Arg, Arg(Me) or D-Arg; and even more preferably Arg or Arg(Me). The following is also preferred as XX09: Arg, Orn, Arg(Me) or Arg(asymMe₂).

In the above formula, XX010 represents 2-naphthylalanine, 2-thienylalanine, tyrosine, phenylalanine which may be substituted or tryptophan which may be substituted. Examples of substituents that may be used here in the phenylalanine which may be substituted or the tryptophan which may be substituted include substituents selected from among the following (referred to collectively as “Substituent Group C”): oxo, a halogen atom (e.g., fluorine, chlorine, bromine, iodine), C₁₋₃ alkylenedioxy (e.g., methylenedioxy, ethylenedioxy), nitro, cyano, C₁₋₆ alkyl which may be substituted, C₂₋₆ alkenyl which may be substituted, C₂₋₆ alkynyl which may be substituted, C₃₋₈ cycloalkyl which may be substituted, C₆₋₁₄ aryl which may be substituted, C₇₋₁₆ aralkyl which may be substituted, C₁₋₆ alkoxy which may be substituted, hydroxy, C₆₋₁₄ aryloxy which may be substituted, C₇₋₁₆ aralkyloxy which may be substituted, mercapto, C₁₋₆ alkylthio which may be substituted, C₆₋₁₄ arylthio which may be substituted, C₇₋₁₆ aralkylthio which may be substituted, amino which may be substituted [e.g., amino, mono- or di-C₁₋₆ alkylamino which may be substituted (e.g., methylamino, dimethylamino, ethylamino, diethylamino, propylamino, isopropylamino), mono- or di-C₂₋₆ alkenylamino which may be substituted (e.g., vinylamino, propenylamino, isopropenylamino), C₂₋₆ alkynylamino which may be substituted (e.g., 2-butyn-1-ylamino, 4-pentyn-1-ylamino, 5-hexyn-1-ylamino), mono- or di-C₃₋₈ cycloalkylamino which may be substituted (e.g., cyclopropylamino, cyclohexylamino), C₆₋₁₄ arylamino which may be substituted (e.g., phenylamino, diphenylamino, naphthylamino), C₁₋₆ alkoxyamino which may be substituted (e.g., methoxyamino, ethoxyamino, propoxyamino, isopropoxyamino), formylamino, C₁₋₆ alkylcarbonylamino which may be substituted (e.g., acetylamino, propionylamino, pivaroylamino), C₃₋₈ cycloalkylcarbonylamino which may be substituted (e.g., cyclopropylcarbonylamino, cyclopentylcarbonylamino, cyclohexylcarbonylamino), C₆₋₁₄ arylcarbonylamino which may be substituted (e.g., benzoylamino, naphthoylamino), C₁₋₆ alkoxycarbonylamino which may be substituted (e.g., methoxycarbonylamino, ethoxycarbonylamino, propoxycarbonylamino, butoxycarbonylamino), C₁₋₆ alkylsulfonylamino which may be substituted (e.g., methylsulfonylamino, ethylsulfonylamino), C₆₋₁₄ arylsulfonylamino which may be substituted (e.g., phenylsulfonylamino, 2-naphthylsulfonylamino, 1-naphthylsulfonylamino)], formyl, carboxy, C₁₋₆ alkylcarbonyl which may be substituted (e.g., acetyl, propionyl, pivaroyl), C₃₋₈ cycloalkylcarbonyl which may be substituted (e.g., cyclopropylcarbonyl, cyclopentylcarbonyl, cyclohexylcarbonyl, 1-methylcyclohexylcarbonyl), C₆₋₁₄ arylcarbonyl which may be substituted (e.g., benzoyl, 1-napthoyl, 2-naphthoyl), C₇₋₁₆ aralkylcarbonyl which may be substituted (e.g., phenylacetyl, 3-phenylpropionyl), optionally substituted 5- to 7-membered heterocyclic carbonyl including, other than carbon atoms, from 1 to 4 heteroatoms of one or two species selected from among nitrogen, sulfur and oxygen atoms (e.g., nicotinoyl, isonicotinoyl, thenoyl, furoyl, morpholinocarbonyl, thiomorpholinocarbonyl, piperazin-1-ylcarbonyl, pyrrolidin-1-ylcarbonyl), carboxyl which may be esterified, carbamoyl which may be substituted, C₁₋₆ alkylsulfonyl which may be substituted (e.g., methylsulfonyl, ethylsulfonyl), C₁₋₆ alkylsulfinyl which may be substituted (e.g., methylsulfinyl, ethylsulfinyl), C₆₋₁₄ arylsulfonyl which may be substituted (e.g., phenylsulfonyl, 1-naphthylsulfonyl, 2-naphthylsulfonyl), C₆₋₁₄ arylsulfinyl which may be substituted (e.g., phenylsulfinyl, 1-naphthylsulfinyl, 2-naphthylsulfinyl), C₁₋₆ alkylcarbonyloxy which may be substituted (e.g., acetoxy, propionyloxy), C₆₋₁₄ arylcarbonyloxy which may be substituted (e.g., benzoyloxy, naphthylcarbonyloxy), C₁₋₆ alkoxylcarbonyloxy which may be substituted (e.g., methoxycarbonyloxy, ethoxycarbonyloxy, propoxycarbonyloxy, butoxycarbonyloxy), mono-C₁₋₆ alkylcarbamoyloxy which may be substituted (e.g., methylcarbamoyloxy, ethylcarbamoyloxy), di-C₁₋₆ alkylcarbamoyloxy which may be substituted (e.g., dimethylcarbamoyloxy, diethylcarbamoyloxy), mono- or di-C₆₋₁₄ arylcarbamoyloxy which may be substituted (e.g., phenylcarbamoyloxy, naphthylcarbamoyloxy), heterocyclic groups which may be substituted, sulfo, sulfamoyl, sulfinamoyl, sulfenamoyl, and radicals to which two or more (e.g., 2 or 3) of these substituents are bonded. The number of substituents is not subject to any particular limitation, although there may be from one to five, and preferably from one to three, at substitutable positions. When the number of substituents is two or more, the respective substituents may be the same or different.

In Substituent Group C, the “carboxyl which may be esterified” is exemplified by C₁₋₆ alkoxycarbonyl which may be substituted (e.g., methoxycarbonyl, ethoxycarbonyl, propoxycarbonyl, tert-butyoxycarbonyl), C₆₋₁₄ aryloxycarbonyl which may be substituted (e.g., phenoxycarbonyl), and C₇₋₁₆ aralkyloxycarbonyl which may be substituted (e.g., benzyloxycarbonyl, phenethyloxycarbonyl).

In Substituent Group C, the “C₁₋₆ alkyl” in the “C₁₋₆ alkyl which may be substituted” is exemplified by methyl, ethyl, propyl, isopropyl, butyl, isobutyl, sec-butyl, tert-butyl, pentyl, isopentyl, neopentyl and hexyl.

In Substituent Group C, the “C₂₋₆ alkenyl” in the “C₂₋₆ alkenyl which may be substituted” is exemplified by vinyl, propenyl, isopropenyl, 2-buten-1-yl, 4-penten-1-yl and 5-hexen-1-yl.

In Substituent Group C, the “C₂₋₆ alkynyl” in the “C₂₋₆ alkynyl which may be substituted” is exemplified by 2-butyn-1-yl, 4-pentyn-1-yl and 5-hexyn-1-yl.

In Substituent Group C, the “C₃₋₈ cycloalkyl” in the “C₃₋₈ cycloalkyl which may be substituted” is exemplified by cyclopropyl, cyclobutyl, cyclopentyl and cyclohexyl.

In Substituent Group C, the “C₆₋₁₄ aryl” in the “C₆₋₁₄ aryl which may be substituted” is exemplified by phenyl, 1-naphthyl, 2-naphthyl, 2-biphenylyl, 3-biphenylyl, 4-biphenylyl and 2-anthryl.

In Substituent Group C, the “C₇₋₁₆ aralkyl” in the “C₇₋₁₆ aralkyl which may be substituted” is exemplified by benzyl, phenethyl, diphenylmethyl, 1-naphthylmethyl, 2-naphthylmethyl, 2,2-diphenylethyl, 3-phenylpropyl, 4-phenylbutyl, 5-phenylpentyl, 2-biphenylylmethyl, 3-biphenylylmethyl and 4-biphenylylmethyl.

In Substituent Group C, the “C₁₋₆ alkoxy” in the “C₁₋₆ alkoxy which may be substituted” is exemplified by methoxy, ethoxy, propoxy, isoporpoxy, butoxy, isobutoxy, sec-butoxy, pentyloxy and hexyloxy.

In Substituent Group C, the “C₆₋₁₄ aryloxy” in the “C₆₋₁₄ aryloxy which may be substituted” is exemplified by phenyloxy, 1-naphthyloxy and 2-naphthyloxy.

In Substituent Group C, the “C₇₋₁₆ aralkyloxy” in the “C₇₋₁₆ aralkyloxy which may be substituted” is exemplified by benzyloxy and phenethyloxy.

In Substituent Group C, the “C₁₋₆ alkylthio” in the “C₁₋₆ alkylthio which may be substituted” is exemplified by methylthio, ethylthio, propylthio, isopropylthio, butylthio, sec-butylthio and tert-butylthio.

In Substituent Group C, the “C₆₋₁₄ arylthio” in the “C₆₋₁₄ arylthio which may be substituted” is exemplified by phenylthio, 1-naphthylthio and 2-naphthylthio.

In Substituent Group C, the “C₇₋₁₆ aralkylthio” in the “C₇₋₁₆ aralkylthio which may be substituted” is exemplified by benzylthio and phenethylthio.

Substituents on these “C₁₋₆ alkoxycarbonyl,” “C₁₋₆ alkyl,” C₂₋₆ alkenyl,” “C₂₋₆ alkynyl, “C₁₋₆ alkoxy,” “C₁₋₆ alkylthio,” “C₁₋₆ alkylamino,” “C₂₋₆ alkenylamino,” “C₂₋₆ alkynylamino,” “C₁₋₆ alkoxyamino,” “C₁₋₆ alkylcarbonyl,” “C₁₋₆ alkylsulfonyl,” “C₁₋₆ alkylsulfinyl,” “C₁₋₆ alkylcarbonylamino,” “C₁₋₆ alkoxycarbonylamino,” “C₁₋₆ alkylsulfonylamino,” “C₁₋₆ alkylcarbonyloxy,” “C₁₋₆ alkoxycarbonyloxy,” “mono-C₁₋₆ alkylcarbamoyloxy” and “di-C₁₋₆ alkylcarbamoyloxy” substituents are exemplified by from one to five substituents selected from among halogens (e.g., fluorine, chlorine, bromine, iodine), carboxy, hydroxy, amino, mono- or di-C₁₋₆ alkylamino, mono- or di-C₆₋₁₄ arylamino, C₃₋₈ cycloalkyl, C₁₋₆ alkoxy, C₁₋₆ alkoxycarbonyl, C₁₋₆ alkylthio, C₁₋₆ alkylsulfinyl, C₁₋₆ alkylsulfonyl, the above-mentioned carboxyl which may be esterified, carbamoyl, thiocarbamoyl, mono-C₁₋₆ alkylcarbamoyl (e.g., methylcarbamoyl, ethylcarbamoyl), di-C₁₋₆ alkylcarbamoyl (e.g., dimethylcarbamoyl, diethylcarbamoyl, ethylmethylcarbamoyl), mono- or di-C₆₋₁₄ arylcarbamoyl (e.g., phenylcarbamoyl, 1-naphthylcarbamoyl, 2-naphthylcarbamoyl) and mono- or di- 5- to 7-membered heterocyclic carbamoyl including, other than carbon atoms, from one to four heteroatoms of one or two species selected from among nitrogen, sulfur and oxygen atoms (e.g., 2-pyridylcarbamoyl, 3-pyridylcarbamoyl, 4-pyridylcarbamoyl, 2-thienylcarbamoyl, 3-thienylcarbamoyl).

In Substituent Group C, substituents on “C₆₋₁₄ aryloxycarbonyl,” “C₇₋₁₆ aralkyloxycarbonyl,” “C₃₋₈ cycloalkyl,” “C₆₋₁₄ aryl,” “C₇₋₁₆ aralkyl,” “C₆₋₁₄ aryloxy,” “C₇₋₁₆ aralkyoxy,” “C₆₋₁₄ arylthio,” “C₇₋₁₆ aralkylthio,” “C₃₋₈ cycloalkylamino,” “C₆₋₁₄ arylamino,” “C₃₋₈ cycloalkylcarbonyl,” “C₆₋₁₄ arylcarbonyl,” “C₇₋₁₆ aralkylcarbonyl,” “5- to 7-membered heterocyclic carbonyl including, other than carbon atoms, from 1 to 4 hetero atoms of one or two species selected from among nitrogen, sulfur and oxygen atoms,” “C₆₋₁₄ arylsulfonyl,” “C₆₋₁₄ arylsulfinyl,” “C₃₋₈ cycloalkylcarbonylamino,” “C₆₋₁₄ arylcarbonylamino,” “C₆₋₁₄ arylsulfonylamino,” “C₆₋₁₄ arylcarbonyloxy” and “mono- or di-C₆₋₁₄ arylcarbamoyloxy” are exemplified by from one to five substituents selected from among halogen atoms, hydroxy, carboxy, nitro, cyano, the above-mentioned C₁₋₆ alkyls which may be substituted, the above-mentioned C₂₋₆ alkenyls which may be substituted, the above-mentioned C₂₋₆ alkynyls which may be substituted, the above-mentioned C₃₋₈ cycloalkyls which may be substituted, the above-mentioned C₁₋₆ alkoxy radicals which may be substituted, the above-mentioned C₁₋₆ alkylthio radicals which may be substituted, the above-mentioned C₁₋₆ alkylsulfinyl radicals which may be substituted, the above-mentioned C₁₋₆ alkylsulfonyl radicals which may be substituted, the above-mentioned carboxyl radicals which may be esterified, carbamoyl, thiocarbamoyl, mono-C₁₋₆ alkylcarbamoyl, di-C₁₋₆ alkylcarbamoyl, mono- or di-C₆₋₁₄ arylcarbamoyl and mono- or di-5- to 7-membered heterocyclic carbamoyl radicals including, other than carbon atoms, from one to four heteroatoms of one or two species selected from among nitrogen, sulfur and oxygen atoms.

In Substituent Group C, examples of “heterocyclic groups which may be substituted” include 5- to 14-membered (monocyclic, bicyclic, or tricyclic) heterocyclic groups including, other than carbon atoms, from one to four heteroatoms of one or two species selected from among nitrogen, sulfur and oxygen atoms, which groups may be substituted with a substituent, including a halogen atom, hydroxy, carboxy, nitro, cyano, the above-mentioned C₁₋₆ alkyl which may be substituted, the above-mentioned C₂₋₆ alkenyl which may be substituted, the above-mentioned C₂₋₆ alkynyl which may be substituted, the above-mentioned C₃₋₈ cycloalkyl which may be substituted, the above-mentioned C₆₋₁₄ aryl which may be substituted, the above-mentioned C₁₋₆ alkoxy which may be substituted, the above-mentioned C₁₋₆ alkylthio which may be substituted, the above-mentioned C₆₋₁₄ arylthio which may be substituted, the above-mentioned C₇₋₁₆ aralkylthio which may be substituted, the above-mentioned C₁₋₆ alkylsulfinyl which may be substituted, the above-mentioned C₆₋₁₄ arylsulfinyl which may be substituted, the above-mentioned C₁₋₆ alkylsulfonyl which may be substituted, the above-mentioned C₆₋₁₄ arylsulfonyl which may be substituted, the above-mentioned carboxyl which may be esterified, carbamoyl, thiocarbamoyl, mono-C₁₋₆ alkylcarbamoyl, di-lower alkylcarbamoyl, mono- or di-C₆₋₁₄ arylcarbamoyl, and mono- or di-5- to 7-membered heterocyclic carbamoyl including, other than carbon atoms, from one to four heteroatoms of one or two species selected from among nitrogen, sulfur and oxygen atoms. Preferred examples include (i) 5- to 14-membered (preferably 5- to 10-membered) aromatic heterocyclic groups, (ii) 5- to 10-membered non-aromatic heterocyclic groups, and (iii) monovalent groups obtained by removing any one hydrogen atom from a 7- to 10-membered heterobridged ring. Of these, a 5-membered aromatic heterocyclic group is especially preferred. Illustrative examples include aromatic heterocyclic groups such as thienyl (e.g., 2-thienyl, 3-thienyl), furyl (e.g., 2-furyl, 3-furyl), pyridyl (e.g., 2-pyridyl, 3-pyridyl, 4-pyridyl), thiazolyl (e.g., 2-thiazolyl, 4-thiazolyl, 5-thiazolyl), oxazolyl (e.g., 2-oxazolyl, 4-oxazolyl), quinolyl (e.g., 2-quinolyl, 3-quinolyl, 4-quinolyl, 5-quinolyl, 8-quinolyl), isoquinolyl (e.g., 1-isoquinolyl, 3-isoquinolyl, 4-isoquinolyl, 5-isoquinolyl), pyrazinyl, pyrimidinyl (e.g., 2-pyrimidinyl, 4-pyrimidinyl), pyrrolyl (e.g., 1-pyrrolyl, 2-pyrrolyl, 3-pyrrolyl), imidazolyl (e.g., 1-imidazolyl, 2-imidazolyl, 4-imidazolyl), pyrazolyl (e.g., 1-pyrazolyl, 3-pyrazolyl, 4-pyrazolyl), pyridazinyl (e.g., 3-pyridazinyl, 4-pyridazinyl), isothiazolyl (e.g., 3-isothiazolyl), isoxazolyl (e.g., 3-isoxazolyl), indolyl (e.g., 1-indolyl, 2-indolyl, 3-indolyl), 2-benzothiazolyl, benzo[b]thienyl (e.g., 2-benzo[b]thienyl, 3-benzo[b]thienyl), benzo[b]furanyl (e.g., 2-benzo[b]furanyl, 3-benzo[b]furanyl); and non-aromatic heterocyclic radicals such as pyrrolidinyl (e.g., 1-pyrrolidinyl, 2-pyrrolidinyl, 3-pyrrolidinyl), oxazolidinyl (e.g., 2-oxazolidinyl), imidazolinyl (e.g., 1-imidazolinyl, 2-imidazolinyl, 4-imidazolinyl), piperidinyl (e.g., 1-piperidinyl, 2-piperidinyl, 3-piperidinyl, 4-piperidinyl), piperazinyl (e.g., 1-piperazinyl, 2-piperazinyl), morpholino and thiomorpholino.

In Substituent Group C, examples of “carbamoyl which may be substituted” include C₁₋₆ alkyl which may be substituted, C₂₋₆ alkenyl which may be substituted, C₂₋₆ alkynyl which may be substituted, C₃₋₈ cycloalkyl which may be substituted, C₆₋₁₄ aryl which may be substituted, and carbamoyl which may be substituted with a heterocyclic group that may be substituted. Illustrative examples include carbamoyl, thiocarbamoyl, mono-C₁₋₆ alkylcarbamoyl (e.g., methylcarbamoyl, ethylcarbamoyl), di-C₁₋₆ alkylcarbamoyl (e.g., dimethylcarbamoyl, diethylcarbamoyl, ethylmethylcarbamoyl), C₁₋₆ alkyl (C₁₋₆ alkoxy) carbamoyl (e.g., methyl(methoxy)carbamoyl, ethyl(methoxy)carbamoyl), mono- or di-C₆₋₁₄ arylcarbamoyl (e.g., phenylcarbamoyl, 1-naphthylcarbamoyl, 2-naphthylcarbamoyl), mono- or di- 5- to 7-membered heterocyclic carbamoyl including, other than carbon atoms, one to four heteroatoms of one or two species selected from among nitrogen, sulfur and oxygen atoms (e.g., 2-pyridylcarbamoyl, 3-pyridylcarbamoyl, 4-pyridylcarbamoyl, 2-thienylcarbamoyl, 3-thienylcarbamoyl), and 5- to 7-membered cyclic carbamoyl (e.g., 1-pyrrolidinylcarbonyl, 1-piperidinylcarbonyl, hexamethyleneiminocarbonyl).

In Substituent Group C, examples of “amino which may be substituted” include aminos which may be substituted with one or two groups such as the above-mentioned C₁₋₆ alkyl which may be substituted, the above-mentioned C₂₋₆ alkenyl which may be substituted, the above-mentioned C₂₋₆ alkynyl which may be substituted, the above-mentioned C₃₋₈ cycloalkyl which may be substituted, the above-mentioned C₆₋₁₄ aryl which may be substituted, the C₁₋₆ alkoxy which may be substituted, formyl, the above-mentioned C₁₋₆ alkylcarbonyl which may be substituted, the above-mentioned C₃₋₈ cycloalkylcarbonyl which may be substituted, the above-mentioned C₆₋₁₄ arylcarbonyl which may be substituted, the above-mentioned C₁₋₆ alkoxycarbonyl which may be substituted, the above-mentioned C₁₋₆ alkylsulfonyl which may be substituted and C₆₋₁₄ arylsulfonyl which may be substituted.

Preferred substituents include halogen atoms, hydroxy, C₁₋₆ alkoxy, C₁₋₆ alkyl which may be halogenated, C₁₋₆ alkoxy which may be halogenated, amino, nitro and cyano.

XX010 represents preferably Phe, Trp, 2-naphthylalanine, 2-thienylalanine, tyrosine or 4-fluorophenylalanine; more preferably Phe or Trp; and even more preferably Trp.

Preferred combinations of the above are metastin derivatives of the formula

XX00-XX02-XX03-XX04-XX05-XX06-AzaGly-XX08-XX09-XX010-NH₂,

or a salt thereof, wherein:
XX00 represents formyl, C₁₋₆ alkanoyl or glycoloyl;
XX02 represents D-Tyr or a valence bond;
XX03 represents Aze(2), Hyp, Gly, Aib, Leu, Lys, Glu, His(3Me), Tyr(PO₃H₂), Hyp(Bzl) or Pro(4F);
XX04 represents Asn or 2-amino-3-ureidopropionic acid;
XX05 represents Ser, Thr or Ala;
XX06 represents Phe, Cha, Phe(2F), Phe(3F), Phe(4F) or Phe(4Cl);
AzaGly represents azaglycine;
XX08 represents Leu or Ala(cPr);
XX09 represents Arg or Arg(Me); and
XX010 represents Phe or Trp.

More preferred combinations of the above are metastin derivatives of the formula

XX00-XX02-XX03-XX04-XX05-XX06-AzaGly-XX08-XX09-XX010-NH₂,

or a salt thereof, wherein:
XX00 represents acetyl or glycoloyl (preferably acetyl);
XX02 represents D-Tyr;
XX03 represents Hyp, Glu, Hyp(Bzl) or Pro(4F);
XX04 represents Asn or 2-amino-3-ureidopropionic acid;
XX05 represents Thr;
XX06 represents Phe, Cha, Phe(3F) or Phe(4F);
AzaGly represents azaglycine;
XX08 represents Leu or Ala(cPr);
XX09 represents Arg or Arg(Me); and
XX010 represents Trp.

Although all compounds in which the above-indicated groups represented by the various symbols are combined in any way may be suitably used as the metastin derivative (III), preferred compounds include those represented as Compound Nos. 708 to 899 in WO 2007/072997. Of these, the compounds represented by the following compound numbers are more preferred.

Compound No. 708:
des(1)-Ac-[D-Tyr2,D-Trp3,Thr5,AzaGly7,D-Arg9,Trp10]MS10
Ac-D-Tyr-D-Trp-Asn-Thr-Phe-AzaGly-Leu-D-Arg-Trp-NH₂
Compound No. 709:
des(1-3)-Ac-[Thr5,AzaGly7,Arg(Me)9,Trp10]MS10
Ac-Asn-Thr-Phe-AzaGly-Leu-Arg(Me)-Trp-NH₂
Compound No. 710:
des(1-3)-Decanoyl-[Thr5,AzaGly7,Arg(Me)9,Trp10]MS10
Decanoyl-Asn-Thr-Phe-AzaGly-Leu-Arg(Me)-Trp-NH₂
Compound No. 712:
des(1-2)-[Acp3, Thr5,AzaGly7,Arg(Me)9,Trp10]MS10
Acp-Asn-Thr-Phe-AzaGly-Leu-Arg(Me)-Trp-NH₂
Compound No. 713:
des(1-2)-Ac-[Acp3,Thr5,AzaGly7,Arg(Me)9,Trp10]MS10
Ac-Acp-Asn-Thr-Phe-AzaGly-Leu-Arg(Me)-Trp-NH₂
Compound No. 714:
des(1)-Ac-[D-Tyr2,D-Trp3,Asp(NHPen)4,Thr5,AzaGly7,Arg(Me)9,Trp10]MS10
Ac-D-Tyr-D-Trp-Asp(NHPen)-Thr-Phe-AzaGly-Leu-Arg(Me)-Trp-NH₂
Compound No. 715:
des(1)-Ac-[D-Tyr2,D-Trp3,Asp(NHcPr)4,Thr5,AzaGly7,Arg(Me)9,Trp10]MS10
Ac-D-Tyr-D-Trp-Asp(NHcPr)-Thr-Phe-AzaGly-Leu-Arg(Me)-Trp-NH₂
Compound No. 716:
des(1)-Ac[D-Tyr2,D-Trp3,Asp(NHBzl)4,Thr5,AzaGly7,Arg(Me)9,Trp10]MS10
Ac-D-Tyr-D-Trp-Asp(NHBzl)-Thr-Phe-AzaGly-Leu-Arg(Me)-Trp-NH₂
Compound No. 717:
des(1)-Ac[-D-Tyr2,D-Trp3,Alb4,Thr5,AzaGly7,Arg(Me)9,Trp10]MS10
Ac-D-Tyr-D-Trp-Alb-Thr-Phe-AzaGly-Leu-Arg(Me)-Trp-NH₂
Compound No. 718:
des(1)-Ac-[D-Tyr2,D-Pya(4)3,Alb4,Thr5,AzaGly7,Arg(Me)9,Trp10]MS10
Ac-D-Tyr-D-Pya(4)-Alb-Thr-Phe-AzaGly-Leu-Arg(Me)-Trp-NH₂
Compound No. 719:
des(1)-Ac-[D-Tyr2,D-Trp3,D-Pro5,AzaGly7,Arg(Me)9,Trp10]MS10
Ac-D-Tyr-D-Trp-Asn-D-Pro-Phe-AzaGly-Leu-Arg(Me)-Trp-NH₂
Compound No. 720:
des(1)-Ac-[D-Tyr2,Aze(2)3,Thr5,AzaGly7,Arg(Me)9,Trp10]MS10
Ac-D-Tyr-Aze(2)-Asn-Thr-Phe-AzaGly-Leu-Arg(Me)-Trp-NH₂
Compound No. 721:
des(1)-Ac-[D-Tyr2,Pic(2)3,Thr5,AzaGly7,Arg(Me)9,Trp10]MS10
Ac-D-Tyr-Pic(2)-Asn-Thr-Phe-AzaGly-Leu-Arg(Me)-Trp-NH₂
Compound No. 722:
des(1)-Ac-[D-Tyr2,Pic(3)3,Thr5,AzaGly7,Arg(Me)9,Trp10]MS10
Ac-D-Tyr-Pic(3 )-Asn-Thr-Phe-AzaGly-Leu-Arg(Me)-Trp-NH₂
Compound No. 723:
des(1)-Ac-[D-Tyr2,Hyp3,Thr5,AzaGly7,Arg(Me)9,Trp10]MS10
Ac-D-Tyr-Hyp-Asn-Thr-Phe-AzaGly-Leu-Arg(Me)-Trp-NH₂
Compound No. 724:
des(1)-Ac-[D-Tyr2,Thz3,Thr5,AzaGly7,Arg(Me)9,Trp10]MS10
Ac-D-Tyr-Thz-Asn-Thr-Phe-AzaGly-Leu-Arg(Me)-Trp-NH₂
Compound No. 725:
des(1)-Ac-[D-Tyr2,NMeAla3,Thr5,AzaGly7,Arg(Me)9,Trp10]MS10
Ac-D-Tyr-NMeAla-Asn-Thr-Phe-AzaGly-Leu-Arg(Me)-Trp-NH₂
Compound No. 726:
des(1)-Ac-[D-Tyr2,Gly3,Thr5,AzaGly7,Arg(Me)9,Trp10]MS10
Ac-D-Tyr-Gly-Asn-Thr-Phe-AzaGly-Leu-Arg(Me)-Trp-NH₂
Compound No. 727:
des(1)-Ac-[D-Tyr2,Alb3,Thr5,AzaGly7,Arg(Me)9,Trp10]MS10
Ac-D-Tyr-Alb-Asn-Thr-Phe-AzaGly-Leu-Arg(Me)-Trp-NH₂
Compound No. 728:
des(1)-Ac-[D-Tyr2,Abz(2)3,Thr5,AzaGly7,Arg(Me)9,Trp10]MS10
Ac-D-Tyr-Abz(2)-Asn-Thr-Phe-AzaGly-Leu-Arg(Me)-Trp-NH₂
Compound No. 730:
des(1)-Ac-[D-Tyr2,Aze(3)3,Thr5,AzaGly7,Arg(Me)9,Trp10]MS10
Ac-D-Tyr-Aze(3)-Asn-Thr-Phe-AzaGly-Leu-Arg(Me)-Trp-NH₂
Compound No. 731:
des(1)-Ac-[D-Tyr2,Sar3,Thr5,AzaGly7,Arg(Me)9,Trp10]MS10
Ac-D-Tyr-Sar-Asn-Thr-Phe-AzaGly-Leu-Arg(Me)-Trp-NH₂
Compound No. 732:
des(1)-Ac-[D-Tyr2,D-NMeAla3,Thr5,AzaGly7,Arg(Me)9,Trp101]MS10
Ac-D-Tyr-D-NMeAla-Asn-Thr-Phe-AzaGly-Leu-Arg(Me)-Trp-NH₂
Compound No. 734:
des(1)-Ac-[D-Tyr2,Izc3,Thr5,AzaGly7,Arg(Me)9,Trp10]MS10
Ac-D-Tyr-Izc-Asn-Thr-Phe-AzaGly-Leu-Arg(Me)-Trp-NH₂
Compound No. 735:
des(1)-Ac[D-Tyr2,D-Asp3,Thr5,AzaGly7,Arg(Me)9,Trp10]MS10
Ac-D-Tyr-D-Asp-Asn-Thr-Phe-AzaGly-Leu-Arg(Me)-Trp-NH₂
Compound No. 736:
des(1)-Ac-[D-Tyr2,D-Dap3,Thr5,AzaGly7,Arg(Me)9,Trp10]MS10
Ac-D-Tyr-D-Dap-Asn-Thr-Phe-AzaGly-Leu-Arg(Me)-Trp-NH₂
Compound No. 737:
des(1)-Ac-[D-Tyr2,D-Ser3,Thr5,AzaGly7,Arg(Me)9,Trp10]MS10
Ac-D-Tyr-D-Ser-Asn-Thr-Phe-AzaGly-Leu-Arg(Me)-Trp-NH₂
Compound No. 738:
des(1)-Ac-[D-Tyr2,D-Gln3,Thr5,AzaGly7,Arg(Me)9,Trp10]MS10
Ac-D-Tyr-D-Gln-Asn-Thr-Phe-AzaGly-Leu-Arg(Me)-Trp-NH₂
Compound No. 739:
des(1)-Ac-[D-Tyr2,D-His3,Thr5,AzaGly7,Arg(Me)9,Trp10]MS10
Ac-D-Tyr-D-His-Asn-Thr-Phe-AzaGly-Leu-Arg(Me)-Trp-NH₂
Compound No. 740:
des(1)-Ac-[D-Tyr2,D-Trp3,Dab4,Thr5,AzaGly7,Arg(Me)9,Trp10]MS10
Ac-D-Tyr-D-Trp-Dab-Thr-Phe-AzaGly-Leu-Arg(Me)-Trp-NH₂
Compound No. 742:
des(1)-Ac-[D-Tyr2,Ala3,Thr5,AzaGly7,Arg(Me)9,Trp10]MS10
Ac-D-Tyr-Ala-Asn-Thr-Phe-AzaGly-Leu-Arg(Me)-Trp-NH₂
Compound No. 743:
des(1)-Ac-[D-Tyr2,Leu3,Thr5,AzaGly7,Arg(Me)9,Trp10]MS10
Ac-D-Tyr-Leu-Asn-Thr-Phe-AzaGly-Leu-Arg(Me)-Trp-NH₂
Compound No. 744:
des(1)-Ac-[D-Tyr2,Ser3,Thr5,AzaGly7,Arg(Me)9,Trp10]MS10
Ac-D-Tyr-Ser-Asn-Thr-Phe-AzaGly-Leu-Arg(Me)-Trp-NH₂
Compound No. 745:
des(1)-Ac-[D-Tyr2,Lys3,Thr5,AzaGly7,Arg(Me)9,Trp10]MS10
Ac-D-Tyr-Lys-Asn-Thr-Phe-AzaGly-Leu-Arg(Me)-Trp-NH₂
Compound No. 746:
des(1)-Ac-[D-Tyr2,Glu3,Thr5,AzaGly7,Arg(Me)9,Trp10]MS10
Ac-D-Tyr-Glu-Asn-Thr-Phe-AzaGly-Leu-Arg(Me)-Trp-NH₂
Compound No. 747:
des(1)-Ac-[D-Tyr2,β-Ala3,Thr5,AzaGly7,Arg(Me)9,Trp10]MS10
Ac-D-Tyr-β-Ala-Asn-Thr-Phe-AzaGly-Leu-Arg(Me)-Trp-NH₂
Compound No. 748:
des(1)-Ac-[D-Tyr2,D-Trp3,Thr5,Phe(4Cl)6,AzaGly7,Arg(Me)9,Trp10]MS10
Ac-D-Tyr-D-Trp-Asn-Thr-Phe(4Cl)-AzaGly-Leu-Arg(Me)-Trp-NH₂
Compound No. 749:
des(1)-Ac-[D-Tyr2,D-Trp3,Thr5,Phe(2F)6,AzaGly7,Arg(Me)9,Trp10]MS10
Ac-D-Tyr-D-Trp-Asn-Thr-Phe(2F)-AzaGly-Leu-Arg(Me)-Trp-NH₂
Compound No. 750:
des(1)-Ac-[D-Tyr2,D-Trp3,Thr5,Phe(3F)6,AzaGly7,Arg(Me)9,Trp10]MS10
Ac-D-Tyr-D-Trp-Asn-Thr-Phe(3F)-AzaGly-Leu-Arg(Me)-Trp-NH₂
Compound No. 754:
des(1)-Ac-[D-Tyr2,Lys3,Thr5,Phe(2F)6,AzaGly7,Arg(Me)9,Trp10]MS10
Ac-D-Tyr-Lys-Asn-Thr-Phe(2F)-AzaGly-Leu-Arg(Me)-Trp-NH₂
Compound No. 755:
des(1)-Ac-[D-Tyr2,Glu3,Thr5,Phe(2F)6,AzaGly7,Arg(Me)9,Trp10]MS10
Ac-D-Tyr-Glu-Asn-Thr-Phe(2F)-AzaGly-Leu-Arg(Me)-Trp-NH₂
Compound No. 756:
des(1)-Ac-[D-Tyr2,Lys3,Thr5,Phe(3F)6,AzaGly7,Arg(Me)9,Trp10]MS10
Ac-D-Tyr-Lys-Asn-Thr-Phe(3F)-AzaGly-Leu-Arg(Me)-Trp-NH₂
Compound No. 757:
des(1)-Ac-[D-Tyr2,Glu3,Thr5,Phe(3F)6,AzaGly7,Arg(Me)9,Trp10]MS10
Ac-D-Tyr-Glu-Asn-Thr-Phe(3F)-AzaGly-Leu-Arg(Me)-Trp-NH₂
Compound No. 758:
des(1)-Ac-[D-Tyr2,Lys3,Thr5,Phe(4Cl)6,AzaGly7,Arg(Me)9,Trp10]MS10
Ac-D-Tyr-Lys-Asn-Thr-Phe(4Cl)-AzaGly-Leu-Arg(Me)-Trp-NH₂
Compound No. 759:
des(1)-Ac-[D-Tyr2,Glu3,Thr5,Phe(4Cl)6,AzaGly7,Arg(Me)9,Trp10]MS10
Ac-D-Tyr-Glu-Asn-Thr-Phe(4Cl)-AzaGly-Leu-Arg(Me)-Trp-NH₂
Compound No. 760:
des(1)-Ac[D-Tyr2,Pzc(2)3,Thr5,AzaGly7,Arg(Me)9,Trp10]MS10
Ac-D-Tyr-Pzc(2)-Asn-Thr-Phe-AzaGly-Leu-Arg(Me)-Trp-NH₂
Compound No. 763:
des(1)-Ac-[D-Tyr2,Hyp3,Thr5,Phe(2F)6,AzaGly7,Arg(Me)9,Trp10]MS10
Ac-D-Tyr-Hyp-Asn-Thr-Phe(2F)-AzaGly-Leu-Arg(Me)-Trp-NH₂
Compound No. 764:
des(1)-Ac-[D-Tyr2,Trp3,Thr5,Phe(2F)6,AzaGly7,Arg(Me)9,Trp10]MS10
Ac-D-Tyr-Trp-Asn-Thr-Phe(2F)-AzaGly-Leu-Arg(Me)-Trp-NH₂
Compound No. 765:
des(1)-Ac-[D-Tyr2,Hyp3,Thr5,Phe(3F)6,AzaGly7,Arg(Me)9,Trp10]MS10
Ac-D-Tyr-Hyp-Asn-Thr-Phe(3F)-AzaGly-Leu-Arg(Me)-Trp-NH₂
Compound No. 766:
des(1)-Ac-[D-Tyr2,Trp3,Thr5,Phe(3F)6,AzaGly7,Arg(Me)9,Trp10]MS10
Ac-D-Tyr-Trp-Asn-Thr-Phe(3F)-AzaGly-Leu-Arg(Me)-Trp-NH₂
Compound No. 767:
des(1)-Ac-[D-Tyr2,Hyp3,Thr5,Phe(4Cl)6,AzaGly7,Arg(Me)9,Trp10]MS10
Ac-D-Tyr-Hyp-Asn-Thr-Phe(4Cl)-AzaGly-Leu-Arg(Me)-Trp-NH₂
Compound No. 768:
des(1)-Ac-[D-Tyr2,Trp3,Thr5,Phe(4Cl)6,AzaGly7,Arg(Me)9,Trp10]MS10
Ac-D-Tyr-Trp-Asn-Thr-Phe(4Cl)-AzaGly-Leu-Arg(Me)-Trp-NH₂
Compound No. 769:
des(1)-Ac-[D-Tyr2,Gly3,Thr5,Phe(4Cl)6,AzaGly7,Arg(Me)9,Trp10]MS10
Ac-D-Tyr-Gly-Asn-Thr-Phe(4Cl)-AzaGly-Leu-Arg(Me)-Trp-NH₂
Compound No. 770:
des(1)-Ac-[D-Tyr2,Alb3,Thr5,Phe(4Cl)6,AzaGly7,Arg(Me)9,Trp10]MS10
Ac-D-Tyr-Alb-Asn-Thr-Phe(4Cl)-AzaGly-Leu-Arg(Me)-Trp-NH₂
Compound No. 771:
des(1)-Ac-[D-Tyr2,Orn3,Thr5,AzaGly7,Arg(Me)9,Trp10]MS10
Ac-D-Tyr-Orn-Asn-Thr-Phe-AzaGly-Leu-Arg(Me)-Trp-NH₂
Compound No. 772:
des(1)-Ac-[D-Tyr2,Thr3,Thr5,AzaGly7,Arg(Me)9,Trp10]MS10
Ac-D-Tyr-Thr-Asn-Thr-Phe-AzaGly-Leu-Arg(Me)-Trp-NH₂
Compound No. 773:
des(1)-Ac-[D-Tyr2,His(3Me)3,Thr5,AzaGly7,Arg(Me)9,Trp10]MS10
Ac-D-Tyr-His(3Me)-Asn-Thr-Phe-AzaGly-Leu-Arg(Me)-Trp-NH₂
Compound No. 774:
des(1)-Ac[D-Tyr2,DL-Ala(Pip)3,Thr5,AzaGly7,Arg(Me)9,Trp10]MS10
Ac-D-Tyr-DL-Ala(Pip)-Asn-Thr-Phe-AzaGly-Leu-Arg(Me)-Trp-NH₂
Compound No. 775:
des(1)-Ac-[D-Tyr2,Tyr(PO₃H₂)3,Thr5,AzaGly7,Arg(Me)9,Trp10]MS10
Ac-D-Tyr-Tyr(PO₃H₂)-Asn-Thr-Phe-AzaGly-Leu-Arg(Me)-Trp-NH₂
Compound No. 776:
des(1)-Glycoloyl[D-Tyr2,Hyp3,Thr5,AzaGly7,Arg(Me)9,Trp10]MS10
Glycoloyl-D-Tyr-Hyp-Asn-Thr-Phe-AzaGly-Leu-Arg(Me)-Trp-NH₂
Compound No. 777:
des(1-2)-Ac-[D-Tyr3,Thr5,AzaGly7,Arg(Me)9,Trp10]MS10
Ac-D-Tyr-Asn-Thr-Phe-AzaGly-Leu-Arg(Me)-Trp-NH₂
Compound No. 780:
des(1)-Ac-[D-Tyr2,Pro(4NH₂)3,Thr5,AzaGly7,Arg(Me)9,Trp10]MS10
Ac-D-Tyr-Pro(4NH₂)-Asn-Thr-Phe-AzaGly-Leu-Arg(Me)-Trp-NH₂
Compound No. 781:
des(1)-Ac-[D-Tyr2,Hyp(Bzl)3,Thr5,AzaGly7,Arg(Me)9,Trp10]MS10
Ac-D-Tyr-Hyp(Bzl)-Asn-Thr-Phe-AzaGly-Leu-Arg(Me)-Trp-NH₂
Compound No. 782:
des(1)-Ac-[D-Tyr2,D-NMePhe3,Thr5,AzaGly7,Arg(Me)9,Trp10]MS10
Ac-D-Tyr-D-NMePhe-Asn-Thr-Phe-AzaGly-Leu-Arg(Me)-Trp-NH₂
Compound No. 783:
des(1)-Ac-[D-Thr5,Phe(2F)6,AzaGly7,Arg(Me)9,Trp10]MS10
Ac-D-Tyr-Gly-Asn-Thr-Phe(2F)-AzaGly-Leu-Arg(Me)-Trp-NH₂
Compound No. 784:
des(1)-Ac-[D-Tyr2,Alb3,Thr5,Phe(2F)6,AzaGly7,Arg(Me)9,Trp10]MS10
Ac-D-Tyr-Alb-Asn-Thr-Phe(2F)-AzaGly-Leu-Arg(Me)-Trp-NH₂
Compound No. 785:
des(1)-Ac-[D-Tyr2,Gly3,Thr5,Phe(3F)6,AzaGly7,Arg(Me)9,Trp10]MS10
Ac-D-Tyr-Gly-Asn-Thr-Phe(3F)-AzaGly-Leu-Arg(Me)-Trp-NH₂
Compound No. 786:
des(1)-Ac-[D-Tyr2,Alb3,Thr5,Phe(3F)6,AzaGly7,Arg(Me)9,Trp10]MS10
Ac-D-Tyr-Alb-Asn-Thr-Phe(3F)-AzaGly-Leu-Arg(Me)-Trp-NH₂
Compound No. 787:
des(1)-Ac-[D-Tyr2,Hyp3,Thr5,Phe(4F)6,AzaGly7,Arg(Me)9,Trp10]MS10
Ac-D-Tyr-Hyp-Asn-Thr-Phe(4F)-AzaGly-Leu-Arg(Me)-Trp-NH₂
Compound No. 788:
des(1)-Ac-[D-Thr5,Phe(4F)6,AzaGly7,Arg(Me)9,Trp10]MS10
Ac-D-Tyr-Glu-Asn-Thr-Phe(4F)-AzaGly-Leu-Arg(Me)-Trp-NH₂
Compound No. 789:
des(1)-Ac-[D-Tyr2,Lys3,Thr5,Phe(4F)6,AzaGly7,Arg(Me)9,Trp10]MS10
Ac-D-Tyr-Lys-Asn-Thr-Phe(4F)-AzaGly-Leu-Arg(Me)-Trp-NH₂
Compound No. 790:
des(1)-Ac-[D-Tyr2,Gly3,Thr5,Phe(4F)6,AzaGly7,Arg(Me)9,Trp10]MS10
Ac-D-Tyr-Gly-Asn-Thr-Phe(4F)-AzaGly-Leu-Arg(Me)-Trp-NH₂
Compound No. 791:
des(1)-Ac-[D-Tyr2,Alb3,Thr5,Phe(4F)6,AzaGly7,Arg(Me)9,Trp10]MS10
Ac-D-Tyr-Alb-Asn-Thr-Phe(-F)-AzaGly-Leu-Arg(Me)-Trp-NH₂
Compound No. 794:
des(1)-Ac-[D-Tyr2,Hyp3,Thr5,D-Phe6,AzaGly7,Arg(Me)9,Trp10]MS10
Ac-D-Tyr-Hyp-Asn-Thr-D-Phe-AzaGly-Leu-Arg(Me)-Trp-NH₂
Compound No. 797:
des(1)-Ac-[D-Tyr2,Hyp3,Thr5,AzaGly7,Trp10]MS10
Ac-D-Tyr-Hyp-Asn-Thr-Phe-AzaGly-Leu-Arg-Trp-NH₂
Compound No. 800:
des(1)-Ac-[D-Tyr2,Hyp3,Alb4,Thr5,AzaGly7,Arg(Me)9,Trp10]MS10
Ac-D-Tyr-Hyp-Alb-Thr-Phe-AzaGly-Leu-Arg(Me)-Trp-NH2
Compound No. 801:
des(1-5)-4-[Bis-(2-Pyridylmethyl)aminomethyl]benzoyl-
[AzaGly7,Arg(Me)9,Trp10]MS10
4-[Bis-(2-Pyridylmethyl)aminomethyl]benzoyl-Phe-AzaGly-
Leu-Arg(Me)-Trp-NH₂
Compound No. 809:
des(1)-Ac-[D-Tyr2,Hyp3,NMeSer5,AzaGly7,Arg(Me)9,Trp10]MS10
Ac-D-Tyr-Hyp-Asn-NMeSer-Phe-AzaGly-Leu-Arg(Me)-Trp-NH₂
Compound No. 810:
des(1)-Ac-[D-Tyr2,Hyp3,Hyp5,AzaGly7,Arg(Me)9,Trp10]MS10
Ac-D-Tyr-Hyp-Asn-Hyp-Phe-AzaGly-Leu-Arg(Me)-Trp-NH₂
Compound No. 813:
des(1)-Ac-[D-Tyr2,Hyp3,Gly5,AzaGly7,Arg(Me)9,Trp10]MS10
Ac-D-Tyr-Hyp-Asn-Gly-Phe-AzaGly-Leu-Arg(Me)-Trp-NH₂
Compound No. 814:
des(1)-Ac-[D-Tyr2,Hyp3,Ala5,AzaGly7,Arg(Me)9,Trp10]MS10
Ac-D-Tyr-Hyp-Asn-Ala-Phe-AzaGly-Leu-Arg(Me)-Trp-NH₂
Compound No. 815:
des(1)-Ac-[D-Tyr2,Hyp3,D-Ala5,AzaGly7,Arg(Me)9,Trp10]MS10
Ac-D-Tyr-Hyp-Asn-D-Ala-Phe-AzaGly-Leu-Arg(Me)-Trp-NH₂
Compound No. 816:
des(1)-Ac-[D-Tyr2,Hyp3,His4,Thr5,AzaGly7,Arg(Me)9,Trp10]MS10
Ac-D-Tyr-Hyp-His-Thr-Phe-AzaGly-Leu-Arg(Me)-Trp-NH₂
Compound No. 843:
des(1)-Ac-[D-Tyr2,Hyp3,Gln4,Thr5,AzaGly7,Arg(Me)9,Trp10]MS10
Ac-D-Tyr-Hyp-Gln-Thr-Phe-AzaGly-Leu-Arg(Me)-Trp-NH₂
Compound No. 844:
des(1)-Ac-[D-Tyr2,Hyp3,D-Asn4,Thr5,AzaGly7,Arg(Me)9,Trp10]MS10
Ac-D-Tyr-Hyp-D-Asn-Thr-Phe-AzaGly-Leu-Arg(Me)-Trp-NH₂
Compound No. 845:
des(1)-Ac-[D-Tyr2,Hyp3,Cit4,Thr5,AzaGly7,Arg(Me)9,Trp10]MS10
Ac-D-Tyr-Hyp-Cit-Thr-Phe-AzaGly-Leu-Arg(Me)-Trp-NH₂
Compound No. 846:
des(1)-Ac-[D-Tyr2,Hyp3,D-Thr5,AzaGly7,Arg(Me)9,Trp10]MS10
Ac-D-Tyr-Hyp-Asn-D-Thr-Phe-AzaGly-Leu-Arg(Me)-Trp-NH₂
Compound No. 856:
des(1)-Ac-[D-Tyr2,Hyp3,Thr5,AzaGly7,Ala(cPr)8,Arg(Me)9,Trp10]MS10
Ac-D-Tyr-Hyp-Asn-Thr-Phe-AzaGly-Ala(cPr)-Arg(Me)-Trp-NH₂
Compound No. 860:
des(1-5)-4-Ureidobenzoyl-[AzaGly7,Arg(Me)9,Trp10]MS10
4-Ureidobenzoyl-Phe-AzaGly-Leu-Arg(Me)-Trp-NH₂
Compound No. 861:
des(1)-Ac-[D-Tyr2,Hyp3,Arg4,Thr5,AzaGly7,Arg(Me)9,Trp10]MS10
Ac-D-Tyr-Hyp-Arg-Thr-Phe-AzaGly-Leu-Arg(Me)-Trp-NH₂
Compound No. 862:
des(1)-Ac-[D-Tyr2,Hyp3,Gly4,Thr5,AzaGly7,Arg(Me)9,Trp10]MS10
Ac-D-Tyr-Hyp-Gly-Thr-Phe-AzaGly-Leu-Arg(Me)-Trp-NH₂
Compound No. 863:
des(1)-Ac-[D-Tyr2,Hyp3,Dap4,Thr5,AzaGly7,Arg(Me)9,Trp10]MS10
Ac-D-Tyr-Hyp-Dap-Thr-Phe-AzaGly-Leu-Arg(Me)-Trp-NH₂
Compound No. 864:
des(1)-Ac-[D-Tyr2,Hyp3,Dab4,Thr5,AzaGly7,Arg(Me)9,Trp10]MS10
Ac-D-Tyr-Hyp-Dab-Thr-Phe-AzaGly-Leu-Arg(Me)-Trp-NH₂
Compound No. 868:
des(1)-Ac-[D-Tyr2,Hyp3,Thr5,αMePhe6,AzaGly7,Arg(Me)9,Trp10]MS10
Ac-D-Tyr-Hyp-Asn-Thr-αMePhe-AzaGly-Leu-Arg(Me)-Trp-NH₂
Compound No. 870:
des(1)-Ac-[D-Tyr2,Hyp3,Thr5,Phe(2Me)6,AzaGly7,Arg(Me)9,Trp10]MS10
Ac-D-Tyr-Hyp-Asn-Thr-Phe(2Me)-AzaGly-Leu-Arg(Me)-Trp-NH₂
Compound No. 872:
des(1)-Ac-[D-Tyr2,Hyp3,Thr5,Phe(3Me)6,AzaGly7,Arg(Me)9,Trp10]MS10
Ac-D-Tyr-Hyp-Asn-Thr-Phe(3Me)-AzaGly-Leu-Arg(Me)-Trp-NH₂
Compound No. 874:
des(1)-Ac-[D-Tyr2,Hyp3,Thr5,Phe(4Me)6,AzaGly7,Arg(Me)9,Trp10]MS10
Ac-D-Tyr-Hyp-Asn-Thr-Phe(4Me)-AzaGly-Leu-Arg(Me)-Trp-NH₂
Compound No. 877:
des(1)-Ac-[D-Tyr2,Hyp3,Thr5,threo-Ser(3Phenyl)6,AzaGly7,Arg(Me)9,Trp10]MS10
Ac-D-Tyr-Hyp-Asn-Thr-threo-Ser(3Phenyl)-AzaGly-Leu-Arg(Me)-Trp-NH₂
Compound No. 882:
des(1)-Ac-[D-Tyr2,Hyp3,Thr5,erythro-Ser(3Phenyl)6,AzaGly7,Arg(Me)9,Trp10]MS10
Ac-D-Tyr-Hyp-Asn-Thr-erythro-Ser(3Phenyl)-AzaGly-Leu-Arg(Me)-Trp-NH₂
Compound No. 886:
des(1)-Ac[D-Tyr2,Hyp3,Nva4,Thr5,AzaGly7,Arg(Me)9,Trp10]MS10
Ac-D-Tyr-Hyp-Nva-Thr-Phe-AzaGly-Leu-Arg(Me)-Trp-NH₂
Compound No. 887:
des(1-2)-Ac-[Hyp3,Thr5,AzaGly7,Arg(Me)9,Trp10]MS10
Ac-Hyp-Asn-Thr-Phe-AzaGly-Leu-Arg(Me)-Trp-NH₂
Compound No. 888:
des(1-2)-3-(p-Hydroxyphenyl)propionyl-[Hyp3,Thr5,AzaGly7,Arg(Me)9,Trp10]MS10
3-(p-Hydroxyphenyl)propionyl-Hyp-Asn-Thr-Phe-AzaGly-Leu-Arg(Me)-Trp-NH₂
Compound No. 889:
des(1-2)-[pGlu3,Thr5,AzaGly7,Arg(Me)9,Trp10]MS10
pGlu-Asn-Thr-Phe-AzaGly-Leu-Arg(Me)-Trp-NH₂
Compound No. 896:
des(1)-Ac-[D-Tyr2,cisHyp3,Thr5,AzaGly7,Arg(Me)9,Trp10]MS10
Ac-D-Tyr-cisHyp-Asn-Thr-Phe-AzaGly-Leu-Arg(Me)-Trp-NH₂
Compound No. 897:
des(1)-Ac-[D-Tyr2,Pro(4F)3,Thr5,AzaGly7,Arg(Me)9,Trp10]MS10
Ac-D-Tyr-Pro(4F)-Asn-Thr-Phe-AzaGly-Leu-Arg(Me)-Trp-NH₂
Compound No. 899:
des(1)-Ac-[Tyr2,Hyp3,Thr5,AzaGly7,Arg(Me)9,Trp10]MS10
Ac-Tyr-Hyp-Asn-Thr-Phe-AzaGly-Leu-Arg(Me)-Trp-NH₂

The compound indicated by the following compound number is especially preferred as the metastin derivative (III) and/or (IV).

(Compound No. 723).
Ac-D-Tyr-Hyp-Asn-Thr-Phe-AzaGly-Leu-Arg(Me)-Trp-NH₂

The metastin derivative (IV) of the present invention (which includes metastin derivatives (I), (II) and (III); abbreviated below as “the inventive compound” or “the compound of the present invention”) may be synthesized in accordance with a method described in WO 2004/063221, WO 2006/001499 or WO 2007/072997.

When the compound of the present invention is present in the form of a configurational isomer, a diastereomer, a conformer, or the like, each can be isolated by the separating and purifying means described above, if desired. In addition, when the compound of the present invention is racemic, it can be separated into an S isomer and an R isomer by the conventional optical resolving means.

When steric isomers exist in the compound of the present invention, the present invention includes both of these isomers alone and the isomers present as a mixture thereof.

In addition, thecompound of the present invention may also be hydrated or non-hydrated. The compound of the present invention may also be labeled with an isotope (e.g., ³H, ¹⁴C, ³⁵S), etc.

Throughout the specification, the peptides are represented in accordance with the conventional way of describing peptides, that is, the N-terminus (amino terminus) at the left hand and the C-terminus (carboxyl terminus) at the right hand. In the peptides, the C-terminus is usually in the form of an amide (—CONH₂), a carboxyl group (—COOH), a carboxylate (—COO⁻), an alkylamide (—CONHR) or an ester (—COOR) and the amide (—CONH₂) is particularly preferred. Examples of R in the ester or alkylamide include a C₁₋₆ alkyl group such as methyl, ethyl, n-propyl, isopropyl, n-butyl, etc.; a C₃₋₈ cycloalkyl group such as cyclopentyl, cyclohexyl, etc.; a C₆₋₁₂ aryl group such as phenyl, α-naphthyl, etc.; a C₇₋₁₄ aralkyl group such as a phenyl-C₁₋₂-alkyl group, e.g., benzyl, phenethyl, etc., or an α-naphthyl-C₁₋₂-alkyl group such as α-naphthylmethyl, etc.; and pivaloyloxymethyl group, which are widely used as an ester for oral use, and the like.

Examples of salts of the compound of the present invention include a metal salt, an ammonium salt, a salt with an organic base, a salt with inorganic acid, a salt with organic acid, a salt with basic or acidic amino acid, and the like. Preferred examples of the metal salts include alkali metal salts such as sodium salts, potassium salts, etc.; alkaline earth metal salts such as calcium salts, magnesium salts, barium salts, etc.; aluminum salts; and the like. Preferred examples of the salts with organic bases include salts with trimethylamine, triethylamine, pyridine, picoline, 2,6-lutidine, ethanolamine, diethanolamine, triethanolamine, cyclohexylamine, dicyclohexylamine, N,N′-dibenzylethylenediamine, etc. Preferred examples of the salts with inorganic acids include salts with hydrochloric acid, hydrobromic acid, nitric acid, sulfuric acid, phosphoric acid, etc. Preferred examples of salts with organic acids include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid, etc. Preferred examples of salts with basic amino acids include salts with arginine, lysine, ornithine, etc., and preferred examples of salts with acidic amino acids include salts with aspartic acid, glutamic acid, etc.

Of these salts, pharmaceutically acceptable salts are preferable. For example, when the compound has an acidic functional group, inorganic salts such as alkali metal salts (e.g., sodium salts, potassium salts, etc.), alkaline earth metal salts (e.g., calcium salts, magnesium salts, barium salts, etc.), ammonium salts, and the like are preferable. When the compound has a basic functional group, salts with inorganic acids such as hydrochloric acid, hydrobromic acid, nitric acid, sulfuric acid, phosphoric acid, and salts with organic acids such as acetic acid, phthalic acid, fumaric acid, oxalic acid, tartaric acid, maleic acid, citric acid, succinic acid, methanesulfonic acid, p-toluenesulfonic acid, etc. are preferable.

The prodrug of the compound of the present invention is used to mean such a metastin derivative that is converted into the compound of the present invention by reactions with an enzyme, a gastric acid, etc., under physiological conditions in vivo. In other words, the prodrug of the present invention refers to the metastin derivative that undergoes enzymatic oxidation, reduction, hydrolysis, etc. to be converted into the compound of the present invention, or the metastin derivative that undergoes hydrolysis, etc. by gastric acid, etc. to be converted into the metastin derivative of the present invention.

Examples of the prodrug of the compound of the present invention include metastin derivatives wherein the amino group in the compound of the present invention is substituted with acyl, alkyl, phosphoric acid, etc. (e.g., metastin derivatives wherein the amino group in the compound of the present invention is substituted with eicosanoyl, alanyl, pentylaminocarbonyl (5-methyl-2-oxo-1,3-dioxolen-4-yl)methoxycarbonyl, tetrahydrofuranyl, pyrrolidylmethyl, pivaloyloxymethyl, tert-butyl, etc); metastin derivatives wherein the hydroxy group in the compound of the present invention is substituted with acyl, alkyl, phosphoric acid, boric acid, etc. (e.g., metastin derivatives wherein the hydroxy group in the compound of the present invention is substituted with acetyl, palmitoyl, propanoyl, pivaloyl, succinyl, fumaryl, alanyl, dimethylaminomethylcarbonyl, etc.); and metastin derivatives wherein the carboxy group in the compound of the present invention is substituted with ester, amide, etc. (e.g., metastin derivatives wherein the carboxy group of the compound of the present invention is converted into the ethyl ester, phenyl ester, carboxymethyl ester, dimethylaminomethyl ester, pivaloyloxymethyl ester, ethoxycarbonyloxyethyl ester, phthalidyl ester, (5-methyl-2-oxo-1,3-dioxolen-4-yl)methyl ester, cyclohexyloxycarbonylethyl ester, methylamide, etc); and the like. These metastin derivatives can be produced from the compound of the present invention by per se known methods.

The prodrugs of the compound of the present invention may be those converted into the compound of the present invention under the physiological conditions as described in “Pharmaceutical Research and Development”, Vol. 7, Drug Design, pages 163-198, published 1990 by Hirokawa Publishing Co.

### 1. Prophylactic/Therapeutic Agent for Androgen-Independent Cancer (Preferably Prostate Cancer)

The inventive compound is highly effective in that, along with suppressing tumor growth in patients with androgen-independent cancer (preferably prostate cancer), it has a low toxicity and few side effects.

Therefore, the inventive compound is useful as a prophylactic/therapeutic agent for androgen-independent cancer (preferably prostate cancer) in mammals (e.g., humans, monkeys, chimpanzees, sheep, dogs, mice and rats; particularly, humans).

In the present invention, “androgen-independent cancer (preferably prostate cancer)” refers to cancer (preferably prostate cancer) which has reacquired an ability to grow following temporary suppression of the tumor growth ability by the inhibition of androgen production or function through some form of therapy, such as orchiectomy or hormone therapy. “Suppression of the tumor growth ability” refers to a state where the suppression of tumor growth or amelioration of ostealgia is observed by a decline in the prostate specific antigen (PSA) concentration in the blood or by a method such as computerized tomography (CT), magnetic resonance imaging (MRI) or ultrasound in a cancer (preferably prostate cancer) patient who has received treatment to inhibit androgen production or function by some form of therapy such as orchiectomy or hormone therapy. A decline in the blood PSA concentration refers herein to a blood PSA concentration of, for example, below 5 ng/mL.

As used herein, “reacquired an ability to grow” signifies a state where tumor growth, the emergence or aggravation of ostealgia, or new sites of metastasis are observed by a sustained rise in the blood PSA concentration or by a method such as CT, MRI or ultrasound in a cancer (preferably prostate cancer) patient in which the tumor growth ability was temporarily suppressed by androgen production or function-inhibiting treatment. “Sustained rise in blood PSA concentration ” refers to a state where the blood PSA concentration is, for example, 5 ng/mL or more, and a sustained rise in the blood PSA concentration is observed in the course of periodic tests.

In the present invention, “androgen-independent cancer (preferably prostate cancer)” includes castration-resistant cancer (preferably prostate cancer). The prophylactic agent for androgen-independent cancer (preferably prostate cancer) of the present invention can also delay the progression from androgen-dependent cancer (preferably prostate cancer) into androgen-independent cancer (preferably prostate cancer).

2. Combination with Concomitant Drug

The prophylactic/therapeutic agent for androgen-independent cancer (preferably prostate cancer) of the present invention can be used in combination with a concomitant drug. By combining the prophylactic/therapeutic agent for androgen-independent cancer (preferably prostate cancer) containing the inventive compound as the active ingredient with a concomitant drug, the androgen-independent cancer (preferably prostate cancer) preventing and treating effects can be further enhanced.

The concomitant drug is not subject to any particular limitation. For example, use may be made of one or more drug selected from among hormonal agents (preferably sex hormones), alkylating agents, metabolic antagonists, anticancer antibiotics, plant alkaloids, immunotherapeutic agents, and drugs which inhibit the action of cell growth factors and receptors of the cell growth factors.

The “hormonal agents” are exemplified by fosfestrol, diethylstilbestrol, chlorotrianisene, medroxyprogesterone acetate, megestrol acetate, chlormadinone acetate, cyproterone acetate, danazol, allylesterenol, gestrinone, mepartricin, raloxifene, ormeloxifene, levormeloxifene, anti-estrogen agents (e.g., tamoxifen citrate, toremifene citrate), pill preparations, mepitiostane, testolactone, aminoglutethimide, LHRH modulator (such as LHRH receptor agonists (e.g., goserelin acetate, buserelin acetate, leuprorelin acetate) and LHRH receptor antagonists (e.g., ganirelix, cetrorelix, abarelix, degarelix)), droloxifene, epitiostanol, ethynylestradiol sulfonate, aromatase inhibitors (e.g., fadrozole hydrochloride, anastrozole, letrozole, exemestane, vorozole, formestane), anti-androgen agents (e.g., flutamide, bicalutamide, nilutamide, RD162, MDV3100), 5α-reductase inhibitors (e.g., finasteride, epristeride, dutasteride), adrenocortical hormone preparations (e.g., cortisol, dexamethasone, prednisolone, betamethasone, triamcinolone), androgen synthesis inhibitors (e.g., abiraterone), retinoid and retinoid metabolism retardants (e.g., liarozole) and ER down-regulators (e.g., fulvestrant (Faslodex®)).

Examples of the “alkylating agents” include nitrogen mustard, nitrogen mustard-N-oxide hydrochloride, chlorambutyl, cyclophosphamide, ifosfamide, thiotepa, carboquone, improsulfan tosylate, busulfan, nimustine hydrochloride, mitobronitol, melphalan, dacarbazine, ranimustine, estramustine sodium phosphate, triethylenemelamine, carmustine, lomustine, streptozocin, pipobroman, etoglucid, carboplatin, cisplatin, miboplatin, nedaplatin, oxaliplatin, satraplatin, altretamine, ambamustine, dibrospidium hydrochloride, fotemustine, prednimustine, pumitepa, ribomustin, temozolomide, treosulphan, trophosphamide, zinostatin stimalamer, carboquone, adozelesin, cystemustine, bizelesin, etc.

Examples of the “metabolic antagonists” include mercaptopurine, 6-mercaptopurine riboside, thioinosine, methotrexate, enocitabine, cytarabine, cytarabine ocfosfate, ancitabine hydrochloride, 5-FU drugs (e.g., fluorouracil, tegafur, UFT, doxifluridine, carmofur, gallocitabine, emmitefur, etc.), aminopterin, leucovorin calcium, tabloid, butocine, folinate calcium, levofolinate calcium, cladribine, fludarabine, gemcitabine, hydroxycarbamide, pentostatin, piritrexim, idoxuridine, mitoguazone, thiazophrine, ambamustine, etc.

Examples of the “anticancer antibiotics” include actinomycin D, actinomycin C, mitomycin C, chromomycin A3, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, neocarzinostatin, mithramycin, sarcomycin, carzinophilin, mitotane, zorubicin hydrochloride, mitoxantrone hydrochloride, idarubicin hydrochloride, geldanamycin, rapamycin etc.

Examples of the “plant alkaloids” include etoposide, etoposide phosphate, vinblastine sulfate, vincristine sulfate, vindesine sulfate, teniposide, paclitaxel, vinorelbine, docetaxel, etc.

Examples of “immunotherapeutic agents” include picibanil, krestin, sizofiran, lentinan, ubenimex, interferons, interleukins, macrophage colony-stimulating factor, granulocyte colony-stimulating factor, erythropoietin, lymphotoxin, BCG vaccine, Corynebacterium parvum, levamisole, polysaccharide K, procodazole, cancer vaccine (GVAX™), Sipuleucel-T (Provenge™), Lapuleucel-T (Neuvenge™), DCVax-Prostate™, ONCOVEX GM-CSF™, PROSTVAC-VF™, and PROMUNE™, etc.

The “cell growth factors” in the “drugs which inhibit the action of cell growth factors and receptors of the cell growth factors ” can be any substance so long as it is a material for stimulating the cell growth and, normally, peptides which have a molecular weight of 40,000 (preferably 20,000) or less and bind to their receptors to exhibit the actions in a lower level can be used as the factors. Specific examples are (1) EGF (epidermal growth factor) or substances having substantially the same activity as EGF [e.g., EGF, heregulin (HER ligand), etc.], (2) insulin or substances having substantially the same activity as insulin [e.g., insulin, IGF (insulin-like growth factor)-1, IGF-2, etc.], (3) FGF (fibroblast growth factor) or substances having substantially the same activity as FGF [e.g., acidic FGF, basic FGF, KGF (keratinocyte growth factor), FGF-10, etc.], (4) other cell growth factors [e.g., CSF(colony stimulating factor), EPO (erythropoietin), IL-2 (interleukin-2), NGF (nerve growth factor), PDGF (platelet-derived growth factor), TGFβ (transforming growth factor β), HGF (hepatocyte growth factor), VEGF (vascular endothelial growth factor), etc.] and the like.

The “receptors of the cell growth factors” can be any receptor as long as it is capable of binding to the cell growth factors described above, and specific examples are EGF receptor, heregulin receptor (HER2), insulin receptor, IGF receptor, FGF receptor-1 or FGF receptor-2, etc.

“Drugs which inhibit the action of cell growth factors” are exemplified by antibodies such as HER2 antibodies (e.g., trastuzumab (Herceptin®)), EGFR antibodies (e.g., cetuximab (Erbitux®)), anti-VEGF antibodies (e.g., bevacizumab (Avastin®)) and VEGFR antibodies; tyrosine kinase inhibitors such as imatinib mesylate, VEGFR inhibitors, EGFR inhibitors (e.g., erlotinib (Tarceva®), gefitinib (Iressa®)), lapatinib (EGF receptor/HER2 tyrosine kinase inhibitor), sunitinib (VEGF receptor-2/PDGF receptor/Kit tyrosine kinase inhibitor), sorafenib (kinase inhibitor for all Raf kinase/VEGF receptors), axitinib (tyrosine kinase inhibitor for all VEGF receptors, PDGF receptor β and c-Kit), and antisense drugs, siRNA drugs, shRNA drugs, miRNA drugs and ribozymes which suppress the expression of cell growth factors and their receptors.

In addition to the above, there are also used L-asparginase, aceglatone, procarbazine hydrochloride, protoporphyrin-cobalt complex, mercury-hematoporphyrin sodium, topoisomerase I inhibitor (e.g., Irinotecan, Topotecan, etc.), topoisomerase II inhibitor (e.g., Sobzoxan, etc.), differentiation-inducing agent (e.g., retinoid, vitamin D group, etc.), angiogenesis inhibitor (e.g., thalidomide, SU11248, etc.), tumor vascular targeting agent (Combretastatin A-4 Prodrug, 5, 6- MeXAA), α-blocker (e.g., tamsulosin hydrochloride, naftopidil, urapidil, alfuzosin, terazosin, prazosin, silodosin, etc.), serine-threonine kinase inhibitor, endothelin receptor antagonist (e.g., atrasentan, Zibotentan etc.), proteasome inhibitor (e.g., bortezomib, etc.), Hsp90 inhibitor (e.g., tanespimycin), spironolactone, minoxidil, 11α-hydroxyprogesterone, bone resorption inhibitor/bone metastasis suppressor (e.g., zoledronic acid, alendronic acid, pamidronic acid, etidronic acid, ibandronic acid, clodronic acid), ispinesib (a kinesin inhibitor), lonafarnib (farnesyltransferase inhibitor), deforolimus (a mTOR inhibitor), RANKL antibodies (denosumab) and CTLA-4 antibodies (ipilimumab), as concomitant drugs.

In the present invention, the concomitant drug is preferably an LHRH modulator such as an LHRH receptor agonist (e.g., goserelin acetate, buserelin acetate, leuprorelin acetate) or an LHRH receptor antagonist (e.g., ganirelix, cetrorelix, abarelix, degarelix); and most preferably an LHRH receptor agonist (preferably, leuprorelin acetate).

When the prophylactic/therapeutic agent of the present invention and a concomitant drug are combined, the dosing times for the inventive agent and the concomitant drug are not subject to any particular limitations. The inventive agent and the concomitant drug may be administered to the subject either concurrently or at different times. The inventive agent and the concomitant drug may be formulated as separated preparations, or may be a combination drug obtained by mixing both together. The dose of the concomitant drug should be in general accordance with the dose that is clinically used, and may be suitably selected according to such factors as the subject to which the drug is to be administered, the route of administration, the disease, and the drug combination.

A mode for administration of the inventive agent and a concomitant drug is not particularly limited, but it is sufficient that the inventive agent is used in combination with the concomitant drug at the time of administration. For such mode of administration, there are, for example, (1) administration of a single dosage form obtained by mixing the inventive agent and the concomitant drug together at the same time, (2) simultaneous administration of two dosage forms prepared separately from the inventive agent and the concomitant drug through the same route for administration, (3) administration of two dosage forms prepared separately from the inventive agent and the concomitant drug at certain time intervals through the same route for administration, (4) simultaneous administration of two dosage forms prepared separately from the inventive agent and the concomitant drug through different routes for administration, (5) administration of two dosage forms prepared separately from the inventive agent and the concomitant drug at certain time intervals (e.g., administration of the inventive agent followed by the administration of the concomitant drug in this order, or administration in a reversed order) through different routes for administration, etc.

The combined use of the inventive agent and a concomitant drug exhibits the following excellent effects.

(1) The dose can be reduced as compared to the dose when the inventive agent or a concomitant drug is administered alone.

(2) A drug concomitantly administered with the inventive agent can be chosen depending on the condition (mild, severe, etc.) of a patient.

(3) A concomitant drug, whose functional mechanism is different from that of the inventive agent, can be chosen so that a treatment period can be set longer.

(4) A concomitant drug, whose functional mechanism is different from that of the c inventive agent, can be chosen so that sustained therapeutic effects can be achieved.

(5) A synergistic effect can be obtained by the concomitant use of the inventive agent and a concomitant drug.

### 3. Drug Preparation

In cases where the inventive prophylactic/therapeutic agent for androgen-independent cancer (preferably prostate cancer) is administered to a patient as a drug preparation, the preparation may be produced entirely from the inventive compound, or may be produced by mixing the inventive compound together with a concomitant drug and a pharmaceutically acceptable carrier. The content of the inventive compound in the drug preparation is generally from 0.1 to 100% (w/w). When a concomitant drug is included in the drug preparation, the content thereof is generally from 0.1 to 100% (w/w).

Suitable examples of the dosage form of the inventive drug when orally administered include solid preparations such as tablets, capsules, granules and powders. Suitable dosage forms when parenterally administered, such as intravenously, subcutaneously or intramuscularly, include injections, suppositories and sublingual tablets. Preferred injections include sustained-release preparations such as microcapsules. Dosage forms that may be used for sublingual, subcutaneous or intramuscular administration include sublingual tablets and sustained-release preparations such as microcapsules.

Types of organic and inorganic carrier substances commonly used as preparation ingredients may be employed as the pharmaceutically acceptable carrier. In solid preparations, suitable amounts of excipients, lubricants, binders, disintegrants and thickeners are typically included. In liquid preparations, suitable amounts of solvents, dispersants, dissolution aids, suspending agents, isotonicity agents, buffers and soothing agents are typically included. Where necessary, additives such as preservatives, antioxidants, colorants and sweeteners may also be added as customary.

Examples of preferred excipients include lactose, saccharose, D-mannitol, starch, crystalline cellulose, light anhydrous silicic acid, etc. Preferred examples of lubricants include magnesium stearate, calcium stearate, talc, colloidal silica, etc.

Examples of preferred binders include crystalline cellulose, saccharose, D-mannitol, dextrin, hydroxypropylcellulose, hydroxypropylmethylcellulose, polyvinylpyrrolidone etc.

Examples of preferred disintegrants include starch, carboxymethylcellulose, carboxymethylcellulose calcium, croscarmellose sodium, sodium carboxymethyl starch, etc.

Examples of preferred thickeners include natural gum, cellulose derivatives, polyacrylic acid polymers, etc.

Examples of preferred solvents include water for injection, alcohol, propylene glycol, Macrogol, sesame oil, corn oil, olive oil, etc.

Examples of preferred dispersants include Tween 80, HCO 60, polyethylene glycol, carboxymethylcellulose and sodium alginate, etc.

Examples of preferred dissolution aids include polyethylene glycol, propylene glycol, D-mannitol, benzyl benzoate, ethanol, trisaminomethane, cholesterol, triethanolamine, sodium carbonate, sodium citrate, etc.

Examples of preferred suspending agents include surfactants such as stearyl triethanolamine, sodium lauryl sulfate, lauryl aminopropionate, lecithin, benzalkonium chloride, benzethonium chloride, glycerin monostearate, etc.; hydrophilic polymers such as polyvinyl alcohol, polyvinyl pyrrolidone, sodium carboxymethylcellulose, methylcellulose, hydroxymethylcellulose, hydroxyethylcellulose, hydroxypropylcellulose, etc.

Examples of preferred isotonicity agents include glucose, D-sorbitol, sodium chloride, glycerin, D-mannitol, etc.

Examples of preferred buffers include buffering solutions of a phosphate, acetate, carbonate, citrate, etc.

Examples of preferred soothing agents include benzyl alcohol, etc.

Examples of preferred preservatives include p-hydroxybenzoates, chlorobutanol, benzyl alcohol, phenethyl alcohol, dehydroacetic acid, sorbic acid, etc. Examples of preferred antioxidants include a sulfite, ascorbic acid, etc.

The drug preparation may be produced by a conventional method. Exemplary methods of preparation include the following.

### (1) Tablets, Powders, Granules:

Preparation may be carried out by adding such ingredients as excipients, disintegrants, binders and lubricants to the inventive compound, then shaping by compression. Following compression, coating may be carried out to mask the taste, improve enteric solubility or make the preparation longer-acting.

### (2) Capsules:

Preparation may be carried out by either filling into capsules, or encapsulating and shaping with a capsule base, the inventive compound which has been rendered into the form of a powder, granules or a liquid. Examples of starting materials for the capsule and capsule base include gelatin and hydroxypropylmethyl cellulose.

### (3) Injections:

Preparation may be carried out by rendering the inventive compound into an aqueous injection together with, for example, dispersants, preservatives and isotonicity agents, or by dissolving, suspending or emulsifying the inventive compound in a vegetable oil (e.g., olive oil, sesame oil, cottonseed oil, corn oil), propylene glycol or the like.

### (4) Suppositories:

Preparation may be carried out by rendering the inventive compound into an oil-based or aqueous solid, semisolid or liquid composition. Examples of the oil base that may used in such compositions include higher fatty acid glycerides (e.g., cocoa butter, Witepsols), medium fatty acids (e.g., Migliols), and vegetable oils (e.g., sesame oil, soybean oil, cottonseed oil). Examples of aqueous gels include natural gums, cellulose derivatives, vinyl polymers and acrylic acid polymers.

### 4. Method of Administration

The method of administering the drug preparation produced in “3. Drug Preparation” above varies according to the type of inventive compound selected, the type of concomitant drug, the animal species selected as the target of administration, the symptoms, the dosage form, and the number of times the preparation is to be given. For example, the daily dose in an adult patient with androgen-independent cancer (preferably prostate cancer) when the drug preparation is given orally, expressed as the effective amount of the inventive compound, is generally from about 0.001 to about 500 mg/kg by body weight, preferably from about 0.1 to about 40 mg/kg by body weight, and even more preferably from about 0.5 to about 20 mg/kg by body weight. When the drug preparation is administered parenterally or the inventive compound and a concomitant drug are used in combination, the daily dose will generally be lower than the foregoing range. For example, the daily dose in an adult patient with androgen-independent cancer (preferably prostate cancer) when the drug preparation is given parenterally, expressed as the effective amount of the inventive compound, is preferably from about 0.01 to about 4 mg/kg by body weight, and more preferably from about 0.03 to about 0.6 mg/kg by body weight. However, the amount of the inventive compound actually administered is determined according to such circumstances as the compound selected, the dosage form, the age, weight and sex of the patient, the severity of the disease, the route of administration, and the dosing period and intervals, and may be changed at any time based on the judgment of the physician.

The route of administration of the above drug preparation is not subject to any particular limitation. For example, administration may be carried out by an oral or a parenteral route. As used herein, “parenteral” includes intravenous, intramuscular, subcutaneous, nasal, intradermal, ophthalmic, intracerebral, rectal, vaginal and intraperitoneal administration.

Although the dosing period and interval for the above drug preparation will vary according to the circumstances and will at all times be at the discretion of the physician, any of a number of different methods of administration may be used, including fractionated administration, daily administration, intermittent administration, short-term high-dose administration and repeated administration. For example, in the case of oral administration, it is desirable to carry administration as divided doses given from once to several times daily (especially two or three times daily). Alternatively, administration as a sustained-release preparation or by drip instillation over an extended period of time (e.g., once a month) is also possible.

In the prevention and treatment of androgen-independent cancer (preferably,prostate cancer), it is also possible to use, together with chemotherapy involving administration of the inventive agent, a treatment modality other than chemotherapy, such as surgical treatment including orchiectomy, thermotherapy or radiation therapy.

### 5. Inventive Medication

The inventive medication is characterized by being composed of a combination of the inventive compound with a concomitant drug.

The concomitant drug is preferably one or more selected from among hormonal agents (preferably, sex hormones), alkylating agents, metabolic antagonists, anticancer antibiotics, plant alkaloids, immunotherapeutic agents, and drugs which inhibit the action of cell growth factors and receptors of the cell growth factors. Illustrative examples include the same concomitant drugs as those mentioned above in “2. Combination with Concomitant Drug.” The concomitant drug is preferably an LHRH modulator such as an LHRH receptor agonist (e.g., goserelin acetate, buserelin acetate, leuprorelin acetate) or an LHRH receptor antagonist (e.g., ganirelix, cetrorelix, abarelix); and most preferably an LHRH receptor agonist (preferably, leuprorelin acetate).

Preferred examples of the inventive medication are medications for preventing or treating prostate cancer or androgen-independent prostate cancer in which the concomitant drug is an LHRH receptor agonist or an LHRH receptor antagonist. The inventive medication is a combination of the inventive compound, or a salt or prodrug thereof, as the first active ingredient, with a concomitant drug (an LHRH receptor agonist or LHRH receptor antagonist) as a second active ingredient.

The medication of the present invention can be obtained by combining the inventive compound with a concomitant drug, and carrying out preparation according to a conventional method. The inventive compound and the concomitant drug serving as the active ingredients may each be separately rendered into preparations, or both may be mixed and prepared as a combination drug. Suitable examples of the dosage form of the inventive medication when orally administered include solid preparations such as tablets, capsules, granules and powders. Suitable dosage forms when parenterally administered, such as intravenously, subcutaneously or intramuscularly, include injections, suppositories and sublingual tablets. Preferred injections include sustained-release preparations such as microcapsules. Dosage forms that may be used for sublingual, subcutaneous or intramuscular administration include sublingual tablets and sustained-release preparations such as microcapsules. Specific methods of preparation that may be used include those described above in “3. Drug Preparation,” and methods in general accordance therewith.

The method of administering the inventive medication to the patient varies according to the type of inventive compound selected, the type of concomitant drug, the animal selected as the target of administration, the symptoms, the dosage form, and the number of times the preparation is to be administered. Specific methods of administration include those described above in “4. Method of Administration,” and methods in general accordance therewith.

The inventive medication, which is a combination of the inventive compound, or a salt or prodrug thereof, with the concomitant drug, is useful as an agent for preventing and treating various diseases, such as prostate cancer, androgen-independent prostate cancer, prostate hypertrophy, virilism, hirsutism, male pattern alopecia, precocious puberty in boys, breast cancer, uterine cancer, ovarian cancer, mastopathy, myometrial tumor, endometriosis, adenomyosis uteri and polycystic ovary syndrome; and particularly as an agent for preventing and treating prostate cancer and androgen-independent prostate cancer.

6. Drug for Administration in Cancer Patients with Tolerance (Resistance) to Therapeutic Agents

The inventive agent for administration in cancer patients who have developed a tolerance (resistance) to therapeutic agents is characterized by being composed of a combination of the inventive compound and a concomitant drug.

The therapeutic agent to which the patient has developed a tolerance is not subject to any particular limitation, and may be, for example, one or more selected from among hormonal agents (preferably, sex hormones), alkylating agents, metabolic antagonists, anticancer antibodies, plant alkaloids, immunotherapeutic agents, and drugs which inhibit the activity of cell growth factors and receptors of cell growth factors. The therapeutic agent may be, in particular, an LHRH modulator such as an LHRH receptor agonist (e.g., goserelin acetate, buserelin acetate, leuprorelin acetate) or an LHRH receptor antagonist (e.g., ganirelix, cetrorelix, abarelix); and especially an LHRH receptor agonist (preferably, leuprorelin acetate).

The cancer is not subject to any particular limitation, and may be, for example, prostate cancer, androgen-independent prostate cancer, prostate hypertrophy, virilism, hirsutism, male pattern alopecia, precocious puberty in boys, breast cancer, uterine cancer, ovarian cancer, mastopathy, myometrial tumor, endometriosis, adenomyosis uteri and polycystic ovary syndrome; and especially prostate cancer or androgen-independent prostate cancer.

“Tolerance to a therapeutic agent” means that the efficacy decreases with repeated use of the therapeutic agent, making it necessary to increase the dose in order to achieve the same effects as when use of the therapeutic agent was begun.

Cancer patients who have developed tolerance to a therapeutic agent include, for example, patients in which cancer recurrence or metastasis has been observed due to the development of tolerance to a therapeutic agent by the tumor, patients in which only the administration of a therapeutic agent has been carried out as treatment for cancer, and patients in which both the administration of a therapeutic agent and other treatment modalities (e.g., surgical therapy, radiation therapy, cryotherapy) have been carried out. When the cancer is prostate cancer or androgen-independent prostate cancer, “cancer patients who have developed tolerance to a therapeutic agent” refers to a state where, following the temporary suppression of tumor growth ability owing to the inhibition of androgen production or function by some form of therapy, tumor growth or the emergence or aggravation of ostealgia or a new site of metastasis is observed by a sustained rise in the blood PSA concentration or by a procedure such as CT, MRI or ultrasound. “Sustained rise in blood PSA concentration ” refers to a state where the blood PSA concentration is, for example, 5 ng/mL or more, and a sustained rise in the blood PSA concentration is observed in the course of periodic tests.

Exemplary concomitant drugs include one or more selected from among hormonal agents (preferably, sex hormones), alkylating agents, metabolic antagonists, anticancer antibiotics, plant alkaloids, immunotherapeutic agents, and drugs which inhibit the action of cell growth factors and receptors of cell growth factors. Illustrative examples include the same concomitant drugs as those mentioned above in “2. Combination with Concomitant Drug.” The concomitant drug is preferably an LHRH modulator such as an LHRH receptor agonist (e.g., goserelin acetate, buserelin acetate, leuprorelin acetate) or an LHRH receptor antagonist (e.g., ganirelix, cetrorelix, abarelix); and most preferably an LHRH receptor agonist.

Medications for administration in cancer patients who have developed tolerance to a therapeutic agent can be obtained by combining the inventive compound and the concomitant drug, and carrying out preparation according to a conventional method. The inventive compound and the concomitant drug serving as the active ingredients may each be separately rendered into preparations, or both may be mixed and prepared as a combination drug. Suitable examples of the dosage form when the inventive drug is to be administered orally include solid preparations such as tablets, capsules, granules and powders. Suitable dosage forms when parenterally administered, such as intravenously, subcutaneously or intramuscularly, include injections, suppositories and sublingual tablets. For sublingual, subcutaneous or intramuscular administration, for example, the dosage form may be a sustained-release preparation such as sublingual tablets or microcapsules. Specific methods of preparation that may be used include those described above in “3. Drug Preparation,” and methods in general accordance therewith.

The method of administering the inventive medication in patients will vary according to, for example, the type of inventive compound selected, the type of concomitant drug, the animal species selected as the target of administration, the symptoms, the dosage form, and the number of times the preparation is to be administered. Specific methods of administration include those described above in “4. Method of Administration,” and methods in general accordance therewith.

The inventive medication for administration in cancer patients who have developed tolerance to a therapeutic agent is a combination of the inventive compound and a concomitant drug, and is useful for administration in patients with various types of cancer, especially patients with prostate cancer or androgen-independent prostate cancer.

### EXAMPLES

The present invention is further illustrated by the following preparation examples and test examples. It is to be understood that the present invention is not limited to these examples, and various changes and modifications may be made in the invention without departing from the spirit and scope thereof.

In the following examples, “room temperature” generally refers to from about 10° C. to about 35° C. As used below, the symbol “%” signifies mol/mol % with respect to yield, vol % with respect to the solvent used in chromatography, and wt % elsewhere. In the proton NMR spectra, results such as for OH and NH protons that were broad and could not be confirmed are not mentioned in the data.

| Abbreviation | Description |
|---|---|
| 10Ψ,CSNH: | The C-terminal-CONH₂ at the 10-position |
| | is substituted with —CSNH₂. |
| 1Ψ2,CH₂NH: | The —CONH— bond between the 1- and 2-positions |
| | is substituted with the —CH₂NH— bond. |
| 2Ψ3,CH₂NH: | The —CONH— bond between the 2- and 3-positions |
| | is substituted with the —CH₂NH— bond. |
| 3Ψ4,CH₂NH: | The —CONH— bond between the 3- and 4-positions |
| | is substituted with the —CH₂NH— bond. |
| 4Ψ5,CH₂NH: | The —CONH— bond between the 4- and 5-positions |
| | is substituted with the —CH₂NH— bond. |
| 6Ψ7,CSNH: | The —CONH— bond between the 6- and 7-positions |
| | is substituted with the —CSNH— bond. |
| 6Ψ7,NHCO: | The —CONH— bond between the 6- and 7-positions |
| | is substituted with the —NHCO— bond. |
| 6Ψ7,CH₂NH: | The —CONH— bond between the 6- and 7-positions |
| | is substituted with the —CH₂NH— bond. |
| 6Ψ7,CH₂O: | The —CONH— bond between the 6- and 7-positions |
| | is substituted with the —CH₂O— bond. |
| 7Ψ8,CH₂NH: | The —CONH— bond between the 7- and 8-positions |
| | is substituted with the —CH₂NH— bond. |
| 8Ψ9,CH₂NH: | The —CONH— bond between the 8- and 9-positions |
| | is substituted with the —CH₂NH— bond. |
| 9Ψ10,CH₂NH: | The —CONH— bond between the 9- and 10-positions |
| | is substituted with the —CH₂NH— bond. |
| Abu: | 2-aminobutanoic acid |
| Ac: | acetyl |
| Acp: | 6-aminocaproic acid |
| AcOEt: | ethyl acetate |
| AcOH: | acetic acid |
| Aib: | α-aminoisobutanoic acid |
| Ala(2-Qui): | 2-quinolylalanine |
| Ala(3-Bzt): | 3-benzothienylalanine |
| Alb: | Albizziin 2-amino-3-ureidopropionic acid |
| Arg(Ac): | N^{ω}-acetylarginine |
| Arg(Boc₂,Me): | N^{ω,ω′}-bis-tert-butoxycarbonyl-N^{ω}-methylarginine |
| Arg(Et): | N^{ω}-ethylarginine |
| Arg(Me): | N^{ω}-methylarginine |
| Arg(asyMe₂) or | asymmetric-N^{ω,ω}-dimethylarginine |
| Arg(Me₂)asym: | |
| Arg(symMe₂) or | symmetric-N^{ω,ω′}-dimethylarginine |
| Arg(Me₂)sym: | |
| Arg(NO₂): | N^{ω}-nitroarginine |
| Arg(n-Pr): | N^{ω}-propylarginine |
| Arg(Tos): | N^{ω}-tosylarginine |
| Asp(NHMe): | N^{ω}-methylasparagine |
| Asp(NMe₂): | N^{ω,ω}-dimethylasparagine |
| AzaGly: | azaglycine |
| AzaPhe: | azaphenylalanine |
| Aze(2): | azetidine-2-carboxylic acid |
| β-Ala: | β-alanine |
| Boc: | tert-butoxycarbonyl |
| Boc₂O: | di-tert-butyl dicarbonate |
| Br-Z: | 2-bromobenzyloxycarbonyl |
| Bu^{t}: | tert-butyl |
| Bzl: | benzyl |
| CDI: | 1,1′-carbonyldiimidazole |
| Cha: | cyclohexylalanine |
| CIP: | 2-chloro-1,3-dimethylimidazolium |
| | tetrafluoroborate |
| Cit: | citrulline |
| Clt resin: | 2-chlorotrytyl resin |
| Cl-Z: | 2-chlorobenzyloxycarbonyl |
| Dab: | 2,4-diaminobutanoic acid |
| Dap: | 2,3-diaminopropionic acid |
| Dap(Ac): | N^{β}-acetyl-β-diaminopropionic acid |
| Dap(For): | N^{β}-formyl-β-diaminopropionic acid |
| Dap(Gly): | N^{β}-glycyl-β-diaminopropionic acid |
| Dap(GnGly): | N^{β}-(N-guanidinoglycyl)-β-diaminopropionic acid |
| DCM: | dichloromethane |
| DEA: | diethylamine |
| DIEA: | N,N-diisopropylethylamine |
| DIPCDI: | 1,3-diisopropylcarbodiimide |
| DMAP: | 4-dimethylaminopyridine |
| DMF: | N,N-dimethylformamide |
| EDT: | 1,2-ethanedithiol |
| Fmoc: | 9-fluorenylmethoxycarbonyl |
| For: | formyl |
| γ-Abu: | 4-aminobutanoic acid |
| γ-MeLeu,: | γ-methylleucine |
| Gn: | guanidino |
| GuAmb: | 4-guanidinomethylbenzoyl |
| Har: | homoarginine |
| Har(Me): | N^{ω} -methylhomoarginine |
| HOAt: | 1-hydroxy-7-azabenzotriazole |
| HOBt: | 1-hydroxybenzotriazole |
| HONB: | N-hydroxy-5-norbornene-2,3-dicarboximide |
| Hph: | homophenylalanine |
| Hyp: | trans-4-hydroxyproline |
| IndPr: | 3-(indole-3-yl)propionyl |
| Lys(Me₂): | N^{ε,ε}-dimethyllysine |
| MBHA: | p-methylbenzhydrylamine |
| MeOH: | methanol |
| Mtt: | 4-methyltrytyl |
| N((CH₂)₃Gn)Gly: | N-(3-guanidinopropyl)glycine |
| Nal(1): | 1-naphthylalanine |
| Nal(2): | 2-naphthylalanine |
| Nar: | norarginine |
| Nar(Me): | N^{ω}-methylnorarginine |
| Nle: | norleucine |
| NMeArg: | N^{α}-methylarginine |
| NMeAsn: | N^{α}-methylasparagine |
| NMeLeu: | N^{α}-methylleucine |
| NMePhe: | N^{α}-methylphenylalanine |
| NMeSer: | N^{α}-methylserine |
| NMeTrp: | N^{α}-methyltryptophan |
| NMeTyr: | N^{α}-methyltyrosine |
| Nva: | norvaline |
| Orn: | ornithine |
| Orn(Mtt): | N^{δ}-(4-methyltrytyl)ornithine |
| PAL: | 5-(4-(9-fluorenylmethoxycarbonyl)aminomethyl- |
| | 3,5-dimethoxyphenoxy)valeric acid |
| Pbf: | 2,2,4,6,7-pentamethyldihydrobenzofuran- |
| | 5-sulfonyl |
| pGlu: | pyroglutamic acid |
| Phe(2Cl): | 2-chlorophenylalanine |
| Phe(2F): | 2-fluorophenylalanine |
| Phe(3,4Cl₂): | 3,4-dichlorophenylalanine |
| Phe(3,4F₂): | 3,4-difluorophenylalanine |
| Phe(3CF₃): | 3-trifluoromethylphenylalanine |
| Phe(3Cl): | 3-chlorophenylalanine |
| Phe(3F): | 3-fluorophenylalanine |
| Phe(4Cl): | 4-chlorophenylalanine |
| Phe(4CN): | 4-cyanophenylalanine |
| Phe(4F): | 4-fluorophenylalanine |
| Phe(4Gn): | 4-guanidinophenylalanine |
| Phe(4NH₂): | 4-aminophenylalanine |
| Phe(4NO₂): | 4-nitrophenylalanine |
| Phe(4CN): | 4-cyanophenylalanine |
| Phe(F₅): | pentafluorophenylalanine |
| PheΨ(CH₂O)Gly: | The —CONH— bond between Phe and Gly is |
| | substituted with the —CH₂O— bond. |
| PheΨ(CSNH)—NH₂: | The C-terminal phenylalanylamide is substituted with |
| | phenylalanylthioamide. |
| Phg: | phenylglycine |
| PhOH: | phenol |
| PhSMe: | thioanisole |
| Pic(2): | pipecolinic acid |
| Pro: | proline |
| Pya(2): | 2-pyridylalanine |
| Pya(3): | 3-pyridylalanine |
| Pya(4): | 4-pyridylalanine |
| PyAOP: | (7-azabenzotriazole-1-yloxy)-tris(pyrrolidino) |
| | phosphonium hexafluorophosphate |
| PyBOP: | (benzotriazol-1-yloxy)-tris(pyrrolidino) |
| | phosphonium hexafluorophosphate |
| PyBrop: | bromo-tris(pyrrolidino) |
| | phosphonium hexafluorophosphate |
| Sar: | N-methylglycine |
| Ser(Ac): | O-acetylserine |
| Ser(Me): | O-methylserine |
| Thi: | 2-thienylalanine |
| Thz: | thioproline |
| Tic: | 1,2,3,4-tetrahydroisoquinoline-2-carboxylic acid |
| TIS: | triisopropylsilane |
| Tle: | tert-leucine |
| Tos: | tosyl |
| Trp(For): | Nⁱⁿ-formyltryptophan |
| Trt: | trytyl |
| Tyr(Me): | O-methyltyrosine |
| TyrΨ(CH₂NH)Asn: | The —CONH— between Tyr and Asn is |
| | substituted with the —CH₂NH— bond. |
| TFA: | trifluoroacetic acid |
| TFE: | trifluoroethanol |
| Z: | benzyloxycarbonyl |
| FCS: | Fetal Calf Serum |
| DCC: | Dextran-Coated Charcoal |
| DMEM: | Dulbecco's Modified Eagle's Medium |
| DPBS: | Dulbecco's Phosphate Buffered Saline |

In the specification and drawings, where the codes of bases and amino acids are denoted by abbreviations, they are based on the abbreviations in accordance with the IUPAC-IUB Commission on Biochemical Nomenclature or the common codes in the art, examples of which are shown below. For amino acids that may have the optical isomer, L form is presented unless otherwise indicated.
DNA: deoxyribonucleic acid
cDNA: complementary deoxyribonucleic acid
A: adenine
T: thymine
G: guanine
C: cytosine
Y: thymine or cytosine
N: thymine, cytosine, adenine or guanine
R: adenine or guanine
M: cytosine or adenine
W: thymine or adenine
S: cytosine or guanine
RNA: ribonucleic acid
mRNA: messenger ribonucleic acid
dATP: deoxyadeno sine triphosphate
dTTP: deoxythymidine triphosphate
dGTP: deoxyguanosine triphosphate
dCTP: deoxycytidine triphosphate
ATP: adenosine triphosphate
EDTA: ethylenediaminetetraacetic acid
SDS: sodium dodecyl sulfate
TFA: trifluoroacetic acid
EIA: enzyme immunoassay
Gly or G: glycine
Ala or A: alanine
Val or V: valine
Leu or L: leucine
Ile or I: isoleucine
Ser or S: serine
Thr or T: threonine
Cys or C: cysteine
Met or M: methionine
Glu or E: glutamic acid
Asp or D: aspartic acid
Lys or K: lysine
Arg or R: arginine
His or H: histidine
Phe or F: phenylalanine
Tyr or Y: tyrosine
Trp or W: tryptophan
Pro or P: proline
Asn or N: asparagine
Gln or Q: glutamine
pGlu: pyroglutamic acid

The sequence identification numbers in the sequence listing of the specification indicates the following sequence, respectively.
[SEQ ID NO: 1]

This shows the amino acid sequence of human-derived metastin (Metastin).
[SEQ ID NO: 2]

This shows the base sequence of DNA encoding human metastin.
[SEQ ID NO: 3]

This shows the amino acid sequence of mouse metastin precursor (A).
[SEQ ID NO: 4]

This shows the base sequence of DNA encoding mouse metastin precursor (A), which is the base sequence in plasmid pCMV-mKiSS-1 harbored on transformant Escherichia coli DH10B/pCMV-mKiSS-1.
[SEQ ID NO: 5]

This shows the amino acid sequence of mouse metastin precursor (B).
[SEQ ID NO: 6]

This shows the base sequence of DNA encoding mouse metastin precursor (B), which is the base sequence in plasmid pCR2.1-mKiSS-1.4A harbored on transformant Escherichia coli DH5α/pCR2.1-mKiSS-1.4A.
[SEQ ID NO: 7]

This shows the amino acid sequence of rat-derived metastin precursor.
[SEQ ID NO: 8]

This shows the base sequence of DNA encoding rat metastin precursor.
[SEQ ID NO: 9]

This shows the amino acid sequence of human OT7T175 (metastin receptor).
[SEQ ID NO: 10]

This shows the base sequence of DNA encoding human OT7T175 (metastin receptor).
[SEQ ID NO: 11]

This shows the amino acid sequence of rat OT7T175 (metastin receptor).
[SEQ ID NO: 12]

This shows the base sequence of DNA encoding rat OT7T175 (metastin receptor).
[SEQ ID NO: 13]

This shows the amino acid sequence of mouse OT7T175 (metastin receptor).
[SEQ ID NO: 14]

This shows the base sequence of DNA encoding mouse OT7T175 (metastin receptor).
[SEQ ID NO: 15]

This shows the amino acid sequence of human metastin 15 (40-54).
[SEQ ID NO: 16]

This shows the amino acid sequence of human metastin 10 (45-54) (MS10).
[SEQ ID NO: 17]

This shows the amino acid sequence of human metastin 9 (46-54).
[SEQ ID NO: 18]

This shows the amino acid sequence of human metastin 8 (47-54).
[SEQ ID NO: 19]

This shows the base sequence of DNA encoding human metastin 15 (40-54).
[SEQ ID NO: 20]

This shows the base sequence of DNA encoding human metastin 10 (45-54).
[SEQ ID NO: 21]

This shows the base sequence of DNA encoding human metastin 9 (46-54).
[SEQ ID NO: 22]

This shows the base sequence of DNA encoding human metastin 8 (47-54)

In the present invention, Tyr-Asn-Trp-Asn-Ser-Phe-Gly-Leu-Arg-Phe-NH₂ (SEQ ID NO: 16) is referred to as Metastin 10, or MS10.

In the subsequent examples, the Tyr position at the N-terminus of MS10 is counted as position 1, and the Phe position at the C-terminus is counted as position 10.

Tyr-Asn-Trp-Asn-Ser-Phe-Gly-Leu-Arg-Phe-NH₂
1 2 3 4 5 6 7 8 9 10

The designation of Compound No. 79 (Example 1) as [Hph10]MS10 means that this is a peptide in which the Phe at the C-terminus (position 10) of MS10 has been substituted with Hph.

The designation of Compound No. 4 as des(1)-MS10 means that this is a peptide in which the Tyr at the N-terminus (position 1) has been deleted.

The designation of Compound No. 53 as des(1-3)-Fmoc-MS10 means that this is a peptide in which the Tyr-Asn-Trp at the N-terminus (positions 1 to 3) have been deleted, and the amino group of Asn at position 4 has been modified with Fmoc.

### Preparation Example 1

| | |
|---|---|
| (1) Compound No. 550 | 10.0 mg |
| (2) Lactose | 60.0 mg |
| (3) Cornstarch | 35.0 mg |
| (4) Gelatin | 3.0 mg |
| (5) Magnesium stearate | 2.0 mg |

A mixture composed of 10.0 mg of Compound No. 550, 60.0 mg of lactose and 35.0 mg of cornstarch was granulated by being passed through a sieve having a mesh size of 1 mm using 0.03 ml of a 10% aqueous solution of gelatin (containing 3.0 mg of gelatin), then dried at 40° C. and again passed through the sieve. The granules thus obtained were mixed with 2.0 mg of magnesium stearate, and compressed. The resulting core tablets were coated with a sugar coat using an aqueous suspension of sucrose, titanium dioxide, talc and gum arabic. The tablets thus coated were then polished with beeswax, thereby giving coated tablets.

### Preparation Example 2

| | |
|---|---|
| (1) Compound No. 550 | 10.0 mg |
| (2) Lactose | 70.0 mg |
| (3) Cornstarch | 50.0 mg |
| (4) Soluble starch | 7.0 mg |
| (5) Magnesium stearate | 3.0 mg |

Compound No. 550 (10.0 mg) and magnesium stearate (3.0 mg) were granulated using 0.07 ml of an aqueous solution of soluble starch (containing 7.0 mg of soluble starch). The granules were then dried, and mixed with 70.0 mg of lactose and 50.0 mg of cornstarch. The mixture was compressed, thereby forming tablets.

### Preparation Example 3

| | |
|---|---|
| (1) Compound No. 550 | 5.0 mg |
| (2) Table salt | 20.0 mg |
| (3) Distilled water | added to a total volume of 2 ml |

Compound No. 550 (5.0 mg) and table salt (20.0 mg) were dissolved in distilled water, and water was added to a total volume of 2.0 ml. The solution was filtered, then filled into 2 ml ampules under sterile conditions. The ampules were sterilized, then sealed, thereby giving a solution for injection.

### Preparation Example 4

| | |
|---|---|
| (1) Compound No. 723 | 10.0 mg |
| (2) Lactose | 60.0 mg |
| (3) Cornstarch | 35.0 mg |
| (4) Gelatin | 3.0 mg |
| (5) Magnesium stearate | 2.0 mg |

A mixture composed of 10.0 mg of Compound No. 723, 60.0 mg of lactose and 35.0 mg of cornstarch was granulated by being passed through a sieve having a mesh size of 1 mm using 0.03 ml of a 10% aqueous solution of gelatin (containing 3.0 mg of gelatin), then dried at 40° C. and again passed through the sieve. The granules thus obtained were mixed with 2.0 mg of magnesium stearate, and compressed. The resulting core tablets were coated with a sugar coat using an aqueous suspension of sucrose, titanium dioxide, talc and gum arabic. The tablets thus coated were then polished with beeswax, thereby giving coated tablets.

### Preparation Example 5

| | |
|---|---|
| (1) Compound No. 723 | 10.0 mg |
| (2) Lactose | 70.0 mg |
| (3) Cornstarch | 50.0 mg |
| (4) Soluble starch | 7.0 mg |
| (5) Magnesium stearate | 3.0 mg |

Compound No. 723 (10.0 mg) and magnesium stearate (3.0 mg) were granulated using 0.07 ml of an aqueous solution of soluble starch (containing 7.0 mg of soluble starch). The granules were then dried, and mixed with 70.0 mg of lactose and 50.0 mg of cornstarch. The mixture was compressed, thereby forming tablets.

### Preparation Example 6

| | |
|---|---|
| (1) Compound No. 723 | 5.0 mg |
| (2) Table salt | 20.0 mg |
| (3) Distilled water | added to a total volume of 2 ml |

Compound No. 723 (5.0 mg) and table salt (20.0 mg) were dissolved in distilled water, and water was added to a total volume of 2.0 ml. The solution was filtered, then filled into 2 ml ampules under sterile conditions. The ampules were sterilized, then sealed, thereby giving a solution for injection.

### Preparation Example 7

409.80 g of a lactic acid-glycolic acid copolymer (Lactic acid/Glycolic acid ratio: 75/25; weight average molecular weight (Mw): 7,800; number average molecular weight (Mn): 3,400; Mw/Mn ratio: 2.3) (Wako Pure Chemical Industries, Ltd.) was dissolved in 757.76 g of dichloromethane. 795.45 g of the solution was weighed and mixed with an aqueous solution which had been obtained by dissolving 35.10 g of Compound No. 723 in 30.60 g of distilled water, and emulsified using a ROBOMIX (manufactured by Tokushukika) to form a W/O emulsion (Rotation speed: about 10,000 rpm, one minute). Then, this W/O emulsion was cooled to about 10° C., poured into 50 liters of a 0.1% (w/w) aqueous polyvinyl alcohol (EG-40, manufactured by Nippon Synthetic Chemical Industry Co., Ltd) solution which had been warmed to about 18° C. in advance, and emulsified using HOMOMIC LINE FLOW (manufactured by Tokushukika) to form a W/O/W emulsion (Turbine rotation speed: about 7,000 rpm; Circulation pump rotation speed: about 2,000 rpm). The obtained W/O/W emulsion was stirred for about 3 hours (water-drying process), filtered through a sieve having 75 μm opening, and microspheres were centrifuged continuously using a centrifuge (H-6005, manufactured by Kokusan Co Ltd.) (Rotation speed: about 2,000 rpm; Flow amount: about 550 ml/min) and collected. The collected microspheres were dispersed again in a small amount of distilled water, filtered through a sieve having 90 μm opening, added with 42.436 g of mannitol, and lyophilized using a lyophilizer (DFM-05A-S, ULVAC) to obtain microcapsule powders. The content of Compound No. 723 in the obtained microcapsule powder was 8.2%.

### Preparation Example 8

1263.2 g of a lactic acid-glycolic acid copolymer (Lactic acid/Glycolic acid ratio: 75/25; weight average molecular weight (Mw): 10,300) (Wako Pure Chemical Industries, Ltd.) was dissolved in 2184.0 g of dichloromethane. 2525.6 g of the solution was weighed and mixed with a solution which had been obtained by dissolving 273.34 g of Compound No. 550 in an mixed solution of 84.00 g of an acetic acid and 280.0 g of methanol to form an Oil phase. Then, this Oil phase was cooled to about 10° C., poured into 200 liters of a 0.1% (w/w) aqueous polyvinyl alcohol (EG-40, manufactured by Nippon Synthetic Chemical Industry Co., Ltd) solution which had been warmed to about 18° C. in advance, and emulsified using HOMOMIC LINE FLOW (manufactured by Tokushukika) to form a O/W emulsion (Turbine rotation speed: about 7,000 rpm; Circulation pump rotation speed: about 2,500 rpm). The obtained O/W emulsion was stirred for about 3 hours (water-drying process), filtered through a sieve having 75 μm opening, and microspheres were centrifuged continuously using a centrifuge (H-1002, manufactured by Kokusan Co Ltd.) (Rotation speed: about 2,000 rpm; Flow amount: about 600 ml/min) and collected. The collected microspheres were dispersed again in a small amount of distilled water, filtered through a sieve having 90 μm opening, added with 168.51 g of mannitol, and lyophilized using a lyophilizer (RL-402BS, manufactured by Kyowa Vacuum Engineering, Ltd.) to obtain microcapsule powders. The content of Compound No. 550 in the obtained microcapsule powder was 16.7%.

### Test Example 1

Dunning R3327-G cells, an androgen-sensitive rat prostate cancer cell line, were implanted into orchiectomized Copenhagen rats, and the androgen-independent antitumor effects by Compound No. 550 and Compound No. 723 were investigated.

R3327-G cells (7×10⁶) were implanted subcutaneously in 10-week-old orchiectomized male Copenhagen rats. Fifty days after implantation, the rats were divided into groups based on the tumor volume, and were then assigned to a Compound 550—50 nmol/kg/W group, a Compound No. 723—50 nmol/kg/W group, and a solution group (each group consisting of 10 animals). The dose was calculated based on the mean weight of the animals on the day that dosing was begun. Administration was carried out by subcutaneous implantation in the dorsal region using an ALZET pump. The tumor diameter was measured on days 50, 65, 71, 78, 91 and 102 following cell implantation (days 0, 15, 21, 28, 41 and 52 following the start of dosing). The tumor volume (mm³) was calculated as follows: major axis×minor axis²÷2. Owing to deaths and the euthanization of animals in the course of the tests, on day 52 following the start of dosing (day 102 after transplantation), only six animals remained in the solution group and only seven animals remained in the Compound No. 723 group.

The results are shown in FIG. 1. On day 52 following the start of dosing, both the administration of 50 nmol/kg/W of Compound No. 550 and the administration of Compound No. 723 showed significant antitumor effects. This demonstrated that Compound No. 550 and Compound No. 723 are useful for treating hormone-independent prostate cancer.

### Test Example 2

Evaluation of the growth-inhibitory activity of the metastin peptide derivative using human prostate cancer cell line VCaP

Regarding Compound No. 723, the inhibitory activity on androgen-independent growth of human prostate cancer cell line VCaP (CRL-2876, American Type Culture Collection) was evaluated. VCaP in the state of three-dimensional culture (spheroid) was used in the evaluation. Specifically, VCaP cells were suspended in a DMEM medium (Ref. No. 11995, Invitrogen) containing 10% FCS (Cat. No. 171012, Cell Culture Bioscience) to set the concentration to 1.5×10⁶ cells/mL. The obtained suspension was spotted on a cover of a tissue culture dish (Ref. No. 353003, FALCON) (20 μL for each) to perform Hanging drop culture (3×10⁴ cells/drop). In order to prevent drying, 10 mL of DPBS (Ref. No. 14190, Invitrogen) was added to the culture dish. The culture was performed at 37° C. under 5% CO₂ and humidified atmosphere for 10 days, and after that, using a pipette tip whose leading edge had been cut off, spheroids formed in each drop were transferred one by one to a low adhesion 96-well plate having a U-shaped bottom (MS-00965, SUMITOMO BAKELITE), and the culture was further performed for another 3 days. After 3 days, the medium was replaced with a phenol red free DMEM medium (Ref. No. 21063, Invitrogen) containing 10% DCC-FCS (FCS subjected to dextran-coated charcoal treatment; the same applies to the following).

Note that the above-described DCC-FCS was prepared as follows: 25 g of charcoal (C-3345, SIGMA) and 250 mg of T70 dextran (17-0280-2, Pharmacia) were added to 500 mL of DPBS and the obtained mixture was autoclaved for sterilization; 25 mL of the resultant suspension was added to 500 mL of FCS and the mixture was shaken for 30 minutes at 45° C.; and then centrifuged at 1700×g for 30 minutes at 4° C., a supernatant thus obtained was filter-sterilized to obtain DCC-FCS.

Immediately after the replacement of the medium, a treatment using Compound No. 723 was started. Firstly, Compound No. 723 was dissolved in a phenol red free DMEM medium to obtain 1 mM Compound No. 723 solution. The Compound No. 723 solution was diluted with a phenol red free DMEM medium containing 10% DCC-FCS, and it was supplied to the culture medium every 12 hours for 8 continuous days so that the final concentration of Compound No. 723 became 1 μM. In the control group, a phenol red free DMEM medium was diluted with a phenol red free DMEM medium containing 10% DCC-FCS in the same way with the Compound No. 723 solution, then supplied to the culture medium.

Cell growth was quantified 9 days after the start of the treatment with Compound No. 723 by measuring the chemical luminescence evoked by cellular ATP using CellTiter-Glo Luminescent Cell Viability Assay (Promega). Wallac 1420 ARVO MX/Light (Parkin Elmer) was used for the measurement of the luminescence intensity. Results are shown below.

| | Luminescence intensity |
|---|---|
| Control | 564458 ± 27628 |
| Compound No. 723 | 507191 ± 73146 * |

* p = 0.00653 (n = 18, Student's t-test)

As is clear from the above table, Compound No. 723 significantly reduced the androgen-independent growth of human prostate cancer cells.

### Test Example 3

Evaluation of the growth-inhibitory activity of the metastin peptide derivative using human prostate cancer cell line 22Rv1

Regarding Compound No. 723, the growth-inhibitory activity on androgen-independent human prostate cancer cell line 22Rv1 (CRL-2505, American Type Culture Collection) was evaluated. VCaP in the state of three-dimensional culture (spheroid) was used in the evaluation. Specifically, 22Rv1 cells were suspended in a RPMI1640 medium (Ref. No. 22400, Invitrogen) containing 10% FCS (Cat. No. 171012, Cell Culture Bioscience) to set the concentration to 1.5×10⁶ cells/mL. The obtained suspension was spotted on a cover of a tissue culture dish (Ref. No. 353003, FALCON) (20 μL for each) to perform Hanging drop culture (3×10³ cells/drop). In order to prevent drying, 10 mL of DPBS (Ref. No. 14190, Invitrogen) was added to the culture dish. The culture was performed at 37° C. under 5% CO₂ and humidified atmosphere for 10 days, and after that, using a pipette tip whose leading edge had been cut off, spheroids formed in each drop were transferred one by one to a low adhesion 96-well plate having a U-shaped bottom (MS-00965, SUMITOMO BAKELITE), and the culture was further performed for another 3 days. After 3 days, the medium was replaced with a phenol red free RPMI1640 medium (Ref. No. 11835, Invitrogen) containing 10% DCC-FCS (FCS subjected to dextran-coated charcoal treatment).

Note that the above-described DCC-FCS was prepared as follows: 25 g of charcoal (C-3345, SIGMA) and 250 mg of T70 dextran (17-0280-2, Pharmacia) were added to 500 mL of DPBS and the obtained mixture was autoclaved for sterilization; 25 mL of the resultant suspension was added to 500 mL of FCS and the mixture was shaken for 30 minutes at 45° C.; and then centrifuged at 1700×g for 30 minutes at 4° C., a supernatant thus obtained was filter-sterilized to obtain DCC-FCS.

Immediately after the replacement of the medium, a treatment using Compound No. 723 was started. Firstly, Compound No. 723 was dissolved in a phenol red free DMEM medium (Ref. No. 21063, Invitrogen) to obtain 1 mM Compound No. 723 solution. The Compound No. 723 solution was diluted with a phenol red free RPMI1640 medium containing 10% DCC-FCS, and it was supplied to the culture medium every 12 hours for 8 continuous days so that the final concentration of Compound No. 723 became 1 μM. In the control group, a phenol red free DMEM medium was diluted with a phenol red free DMEM medium containing 10% DCC-FCS in the same way with the Compound No. 723 solution, then supplied to the culture medium.

Cell growth was quantified 9 days after the start of the treatment with Compound No. 723 by measuring the chemical luminescence evoked by cellular ATP using CellTiter-Glo Luminescent Cell Viability Assay (Promega). Wallac 1420 ARVO MX/Light (Parkin Elmer) was used for the measurement of the luminescence intensity. Results are shown below.

| | Luminescence intensity |
|---|---|
| Control | 483469 ± 55917 |
| Compound No. 723 | 401291 ± 40565 * |

* p = 0.00003 (n = 18, Student's t-test)

As is clear from the above table, Compound No. 723 significantly reduced the growth of androgen-independent human prostate cancer cells in the absence of androgen.

### Test Example 4

Evaluation of antitumor activity of the metastin peptide derivative in DU145 tumor-bearing male rat model

Antitumor activity of Compound No. 550 and Compound No. 723 against a DU145 tumor-bearing male rat model was evaluated. Specifically, 1×10⁶ cells/100 μL suspension of DU145 (androgen-independent cell line, GPR54 highly expressing cell line, ATCC) was mixed with 100 μL of basement membrane matrix: Matrigel (trade name, BD Biosciences), and the obtained mixture was transplanted under the abdominal skin of 7-week-old F344/NJc1-rnu/rnu male rats (CLEA Japan, Inc.) which had been etherized. 10 days after the transplantation, the rats in which the volume of subcutaneous tumor reached 200 mm³ were divided into 4 groups (Groups A-D, 10 rats for each group). In each group, the rats were etherized, and then subcutaneously injected with the following suspensions: Group A: about 2 ml of suspension of dispersion medium of the microcapsule powder obtained in Preparation Example 8 (containing 10 mg of microcapsule powder per 1 ml of dispersion medium) (the dose of Compound No. 550: 10mg/kg body weight); Group B: about 0.2 ml of suspension of dispersion medium of the microcapsule powder obtained in Preparation Example 7 (containing 1 mg of microcapsule powder per 1 ml of dispersion medium) (the dose of Compound No. 723: 1 mg/kg body weight); and Group C: about 2 ml of suspension of dispersion medium of the microcapsule powder obtained in Preparation Example 7 (containing 1 mg of microcapsule powder per 1 ml of dispersion medium) (the dose of Compound No. 723: 10 mg/kg body weight). In this context, the dispersion medium was a suspension obtained by suspending D-mannitol, carmellose sodium and Polysorbate 80 in water for injection, and 50 mg of D-mannitol, 5 mg of carmellose sodium and 1 mg of Polysorbate 80 were contained in 1 ml of suspension.

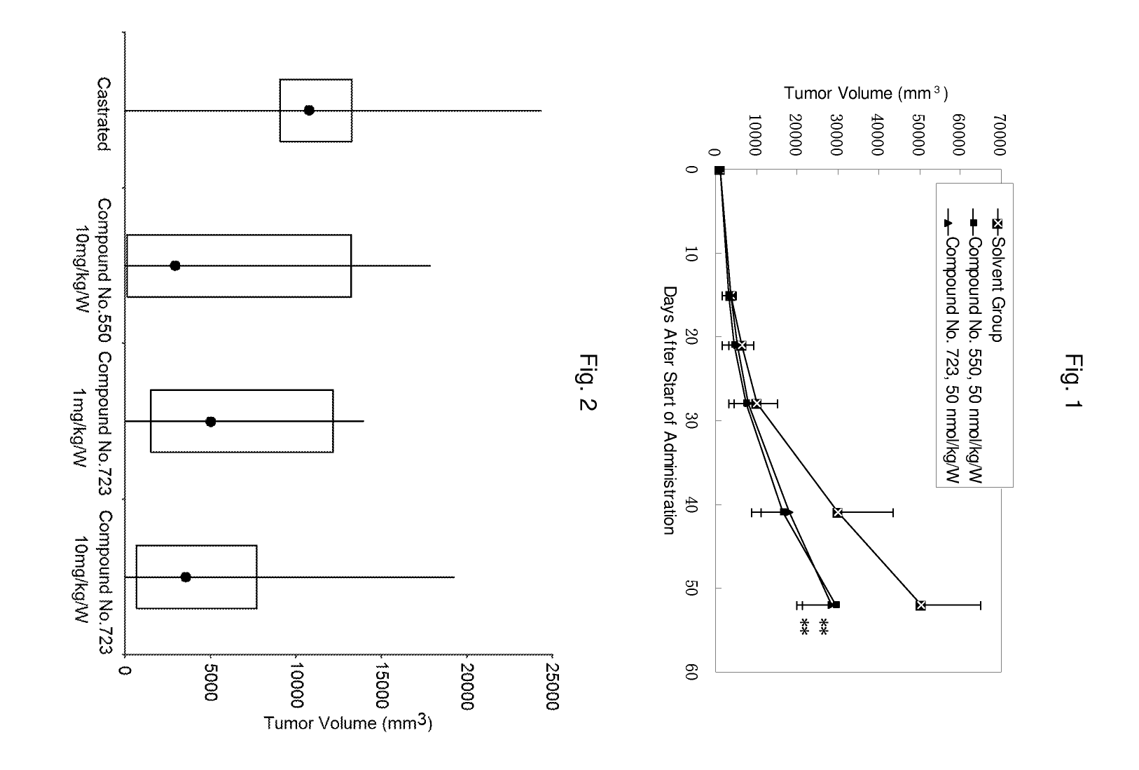

Regarding Group D, the rats were surgically castrated and used as the negative control group. The rats in Groups A to D were reared under ordinary rearing conditions. 74 days after the transplantation of DU145 cells, the tumor volume (longer diameter×shorter diameter×shorter diameter/2) was measured. In addition, Compound No. 550 or Compound No. 723 was administered to the rats in Groups A to C 30 days and 60 days after the transplantation of DU145 cells in the same manner as described above. Results are shown in FIG. 2. This test clearly indicates that Compound No. 550 and Compound No. 723 exhibit antitumor effects more than the surgical castration.

### INDUSTRIAL APPLICABILITY

The prophylactic/therapeutic agents for androgen-independent cancer (preferably prostate cancer) of the present invention are useful because they can be administrated to patients with androgen-independent cancer (preferably prostate cancer), which has posed a challenge in the clinical setting. Moreover, the medication according to the present invention is a combination of the inventive compound and a concomitant drug, and is particularly useful as a prophylactic/therapeutic agent for prostate cancer and androgen-independent prostate cancer. The inventive medication is also useful for administration in cancer patients who have developed a tolerance to therapeutic agents.

### Caracteristics of the Sequences

SEQ ID NO: 15: C terminus is amidated.
SEQ ID NO: 16: C terminus is amidated.
SEQ ID NO: 17: C terminus is amidated.
SEQ ID NO: 18: C terminus is amidated.

1. A prophylactic/therapeutic agent for androgen-independent cancer, comprising a metastin derivative (IV) of the following general formula, or a salt or prodrug thereof, 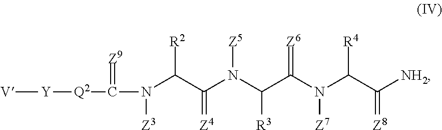
wherein V′ is a group of the formula 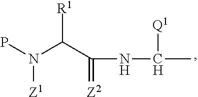
a group of the formula 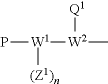
or a group of the formula 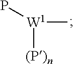
n represents 0 or 1;
W¹ represents N, CH or O (provided that when W¹ is N or CH, n represents 1 and when W¹ is O, n represents 0);
W² represents N or CH;
Z¹, Z³, Z⁵ and Z⁷ each represents hydrogen atom or a C₁₋₃ alkyl group;
Z², Z⁴, Z⁶ and Z⁸ each represents hydrogen atom, O or S;
R¹ represents (1) a hydrogen atom, (2) a C₁₋₈ alkyl optionally substituted with a substituent selected from the group consisting of an optionally substituted carbamoyl group, an optionally substituted hydroxyl group and an optionally substituted aromatic cyclic group, (3) a cyclic or linear C₁₋₁₀ alkyl group, (4) a C₁₋₁₀ alkyl group consisting of a cyclic alkyl group and a linear alkyl group or (5) an optionally substituted aromatic cyclic group;
R² represents (1) hydrogen atom or (2) a cyclic or linear C₁₋₁₀ alkyl group, (3) a C₁₋₁₀ alkyl group consisting of a cyclic alkyl group and a linear alkyl group, or (4) a C₁₋₈ alkyl group optionally substituted with a substituent selected from the group consisting of an optionally substituted carbamoyl group, an optionally substituted hydroxyl group and an optionally substituted aromatic cyclic group;
R³ represents (1) a C₁₋₈ alkyl group having an optionally substituted basic group and optionally having an additional substituent, (2) an aralkyl group having an optionally substituted basic group and optionally having an additional substituent, (3) a C₁₋₄ alkyl group having a non-aromatic cyclic hydrocarbon group of carbon atoms not greater than 7 having an optionally substituted basic group, and optionally having an additional substituent, or (4) a C₁₋₄ alkyl group having a non-aromatic heterocyclic group of carbon atoms not greater than 7 having an optionally substituted basic group, and optionally having an additional substituent;
R⁴ represents a C₁₋₄ alkyl group, which may optionally be substituted with a substituent selected from the group consisting of (1) an optionally substituted C₆₋₁₂ aromatic hydrocarbon group, (2) an optionally substituted 5- to 14-membered aromatic heterocyclic group consisting of 1 to 7 carbon atoms and hetero atoms selected from the group consisting of nitrogen, oxygen and sulfur atoms, (3) an optionally substituted C₈₋₁₄ aromatic fused-ring group, (4) an optionally substituted 5- to 14-membered aromatic fused heterocyclic group consisting of 3 to 11 carbon atoms and hetero atoms selected from the group consisting of nitrogen, oxygen and sulfur atoms, (5) an optionally substituted non-aromatic cyclic hydrocarbon group having carbon atoms not greater than 7, and (6) an optionally substituted non-aromatic heterocyclic group having carbon atoms not greater than 7;
Q¹ represents a C₁₋₄ alkyl group, which may optionally be substituted with a substituent selected from the group consisting of (1) an optionally substituted C₆₋₁₂ aromatic hydrocarbon group, (2) an optionally substituted 5- to 14-membered aromatic heterocyclic group consisting of 1 to 7 carbon atoms and hetero atoms selected from the group consisting of nitrogen, oxygen and sulfur atoms, (3) an optionally substituted C₈₋₁₄ aromatic fused-ring group, (4) an optionally substituted 5- to 14-membered aromatic fused heterocyclic group consisting of 3 to 11 carbon atoms and hetero atoms selected from the group consisting of nitrogen, oxygen and sulfur atoms, (5) an optionally substituted non-aromatic cyclic hydrocarbon group having carbon atoms not greater than 7, and (6) an optionally substituted non-aromatic heterocyclic group having carbon atoms not greater than 7;
Q² represents (1) CH₂, which may optionally be substituted with an optionally substituted C₁₋₄ alkyl group with a substituent selected from the group consisting of carbamoyl group and hydroxyl group, (2) NH, which may optionally be substituted with an optionally substituted C₁₋₄ alkyl group with a substituent selected from the group consisting of carbamoyl group and hydroxyl group, or (3) O;
Y represents a group represented by formula: —CONH—, —CSNH—, —CH₂NH—, —NHCO—, —CH₂O—, —CH₂S—, —COO—, —CSO—, —CH₂CH₂—, or —CH═CH—, which may optionally be substituted with a C₁₋₆ alkyl group; and,
Z⁹ represents hydrogen atom, O or S; and,
P and P′, which may be the same or different, each may form a ring by combining P and P′ or P and Q¹ together and represents:
(1) hydrogen atom;
(2) an optional amino acid residue continuously or discontinuously bound from the C terminus of the 1-48 amino acid sequence in the amino acid sequence represented by SEQ ID NO: 1;
(3) a group represented by formula:
J¹-J²-C(J³)(Q³)Y¹C(J⁴)(Q⁴)Y²C(J⁵)(Q⁵)Y³C(J⁶)(Q⁶)C(═Z¹⁰)—
(wherein:
J¹ represents (a) hydrogen atom or (b) (i) a C₁₋₁₅ acyl group, (ii) a C₁₋₁₅ alkyl group, (iii) a C₆₋₁₄ aryl group, (iv) carbamoyl group, (v) carboxyl group, (vi) sulfino group, (vii) amidino group, (viii) glyoxyloyl group or (ix) amino group, which groups may optionally be substituted with a substituent containing an optionally substituted cyclic group;
J² represents (1) NH optionally substituted with a C₁₋₆ alkyl group, (2) CH₂ optionally substituted with a C₁₋₆ alkyl group, (3) O or (4) S;
J³ through J⁶ each represents hydrogen atom or a C₁₋₃ alkyl group;
Q³ through Q⁶ each represents a C₁₋₄ alkyl group, which may optionally have a substituent selected from the group consisting of:
(1) an optionally substituted C₆₋₁₂ aromatic hydrocarbon group,
(2) an optionally substituted 5- to 14-membered aromatic heterocyclic group consisting of 1 to 7 carbon atoms and hetero atoms selected from the group consisting of nitrogen, oxygen and sulfur atoms,
(3) an optionally substituted C₈₋₁₄ aromatic fused-ring group,
(4) an optionally substituted 5- to 14-membered aromatic fused heterocyclic group consisting of 3 to 11 carbon atoms and hetero atoms selected from the group consisting of nitrogen, oxygen and sulfur atoms,
(5) an optionally substituted non-aromatic cyclic hydrocarbon group having carbon atoms not greater than 7,
(6) an optionally substituted non-aromatic heterocyclic group having carbon atoms not greater than 7,
(7) an optionally substituted amino group,
(8) an optionally substituted guanidino group,
(9) an optionally substituted hydroxyl group,
(10) an optionally substituted carboxyl group,
(11) an optionally substituted carbamoyl group, and
(12) an optionally substituted sulfhydryl group, or hydrogen atom;
J³ and Q³, J⁴ and Q⁴, J⁵ and Q⁵ or J⁶ and Q⁶ may be combined together, or, Z¹ and R¹, J² and Q³, Y¹ and Q⁴, Y² and Q⁵, or Y³ and Q⁶ may be combined together, to form a ring;
Y¹ through Y³ each represents a group represented by formula:
—CON(J¹³)-, —CSN(J¹³)-, —C(J¹⁴)N(J¹³)- or —N(J¹³)CO— (wherein J¹³ and J¹⁴ each represents hydrogen atom or a C₁₋₃ alkyl group); and,
Z¹⁰ represents hydrogen atom, O or S);
(4) a group represented by formula:
J¹-J²-C(J⁷)(Q⁷)Y²C(J⁸)(Q⁸)Y³C(J⁹)(Q⁹)C(═Z¹⁰)—
(wherein:
J¹ and J², each has the same significance as defined above;
J⁷ through J⁹ have the same significance as for J³;
Q⁷ through Q⁹ have the same significance as for Q³;
Y² and Y³ each has the same significance as defined above;
Z¹⁰ has the same significance as defined above;
J⁷ and Q⁷, J⁸ and Q⁸ or J⁹ and Q⁹ may be combined together, or, J² and Q⁷, Y² and Q⁸ or Y³ and Q⁹ may be combined together, to form a ring);
(5) a group represented by formula:
J¹-J²-C(J¹⁰)(Q¹⁰)Y³C(J¹¹)(Q¹¹)C(═Z¹⁰)—
(wherein:
J¹ and J² have the same significance as defined above represents;
J¹⁰ and J¹¹ have the same significance as for J³;
Q¹⁰ and Q¹¹ have the same significance as for Q³;
Y³ has the same significance as defined above;
Z¹⁰ has the same significance as defined above; and, J¹⁰ and Q¹⁰ or J¹¹ and Q¹¹ may be combined together, or J² and Q¹⁰ or Y³ and Q¹¹ may be combined together, to form a ring);
(6) a group represented by formula:
J¹-J²-C(J¹²)(Q¹²)C(═Z¹⁰)—
(wherein;
J¹ and J² have the same significance as defined above;
J¹² has the same significance as for J³;
Q¹² has the same significance as for Q³;
Z¹⁰ has the same significance as defined above; and,
J¹² and Q¹² may be combined together, or J² and Q¹² may be combined together, to form a ring); or,
(7) a group represented by formula:
J¹ (where J¹ has the same significance as defined above).

2. The agent of claim 1, wherein the androgen-independent cancer is androgen-independent prostate cancer.

3. The agent of claim 1, wherein the metastin derivative (IV) is Ac-D-Tyr-Hyp-Asn-Thr-Phe-AzaGly-Leu-Arg(Me)-Trp-NH₂ (Compound No. 723) or a salt thereof.

4. The agent of claim 1, wherein the metastin derivative (IV) is Ac-D-Tyr-D-Trp-Asn-Thr-Phe-AzaGly-Leu-Arg(Me)-Trp-NH₂ (Compound No. 550) or a salt thereof.

5. A prophylactic/therapeutic agent for androgen-independent cancer, comprising;
Ac-D-Tyr-D-Trp-Asn-Thr-Phe-AzaGly-Leu-Arg(Me)-Trp-NH₂ (Compound No. 550), or
a salt thereof.

6. The agent of claim 5, wherein the androgen-independent cancer is androgen-independent prostate cancer.

7. A prophylactic/therapeutic agent for androgen-independent cancer, comprising;
Ac-D-Tyr-Hyp-Asn-Thr-Phe-AzaGly-Leu-Arg(Me)-Trp-NH₂ (Compound No. 723), or a salt thereof.

8. The agent of claim 7, wherein the androgen-independent cancer is androgen-independent prostate cancer.

9. A prophylactic/therapeutic method for androgen-independent cancer in mammals, the method comprising: administering an effective dose of a metastin derivative (IV) of the following general formula, or a salt or prodrug thereof, 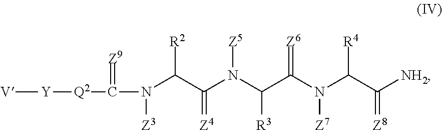
wherein V′ is a group of the formula 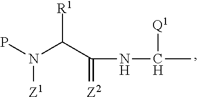
a group of the formula 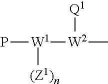
or a group of the formula 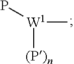
n represents 0 or 1;
W¹ represents N, CH or O (provided that when W¹ is N or CH, n represents 1 and when W¹ is O, n represents 0);
W² represents N or CH;
Z¹, Z³, Z⁵ and Z⁷ each represents hydrogen atom or a C₁₋₃ alkyl group;
Z², Z⁴, Z⁶ and Z⁸ each represents hydrogen atom, O or S;
R¹ represents (1) a hydrogen atom, (2) a C₁₋₈ alkyl optionally substituted with a substituent selected from the group consisting of an optionally substituted carbamoyl group, an optionally substituted hydroxyl group and an optionally substituted aromatic cyclic group, (3) a cyclic or linear C₁₋₁₀ alkyl group, (4) a C₁₋₁₀ alkyl group consisting of a cyclic alkyl group and a linear alkyl group or (5) an optionally substituted aromatic cyclic group;
R² represents (1) hydrogen atom or (2) a cyclic or linear C₁₋₁₀ alkyl group, (3) a C₁₋₁₀ alkyl group consisting of a cyclic alkyl group and a linear alkyl group, or (4) a C₁₋₈ alkyl group optionally substituted with a substituent selected from the group consisting of an optionally substituted carbamoyl group, an optionally substituted hydroxyl group and an optionally substituted aromatic cyclic group;
R³ represents (1) a C₁₋₈ alkyl group having an optionally substituted basic group and optionally having an additional substituent, (2) an aralkyl group having an optionally substituted basic group and optionally having an additional substituent, (3) a C₁₋₄ alkyl group having a non-aromatic cyclic hydrocarbon group of carbon atoms not greater than 7 having an optionally substituted basic group, and optionally having an additional substituent, or (4) a C₁₋₄ alkyl group having a non-aromatic heterocyclic group of carbon atoms not greater than 7 having an optionally substituted basic group, and optionally having an additional substituent;
R⁴ represents a C₁₋₄ alkyl group, which may optionally be substituted with a substituent selected from the group consisting of (1) an optionally substituted C₆₋₁₂ aromatic hydrocarbon group, (2) an optionally substituted 5- to 14-membered aromatic heterocyclic group consisting of 1 to 7 carbon atoms and hetero atoms selected from the group consisting of nitrogen, oxygen and sulfur atoms, (3) an optionally substituted C₈₋₁₄ aromatic fused-ring group, (4) an optionally substituted 5- to 14-membered aromatic fused heterocyclic group consisting of 3 to 11 carbon atoms and hetero atoms selected from the group consisting of nitrogen, oxygen and sulfur atoms, (5) an optionally substituted non-aromatic cyclic hydrocarbon group having carbon atoms not greater than 7, and (6) an optionally substituted non-aromatic heterocyclic group having carbon atoms not greater than 7;
Q¹ represents a C₁₋₄ alkyl group, which may optionally be substituted with a substituent selected from the group consisting of (1) an optionally substituted C₆₋₁₂ aromatic hydrocarbon group, (2) an optionally substituted 5- to 14-membered aromatic heterocyclic group consisting of 1 to 7 carbon atoms and hetero atoms selected from the group consisting of nitrogen, oxygen and sulfur atoms, (3) an optionally substituted C₈₋₁₄ aromatic fused-ring group, (4) an optionally substituted 5- to 14-membered aromatic fused heterocyclic group consisting of 3 to 11 carbon atoms and hetero atoms selected from the group consisting of nitrogen, oxygen and sulfur atoms, (5) an optionally substituted non-aromatic cyclic hydrocarbon group having carbon atoms not greater than 7, and (6) an optionally substituted non-aromatic heterocyclic group having carbon atoms not greater than 7;
Q² represents (1) CH₂, which may optionally be substituted with an optionally substituted C₁₋₄ alkyl group with a substituent selected from the group consisting of carbamoyl group and hydroxyl group, (2) NH, which may optionally be substituted with an optionally substituted C₁₋₄ alkyl group with a substituent selected from the group consisting of carbamoyl group and hydroxyl group, or (3) O;
Y represents a group represented by formula: —CONH—, —CSNH—, —CH₂NH—, —NHCO—, —CH₂O—, —CH₂S—, —COO—, —CSO—, —CH₂CH₂—, or —CH═CH—, which may optionally be substituted with a C₁₋₆ alkyl group; and,
Z⁹ represents hydrogen atom, O or S; and,
P and P′, which may be the same or different, each may form a ring by combining P and P′ or P and Q¹ together and represents:
(1) hydrogen atom;
(2) an optional amino acid residue continuously or discontinuously bound from the C terminus of the 1-48 amino acid sequence in the amino acid sequence represented by SEQ ID NO: 1;
(3) a group represented by formula:
J¹-J²-C(J³)(Q³)Y¹C(J⁴)(Q⁴)Y²C(J⁵)(Q⁵)Y³C(J⁶)(Q⁶)C(═Z¹⁰)—
(wherein:
J¹ represents (a) hydrogen atom or (b) (i) a C₁₋₁₅ acyl group, (ii) a C₁₋₁₅ alkyl group, (iii) a C₆₋₁₄ aryl group, (iv) carbamoyl group, (v) carboxyl group, (vi) sulfino group, (vii) amidino group, (viii) glyoxyloyl group or (ix) amino group, which groups may optionally be substituted with a substituent containing an optionally substituted cyclic group;
J² represents (1) NH optionally substituted with a C₁₋₆ alkyl group, (2) CH₂ optionally substituted with a C₁₋₆ alkyl group, (3) O or (4) S;
J³ through J⁶ each represents hydrogen atom or a C₁₋₃ alkyl group;
Q³ through Q⁶ each represents a C₁₋₄ alkyl group, which may optionally have a substituent selected from the group consisting of:
(1) an optionally substituted C₆₋₁₂ aromatic hydrocarbon group,
(2) an optionally substituted 5- to 14-membered aromatic heterocyclic group consisting of 1 to 7 carbon atoms and hetero atoms selected from the group consisting of nitrogen, oxygen and sulfur atoms,
(3) an optionally substituted C₈₋₁₄ aromatic fused-ring group,
(4) an optionally substituted 5- to 14-membered aromatic fused heterocyclic group consisting of 3 to 11 carbon atoms and hetero atoms selected from the group consisting of nitrogen, oxygen and sulfur atoms,
(5) an optionally substituted non-aromatic cyclic hydrocarbon group having carbon atoms not greater than 7,
(6) an optionally substituted non-aromatic heterocyclic group having carbon atoms not greater than 7,
(7) an optionally substituted amino group,
(8) an optionally substituted guanidino group,
(9) an optionally substituted hydroxyl group,
(10) an optionally substituted carboxyl group,
(11) an optionally substituted carbamoyl group, and
(12) an optionally substituted sulfhydryl group, or hydrogen atom;
J³ and Q³, J⁴ and Q⁴, J⁵ and Q⁵ or J⁶ and Q⁶ may be combined together, or, Z¹ and R¹, J² and Q³, Y¹ and Q⁴, Y² and Q⁵, or Y³ and Q⁶ may be combined together, to form a ring;
Y¹ through Y³ each represents a group represented by formula:
—CON(J¹³)—, —CSN(J¹³)—, —C(J¹⁴)N(J¹³)— or —N(J¹³)CO— (wherein J¹³ and J¹⁴ each represents hydrogen atom or a C₁₋₃ alkyl group); and,
Z¹⁰ represents hydrogen atom, O or S);
(4) a group represented by formula:
J¹-J²-C(J⁷)(Q⁷)Y²C(J⁸)(Q⁸)Y³C(J⁹)(Q⁹)C(═Z¹⁰)—
(wherein:
J¹ and J², each has the same significance as defined above;
J⁷ through J⁹ have the same significance as for J³;
Q⁷ through Q⁹ have the same significance as for Q³;
Y² and Y³ each has the same significance as defined above;
Z¹⁰ has the same significance as defined above;
J⁷ and Q⁷, J⁸ and Q⁸ or J⁹ and Q⁹ may be combined together, or, J² and Q⁷, Y² and Q⁸ or Y³ and Q⁹ may be combined together, to form a ring);
(5) a group represented by formula:
J¹-J²-C(J¹⁰)(Q¹⁰)Y³C(J¹¹)(Q¹¹)C(═Z¹⁰)—
(wherein:
J¹ and J² have the same significance as defined above represents;
J¹⁰ and J¹¹ have the same significance as for J³;
Q¹⁰ and Q¹¹ have the same significance as for Q³;
Y³ has the same significance as defined above;
Z¹⁰ has the same significance as defined above; and,
J¹⁰ and Q¹⁰ or J¹¹ and Q¹¹ may be combined together, or J² and Q¹⁰ or Y³ and Q¹¹ may be combined together, to form a ring);
(6) a group represented by formula:
J¹-J²-C(J¹²)(Q¹²)C(═Z¹⁰)—
(wherein;
J¹ and J² have the same significance as defined above;
J¹² has the same significance as for J³;
Q¹² has the same significance as for Q³;
Z¹⁰ has the same significance as defined above; and,
J¹² and Q¹² may be combined together, or J² and Q¹² may be combined together, to form a ring); or,
(7) a group represented by formula:
J¹ (where J¹ has the same significance as defined above).

10. Use of a metastin derivative (IV) of the following general formula, or a salt or prodrug thereof for producing prophylactic/therapeutic agent for androgen-independent cancer, 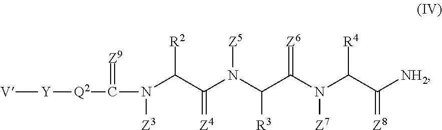
wherein V′ is a group of the formula 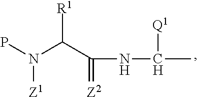
a group of the formula 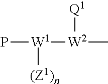
or a group of the formula 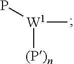
n represents 0 or 1;
W¹ represents N, CH or O (provided that when W¹ is N or CH, n represents 1 and when W¹ is O, n represents 0);
W² represents N or CH;
Z¹, Z³, Z⁵ and Z⁷ each represents hydrogen atom or a C₁₋₃ alkyl group;
Z², Z⁴, Z⁶ and Z⁸ each represents hydrogen atom, O or S;
R¹ represents (1) a hydrogen atom, (2) a C₁₋₈ alkyl optionally substituted with a substituent selected from the group consisting of an optionally substituted carbamoyl group, an optionally substituted hydroxyl group and an optionally substituted aromatic cyclic group, (3) a cyclic or linear C₁₋₁₀ alkyl group, (4) a C₁₋₁₀ alkyl group consisting of a cyclic alkyl group and a linear alkyl group or (5) an optionally substituted aromatic cyclic group;
R² represents (1) hydrogen atom or (2) a cyclic or linear C₁₋₁₀ alkyl group, (3) a C₁₋₁₀ alkyl group consisting of a cyclic alkyl group and a linear alkyl group, or (4) a C₁₋₈ alkyl group optionally substituted with a substituent selected from the group consisting of an optionally substituted carbamoyl group, an optionally substituted hydroxyl group and an optionally substituted aromatic cyclic group;
R³ represents (1) a C₁₋₈ alkyl group having an optionally substituted basic group and optionally having an additional substituent, (2) an aralkyl group having an optionally substituted basic group and optionally having an additional substituent, (3) a C₁₋₄ alkyl group having a non-aromatic cyclic hydrocarbon group of carbon atoms not greater than 7 having an optionally substituted basic group, and optionally having an additional substituent, or (4) a C₁₋₄ alkyl group having a non-aromatic heterocyclic group of carbon atoms not greater than 7 having an optionally substituted basic group, and optionally having an additional substituent;
R⁴ represents a C₁₋₄ alkyl group, which may optionally be substituted with a substituent selected from the group consisting of (1) an optionally substituted C₆₋₁₂ aromatic hydrocarbon group, (2) an optionally substituted 5- to 14-membered aromatic heterocyclic group consisting of 1 to 7 carbon atoms and hetero atoms selected from the group consisting of nitrogen, oxygen and sulfur atoms, (3) an optionally substituted C₈₋₁₄ aromatic fused-ring group, (4) an optionally substituted 5- to 14-membered aromatic fused heterocyclic group consisting of 3 to 11 carbon atoms and hetero atoms selected from the group consisting of nitrogen, oxygen and sulfur atoms, (5) an optionally substituted non-aromatic cyclic hydrocarbon group having carbon atoms not greater than 7, and (6) an optionally substituted non-aromatic heterocyclic group having carbon atoms not greater than 7;
Q¹ represents a C₁₋₄ alkyl group, which may optionally be substituted with a substituent selected from the group consisting of (1) an optionally substituted C₆₋₁₂ aromatic hydrocarbon group, (2) an optionally substituted 5- to 14-membered aromatic heterocyclic group consisting of 1 to 7 carbon atoms and hetero atoms selected from the group consisting of nitrogen, oxygen and sulfur atoms, (3) an optionally substituted C₈₋₁₄ aromatic fused-ring group, (4) an optionally substituted 5- to 14-membered aromatic fused heterocyclic group consisting of 3 to 11 carbon atoms and hetero atoms selected from the group consisting of nitrogen, oxygen and sulfur atoms, (5) an optionally substituted non-aromatic cyclic hydrocarbon group having carbon atoms not greater than 7, and (6) an optionally substituted non-aromatic heterocyclic group having carbon atoms not greater than 7;
Q² represents (1) CH₂, which may optionally be substituted with an optionally substituted C₁₋₄ alkyl group with a substituent selected from the group consisting of carbamoyl group and hydroxyl group, (2) NH, which may optionally be substituted with an optionally substituted C₁₋₄ alkyl group with a substituent selected from the group consisting of carbamoyl group and hydroxyl group, or (3) O;
Y represents a group represented by formula: —CONH—, —CSNH—, —CH₂NH—, —NHCO—, —CH₂O—, —CH₂S—, —COO—, —CSO—, —CH₂CH₂—, or —CH═CH—, which may optionally be substituted with a C₁₋₆ alkyl group; and,
Z⁹ represents hydrogen atom, O or S; and,
P and P′, which may be the same or different, each may form a ring by combining P and P′ or P and Q¹ together and represents:
(1) hydrogen atom;
(2) an optional amino acid residue continuously or discontinuously bound from the C terminus of the 1-48 amino acid sequence in the amino acid sequence represented by SEQ ID NO: 1;
(3) a group represented by formula:
J¹-J²-C(J³)(Q³)Y¹C(J⁴)(Q⁴)Y²C(J⁵)(Q⁵)Y³C(J⁶)(Q⁶)C(═Z¹⁰)—
(wherein:
J¹ represents (a) hydrogen atom or (b) (i) a C₁₋₁₅ acyl group, (ii) a C₁₋₁₅ alkyl group, (iii) a C₆₋₁₄ aryl group, (iv) carbamoyl group, (v) carboxyl group, (vi) sulfino group, (vii) amidino group, (viii) glyoxyloyl group or (ix) amino group, which groups may optionally be substituted with a substituent containing an optionally substituted cyclic group;
J² represents (1) NH optionally substituted with a C₁₋₆ alkyl group, (2) CH₂ optionally substituted with a C₁₋₆ alkyl group, (3) O or (4) S;
J³ through J⁶ each represents hydrogen atom or a C₁₋₃ alkyl group;
Q³ through Q⁶ each represents a C₁₋₄ alkyl group, which may optionally have a substituent selected from the group consisting of:
(1) an optionally substituted C₆₋₁₂ aromatic hydrocarbon group,
(2) an optionally substituted 5- to 14-membered aromatic heterocyclic group consisting of 1 to 7 carbon atoms and hetero atoms selected from the group consisting of nitrogen, oxygen and sulfur atoms,
(3) an optionally substituted C₈₋₁₄ aromatic fused-ring group,
(4) an optionally substituted 5- to 14-membered aromatic fused heterocyclic group consisting of 3 to 11 carbon atoms and hetero atoms selected from the group consisting of nitrogen, oxygen and sulfur atoms,
(5) an optionally substituted non-aromatic cyclic hydrocarbon group having carbon atoms not greater than 7,
(6) an optionally substituted non-aromatic heterocyclic group having carbon atoms not greater than 7,
(7) an optionally substituted amino group,
(8) an optionally substituted guanidino group,
(9) an optionally substituted hydroxyl group,
(10) an optionally substituted carboxyl group,
(11) an optionally substituted carbamoyl group, and
(12) an optionally substituted sulfhydryl group, or hydrogen atom;
J³ and Q³, J⁴ and Q⁴, J⁵ and Q⁵ or J⁶ and Q⁶ may be combined together, or, Z¹ and R¹, J² and Q³, Y¹ and Q⁴, Y² and Q⁵, or Y³ and Q⁶ may be combined together, to form a ring;
Y¹ through Y³ each represents a group represented by formula:
—CON(J¹³)-, —CSN(J¹³)-, —C(J¹⁴)N(J¹³)- or —N(J¹³)CO— (wherein J¹³ and J¹⁴ each represents hydrogen atom or a C₁₋₃ alkyl group); and,
Z¹⁰ represents hydrogen atom, O or S);
(4) a group represented by formula:
J¹-J²-C(J⁷)(Q⁷)Y²C(J⁸)(Q⁸)Y³C(J⁹)(Q⁹)C(═Z¹⁰)—
(wherein:
J¹ and J², each has the same significance as defined above;
J⁷ through J⁹ have the same significance as for J³;
Q⁷ through Q⁹ have the same significance as for Q³;
Y² and Y³ each has the same significance as defined above;
Z¹⁰ has the same significance as defined above;
J⁷ and Q⁷, J⁸ and Q⁸ or J⁹ and Q⁹ may be combined together, or, J² and Q⁷, Y² and Q⁸ or Y³ and Q⁹ may be combined together, to form a ring);
(5) a group represented by formula:
J¹-J²-C(J¹⁰)(Q¹⁰)Y³C(J¹¹)(Q¹¹)C(═Z10)—
(wherein:
J¹ and J² have the same significance as defined above represents;
J¹⁰ and J¹¹ have the same significance as for J³;
Q¹⁰ and Q¹¹ have the same significance as for Q³;
Y³ has the same significance as defined above;
Z¹⁰ has the same significance as defined above; and,
J¹⁰ and Q¹⁰ or J¹¹ and Q¹¹ may be combined together, or J² and Q¹⁰ or Y³ and Q¹¹ may be combined together, to form a ring);
(6) a group represented by formula:
J¹-J²-C(J¹²)(Q¹²)C(═Z¹⁰)—
(wherein;
J¹ and J² have the same significance as defined above;
J¹² has the same significance as for J³;
Q¹² has the same significance as for Q³;
Z¹⁰ has the same significance as defined above; and,
J¹² and Q¹² may be combined together, or J² and Q¹² may be combined together, to form a ring); or,
(7) a group represented by formula:
J¹ (where J¹ has the same significance as defined above).


SEQUENCE LISTING (22 sequences)

SEQ ID NO: 1 - protein, 54 aa, Homo sapiens
GTSLSPPPESSGSRQQPGLSAPHSRQIPAPQGAVLVQREKDLPNYNWNSFGLRF

SEQ ID NO: 2 - DNA, 162 bp, Homo sapiens
ggtacttctctgtctccgccgccggaatcttctggttctcgtcagcagccgggtctgtctgctccgcactctcgtcagatcccggctccgcagggtgctgttctggttcagcgtgaaaaagacctgccgaactacaactggaactctttcggtctgcgtttc

SEQ ID NO: 3 - protein, 152 aa, Mus musculus
MYLRFGVDVCSLSPWKETVDLPLPPRMISMASWQLLLLLCVATYGEPLAKVAPGSTGQQSGPQELVNAWEKESRYAESKPGSAGLRARRSSPCPPVEGPAGRQRPLCASRSRLIPAPRGAVLVQREKDLSTYNWNSFGLRYGRRQAARAARG

SEQ ID NO: 4 - DNA, 456 bp, Mus musculus
atgtatctgagatttggcgttgatgtctgcagcctgagtccctggaaggagactgtagacctgccccttcctcccagaatgatctcaatggcttcttggcagctgctgcttctcctctgtgtcgccacctatggggagccgctggcaaaagtgaagcctggatccacaggccagcagtccggaccccaggaactcgttaatgcctgggaaaaggaatcgcggtatgcagagagcaagcctgggtctgcagggctgcgcgctcgtaggtcgtcgccatgcccgccggttgagggccccgcggggcgccagcggcccctgtgtgcctcccgcagtcgcctgatccctgcgccccgcggagcggtgctggtgcagcgggagaaggacctgtccacctacaactggaactccttcggcctgcgctacggcaggaggcaggcggcgcgggcagcacggggc

SEQ ID NO: 5 - protein, 156 aa, Mus musculus
MYLRFGVDVCSLSPWKETVDLPLPPRMISMASWQLLLLLCVATYGEPLAKVAPLVKPGSTGQQSGPQELVNAWEKESRYAESKPGSAGLRARRSSPCPPVEGPAGRQRPLCASRSRLIPAPRGAVLVQREKDLSTYNWNSFGLRYGRRQAARAARG

SEQ ID NO: 6 - DNA, 468 bp, Mus musculus
atgtatctgagatttggcgttgatgtctgcagcctgagtccctggaaggagactgtagacctgccccttcctcccagaatgatctcaatggcttcttggcagctgctgcttctcctctgtgtcgccacctatggggagccgctggcaaaagtggcacctttggtgaagcctggatccacaggccagcagtccggaccccaggaactcgttaatgcctgggaaaaggaatcgcggtatgcagagagcaagcctgggtctgcagggctgcgcgctcgtaggtcgtcgccatgcccgccggttgagggccccgcggggcgccagcggcccctgtgtgcctcccgcagtcgcctgatccctgcgccccgcggagcggtgctggtgcagcgggagaaggacctgtccacctacaactggaactccttcggcctgcgctacggcaggaggcaggcggcgcgggcagcacggggc

SEQ ID NO: 7 - protein, 130 aa, Rattus sp.
MTSLASWQLLLLLCVASFGEPLAKMAPVVNPEPTGQQSGPQELVNAWQKGPRYAESKPGAAGLRARRTSPCPPVENPTGHQRPPCATRSRLIPAPRGSVLVQREKDMSAYNWNSFGLRYGRRQVARAARG

SEQ ID NO: 8 - DNA, 390 bp, Rattus sp.
atgacctcgctggcttcttggcagctgctgcttctcctctgtgtggcctcttttggggagccactggcaaaaatggcacctgtggtgaaccctgaacccacaggccaacagtccggaccccaggaactcgttaatgcctggcaaaagggcccgcggtatgcagagagcaagcctggggctgcaggactgcgcgctcgccgaacatcgccatgcccgccggtggagaaccccacggggcaccagcggcccccgtgtgccacccgcagtcgcctgatccctgcgccccgcggatcggtgctggtgcagcgcgagaaggacatgtcagcctacaactggaactcctttggcctgcgctacggcaggaggcaggtggcgcgggcggcacggggc

SEQ ID NO: 9 - protein, 398 aa, Homo sapiens
MHTVATSGPNASWGAPANASGCPGCGANASDGPVPSPRAVDAWLVPLFFAALMLLGLVGNSLVIYVICRHKPMRTVTNFYIANLAATDVTFLLCCVPFTALLYPLPGWVLGDFMCKFVNYIQQVSVQATCATLTAMSVDRWYVTVFPLRALHRRTPRLALAVSLSIWVGSAAVSAPVLALHRLSPGPRAYCSEAFPSRALERAFALYNLLALYLLPLLATCACYAAMLRHLGRVAVRPAPADSALQGQVLAERAGAVRAKVSRLVAAVVLLFAACWGPIQLFLVLQALGPAGSWHPRSYAAYALKTWAHCMSYSNSALNPLLYAFLGSHFRQAFRRVCPCAPRRPRRPRRPGPSDPAAPHAELHRLGSHPAPARAQKPGSSGLAARGLCVLGEDNAPL

SEQ ID NO: 10 - DNA, 1194 bp, Homo sapiens
atgcacaccgtggctacgtccggacccaacgcgtcctggggggcaccggccaacgcctccggctgcccgggctgtggcgccaacgcctcggacggcccagtcccttcgccgcgggccgtggacgcctggctcgtgccgctcttcttcgcggcgctgatgctgctgggcctggtggggaactcgctggtcatctacgtcatctgccgccacaagccgatgcggaccgtgaccaacttctacatcgccaacctggcggccacggacgtgaccttcctcctgtgctgcgtccccttcacggccctgctgtacccgctgcccggctgggtgctgggcgacttcatgtgcaagttcgtcaactacatccagcaggtctcggtgcaggccacgtgtgccactctgaccgccatgagtgtggaccgctggtacgtgacggtgttcccgttgcgcgccctgcaccgccgcacgccccgcctggcgctggctgtcagcctcagcatctgggtaggctctgcggcggtgtctgcgccggtgctcgccctgcaccgcctgtcacccgggccgcgcgcctactgcagtgaggccttccccagccgcgccctggagcgcgccttcgcactgtacaacctgctggcgctgtacctgctgccgctgctcgccacctgcgcctgctatgcggccatgctgcgccacctgggccgggtcgccgtgcgccccgcgcccgccgatagcgccctgcaggggcaggtgctggcagagcgcgcaggcgccgtgcgggccaaggtctcgcggctggtggcggccgtggtcctgctcttcgccgcctgctggggccccatccagctgttcctggtgctgcaggcgctgggccccgcgggctcctggcacccacgcagctacgccgcctacgcgcttaagacctgggctcactgcatgtcctacagcaactccgcgctgaacccgctgctctacgccttcctgggctcgcacttccgacaggccttccgccgcgtctgcccctgcgcgccgcgccgcccccgccgcccccgccggcccggaccctcggaccccgcagccccacacgcggagctgcaccgcctggggtcccacccggcccccgccagggcgcagaagccagggagcagtgggctggccgcgcgcgggctgtgcgtcctgggggaggacaacgcccctctc

SEQ ID NO: 11 - protein, 396 aa, Rattus sp.
MAAEATLGPNVSWWAPSNASGCPGCGVNASDGPGSAPRPLDAWLVPLFFAALMLLGLVGNSLVIFVICRHKHMQTVTNFYIANLAATDVTFLLCCVPFTALLYPLPTWVLGDFMCKFVNYIQQVSVQATCATLTAMSVDRWYVTVFPLRALHRRTPRLALTVSLSIWVGSAAVSAPVLALHRLSPGPHTYCSEAFPSRALERAFALYNLLALYLLPLLATCACYGAMLRHLGRAAVRPAPTDGALQGQLLAQRAGAVRTKVSRLVAAVVLLFAACWGPIQLFLVLQALGPSGAWHPRSYAAYALKIWAHCMSYSNSALNPLLYAFLGSHFRQAFCRVCPCGPQRQRRPHASAHSDRAAPHSVPHSRAAHPVRVRTPEPGNPVVRSPSVQDEHTAPL

SEQ ID NO: 12 - DNA, 1188 bp, Rattus sp.
atggccgcagaggcgacgttgggtccgaacgtgagctggtgggctccgtccaacgcttcgggatgcccgggctgcggtgtcaatgcctcggatggcccaggctccgcgccaaggcccctggatgcctggctggtgcccctgtttttcgctgccctaatgttgctggggctagtcgggaactcactggtcatcttcgttatctgccgccacaagcacatgcagaccgtcaccaatttctacatcgctaacctggcggccacagatgtcactttccttctgtgctgcgtacccttcaccgcgctcctctatccgctgcccacctgggtgctgggagacttcatgtgcaaattcgtcaactacatccagcaggtctcggtgcaagccacatgtgccactttgacagccatgagtgtggaccgctggtacgtgactgtgttcccgctgcgtgcacttcaccgccgcactccgcgcctggccctgactgtcagccttagcatctgggtgggttccgcagctgtttccgccccggtgctggctctgcaccgcctgtcgcccgggcctcacacctactgcagtgaggcgtttcccagccgtgccctggagcgcgctttcgcgctctacaacctgctggccctatacctgctgccgctgctcgccacctgcgcctgctacggtgccatgctgcgccacctgggccgcgccgctgtacgccccgcacccactgatggcgccctgcaggggcagctgctagcacagcgcgctggagcagtgcgcaccaaggtctcccggctggtggccgctgtcgtcctgctcttcgccgcctgctggggcccgatccagctgttcctggtgcttcaagccctgggcccctcgggggcctggcaccctcgaagctatgccgcctacgcgctcaagatctgggctcactgcatgtcctacagcaattctgcgctcaacccgctgctctatgccttcctgggttcccacttcagacaggccttctgccgcgtgtgcccctgcggcccgcaacgccagcgtcggccccacgcgtcagcgcactcggaccgagccgcaccccatagtgtgccgcacagccgggctgcgcaccctgtccgggtcaggacccccgagcctgggaaccctgtggtgcgctcgccctctgttcaggatgaacacactgccccactc

SEQ ID NO: 13 - protein, 396 aa, Mus musculus
MATEATLAPNVTWWAPSNASGCPGCGVNASDDPGSAPRPLDAWLVPLFFATLMLLGLVGNSLVIYVICRHKHMQTVTNFYIANLAATDVTFLLCCVPFTALLYPLPAWVLGDFMCKFVNYIQQVSVQATCATLTAMSVDRWYVTVFPLRALHRRTPRLALAVSLSIWVGSAAVSAPVLALHRLSPGPRTYCSEAFPSRALERAFALYNLLALYLLPLLATCACYGAMLRHLGRAAVRPAPTDGALQGQLLAQRAGAVRTKVSRLVAAVVLLFAACWGPIQLFLVLQALGPSGAWHPRSYAAYAVKIWAHCMSYSNSALNPLLYAFLGSHFRQAFCRVCPCCRQRQRRPHTSAHSDRAATHTVPHSRAAHPVRIRSPEPGNPVVRSPCAQSERTASL

SEQ ID NO: 14 - DNA, 1188 bp, Mus musculus
atggccaccgaggcgacattggctcccaatgtgacctggtgggctccgtccaacgcttcaggatgcccaggctgcggtgtcaacgcctcggatgacccaggctctgcgccaaggcccctggatgcctggctggttcccctgtttttcgctacactcatgttgcttgggctggtcggaaactcattggtcatctacgttatctgccgccacaagcacatgcagacagttaccaacttctacatcgctaacctggctgccacagacgtcactttcctactgtgctgcgtgcccttcaccgcactcctctacccgctgcccgcctgggtgctgggagacttcatgtgcaaattcgtcaactacatccagcaggtctcggtgcaagccacatgtgccactctgacggccatgagtgtggaccgctggtatgtgactgtgttcccgctgcgtgcacttcaccgccgcactccgcgcctggccctggctgtcagcctcagcatctgggtggggtcagcagctgtgtccgccccggtgctggccctgcaccgcctgtcgccagggcctcgcacctactgcagcgaggcgtttcccagccgcgccctggagcgcgccttcgcgctctacaacctgctggctctatatctgctgccgctgctcgccacctgcgcctgctacggcgccatgctgcgccacctgggccgtgcggctgtacgccccgcacccactgacggcgccctgcagggacagctgctagcacagcgcgccggagcagtgcgcaccaaggtctcccggctggtggccgctgtcgtcctgctcttcgccgcctgctggggcccgatccagctgttcctggtgcttcaagccctgggcccctcgggggcctggcaccctcgaagctatgccgcctacgcggtcaagatctgggctcactgcatgtcctacagcaactcggcgctcaatccgctgctctatgccttcctgggttcacacttcagacaggccttctgccgcgtgtgcccctgctgccggcaacgccagcgccggccccacacgtcagcgcactcggaccgagctgcaactcacactgtgccgcacagccgtgctgcgcaccctgtgcggatcaggagcccggagcctgggaaccctgtggtgcgctcgccctgcgctcagagtgaacgcactgcctcactc

SEQ ID NO: 15 - protein, 15 aa, Artificial Sequence
  Description of Artificial Sequence Synthetic peptide
KDLPNYNWNSFGLRF

SEQ ID NO: 16 - protein, 10 aa, Artificial Sequence
  Description of Artificial Sequence Synthetic peptide
YNWNSFGLRF

SEQ ID NO: 17 - protein, 9 aa, Artificial Sequence
  Description of Artificial Sequence Synthetic peptide
NWNSFGLRF

SEQ ID NO: 18 - protein, 8 aa, Artificial Sequence
  Description of Artificial Sequence Synthetic peptide
WNSFGLRF

SEQ ID NO: 19 - DNA, 45 bp, Homo sapiens
aaggacctgccgaactacaactggaactccttcggcctgcgcttc

SEQ ID NO: 20 - DNA, 30 bp, Homo sapiens
tacaactggaactccttcggcctgcgcttc

SEQ ID NO: 21 - DNA, 27 bp, Homo sapiens
aactggaactccttcggcctgcgcttc

SEQ ID NO: 22 - DNA, 24 bp, Homo sapiens
tggaactccttcggcctgcgcttc